(19) United States
(12) Patent Application Publication
(10) Pub. No.: US 2024/0400560 A1
(43) Pub. Date: Dec. 5, 2024

(54) BICYCLIC HETEROCYCLIC DERIVATIVE HAVING VIRAL GROWTH INHIBITORY ACTIVITY AND PHARMACEUTICAL COMPOSITION CONTAINING SAME

(71) Applicants: Shionogi & Co., Ltd., Osaka (JP); NATIONAL UNIVERSITY CORPORATION HOKKAIDO UNIVERSITY, Hokkaido (JP)

(72) Inventors: Yoshiyuki TAODA, Osaka (JP); Shota UEHARA, Osaka (JP); Yuseke SAKO, Osaka (JP); Keiichiro HIRAI, Osaka (JP)

(73) Assignees: Shionogi & Co., Ltd., Osaka (JP); NATIONAL UNIVERSITY CORPORATION HOKKAIDO UNIVERSITY, Hokkaido (JP)

(21) Appl. No.: 18/692,022

(22) PCT Filed: Sep. 15, 2022
(86) PCT No.: PCT/JP2022/034552

§ 371 Date: Mar. 14, 2024

(30) Foreign Application Priority Data
Sep. 17, 2021 (JP) 2021-152086
Nov. 26, 2021 (JP) 2021-191637
Jun. 8, 2022 (JP) 2022-093120

(51) Int. Cl. C07D 471/04; A61K 31/517; A61K 31/519; A61K 31/5386; A61P 31/14; C07D 401/06; C07D 403/06; C07D 487/08; C07D 491/048; C07D 519/00

(52) U.S. Cl. CPC C07D 471/04; A61K 31/517; A61K 31/519; A61K 31/5386; A61P 31/14; C07D 401/06; C07D 403/06; C07D 487/08; C07D 491/048; C07D 519/00

(57) ABSTRACT

The present invention provides a compound exhibiting coronavirus 3CL protease inhibitory activity or a pharmaceutically acceptable salt thereof, and a pharmaceutical composition comprising the same.

Provided is a compound represented by Formula (I):

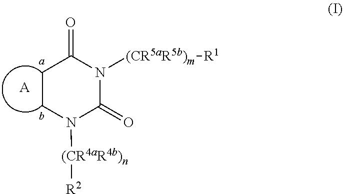

wherein carbon atom a and carbon atom b are each a carbon atom constituting ring A, the ring A is substituted aromatic carbocycle or the like, R¹ is substituted or unsubstituted aromatic heterocyclyl or the like, R² is substituted or unsubstituted 6-membered aromatic carbocyclyl or the like, m is 1 or the like, R^{5a}s are each independently a hydrogen atom or the like, R^{5b}s are each independently a hydrogen atom or the like, n is 1 or the like, R^{4a} is a hydrogen atom or the like, and R^{4a} is a hydrogen atom or the like. Alternatively, provided is a pharmaceutically acceptable salt of the compound.

### TECHNICAL FIELD

The present invention relates to a compound exhibiting coronavirus 3CL protease inhibitory activity and a pharmaceutical composition comprising a compound exhibiting coronavirus 3CL protease inhibitory activity.

### BACKGROUND ART

Coronaviruses, which belong to the order Nidovirales, family Coronaviridae, and the subfamily Coronavirinae, are positive-sense single-stranded RNA viruses that have a genome size of about 30 kilobases and are the largest among the known RNA viruses. Coronaviruses are classified into four genera, namely, the genus Alphacoronavirus, the genus Betacoronavirus, Gammacoronavirus, and Deltacoronavirus, and a total of seven types of coronaviruses, including two kinds in the genus Alphacoronavirus (HCoV-229E and HCoV-NL63) and five kinds in the genus Betacoronavirus (HCoV-HKU1, HCoV-OC43, SARS-CoV, MERS-CoV, and SARS-CoV-2), are known as coronaviruses that infect humans. Among these, four kinds (HCoV-229E, HCoV-NL63, HCoV-HKU1, and HCoV-OC43) are pathogens of common cold, while the other three kinds are severe acute respiratory syndrome (SARS) coronavirus (SARS-CoV), Middle East respiratory syndrome (MERS) coronavirus (MERS-CoV), and a novel coronavirus (SARS-CoV-2), all of which cause severe pneumonia.

Novel coronavirus infections (COVID-19) that occurred in Wuhan, China, in December 2019, rapidly spread to the international community, and the pandemic was announced by the WHO on Mar. 11, 2020. The number of infected people confirmed as of Sep. 6, 2022, reached 600 million or more, and the number of deaths reached more than 6.5 million or more (Non-patent Document 1). Droplet infection, contact infection, and aerosol infection have been reported as main routes of infection of SARS-CoV-2, and it has been confirmed that SARS-CoV-2 continues to drift in air together with aerosols and maintains infectivity for about 3 hours (Non-patent Document 2). The incubation period is about 2 to 14 days, and cold-like symptoms such as fever (87.9%), dry cough (67.7%), malaise (38.1%), and phlegm (33.4%) are typical (Non-patent Document 3). In severe cases, respiratory failure due to acute respiratory distress syndrome, acute lung injury, interstitial pneumonia, and the like occurs. Furthermore, multiple organ failure such as renal failure and hepatic failure has also been reported.

In Japan, as a result of drug repositioning of existing drugs, remdesivir, which is an antiviral drug, dexamethasone, which is an anti-inflammatory drug, and baricitinib, which is an antirheumatic drug, have been approved as therapeutic agents against COVID-19, and in January 2022, tocilizumab, which is an anti-IL-6 receptor antibody, have been received additional approval. Additionally, ronapreve (casirivimab/imdevimab), which is an antibody cocktail therapy, was approved as special case approval in July 2021, sotrovimab was approved as special case approval in September 2021, molnupiravir was approved as special case approval in December 2021, and Evusheld (tixagevimab/cilgavimab) was approved as special case approval in August 2022. However, among these drugs, there are cases where clinical use is refrained due to drug interaction, contraindications to pregnant women, and the like, and development of therapeutic drugs as new therapeutic options is awaited.

Upon infection of cells, coronaviruses synthesize two polyproteins. In these two polyproteins, replication complexes producing viral genomes, and two proteases are included. Proteases play an indispensable role for cleaving the polyproteins synthesized by viruses and causing each of the proteins to function. Between these two proteases, 3CL protease (main protease) bears most of the cleavage of the polyproteins (Non-patent Document 4).

Regarding COVID-19 therapeutic agents targeting 3CL proteases, it was published in ClinicalTrials.gov that Phase 1b trials for Lufotrelvir (PF-07304814), which is a prodrug of PF-00835231, have completed by Pfizer Inc (NCT04535167). Furthermore, in March 2021, Pfizer Inc. announced that Phase 1 trials for PF-07321332, a therapeutic agent against novel coronavirus infections, will be initiated. The structural formulae of PF-00835231, Lufotrelvir and PF-07321332 are as shown below, and these agents are different from the compound of the present invention in chemical structure (Non-patent Documents 5, 10 and 11 and Patent Documents 1 and 2).

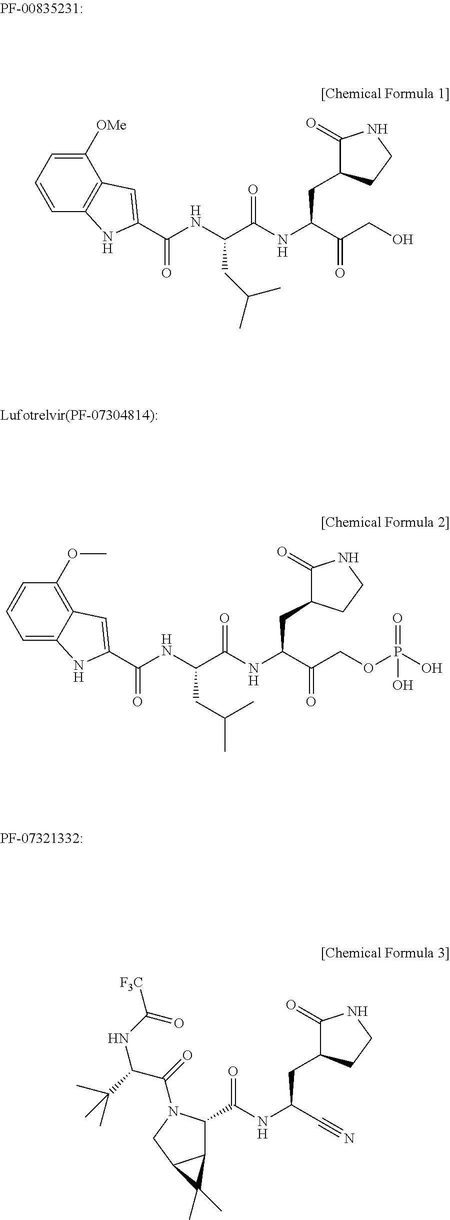 [SMILES: COc1cccc2[nH]c(C(=O)N[C@@H](CC(C)C)C(=O)N[C@@H](C[C@@H]3CCNC3=O)C(=O)CO)cc12 ; COc1cccc2[nH]c(C(=O)N[C@@H](CC(C)C)C(=O)N[C@@H](C[C@@H]3CCNC3=O)C(=O)COP(=O)(O)O)cc12 ; CC(C)(C)[C@H](NC(=O)C(F)(F)F)C(=O)N1C[C@H]2[C@@H]([C@H]1C(=O)N[C@H](C#N)C[C@@H]1CCNC1=O)C2(C)C]

In December 2021, PAXLOVID™ was approved for emergency use in the United States, and on Feb. 10, 2022, the PAXLOVID (registered trademark) PACK was approved as special case approval in Japan.

Furthermore, regarding COVID-19 therapeutic agents targeting 3CL proteases, the start of Phase 1 trials for PBI-0451 by Pardes Biosciences was reported in ClinicalTrials.gov on August 2021 (NCT05011812). The structural formula of PBI-0451 is as shown below, and these agents are different from the compound of the present invention in chemical structure (Non-patent Document 12).

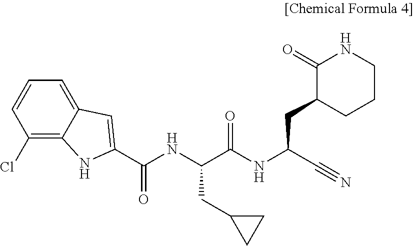 [SMILES: N#C[C@H](C[C@@H]1CCCNC1=O)NC(=O)[C@H](CC1CC1)NC(=O)c1cc2cccc(Cl)c2[nH]1]

No sufficient evidence has been obtained for mutations resistant to COVID-19 therapeutic agents targeting 3CL proteases.

Compounds having 3CL protease inhibitory activity are disclosed in Non-patent Documents 5 to 8 and 13 to 16; however, the compounds related to the present invention are neither described nor suggested in any of the documents.

Although derivatives having P2X₃ receptor inhibitory activity are disclosed in Patent Document 1, 3CL protease inhibitory activity and antiviral effect have neither been described nor suggested. In addition, Non-patent Documents 9 and Patent Documents 2, and 5 to 8 describe compounds having structures similar to those of the compounds of the present invention, but none of them describes or suggests the 3CL protease inhibitory activity and the antiviral effect.

### PRIOR ART REFERENCES

### Patent Document

[Patent Document 1] International Publication WO 2012/020742A
[Patent Document 2] International Publication WO 2018/107112A
[Patent Document 3] International Publication WO 2021/205298A
[Patent Document 4] International Publication WO 2021/250648A
[Patent Document 5] International Publication WO 2015/003879A
[Patent Document 6] International Publication WO 2020/261114A
[Patent Document 7] US Patent Publication U.S. Pat. No. 6,242,452
[Patent Document 8] JP 2002-308774 A

### Non-Patent Document

[Non-patent Document 1] “COVID-19 Dashboard by the Center for Systems Science and Engineering at Johns Hopkins University”, [online], Johns Hopkins University, [retrieved on Sep. 6, 2022], Internet <URL: https://coronavirus.jhu.edu/map.html>.
[Non-patent Document 2] The NEW ENGLAND JOURNAL of MEDICINE (2020), Vol. 382, pp. 1564-1567.
[Non-patent Document 3] “Report of the WHO-China Joint Mission on Coronavirus disease 2019 (COVID-19)”, [online], Feb. 28, 2020, WHO, [retrieved on Feb. 8, 2021], Internet <URL: https://www.who.int/docs/default-source/coronaviruse/who-china-joint-mission-on-covid-19-final-report.pdf>
[Non-patent Document 4] Science (2003), Vol. 300, pp. 1763-1767.
[Non-patent Document 5] “A comparative analysis of SARS-CoV-2 antivirals characterizes 3CLpro inhibitor PF-00835231 as a potential new treatment for COVID-19”, Journal of Virology, [online], Apr. 26, 2021, [retrieved on Feb. 15, 2022], Internet <URL: https://journals.asm.org/doi/10.1128/JVI.01819-20><doi: 10.1128/JVI.01819-20>.
[Non-patent Document 6] Cell Research (2020), Vol. 30, pp. 678-692.
[Non-patent Document 7] Science (2020), Vol. 368, pp. 409-412.
[Non-patent Document 8] ACS Central Science (2021), Vol. 7, No. 3, pp. 467-475.
[Non-patent Document 9] European Journal of Medicinal Chemistry (2019), Vol. 172, pp. 109-130
[Non-patent Document 10] 261st Am Chem Soc (ACS) Natl Meet⋅2021 Apr. 5/2021 Apr. 16 Virtual, Abst 243
[Non-patent Document 11] Science (2021), Vol. 374, pp. 1586-1593.
[Non-patent Document 12] “Discovery and Development of PBI-0451”, [online], Mar. 24, 2022, 35th International Conference on Antiviral Research (ICAR), [retrieved on Apr. 5, 2022], Internet <URL: https://ir.pardesbio.com/static-files/fc7c4f8c-e0bd-4b97-8c9c-eff09bafd4db>
[Non-patent Document 13] Molecules (2020), vol. 25, p. 3193
[Non-patent Document 14] Molecules (2020), vol. 25, p. 3920
[Non-patent Document 15] European Journal of Medicinal Chemistry (2020), Vol. 206, p. 112711
[Non-patent Document 16] Journal of the American Chemical Society (2022), vol. 144, pp. 2905-2920.

### SUMMARY OF THE INVENTION

### Problems to be Solved by the Invention

An object of the present invention is to provide a compound having coronavirus 3CL protease inhibitory activity. Preferably, the present invention provides a compound having an antiviral activity, particularly a coronavirus replication inhibitory activity, and a medicament comprising the compound.

### Means for Solving the Problem

The present invention relates to the following.
(1′″) A compound represented by Formula (I):

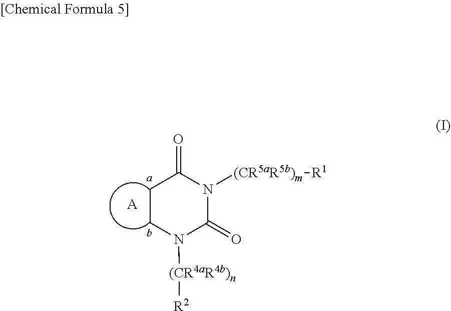

wherein carbon atom a and carbon atom b are each a carbon atom constituting ring A,
the ring A is a ring represented by:

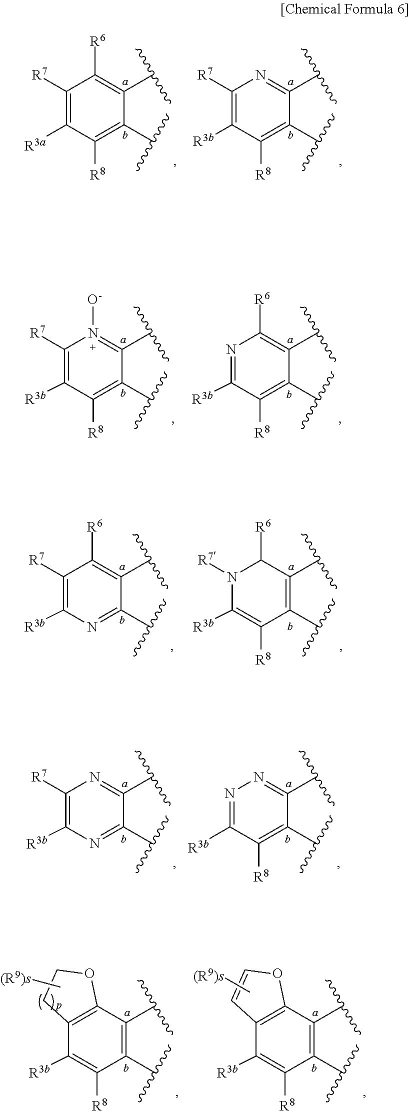

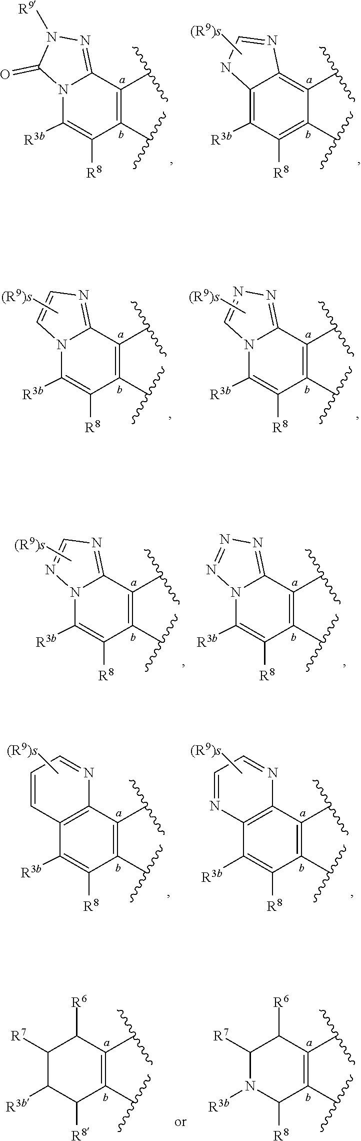

wherein carbon atom a represents the carbon atom a in Formula (I);
carbon atom b represents the carbon atom b in Formula (I);
R^{3a} is a hydrogen atom, substituted or unsubstituted aromatic carbocyclyl, substituted or unsubstituted aromatic heterocyclyl, substituted or unsubstituted non-aromatic heterocyclyl, substituted or unsubstituted alkyloxy, substituted amino, halogen, substituted or unsubstituted carbamoyl, substituted or unsubstituted sulfamoyl, substituted or unsubstituted non-aromatic carbocyclyloxy, or substituted or unsubstituted non-aromatic heterocyclyloxy;
R^{3b} and R^{3b′} are each independently a hydrogen atom, substituted or unsubstituted aromatic carbocyclyl, substituted or unsubstituted aromatic heterocyclyl, substituted or unsubstituted non-aromatic heterocyclyl, substituted or unsubstituted alkyloxy, substituted amino, halogen, cyano, substituted or unsubstituted alkyl, substituted or unsubstituted alkenyl, substituted or unsubstituted carbamoyl, substituted or unsubstituted sulfamoyl, substituted or unsubstituted non-aromatic carbocyclyloxy, or substituted or unsubstituted non-aromatic heterocyclyloxy;
R⁸ and R^{8′} are each independently a hydrogen atom, halogen, substituted or unsubstituted alkyl, or substituted or unsubstituted alkyloxy; or
R^{3a} and R⁸, and R^{3b} and R⁸ may be taken together with a carbon atom to which they are each bonded to form a substituted or unsubstituted aromatic carbocycle, or a substituted or unsubstituted aromatic heterocycle;
R⁶ is a hydrogen atom, halogen, substituted or unsubstituted alkyloxy, hydroxy, or cyano;
R⁷ is a hydrogen atom, halogen, substituted or unsubstituted alkyl, substituted or unsubstituted alkenyl, substituted or unsubstituted alkynyl, substituted or unsubstituted alkyloxy, substituted or unsubstituted non-aromatic carbocyclyl, substituted or unsubstituted non-aromatic heterocyclyl, substituted or unsubstituted non-aromatic carbocyclyloxy, substituted or unsubstituted amino, substituted or unsubstituted alkylsulfoxy, substituted or unsubstituted carbamoyl, hydroxy, carboxy, formyl, or cyano;
R^{7′} and R^{9′} are each independently a hydrogen atom, or substituted or unsubstituted alkyl;
R^{3b} and R^{7′} may be taken together with an atom to which they are each bonded to form a substituted or unsubstituted non-aromatic heterocycle;
R⁹s are each independently halogen, hydroxy, substituted or unsubstituted alkyl, or substituted or unsubstituted alkyloxy;
s is 0, 1, or 2;
p is 1, 2, or 3;
R¹ is substituted or unsubstituted nitrogen-containing aromatic heterocyclyl, substituted or unsubstituted nitrogen-containing non-aromatic heterocyclyl, or substituted or unsubstituted carbamoyl;
R² is substituted or unsubstituted 6-membered aromatic carbocyclyl, or substituted or unsubstituted 6-membered aromatic heterocyclyl;
n is 0 or 1;
R^{4a} is a hydrogen atom, or substituted or unsubstituted alkyl;
R^{4b} is a hydrogen atom, or substituted or unsubstituted alkyl; or R^{4a} and R^{4b} may be taken together with a carbon atom to which they are each bonded to form a substituted or unsubstituted non-aromatic carbocycle, or a substituted or unsubstituted non-aromatic heterocycle;
m is 0, 1, or 2;
R^{5a}s are each independently a hydrogen atom, or substituted or unsubstituted alkyl;
R^{5b}s are each independently a hydrogen atom, or substituted or unsubstituted alkyl,
provided that the compound in which R¹ is substituted or unsubstituted carbamoyl, and R^{3a} and R^{3b} are substituted or unsubstituted oxazolyl, or substituted or unsubstituted thiazolyl are excluded,
or a pharmaceutically acceptable salt thereof.
(2′″) The compound according to the above item (1′″), wherein the ring A is a ring represented by:

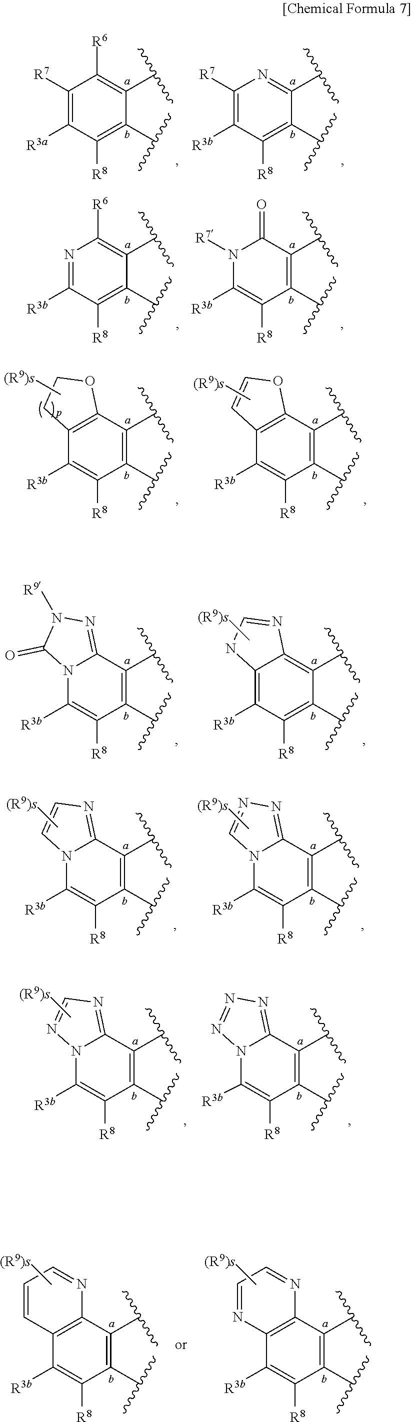

wherein carbon atom a, carbon atom b, R^{3a}, R^{3b}, R⁶, R⁷, R^{7′}, R⁸, R⁹, R⁹ s, and p have the same meanings as those in the above item (1′″),
or a pharmaceutically acceptable salt thereof.
(3′″) The compound according to the above item (1′″) or (2′″),
wherein R^{3a} and R^{3b} are each independently a group represented by:

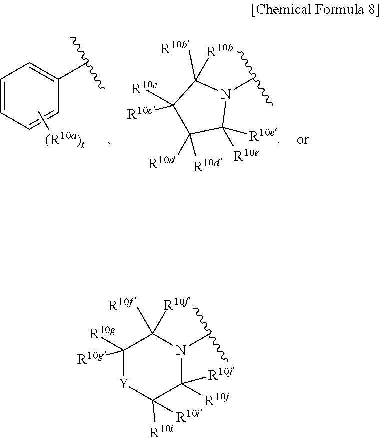

wherein Y is CR^{10h}R^{10h′} or O;
t is an integer of 0 to 5;
R^{10a}s are each independently halogen, cyano, carboxy, substituted or unsubstituted alkyl, substituted or unsubstituted alkyloxy, substituted or unsubstituted alkylcarbonyl, substituted or unsubstituted alkyloxycarbonyl, substituted or unsubstituted alkylsulfonyl, substituted or unsubstituted sulfamoyl, substituted or unsubstituted carbamoyl, substituted or unsubstituted amino, substituted or unsubstituted aromatic heterocyclyl, substituted or unsubstituted non-aromatic heterocyclyl, substituted or unsubstituted non-aromatic heterocyclylcarbonyl, substituted or unsubstituted sulfoxyimino, or substituted or unsubstituted iminosulfino; or
two R^{10a}s bonded to an adjacent carbon atom may be taken together to form substituted or unsubstituted aromatic heterocycle, or substituted or unsubstituted non-aromatic heterocycle;
R^{10b}, R^{10b′}, R^{10c}, R^{10c′}, R^{10d}, R^{10d′}, R^{10e}, R^{10e′}, R^{10f}, R^{10f′}, R^{10g}, R^{10g′}, R^{10h}, R^{10h′}, R¹⁰ⁱ, R^{10i′}, R^{10j}, and R^{10j′} are each independently a hydrogen atom, hydroxy, halogen, substituted or unsubstituted alkyl, substituted or unsubstituted amino, substituted or unsubstituted carbamoyl, substituted or unsubstituted alkyloxy, substituted or unsubstituted alkyloxycarbonyl, or substituted or unsubstituted aromatic heterocyclyl;
R^{10b} and R^{10b′}, R^{10c} and R^{10c′}, R^{10d} and R^{10d′}, R^{10e} and R^{10e′}, R^{10f} and R^{10f′}, R^{10g} and R^{10g′}, R^{10h} and R^{10h′}, R¹⁰ⁱ and R^{10i′}, and R^{10j} and R^{10j′} may be each independently taken together with a carbon atom to which they are bonded to form oxo, a substituted or unsubstituted non-aromatic carbocycle, or a substituted or unsubstituted non-aromatic heterocycle;
R^{10b} and R^{10c}, R^{10c} and R^{10d}, R^{10d} and R^{10e}, R^{10f} and R^{10g}, R^{10g} and R^{10h}, R^{10h} and R¹⁰ⁱ, and R¹⁰ⁱ and R^{10j} may be each independently taken together with a carbon atom to which they are each bonded to form a substituted or unsubstituted non-aromatic heterocycle, a substituted or unsubstituted aromatic carbocycle, or a substituted or unsubstituted non-aromatic carbocycle;
R^{10b} and R^{10d}, R^{10b} and R^{10e}, R^{10c} and R^{10e}, R^{10f} and R^{10h}, R^{10f} and R¹⁰ⁱ, R^{10f} and R^{10j}, R^{10g} and R¹⁰ⁱ, and R^{10g} and R^{10j} may each independently together each form a (C1-C4) bridge, in which one of the carbon atoms that constitute the bridge may be replaced by an oxygen atom or a nitrogen atom; the carbon atoms constituting the bridge are each independently substituted with a substituent selected from R^{a}, and the nitrogen atom constituting the bridge is substituted with a substituent selected from R^{b};
wherein R^{a} is a hydrogen atom, halogen, substituted or unsubstituted alkyl, or substituted or unsubstituted alkyloxy; and
R^{b} is a hydrogen atom, or substituted or unsubstituted alkyl, or
R^{3a} and R^{3b} are each independently substituted amino,
or a pharmaceutically acceptable salt thereof.
(4′″) The compound according to any one of the above items (1′″) to (3′″),
wherein
R² is 6-membered aromatic carbocyclyl which is substituted with one halogen and is further substituted with one, two, three, or four substituents selected from substituent group G, or 6-membered aromatic heterocyclyl which is substituted with one halogen and is further substituted with one or two substituents selected from substituent group G,
wherein the substituent group G is the group consisting of halogen, cyano, alkyl, alkenyl, alkynyl, haloalkyl, alkyloxy, alkenyloxy, alkynyloxy, and haloalkyloxy,
or a pharmaceutically acceptable salt thereof.
(5′″) The compound according to any one of the above items (1′″) to (4′″), wherein R² is 6-membered aromatic carbocyclyl which is substituted with one halogen and is further substituted with one, two, three, or four substituents selected from the substituent group G (having the same meanings as those in the above item (4′″)), or a pharmaceutically acceptable salt thereof.
(6′″) The compound according to any one of the above items (1′″) to (5′″), wherein R¹ is substituted or unsubstituted nitrogen-containing aromatic heterocyclyl, or substituted or unsubstituted nitrogen-containing non-aromatic heterocyclyl, or a pharmaceutically acceptable salt thereof.
(7′″) The compound according to any one of the above items (1′″) to (6′″), wherein the ring A is a ring represented by:

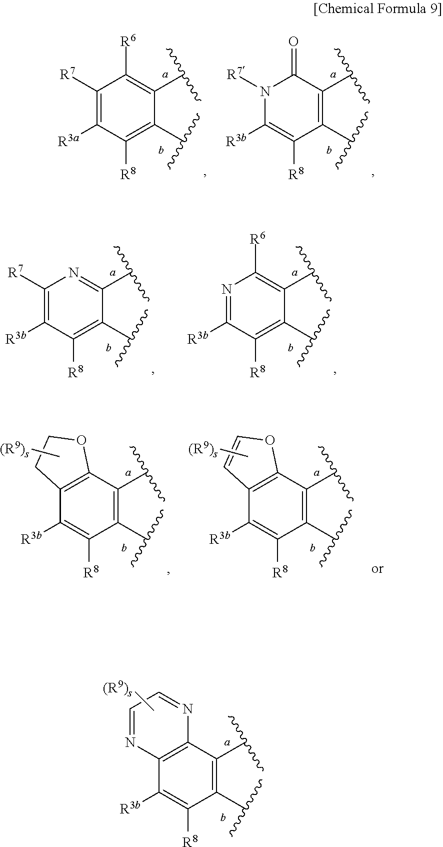

wherein carbon atom a, carbon atom b, R^{3a}, R^{3b}, R⁶, R⁷, R^{7′}, R⁸, R⁹ and s have the same meanings as those in the above item (1′″),
or a pharmaceutically acceptable salt thereof.
(8′″) The compound according to any one of the above items (1′″) to (7′″), wherein the ring A is a ring represented by:

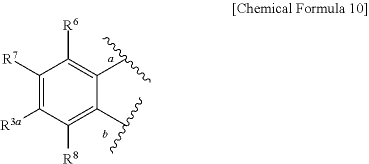

wherein carbon atom a, carbon atom b, R^{3a}, R⁶, and R⁸ have the same meanings as those in the above item (1′″);
R⁷ is a hydrogen atom, halogen, or substituted or unsubstituted alkyl,
or a pharmaceutically acceptable salt thereof.
(9′″) The compound according to any one of the above items (1′″) to (7′″), wherein the ring A is a ring represented by:

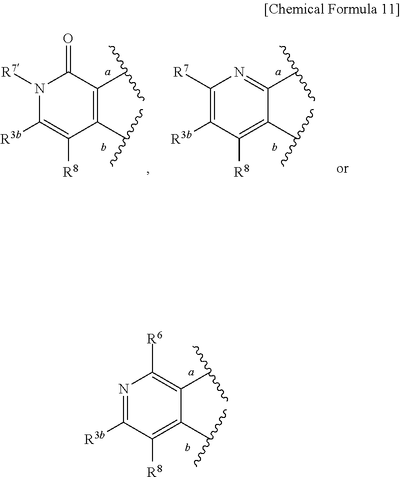

wherein carbon atom a, carbon atom b, R^{3b}, R⁶, R⁷, R^{7′}, and R⁸ have the same meanings as those in the above item (1′),
or a pharmaceutically acceptable salt thereof.
(10′″) The compound according to any one of the above items (1′″) to (9′″), wherein m is 0 or 1,
or a pharmaceutically acceptable salt thereof.
(11′″) The compound according to any one of the above items (1′″) to (10′″), wherein R^{5a}s are each independently a hydrogen atom, and R^{5b}s are each independently a hydrogen atom,
or a pharmaceutically acceptable salt thereof.
(12′″) The compound according to any of the above items (1′″) to (11′″), wherein R⁶ is halogen, substituted or unsubstituted alkyloxy, or hydroxy, or a pharmaceutically acceptable salt thereof.
(13′″) The compound according to any one of the above items (1′″) to (12′″), wherein R^{4a} is a hydrogen atom, and R^{4b} is a hydrogen atom, or a pharmaceutically acceptable salt thereof.
(14′″) The compound according to the above item (1′″), selected from the group consisting of compounds I-0174, I-0414, I-0417, I-0437, I-0699, I-0934, I-0955, I-0957, I-0978, I-0990, and I-1005, or a pharmaceutically acceptable salt thereof.
(15′″) A pharmaceutical composition comprising the compound according to any one of the above items (1′″) to (14′″) or a pharmaceutically acceptable salt thereof.
(16′″) A coronavirus 3CL protease inhibitor comprising the compound according to any one of the above items (1′″) to (14′″) or a pharmaceutically acceptable salt thereof.
(17′″) A coronavirus replication inhibitor comprising the compound according to any one of the above items (1′″) to (14′″) or a pharmaceutically acceptable salt thereof.
(18′″) The coronavirus replication inhibitor according to the above item (17′″), wherein the coronavirus is an alphacoronavirus and/or betacoronavirus.
(19′″) The coronavirus replication inhibitor according to the above item (17′″), wherein the coronavirus is SARS-CoV-2.

Furthermore, the present invention relates to the following.
(1″) A compound represented by Formula (I):

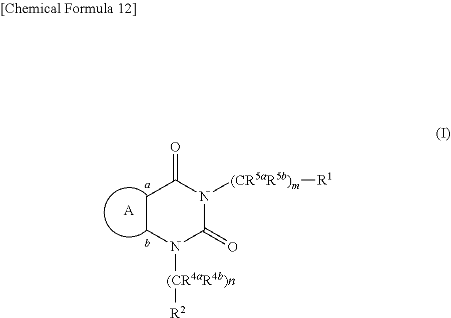

wherein carbon atom a and carbon atom b are each a carbon atom constituting ring A,
the ring A is a ring represented by:

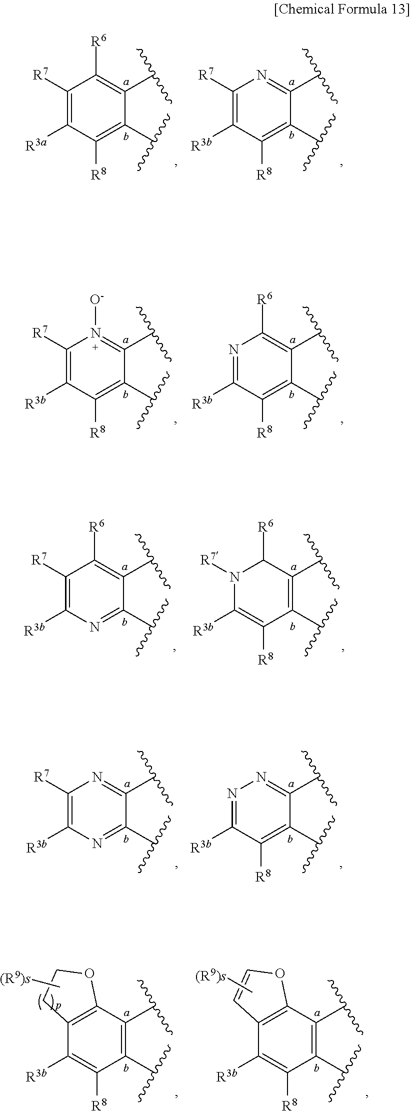

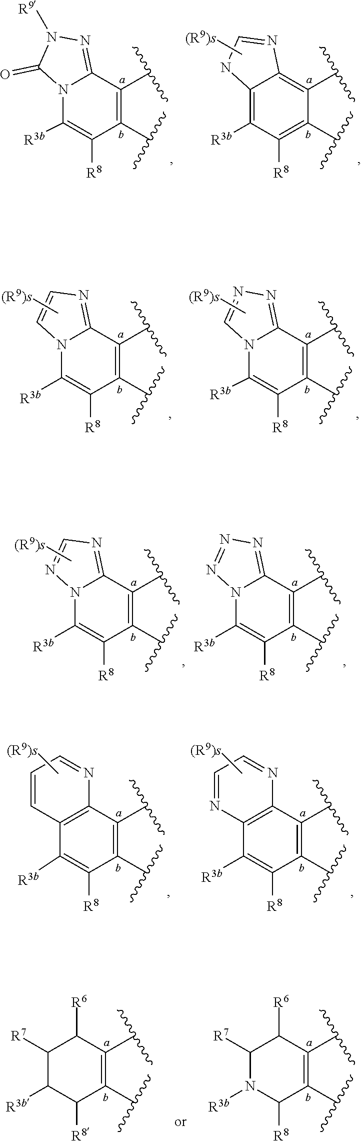

wherein carbon atom a represents the carbon atom a in Formula (I);
the carbon atom b represents the carbon atom b in Formula (I);
R^{3a} is a hydrogen atom, substituted or unsubstituted aromatic carbocyclyl, substituted or unsubstituted aromatic heterocyclyl, substituted or unsubstituted non-aromatic heterocyclyl, substituted or unsubstituted alkyloxy, substituted amino, halogen, substituted or unsubstituted carbamoyl, substituted or unsubstituted sulfamoyl, substituted or unsubstituted non-aromatic carbocyclyloxy, or substituted or unsubstituted non-aromatic heterocyclyloxy;
R^{3b} and R^{3b′} are each independently a hydrogen atom, substituted or unsubstituted aromatic carbocyclyl, substituted or unsubstituted aromatic heterocyclyl, substituted or unsubstituted non-aromatic heterocyclyl, substituted or unsubstituted alkyloxy, substituted amino, halogen, substituted or unsubstituted alkyl, substituted or unsubstituted carbamoyl, substituted or unsubstituted sulfamoyl, substituted or unsubstituted non-aromatic carbocyclyloxy, or substituted or unsubstituted non-aromatic heterocyclyloxy;
R⁸ and R^{8′} are each independently a hydrogen atom, halogen, substituted or unsubstituted alkyl, or substituted or unsubstituted alkyloxy; or
R^{3a} and R⁸, and R^{3b} and R⁸ may be taken together with a carbon atom to which they are each bonded to form a substituted or unsubstituted aromatic carbocycle, or a substituted or unsubstituted aromatic heterocycle;
R⁶ is a hydrogen atom, halogen, substituted or unsubstituted alkyloxy, hydroxy, or cyano;
R⁷ is a hydrogen atom, halogen, substituted or unsubstituted alkyl, substituted or unsubstituted alkenyl, substituted or unsubstituted alkynyl, substituted or unsubstituted alkyloxy, substituted or unsubstituted non-aromatic carbocycle, substituted or unsubstituted non-aromatic heterocycle, substituted or unsubstituted non-aromatic carbocyclyloxy, substituted or unsubstituted amino, substituted or unsubstituted alkylsulfoxy, substituted or unsubstituted carbamoyl, hydroxy, carboxy, formyl, or cyano;
R^{7′} and R^{9′} are each independently a hydrogen atom, or substituted or unsubstituted alkyl;
R^{3b} and R^{7′} may be taken together with an atom to which they are each bonded to form a substituted or unsubstituted non-aromatic heterocycle;
R⁹ is halogen, hydroxy, substituted or unsubstituted alkyl, or substituted or unsubstituted alkyloxy;
s is 0, 1, or 2;
p is 1, 2, or 3;
R¹ is substituted or unsubstituted nitrogen-containing aromatic heterocyclyl, substituted or unsubstituted nitrogen-containing non-aromatic heterocyclyl, or substituted or unsubstituted carbamoyl;
R² is substituted or unsubstituted 6-membered aromatic carbocyclyl, or substituted or unsubstituted 6-membered aromatic heterocyclyl;
n is 0 or 1;
R^{4a}s are each independently a hydrogen atom, or substituted or unsubstituted alkyl;
R^{4b}s are each independently a hydrogen atom, or substituted or unsubstituted alkyl; or R^{4a} and R^{4b} may be taken together with a carbon atom to which they are each bonded to form a substituted or unsubstituted non-aromatic carbocycle, or a substituted or unsubstituted non-aromatic heterocycle;
m is 0, 1, or 2;
R^{5a}s are each independently a hydrogen atom, or substituted or unsubstituted alkyl;
R^{5b}s are each independently a hydrogen atom, or substituted or unsubstituted alkyl,
provided that the compound in which R¹ is substituted or unsubstituted carbamoyl, and R^{3a} and R^{3b} are substituted or unsubstituted oxazolyl, or substituted or unsubstituted thiazolyl are excluded,
or a pharmaceutically acceptable salt thereof.
(2″) The compound according to the above item (1″), wherein the ring A is a ring represented by:

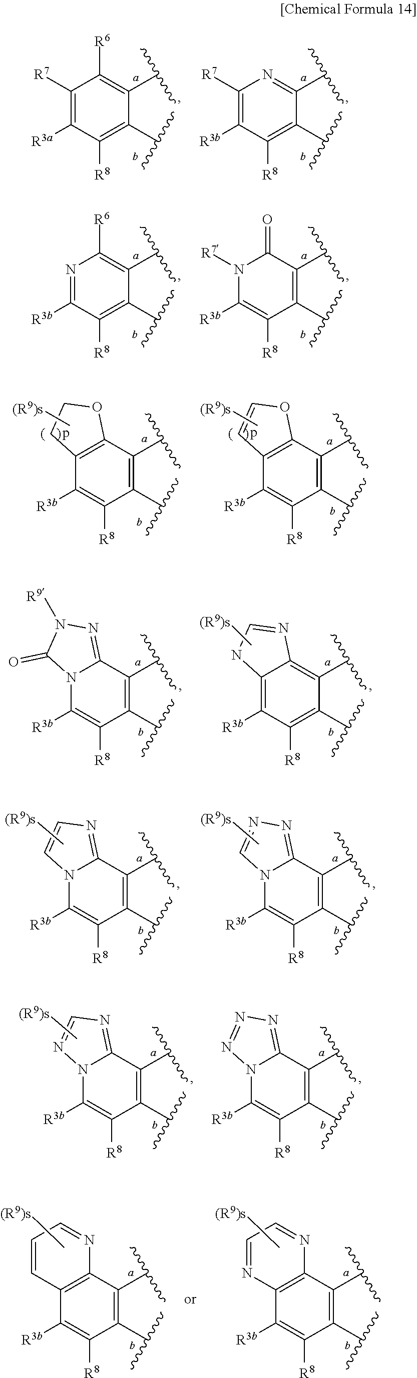

wherein carbon atom a, carbon atom b, R^{3a}, R^{3b}, R⁶, R⁷, R^{7′}, R⁸, R⁹, R^{9′}, s, and p have the same meanings as those in the above item (1″),
or a pharmaceutically acceptable salt thereof.
(3″) The compound according to the above item (1″) or (2″),
wherein R^{3a} and R^{3b} are each independently a group represented by:

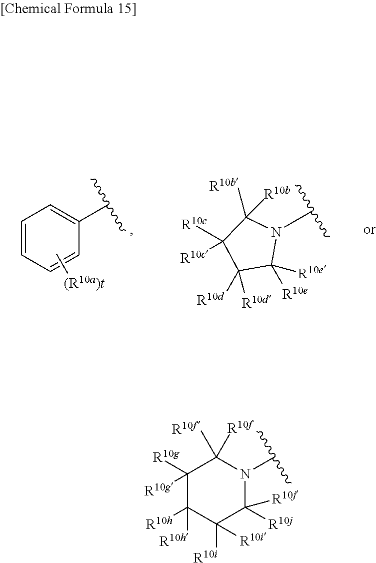

wherein
t is an integer of 0 to 5;
R^{10a}s are each independently halogen, cyano, carboxy, substituted or unsubstituted alkyl, substituted or unsubstituted alkyloxy, substituted or unsubstituted alkylcarbonyl, substituted or unsubstituted alkyloxycarbonyl, substituted or unsubstituted alkylsulfonyl, substituted or unsubstituted sulfamoyl, substituted or unsubstituted carbamoyl, substituted or unsubstituted amino, substituted or unsubstituted aromatic heterocyclyl, substituted or unsubstituted non-aromatic heterocyclyl, substituted or unsubstituted non-aromatic heterocyclylcarbonyl, substituted or unsubstituted sulfoxyimino, or substituted or unsubstituted iminosulfino; or
two R^{10a}s bonded to an adjacent carbon atom may be taken together to form substituted or unsubstituted aromatic heterocycle, or substituted or unsubstituted non-aromatic heterocycle;
R^{10b}, R^{10b′}, R^{10c}, R^{10c′}, R^{10d}, R^{10d′}, R^{10e}, R^{10e′}, R^{10f}, R^{10f′}, R^{10g}, R^{10g′}, R^{10h}, R^{10h′}, R¹⁰ⁱ, R^{10i′}, R^{10j}, and R^{10j′} are each independently a hydrogen atom, hydroxy, halogen, substituted or unsubstituted alkyl, substituted or unsubstituted amino, substituted or unsubstituted carbamoyl, substituted or unsubstituted alkyloxy, substituted or unsubstituted alkyloxycarbonyl, or substituted or unsubstituted aromatic heterocyclyl;
R^{10b} and R^{10b′}, R^{10c} and R^{10c′}, R^{10d} and R^{10d′}, R^{10e} and R^{10e′}, R^{10f} and R^{10f′}, R^{10g} and R^{10g′}, R^{10h} and R^{10h′}, R¹⁰ⁱ and R^{10i′}, and R^{10j} and R^{10j′} may be each independently taken together with a carbon atom to which they are bonded to form oxo, a substituted or unsubstituted non-aromatic carbocycle, or a substituted or unsubstituted non-aromatic heterocycle;
R^{10b} and R^{10c}, R^{10c} and R^{10d}, R^{10d} and R^{10e}, R^{10f} and R^{10g}, R^{10g} and R^{10h}, R^{10h} and R¹⁰ⁱ, and R¹⁰ⁱ and R^{10j} may be each independently taken together with a carbon atom to which they are each bonded to form a substituted or unsubstituted non-aromatic heterocycle, a substituted or unsubstituted aromatic carbocycle, or a substituted or unsubstituted non-aromatic carbocycle;
R^{10b} and R^{10d}, R^{10b} and R^{10e}, R^{10c} and R^{10e}, R^{10f} and R^{10h}, R^{10f} and R¹⁰ⁱ, R^{10f} and R^{10j}, R^{10g} and R¹⁰ⁱ, and R^{10g} and R^{10j} may each independently together each form a (C1-C4) bridge, in which one of the carbon atoms that constitute the bridge may be replaced by an oxygen atom or a nitrogen atom; the carbon atoms constituting the bridge are each independently substituted with a substituent selected from R^{a}, and the nitrogen atom constituting the bridge is substituted with a substituent selected from R^{b};
wherein R^{a} is a hydrogen atom, halogen, substituted or unsubstituted alkyl, or substituted or unsubstituted alkyloxy; and
R^{b} is a hydrogen atom, or substituted or unsubstituted alkyl, or
R^{3a} and R^{3b} are each independently substituted amino,
or a pharmaceutically acceptable salt thereof.
(4″) The compound according to any one of the above items (1″) to (3″),
wherein
R² is 6-membered aromatic carbocyclyl which is substituted with one halogen and is further substituted with one, two, three, or four substituents selected from substituent group G, or 6-membered aromatic heterocyclyl which is substituted with one halogen and is further substituted with one or two substituents selected from substituent group G,
wherein the substituent group G is the group consisting of halogen, cyano, alkyl, alkenyl, alkynyl, haloalkyl, alkyloxy, alkenyloxy, alkynyloxy, and haloalkyloxy,
or a pharmaceutically acceptable salt thereof.
(5″) The compound according to any one of the above items (1″) to (4″), wherein R² is 6-membered aromatic carbocyclyl which is substituted with one halogen and is further substituted with one, two, three, or four substituents selected from the substituent group G (having the same meanings as those in the above item (4″)), or a pharmaceutically acceptable salt thereof.
(6″) The compound according to any one of the above items (1″) to (5″), wherein R¹ is substituted or unsubstituted nitrogen-containing aromatic heterocyclyl, or substituted or unsubstituted nitrogen-containing non-aromatic heterocyclyl, or a pharmaceutically acceptable salt thereof.
(7″) The compound according to any one of the above items (1″) to (6″), wherein the ring A is a ring represented by:

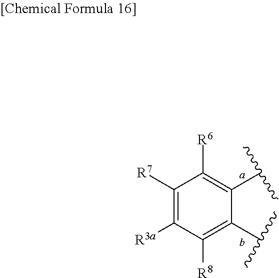

wherein carbon atom a, carbon atom b, R^{3a}, R⁶, and R⁸ have the same meanings as those in the above item (1″);
R⁷ is a hydrogen atom, halogen, or substituted or unsubstituted alkyl,
or a pharmaceutically acceptable salt thereof.
(8″) The compound according to any one of the above items (1″) to (6″), wherein the ring A is a ring represented by:

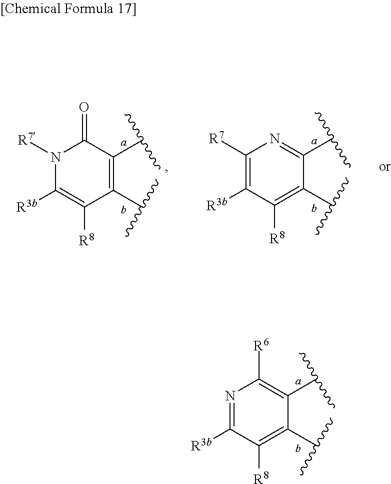

wherein carbon atom a, carbon atom b, R^{3b}, R⁶, R⁷, R^{7′}, and R⁸ have the same meanings as those in the above item (1″),
or a pharmaceutically acceptable salt thereof.
(9″) The compound according to any one of the above items (1″) to (8″), wherein m is 0 or 1,
or a pharmaceutically acceptable salt thereof.
(10″) The compound according to any one of the above items (1″) to (9″), wherein R^{5a}s are each independently a hydrogen atom, and R^{5b}s are each independently a hydrogen atom, or a pharmaceutically acceptable salt thereof.
(11″) The compound according to any of the above items (1″) to (10″), wherein each R⁶ is halogen, substituted or unsubstituted alkyloxy, or hydroxy, or a pharmaceutically acceptable salt thereof.
(12″) The compound according to any one of the above items (1″) to (11″), wherein R^{4a}s are each independently a hydrogen atom, and R^{4b}s are each independently a hydrogen atom, or a pharmaceutically acceptable salt thereof.
(13″) A pharmaceutical composition comprising the compound according to any one of the above items (1″) to (12″) or a pharmaceutically acceptable salt thereof.
(14″) A coronavirus 3CL protease inhibitor comprising the compound according to any one of the above items (1″) to (12″) or a pharmaceutically acceptable salt thereof.
(15″) A coronavirus replication inhibitor comprising the compound according to any one of the above items (1″) to (12″) or a pharmaceutically acceptable salt thereof.
(16″) The coronavirus replication inhibitor according to the above item (15″), wherein the coronavirus is an alphacoronavirus and/or betacoronavirus.
(17″) The coronavirus replication inhibitor according to the above item (15″), wherein the coronavirus is SARS-CoV-2.

Furthermore, the present invention relates to the following.
(1′) A compound re resented by Formula (I):

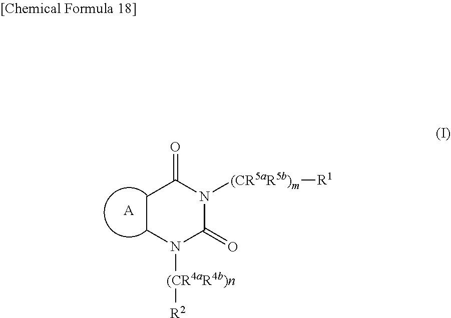

wherein ring A is a ring represented by:

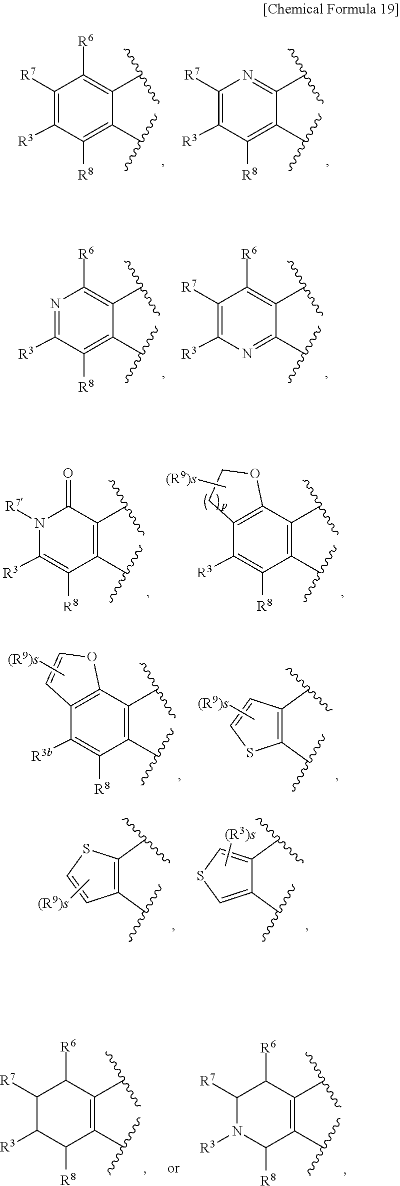

wherein
R³ is a hydrogen atom, substituted or unsubstituted aromatic carbocyclyl, substituted or unsubstituted aromatic heterocyclyl, substituted or unsubstituted non-aromatic heterocyclyl, substituted or unsubstituted alkyloxy, substituted or unsubstituted amino, halogen, substituted or unsubstituted alkyl, or substituted or unsubstituted non-aromatic heterocyclyloxy;
R⁸ is a hydrogen atom or halogen; or
R³ and R⁸ may be taken together with a carbon atom to which they are each bonded to form a substituted or unsubstituted non-aromatic carbocycle, or a substituted or unsubstituted aromatic heterocycle;
R⁶ is a hydrogen atom, halogen, substituted or unsubstituted alkyloxy, or hydroxy;
R⁷ is a hydrogen atom, halogen, substituted or unsubstituted alkyl, substituted or unsubstituted alkyloxy, or substituted or unsubstituted amino or hydroxy;
R^{7′} is a hydrogen atom, or substituted or unsubstituted alkyl;
R⁹ is halogen, substituted or unsubstituted alkyl, or substituted or unsubstituted alkyloxy;
s is 0, 1, or 2;
p is 1, 2, or 3,
R¹ is substituted or unsubstituted aromatic heterocyclyl, substituted or unsubstituted non-aromatic heterocyclyl, substituted or unsubstituted carbamoyl, or substituted or unsubstituted amino;
R² is substituted or unsubstituted aromatic carbocyclyl, substituted or unsubstituted non-aromatic carbocyclyl, substituted or unsubstituted aromatic heterocyclyl, substituted or unsubstituted non-aromatic heterocyclyl, or substituted or unsubstituted alkyl;
n is 0, 1, or 2;
R^{4a}s is each independently a hydrogen atom, or substituted or unsubstituted alkyl;
R^{4b}s are each independently a hydrogen atom, or substituted or unsubstituted alkyl; or R^{4a} and R^{4b} may be taken together with a carbon atom to which they are each bonded to form a substituted or unsubstituted non-aromatic carbocycle, or a substituted or unsubstituted non-aromatic heterocycle;
m is 0, 1, or 2;
R^{5a}s are each independently a hydrogen atom, or substituted or unsubstituted alkyl;
R^{5b}s are each independently a hydrogen atom, or substituted or unsubstituted alkyl,
provided that the following compounds are excluded:

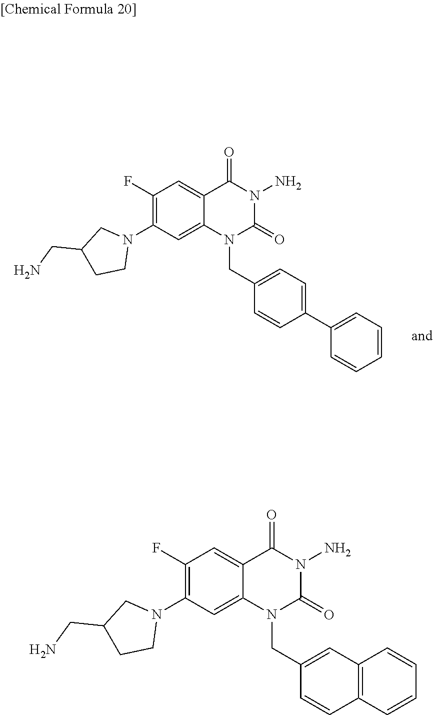 [SMILES: NCC1CCN(c2cc3c(cc2F)c(=O)n(N)c(=O)n3Cc2ccc(-c3ccccc3)cc2)C1 ; NCC1CCN(c2cc3c(cc2F)c(=O)n(N)c(=O)n3Cc2ccc3ccccc3c2)C1]

or a pharmaceutically acceptable salt thereof.
(2′) The compound according to the above item (1′), wherein the ring A is a ring represented by:

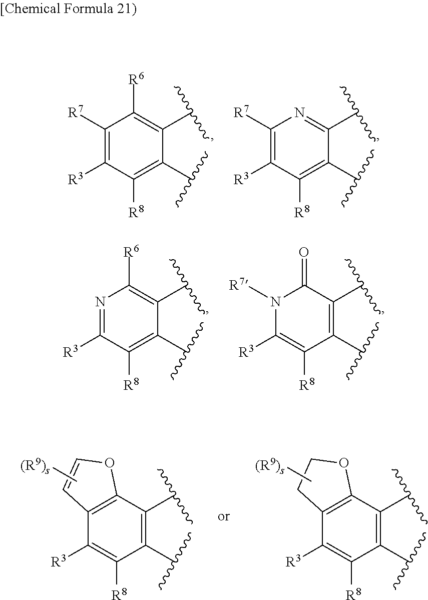

wherein R⁶, R⁷, R⁹, and s have the same meanings as those in the above item (1′);
R⁸ is a hydrogen atom or halogen;
R³ is a cyclic group represented by:

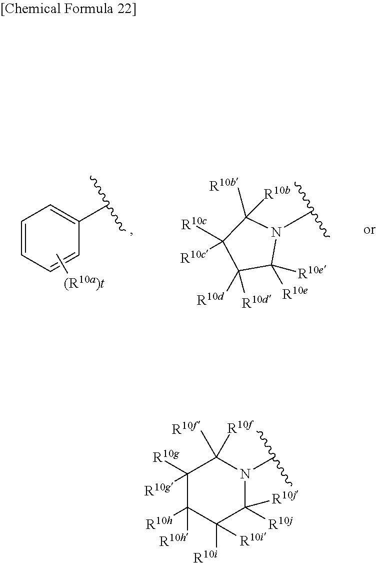

wherein
t is an integer of 0 to 5;
R^{10a}s are each independently halogen, cyano, carboxy, substituted or unsubstituted alkyl, substituted or unsubstituted alkyloxy, substituted or unsubstituted alkylcarbonyl, substituted or unsubstituted alkyloxycarbonyl, substituted or unsubstituted alkylsulfonyl, substituted or unsubstituted sulfamoyl, substituted or unsubstituted carbamoyl substituted or unsubstituted amino, substituted or unsubstituted aromatic heterocyclyl, substituted or unsubstituted non-aromatic heterocyclyl, substituted or unsubstituted non-aromatic heterocyclylcarbonyl, substituted or unsubstituted sulfoxyimino, or substituted or unsubstituted iminosulfino; or
two R^{10a}s bonded to an adjacent carbon atom may be taken together to form substituted or unsubstituted aromatic heterocycle, or substituted or unsubstituted non-aromatic heterocycle;
R^{10b}, R^{10b′}, R^{10c}, R^{10c′}, R^{10d}, R^{10d′}, R^{10e}, R^{10e′}, R^{10f}, R^{10f′}, R^{10g}, R^{10g′}, R^{10h}, R^{10h′}, R¹⁰ⁱ, R^{10i′}, R^{10j}, and R^{10j′} are each independently a hydrogen atom, hydroxy, halogen, substituted or unsubstituted alkyl, substituted or unsubstituted amino, substituted or unsubstituted carbamoyl, substituted or unsubstituted alkyloxy, substituted or unsubstituted alkyloxycarbonyl, or substituted or unsubstituted aromatic heterocyclyl;
R^{10b} and R^{10b′}, R^{10c} and R^{10c′}, R^{10d} and R^{10d′}, R^{10e} and R^{10e′}, R^{10f} and R^{10f′}, R^{10g} and R^{10g′}, R^{10h} and R^{10h′}, R¹⁰ⁱ and R^{10i′}, and R^{10j} and R^{10j′} may be each independently taken together with a carbon atom to which they are each bonded to form oxo, a substituted or unsubstituted non-aromatic carbocycle, or a substituted or unsubstituted non-aromatic heterocycle;
R^{10b} and R^{10c}, R^{10c} and R^{10d}, R^{10d} and R^{10e}, R^{10f} and R^{10g}, R^{10g} and R^{10h}, R^{10h} and R¹⁰ⁱ, and R¹⁰ⁱ and R^{10j} may be each independently taken together with a carbon atom to which they are each bonded to form a substituted or unsubstituted non-aromatic heterocycle, a substituted or unsubstituted aromatic carbocycle, or a substituted or unsubstituted non-aromatic carbocycle;
R^{10b} and R^{10d}, R^{10b} and R^{10e}, R^{10c} and R^{10e}, R^{10f} and R^{10h}, R^{10f} and R¹⁰ⁱ, R^{10f} and R^{10j}, R^{10g} and R¹⁰ⁱ, and R^{10g} and R¹⁰ⁱ may each independently together each form a (C1-C4) bridge, in which one of the carbon atoms that constitute the bridge may be replaced by an oxygen atom or a nitrogen atom; the carbon atoms constituting the bridge are each independently substituted with a substituent selected from R^{a}, and the nitrogen atom constituting the bridge is substituted with a substituent selected from R^{b};
wherein R^{a} is a hydrogen atom, halogen, substituted or unsubstituted alkyl, or substituted or unsubstituted alkyloxy; and
R^{b} is a hydrogen atom, or substituted or unsubstituted alkyl; or alternatively, R³ is substituted or unsubstituted alkyloxy;
R¹ is substituted or unsubstituted aromatic heterocyclyl, substituted or unsubstituted non-aromatic heterocyclyl, or substituted or unsubstituted amino;
R² is substituted or unsubstituted aromatic carbocyclyl, or substituted or unsubstituted aromatic heterocyclyl,
or a pharmaceutically acceptable salt thereof.
(3′) The compound according to any one of the above item (1′) or (2′),
wherein
R² is 6-membered aromatic carbocyclyl which is substituted with one halogen and is further substituted with one, two, three, or four substituents selected from substituent group G, or 6-membered aromatic heterocyclyl which is substituted with one halogen and is further substituted with one or two substituents selected from substituent group G,
wherein the substituent group G is the group consisting of halogen, cyano, alkyl, alkenyl, alkynyl, haloalkyl, alkyloxy, alkenyloxy, alkynyloxy, and haloalkyloxy,
or a pharmaceutically acceptable salt thereof.
(4′) The compound according to any one of the above items (1′) to (3′), wherein R² is 6-membered aromatic carbocyclyl which is substituted with one halogen and is further substituted with one, two, three, or four substituents selected from the substituent group G (having the same meanings as those in the above item (3′)), or a pharmaceutically acceptable salt thereof.
(5′) The compound according to any one of the above items (1′) to (4′), wherein R¹ is substituted or unsubstituted aromatic heterocyclyl, or substituted or unsubstituted non-aromatic heterocyclyl, or a pharmaceutically acceptable salt thereof.
(6′) The compound according to any one of the above items (1′) to (5′), wherein the ring A is a ring represented by:

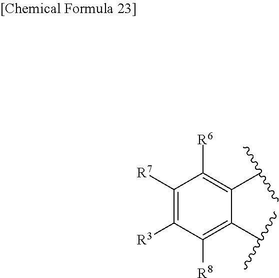

wherein R⁶ and R⁸ have the same meanings as those in the above item (1′);
R³ has the same meaning as that in the above item (2′);
R⁷ is a hydrogen atom, halogen, or substituted or unsubstituted alkyl,
or a pharmaceutically acceptable salt thereof.
(7′) The compound according to any one of the above items (1′) to (5′), wherein the ring A is a ring represented by:

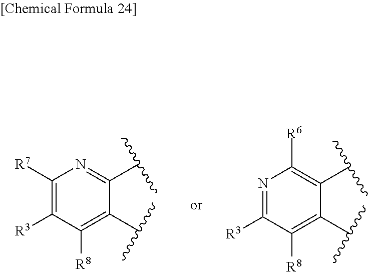

wherein R⁶, R⁷, and R⁸ have the same meanings as those in the above item (1′);
R³ has the same meaning as that in the above item (2′),
or a pharmaceutically acceptable salt thereof.
(8′) The compound according to any one of the above items (1′) to (7′), wherein m is 0 or 1, or a pharmaceutically acceptable salt thereof.
(9′) The compound according to any one of the above items (1′) to (8′), wherein R^{5a}s are each independently a hydrogen atom, and R^{5b}s are each independently a hydrogen atom, or a pharmaceutically acceptable salt thereof.
(10′) The compound according to any one of the above items (1′) to (9′), wherein n is 0 or 1,
or a pharmaceutically acceptable salt thereof.
(11′) The compound according to any one of the above items (1′) to (10′), wherein R^{4a}s are each independently a hydrogen atom, and R^{4b}s are each independently a hydrogen atom, or a pharmaceutically acceptable salt thereof.
(12′) A pharmaceutical composition comprising the compound according to any one of the above items (1′) to (11′) or a pharmaceutically acceptable salt thereof.
(13′) A coronavirus 3CL protease inhibitor comprising the compound according to any one of the above items (1′) to (11′) or a pharmaceutically acceptable salt thereof.
(14′) A coronavirus replication inhibitor comprising the compound according to any one of the above items (1′) to (11′) or a pharmaceutically acceptable salt thereof.
(15′) The coronavirus replication inhibitor according to the above item (14′), wherein the coronavirus is an alphacoronavirus and/or beta coronavirus.
(16′) The coronavirus replication inhibitor according to the above item (14′), wherein the coronavirus is SARS-CoV-2.

Furthermore, the present invention relates to the following.
(1) A compound represented by Formula (I):

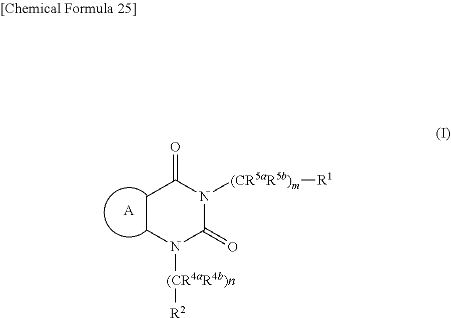

wherein ring A is a ring represented by:

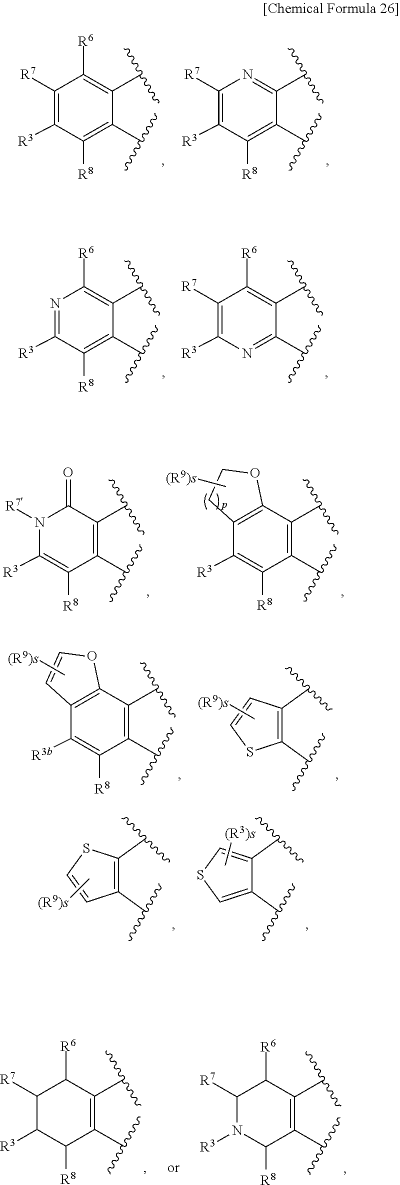

wherein
R³ is a hydrogen atom, substituted or unsubstituted aromatic carbocyclyl, substituted or unsubstituted aromatic heterocyclyl, substituted or unsubstituted non-aromatic heterocyclyl, substituted or unsubstituted alkyloxy, or substituted or unsubstituted amino;
R⁸ is a hydrogen atom or halogen; or
R³ and R⁸ may be taken together with a carbon atom to which they are each bonded to form a substituted or unsubstituted aromatic carbocycle, or a substituted or unsubstituted aromatic heterocycle;
R⁶ is a hydrogen atom, halogen, or substituted or unsubstituted alkyloxy;
R⁷ is a hydrogen atom, halogen, substituted or unsubstituted alkyl, substituted or unsubstituted alkyloxy, or substituted or unsubstituted amino;
R^{7′} is a hydrogen atom, or substituted or unsubstituted alkyl;
R⁹ is halogen, substituted or unsubstituted alkyl, or substituted or unsubstituted alkyloxy;
s is 0, 1, or 2;
p is 1, 2, or 3,
R¹ is substituted or unsubstituted aromatic heterocyclyl, substituted or unsubstituted non-aromatic heterocyclyl, substituted or unsubstituted carbamoyl, or substituted or unsubstituted amino;
R² is substituted or unsubstituted aromatic carbocyclyl, substituted or unsubstituted non-aromatic carbocyclyl, substituted or unsubstituted aromatic heterocyclyl, substituted or unsubstituted non-aromatic heterocyclyl, or substituted or unsubstituted alkyl;
n is 0, 1, or 2;
R^{4a}s are each independently a hydrogen atom, or substituted or unsubstituted alkyl;
R^{4b}s are each independently a hydrogen atom, or substituted or unsubstituted alkyl; or R^{4a} and R^{4b} may be taken together with a carbon atom to which they are each bonded to form a substituted or unsubstituted non-aromatic carbocycle, or a substituted or unsubstituted non-aromatic heterocycle;
m is 0, 1, or 2;
R^{5a}s are each independently a hydrogen atom, or substituted or unsubstituted alkyl;
R^{5b}s are each independently a hydrogen atom, or substituted or unsubstituted alkyl,
provided that the following compounds are excluded:

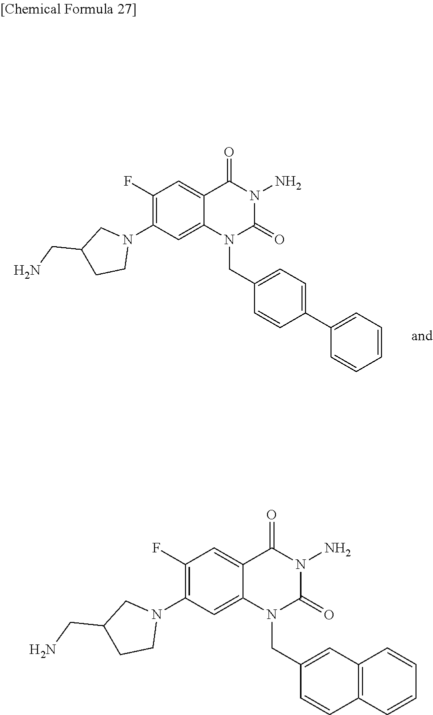 [SMILES: NCC1CCN(c2cc3c(cc2F)c(=O)n(N)c(=O)n3Cc2ccc(-c3ccccc3)cc2)C1 ; NCC1CCN(c2cc3c(cc2F)c(=O)n(N)c(=O)n3Cc2ccc3ccccc3c2)C1]

or a pharmaceutically acceptable salt thereof.
(2) The compound according to the above item (1), wherein the ring A is a ring represented by:

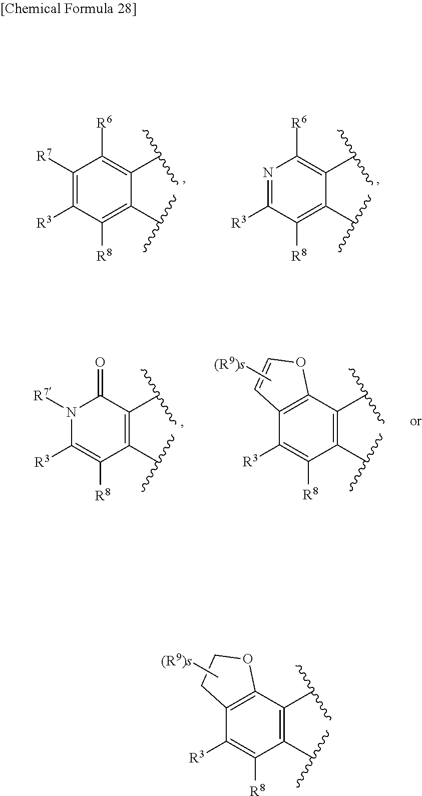

wherein R⁶, R⁷, R⁹, and s have the same meanings as those in the above item (1);
R⁸ is a hydrogen atom or halogen;
R³ is a cyclic group represented by:

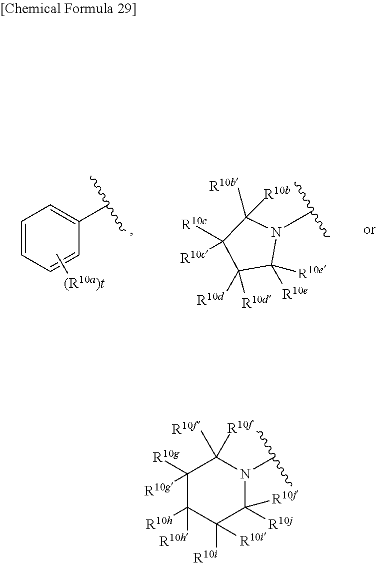

wherein
t is an integer of 0 to 5;
R^{10a}s are each independently halogen, cyano, carboxy, substituted or unsubstituted alkyl, substituted or unsubstituted alkyloxy, substituted or unsubstituted alkylcarbonyl, substituted or unsubstituted alkyloxycarbonyl, substituted or unsubstituted alkylsulfonyl, substituted or unsubstituted sulfamoyl, substituted or unsubstituted carbamoyl, substituted or unsubstituted amino, substituted or unsubstituted aromatic heterocyclyl, substituted or unsubstituted non-aromatic heterocyclyl, substituted or unsubstituted non-aromatic heterocyclylcarbonyl, substituted or unsubstituted sulfoxyimino, or substituted or unsubstituted iminosulfino; or
two R^{10a}s bonded to an adjacent carbon atom may be taken together to form substituted or unsubstituted aromatic heterocycle, or substituted or unsubstituted non-aromatic heterocycle;
R^{10b}, R^{10b′}, R_{10c}, R^{10c′}, R^{10d}, R^{10d′}, R^{10e}, R^{10e′}, R^{10f}, R^{10f′}, R^{10g}, R^{10g′}, R^{10h}, R^{10h′}, R¹⁰ⁱ, R^{10i′}, R^{10j}, and R^{10j′} are each independently a hydrogen atom, hydroxy, halogen, substituted or unsubstituted alkyl, substituted or unsubstituted amino, substituted or unsubstituted carbamoyl, substituted or unsubstituted alkyloxy, substituted or unsubstituted alkyloxycarbonyl, or substituted or unsubstituted aromatic heterocyclyl;
R^{10b} and R^{10b′}, R^{10c} and R^{10c′}, R^{10d} and R^{10d′}, R^{10e} and R^{10e′}, R^{10f} and R^{10f′}, R^{10g} and R^{10g′}, R^{10h} and R^{10h′}, R¹⁰ⁱ and R^{10i′}, and R^{10j} and R^{10j′} may be each independently taken together with a carbon atom to which they are bonded to form oxo, a substituted or unsubstituted non-aromatic carbocycle, or a substituted or unsubstituted non-aromatic heterocycle;
R^{10b} and R^{10c}, R^{10c} and R^{10d}, R^{10d} and R^{10e}, R^{10f} and R^{10g}, R^{10g} and R^{10h}, R^{10h} and R¹⁰ⁱ, and R¹⁰ⁱ and R^{10j} may be each independently taken together with a carbon atom to which they are each bonded to form a substituted or unsubstituted non-aromatic heterocycle, a substituted or unsubstituted aromatic carbocycle, or a substituted or unsubstituted non-aromatic carbocycle;
R^{10b} and R^{10d}, R^{10b} and R^{10e}, R^{10c} and R^{10e}, R^{10f} and R^{10h}, R^{10f} and R¹⁰ⁱ, R^{10f} and R^{10j}, R^{10g} and R¹⁰ⁱ, and R^{10g} and R^{10j} may each independently together each form a (C1-C4) bridge, in which one of the carbon atoms that constitute the bridge may be replaced by an oxygen atom or a nitrogen atom; the carbon atoms constituting the bridge are each independently substituted with a substituent selected from R^{a}, and the nitrogen atom constituting the bridge is substituted with a substituent selected from R^{b};
wherein R^{a} is a hydrogen atom, halogen, substituted or unsubstituted alkyl, or substituted or unsubstituted alkyloxy;
R^{b} is a hydrogen atom, or substituted or unsubstituted alkyl;
or alternatively, R³ is substituted or unsubstituted alkyloxy;
R¹ is substituted or unsubstituted aromatic heterocyclyl, substituted or unsubstituted non-aromatic heterocyclyl, or substituted or unsubstituted amino;
R² is substituted or unsubstituted aromatic carbocyclyl, or substituted or unsubstituted aromatic heterocyclyl,
or a pharmaceutically acceptable salt thereof.
(3) The compound according to any one of the above item (1) or (2),
wherein
R² is 6-membered aromatic carbocyclyl which is substituted with one halogen and is further substituted with one, two, three, or four substituents selected from substituent group G, or 6-membered aromatic heterocyclyl which is substituted with one halogen and is further substituted with one or two substituents selected from substituent group G,
wherein the substituent group G is the group consisting of halogen, cyano, alkyl, alkenyl, alkynyl, haloalkyl, alkyloxy, alkenyloxy, alkynyloxy, and haloalkyloxy,
or a pharmaceutically acceptable salt thereof.
(4) The compound according to any one of the above items (1) to (3), wherein R² is 6-membered aromatic carbocyclyl which is substituted with one halogen and is further substituted with one, two, three, or four substituents selected from the substituent group G (having the same meanings as those in the above item (3)), or a pharmaceutically acceptable salt thereof.
(5) The compound according to any one of the above items (1) to (4), wherein R¹ is substituted or unsubstituted aromatic heterocyclyl, or substituted or unsubstituted non-aromatic heterocyclyl, or a pharmaceutically acceptable salt thereof.
(6) The compound according to any one of the above items (1) to (5), wherein the ring A is a ring represented by:

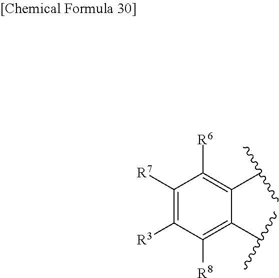

wherein R⁶ and R⁸ have the same meanings as those in the above item (1);
R³ has the same meaning as that in the above item (2); and
R⁷ is a hydrogen atom, halogen, or substituted or unsubstituted alkyl,
or a pharmaceutically acceptable salt thereof.
(7) The compound according to any one of the above items (1) to (6), wherein m is 0 or 1, or a pharmaceutically acceptable salt thereof.
(8) The compound according to any one of the above items (1) to (7), wherein R^{5a}s are each independently a hydrogen atom, and R^{5b}s are each independently a hydrogen atom, or a pharmaceutically acceptable salt thereof.
(9) The compound according to any one of the above items (1) to (8), wherein n is 0 or 1,
or a pharmaceutically acceptable salt thereof.
(10) The compound according to any one of the above items (1) to (9), wherein R^{4a}s are each independently a hydrogen atom, and R^{4b}s are each independently a hydrogen atom, or a pharmaceutically acceptable salt thereof.
(11) A pharmaceutical composition comprising the compound according to any one of the above items (1) to (10) or a pharmaceutically acceptable salt thereof.
(12) A coronavirus 3CL protease inhibitor comprising the compound according to any one of the above items (1) to (10) or a pharmaceutically acceptable salt thereof.
(13) A coronavirus replication inhibitor comprising the compound according to any one of the above items (1) to (10) or a pharmaceutically acceptable salt thereof.
(14) The coronavirus replication inhibitor according to the above item (13), wherein the coronavirus is an alphacoronavirus and/or betacoronavirus.
(15) The coronavirus replication inhibitor according to the above item (13), wherein the coronavirus is SARS-CoV-2.
(16) A pharmaceutical composition for preventing and/or treating coronavirus infections, the pharmaceutical composition containing the compound according to any one of the above items (1) to (10), (1′) to (11′), (1″) to (12″), and (1′″) to (14′″) or a pharmaceutically acceptable salt thereof.
(17) The pharmaceutical composition according to the above item (16), for preventing and/or treating novel coronavirus infections (COVID-19).
(18) The pharmaceutical composition according to the above item (16), for preventing and/or treating infections caused by SARS-CoV-2.
(19) A method for inhibiting replication of coronavirus, the method comprising administering the compound according to any one of the above items (1) to (10), (1′) to (11′), (1″) to (12″), and (1′″) to (14′″) or a pharmaceutically acceptable salt thereof.
(20) The method for inhibiting replication according to the above item (19), wherein the coronavirus is an alphacoronavirus and/or betacoronavirus.
(21) The method for inhibiting replication according to the above item (19), wherein the coronavirus is SARS-CoV-2.
(22) A method for treating and/or preventing a disease associated with coronavirus 3CL proteases, the method comprising administering the compound according to any one of the above items (1) to (10), (1′) to (11′), (1″) to (12″), and (1′″) to (14′″) or a pharmaceutically acceptable salt thereof.
(23) A method for treating and/or preventing coronavirus infections, characterized by administering the compound according to any one of the above items (1) to (10), (1′) to (11′), (1″) to (12″), and (1′″) to (14′″) or a pharmaceutically acceptable salt thereof.
(24) The method for preventing and/or treating according to the above item (23), wherein the coronavirus infections are novel coronavirus infections (COVID-19).
(25) The method for preventing and/or treating according to the above item (23), wherein the coronavirus infections are infections caused by SARS-CoV-2.
(26) Use of the compound according to any one of the above-described items (1) to (10), (1′) to (11′), (1″) to (12″), and (1′″) to (14′″) or a pharmaceutically acceptable salt thereof, for producing a therapeutic and/or prophylactic agent for a disease associated with coronavirus 3CL protease.
(27) Use of the compound according to any one of the above items (1) to (10), (1′) to (11′), (1″) to (12″), and (1′″) to (14′″) or a pharmaceutically acceptable salt thereof, for producing a replication inhibitor for coronavirus.
(28) The use according to the above item (27), wherein the coronavirus is an alphacoronavirus and/or betacoronavirus.
(29) The use according to the above item (27), wherein the coronavirus is SARS-CoV-2.
(30) Use of the compound according to any one of the above items (1) to (10), (1′) to (11′), (1″) to (12″), and (1′″) to (14′″) or a pharmaceutically acceptable salt thereof, for producing a therapeutic and/or prophylactic agent for coronavirus infections.
(31) The use according to the above item (30), wherein the coronavirus infections are novel coronavirus infections (COVID-19).
(32) The use according to the above item (30), wherein the coronavirus infections are infections caused by SARS-CoV-2.
(33) The compound according to any one of the above items (1) to (10), (1′) to (11′), (1″) to (12″), and (1′″) to (14′″) or a pharmaceutically acceptable salt thereof, for use in the treatment and/or prevention of a disease associated with coronavirus 3CL proteases.
(34) The compound according to any one of the above items (1) to (10), (1′) to (11′), (1″) to (12″), and (1′″) to (14′″) or a pharmaceutically acceptable salt thereof, for use in the inhibition of replication of coronavirus.
(35) The compound according to the above item (34), or a pharmaceutically acceptable salt thereof, wherein the coronavirus is an alphacoronavirus and/or betacoronavirus.
(36) The compound according to the above item (34), or a pharmaceutically acceptable salt thereof, wherein the coronavirus is SARS-CoV-2.
(37) The compound according to any one of the above items (1) to (10), (1′) to (11′), (1″) to (12″), and (1′″) to (14′″) or a pharmaceutically acceptable salt thereof, for use in treating and/or preventing coronavirus infections.
(38) The compound according to the above item (37), or a pharmaceutically acceptable salt thereof, wherein the coronavirus infections are novel coronavirus infections (COVID-19).
(39) The compound according to the above item (37), or a pharmaceutically acceptable salt thereof, wherein the coronavirus infections are infections caused by SARS-CoV-2.

### Effect of the Invention

The compound of the present invention has inhibitory activity against coronavirus 3CL proteases and is useful as a therapeutic (treating) agent and/or prophylactic (preventing) agent for coronavirus infections.

### MODE FOR CARRYING OUT THE INVENTION

Hereinafter, the meaning of each term used in the present specification will be described. Unless particularly stated otherwise, each term is used in the same sense, either alone or in combination with other terms.

The term “consist of” means having only the constituent elements.

The term “comprise” means that elements are not limited to the constituent elements, and elements that are not described are not excluded.

Hereinafter, the present invention will be described while showing exemplary embodiments. Throughout the present specification, it should be understood that, unless particularly stated otherwise, an expression of a singular form also includes the concept of a plural form thereof. Therefore, it should be understood that, unless particularly stated otherwise, an article for a singular form (for example, in the case of English, “a”, “an”, “the”, or the like) also includes the concept of a plural form thereof.

Furthermore, it should be understood that, unless particularly stated otherwise, the terms used in the present specification are used in the meanings normally used in the above-described art. Accordingly, unless otherwise defined, all terminologies and scientific and technical terms used in the present specification have the same meanings as commonly understood by those having ordinary skill in the art to which the present invention belongs. In a case of contradiction, priority is given to the present specification (including definitions).

“Halogen” includes a fluorine atom, a chlorine atom, a bromine atom, and an iodine atom. Particularly, a fluorine atom and a chlorine atom are preferred.

“Alkyl” includes linear or branched hydrocarbon groups each having 1 to 15 carbon atoms, preferably 1 to 10 carbon atoms, more preferably 1 to 6 carbon atoms, and even more preferably 1 to 4 carbon atoms. Examples include methyl, ethyl, n-propyl, isopropyl, n-butyl, isobutyl, sec-butyl, tert-butyl, n-pentyl, isopentyl, neopentyl, n-hexyl, isohexyl, n-heptyl, isoheptyl, n-octyl, isooctyl, n-nonyl, and n-decyl.

Preferred embodiments of “alkyl” include methyl, ethyl, n-propyl, isopropyl, n-butyl, isobutyl, sec-butyl, tert-butyl, and n-pentyl. More preferred embodiments include methyl, ethyl, n-propyl, isopropyl, and tert-butyl.

“Alkenyl” includes linear or branched hydrocarbon groups each having one or more double bonds at any position and having 2 to 15 carbon atoms, preferably 2 to 10 carbon atoms, more preferably 2 to 6 carbon atoms, and even more preferably 2 to 4 carbon atoms. Examples include vinyl, allyl, propenyl, isopropenyl, butenyl, isobutenyl, prenyl, butadienyl, pentenyl, isopentenyl, pentadienyl, hexenyl, isohexenyl, hexadienyl, heptenyl, octenyl, nonenyl, decenyl, undecenyl, dodecenyl, tridecenyl, tetradecenyl, and pentadecenyl.

Preferred embodiments of “alkenyl” include vinyl, allyl, propenyl, isopropenyl, and butenyl. More preferred embodiments include ethenyl and n-propenyl.

“Alkynyl” includes linear or branched hydrocarbon groups each having one or more triple bonds at any position and having 2 to 10 carbon atoms, preferably 2 to 8 carbon atoms, more preferably 2 to 6 carbon atoms, and even more preferably 2 to 4 carbon atoms. Alkynyl may further have a double bond at any position. Examples include ethynyl, propynyl, butynyl, pentynyl, hexynyl, heptynyl, octynyl, nonynyl, and decynyl.

Preferred embodiments of “alkynyl” include ethynyl, propynyl, butynyl, and pentynyl. More preferred embodiments include ethynyl and propynyl.

“Aromatic carbocyclyl” means a cyclic aromatic hydrocarbon group having a single ring or two or more rings. Examples include phenyl, naphthyl, anthryl, and phenanthryl. Examples of 6-membered aromatic carbocyclyl include phenyl.

Preferred embodiments of the “aromatic carbocyclyl” include phenyl.

“Aromatic carbocycle” means a ring derived from the above-described “aromatic carbocyclyl”.

In Formula (I) above, examples of “substituted or unsubstituted aromatic carbocycle formed by R³ and R⁸ taken together with a carbon atom to which they are each bonded”, and the “substituted or unsubstituted aromatic carbocycles formed by R^{3a} and R⁸, and R^{3b} and R⁸ taken together with a carbon atom to which they are each bonded” are, for example, the following ring:

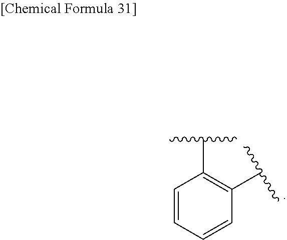 [SMILES: *c1ccccc1*]

In Formula (I) above, examples of “substituted or unsubstituted aromatic carbocycle formed by R^{10b} and R^{10c}, R^{10c} and R^{10d}, R^{10d} and R^{10e}, R^{10f} and R^{10g}, R^{10g} and R^{10h}, R^{10h} and R¹⁰ⁱ, and R¹⁰ⁱ and R^{10j} each independently taken together with a carbon atom to which they are each bonded” include the following ring:

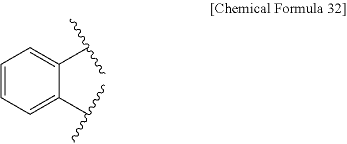 [SMILES: *c1ccccc1*]

“Non-aromatic carbocyclyl” means a cyclic saturated hydrocarbon group or a cyclic non-aromatic unsaturated hydrocarbon group, both having a single ring or two or more rings. The “non-aromatic carbocyclyl” having two or more rings also includes a non-aromatic carbocyclyl having a single ring or two or more rings, to which the ring in the “aromatic carbocyclyl” is fused.

Furthermore, the “non-aromatic carbocyclyl” also includes a bridged group or a group forming a spiro ring, such as follows.

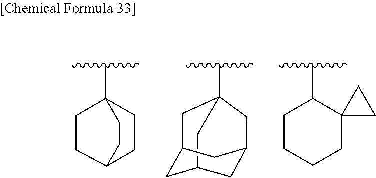

A non-aromatic carbocyclyl having a single ring preferably has 3 to 16 carbon atoms, more preferably 3 to 12 carbon atoms, and even more preferably 4 to 8 carbon atoms. Examples include cyclopropyl, cyclobutyl, cyclopentyl, cyclohexyl, cycloheptyl, cyclooctyl, cyclononyl, cyclodecyl, cyclopropenyl, cyclobutenyl, cyclopentenyl, cyclohexenyl, cycloheptenyl, and cyclohexadienyl.

A non-aromatic carbocyclyl having two or more rings preferably has 8 to 20 carbon atoms, and more preferably 8 to 16 carbon atoms. Examples include indanyl, indenyl, acenaphthyl, tetrahydronaphthyl, and fluorenyl.

“Non-aromatic carbocycle” means a ring derived from the above-described “non aromatic carbocyclyl”.

When n is 1 in Formula (I) above, examples of “substituted or unsubstituted non-aromatic carbocycle formed by R^{4a} and R^{4b} taken together with a carbon atom to which they are each bonded” include the following rings.

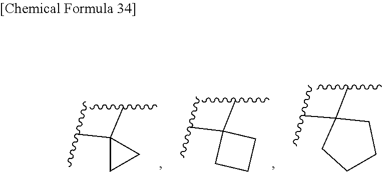 [SMILES: *C1(*)CC1 ; *C1(*)CCC1 ; *C1(*)CCCC1]

In Formula (I) above, examples of “substituted or unsubstituted non-aromatic carbocycle formed by R^{10b} and R^{10b′}, R^{10c} and R^{10c′}, R^{10d} and R^{10d′}, R^{10e} and R^{10e′}, R^{10f} and R^{10f′}, R^{10g} and R^{10g′}, R^{10h} and R^{10h′}, R¹⁰ⁱ and R^{10i′}, and R^{10j} and R^{10j′} each independently taken together with a carbon atom to which they are bonded” include the following rings:

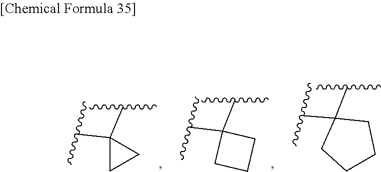 [SMILES: *C1(*)CC1 ; *C1(*)CCC1 ; *C1(*)CCCC1]

In Formula (I) above, examples of “substituted or unsubstituted non-aromatic carbocycle formed by R^{10b} and R^{10c}, R^{10c} and R^{10d}, R^{10d} and R^{10e}, R^{10f} and R^{10g}, R^{10g} and R^{10h}, R^{10h} and R¹⁰ⁱ, and R¹⁰ⁱ and R^{10j} each independently taken together with a carbon atom to which they are each bonded” include the following rings:

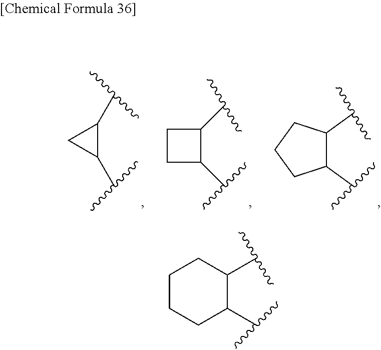 [SMILES: *C1CC1* ; *C1CCC1* ; *C1CCCC1* ; *C1CCCCC1*]

“Aromatic heterocyclyl” means an aromatic cyclyl having a single ring or two or more rings, which has one or more identical or different heteroatoms optionally selected from O, S, and N in the ring(s).

An aromatic heterocyclyl having two or more rings also includes an aromatic heterocyclyl having a single ring or two or more rings, to which a ring in the “aromatic carbocyclyl” is fused, and the linking bond may be carried by any of the rings.

The aromatic heterocyclyl having a single ring is preferably a 5- to 8-membered ring, and more preferably a 5-membered or 6-membered ring. Examples of 5-membered aromatic heterocyclyl include pyrrolyl, imidazolyl, pyrazolyl, triazolyl, tetrazolyl, furyl, thienyl, isoxazolyl, oxazolyl, oxadiazolyl, isothiazolyl, thiazolyl, and thiadiazolyl. Examples of 6-membered aromatic heterocyclyl include pyridyl, pyridazinyl, pyrimidinyl, pyrazinyl, and triazinyl.

The aromatic heterocyclyl having two rings is preferably an 8- to 10-membered ring, and more preferably a 9-membered or 10-membered ring. Examples include indolyl, isoindolyl, indazolyl, indolizinyl, quinolinyl, isoquinolinyl, cinnolinyl, phthalazinyl, quinazolinyl, naphthyridinyl, quinoxalinyl, purinyl, pteridinyl, benzimidazolyl, benzisoxazolyl, benzoxazolyl, benzoxadiazolyl, benzisothiazolyl, benzothiazolyl, benzothiadiazolyl, benzofuryl, isobenzofuryl, benzothienyl, benzotriazolyl, imidazopyridyl, triazolopyridyl, imidazothiazolyl, pyrazinopyridazinyl, oxazolopyridyl, and thiazolopyridyl. Examples of 9-membered aromatic heterocyclyl include indolyl, isoindolyl, indazolyl, indolizinyl, purinyl, benzimidazolyl, benzisoxazolyl, benzoxazolyl, benzoxadiazolyl, benzisothiazolyl, benzothiazolyl, benzothiadiazolyl, benzotriazolyl, benzofuranyl, imidazopyridyl, triazolopyridyl, oxazolopyridyl, and thiazolopyridyl. Examples of 10-membered aromatic heterocyclyl include quinolinyl, isoquinolinyl, cinnolinyl, phthalazinyl, quinazolinyl, naphthyridinyl, quinoxalinyl, pteridinyl, and pyrazinopyridazinyl.

An aromatic heterocyclyl having three or more rings is preferably a 13- to 15-membered group. Examples include carbazolyl, acridinyl, xanthenyl, phenothiazinyl, phenoxathiinyl, phenoxazinyl, and dibenzofuryl.

The term “nitrogen-containing aromatic heterocyclyl” means an aromatic heterocyclyl that is monocyclic or polycyclic having two or more rings, containing one or more nitrogen atoms in the ring(s).

“Aromatic heterocycle” means a ring derived from the above-described “aromatic heterocyclyl”.

In Formula (I) above, examples of “substituted or unsubstituted aromatic heterocycle formed by R³ and R⁸ taken together with a carbon atom to which they are each bonded”, and the “substituted or unsubstituted aromatic heterocycle formed by R^{3a} and R⁸, and R^{3b} and R⁸ taken together with a carbon atom to which they are each bonded” include the following rings:

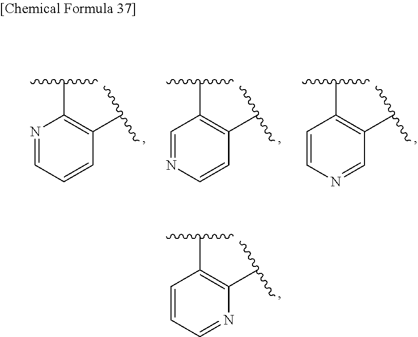 [SMILES: *c1cccnc1* ; *c1ccncc1* ; *c1ccncc1* ; *c1cccnc1*]

In Formula (I) above, examples of “substituted or unsubstituted aromatic heterocycle formed by two R^{10a}s bonded to an adjacent carbon atom” include the following rings.

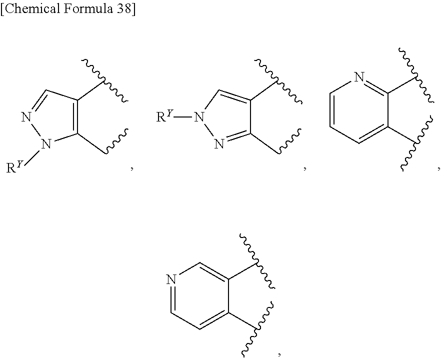

wherein R^{Y} is a hydrogen atom, substituted or unsubstituted alkyl, or the like.

“Non-aromatic heterocyclyl” means a non-aromatic cyclyl having a single ring or two or more rings, which has one or more identical or different heteroatoms optionally selected from O, S, and N in the ring(s). A non-aromatic heterocyclyl having two or more rings also includes a non-aromatic heterocyclyl having a single ring or two or more rings, to which a ring in each of the “aromatic carbocyclyl”, “non-aromatic carbocyclyl”, and/or “aromatic heterocyclyl” is fused, as well as a non-aromatic carbocyclyl having a single ring or two or more rings, to which a ring in the “aromatic heterocyclyl” is fused, and the linking bond may be carried by any of the rings.

Furthermore, the “non-aromatic heterocyclyl” also includes a bridged group or a group forming a spiro ring, such as follows.

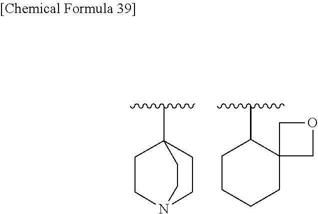

The non-aromatic heterocyclyl having a single ring is preferably a 3- to 8-membered ring, and more preferably a 5-membered or 6-membered ring.

Examples of 3-membered non-aromatic heterocyclyl include thiiranyl, oxiranyl, and aziridinyl. Examples of 4-membered non-aromatic heterocyclyl include oxetanyl and azetidinyl. Examples of 5-membered non-aromatic heterocyclyl include oxathiolanyl, thiazolidinyl, pyrrolidinyl, pyrrolinyl, imidazolidinyl, imidazolinyl, pyrazolidinyl, pyrazolinyl, tetrahydrofuryl, dihydrothiazolyl, tetrahydroisothiazolyl, dioxolanyl, dioxolyl, and thiolanyl. Examples of 6-membered non-aromatic heterocyclyl include dioxanyl, thianyl, piperidyl, piperazinyl, morpholinyl, morpholino, thiomorpholinyl, thiomorpholino, dihydropyridyl, tetrahydropyridyl, tetrahydropyranyl, dihydroxazinyl, tetrahydropyridazinyl, hexahydropyrimidinyl, dioxazinyl, thiinyl, and thiazinyl. Examples of 7-membered non-aromatic heterocyclyl include hexahydroazepinyl, tetrahydrodiazepinyl, and oxepanyl.

The non-aromatic heterocyclyl having two or more rings is preferably an 8- to 20-membered ring, more preferably an 8- to 13-membered ring, and even more preferably an 8- to 10-membered ring. Examples include indolinyl, isoindolinyl, chromanyl, and isochromanyl.

The term “nitrogen-containing non-aromatic heterocyclyl” means a non-aromatic heterocyclyl that is monocyclic or polycyclic having two or more rings, containing one or more nitrogen atoms in the ring(s). The non-aromatic nitrogen-containing heterocyclyl, which is polycyclic having two or more rings, includes a fused ring group wherein a nitrogen-containing non-aromatic heterocyclyl that is monocyclic or polycyclic having two or more rings is fused with a ring of the above “aromatic carbocyclyl”, “non-aromatic carbocyclyl” and/or “aromatic heterocyclyl”, and the linking bond may be held in any ring. A non-aromatic heterocyclyl having two or more rings also includes a non-aromatic carbocyclyl having a single ring or two or more rings, to which each ring in the “nitrogen-containing aromatic carbocyclyl” is fused, and the linking bond may be held in any ring.

Examples of the same include the following groups.

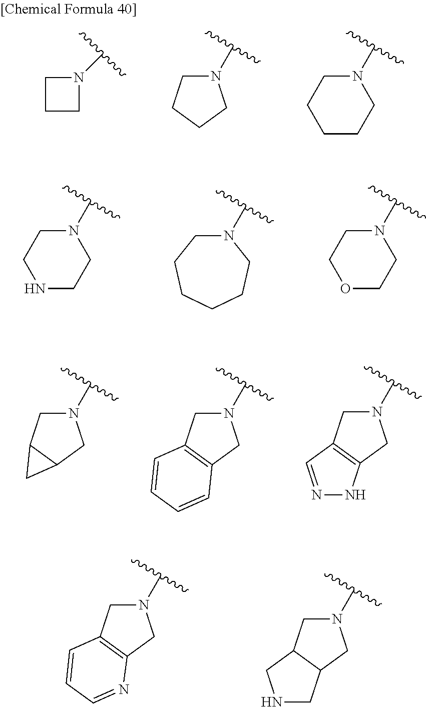

Furthermore, the “nitrogen-containing non-aromatic heterocyclyl” also includes a bridged group or a group forming a spiro ring, such as follows:

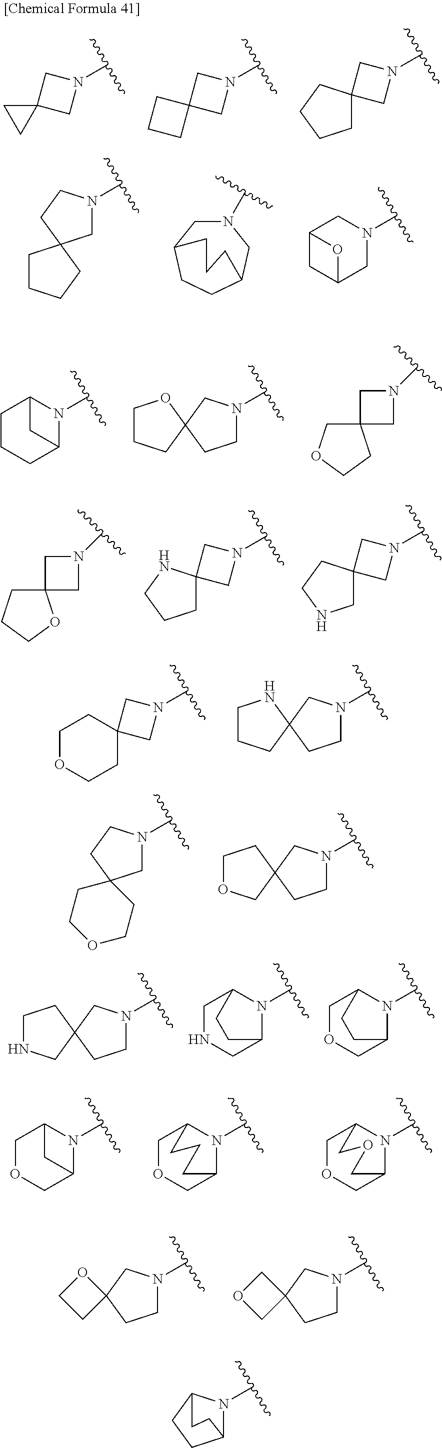

“Non-aromatic heterocycle” means a ring derived from the above-described “non-aromatic heterocyclyl”.

When n is 1 in Formula (I) above, examples of “substituted or unsubstituted non-aromatic heterocycle formed by R^{4a} and R^{4b} together with a carbon atom to which they are each bonded” include the following rings.

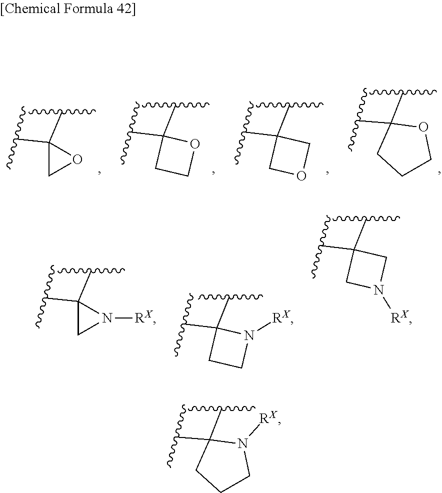

wherein R^{x} is substituted or unsubstituted alkyl, or the like.

In Formula (I) above, examples of “substituted or unsubstituted non-aromatic heterocycle formed by two R^{10a}s bonded to an adjacent carbon atom” include the following rings.

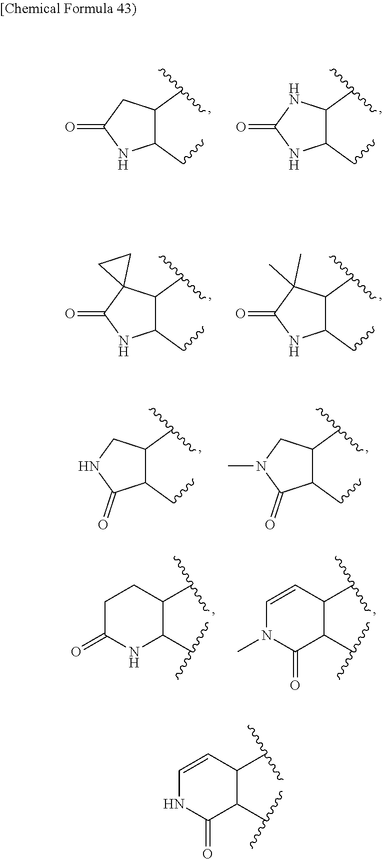

In Formula (I) above, examples of “substituted or unsubstituted non-aromatic heterocycle formed by R^{10b} and R^{10b′}, R^{10c} and R^{10c′}, R^{10d} and R^{10d′}, R^{10e} and R^{10e′}, R^{10f} and R^{10f′}, R^{10g} and R^{10g′}, R^{10h} and R^{10h′}, R¹⁰ⁱ and R^{10i′}, and R^{10j} and R^{10j′} each independently taken together with a carbon atom to which they are bonded” include the following rings:

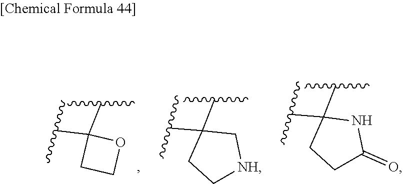 [SMILES: *C1(*)CCO1 ; *C1(*)CCNC1 ; *C1(*)CCC(=O)N1]

In Formula (I) above, examples of “substituted or unsubstituted non-aromatic heterocycle formed by R^{10b} and R^{10c}, R^{10c} and R^{10d}, R^{10d} and R^{10e}, R^{10f} and R^{10g}, R^{10g} and R^{10h}, R^{10h} and R¹⁰ⁱ, and R¹⁰ⁱ and R^{10j} each independently taken together with a carbon atom to which they are each bonded” include the following rings:

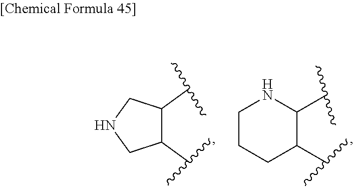 [SMILES: *C1CNCC1* ; *C1CCCNC1*]

In Formula (I) above, examples of “substituted or unsubstituted non-aromatic heterocycle formed by R^{3b} and R^{7′} taken together with an atom to which they are each bonded” include the following ring.

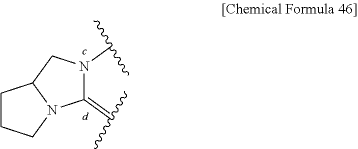 [SMILES: *N1CC2CCCN2C1=*]

wherein the nitrogen atom c represents a nitrogen atom to which R^{7′} is bonded in Formula (I) above;
the carbon atom d represents a carbon atom to which R^{3b} is bonded in Formula (I) above.

“Trialkylsilyl” means a group in which three moieties of the above-described “alkyl” are bonded to a silicon atom. The three alkyl groups may be identical or different. Examples include trimethylsilyl, triethylsilyl, and tert-butyldimethylsilyl.

“Iminosulfino” means a group represented by the following formula:

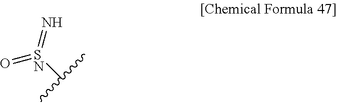

One or two hydrogen atoms at any position may be substituted. Examples of a substituent of the “substituted iminosulfino” include alkyl, or the like.

In the present specification, the phrase “may be substituted with substituent group α” means that “may be substituted with one or more group(s) selected from substituent group α”. The same also applies to substituent groups β, γ, and γ′.

Substituent for “substituted alkyl”, “substituted alkenyl”, “substituted alkynyl”, “substituted alkyloxy”, “substituted alkenyloxy”, “substituted alkynyloxy”, “substituted alkylcarbonyloxy”, “substituted alkenylcarbonyloxy”, “substituted alkynylcarbonyloxy”, “substituted alkylcarbonyl”, “substituted alkenylcarbonyl”, “substituted alkynylcarbonyl”, “substituted alkyloxycarbonyl”, “substituted alkenyloxycarbonyl”, “substituted alkynyloxycarbonyl”, “substituted alkylsulfanyl”, “substituted alkenylsulfanyl”, “substituted alkynylsulfanyl”, “substituted alkylsulfinyl”, “substituted alkenylsulfinyl”, “substituted alkynylsulfinyl”, “substituted alkylsulfonyl”, “substituted alkenylsulfonyl” “substituted alkynylsulfonyl”, and the like include the following substituent group A. A carbon atom at any position may be bonded to one or more group(s) selected from the following substituent group A.

Substituent group A: halogen, hydroxy, carboxy, formyl, formyloxy, sulfanyl, sulfino, sulfo, thioformyl, thiocarboxy, dithiocarboxy, thiocarbamoyl, cyano, nitro, nitroso, azide, hydrazino, ureido, amidino, guanidino, pentafluorothio, trialkylsilyl, alkyloxy which may be substituted with substituent group α, alkenyloxy which may be substituted with substituent group α, alkynyloxy which may be substituted with substituent group α, alkylcarbonyloxy which may be substituted with substituent group α, alkenylcarbonyloxy which may be substituted with substituent group α, alkynylcarbonyloxy which may be substituted with substituent group α, alkylcarbonyl which may be substituted with substituent group α, alkenylcarbonyl which may be substituted with substituent group α, alkynylcarbonyl which may be substituted with substituent group α, alkyloxycarbonyl which may be substituted with substituent group α, alkenyloxycarbonyl which may be substituted with substituent group α, alkynyloxycarbonyl which may be substituted with substituent group α, alkylsulfanyl which may be substituted with substituent group α, alkenylsulfanyl which may be substituted with substituent group α, alkynylsulfanyl which may be substituted with substituent group α, alkylsulfinyl which may be substituted with substituent group α, alkenylsulfinyl which may be substituted with substituent group α, alkynylsulfinyl which may be substituted with substituent group α, alkylsulfonyl which may be substituted with substituent group α, alkenylsulfonyl which may be substituted with substituent group α, alkynylsulfonyl which may be substituted with substituent group α, amino which may be substituted with substituent group β, imino which may be substituted with substituent group β, carbamoyl which may be substituted with substituent group β, sulfamoyl which may be substituted with substituent group β, aromatic carbocyclyl which may be substituted with substituent group γ, non-aromatic carbocyclyl which may be substituted with substituent group γ′, aromatic heterocyclyl which may be substituted with substituent group γ, non-aromatic heterocyclyl which may be substituted with substituent group γ′, aromatic carbocyclyloxy which may be substituted with substituent group γ, non-aromatic carbocyclyloxy which may be substituted with substituent group γ′, aromatic heterocyclyloxy which may be substituted with substituent group γ, non-aromatic heterocyclyloxy which may be substituted with substituent group γ′, aromatic carbocyclylcarbonyloxy which may be substituted with substituent group γ, non-aromatic carbocyclylcarbonyloxy which may be substituted with substituent group γ′, aromatic heterocyclylcarbonyloxy which may be substituted with substituent group γ, non-aromatic heterocyclylcarbonyloxy which may be substituted with substituent group γ′, aromatic carbocyclylcarbonyl which may be substituted with substituent group γ, non-aromatic carbocyclylcarbonyl which may be substituted with substituent group γ′, aromatic heterocyclylcarbonyl which may be substituted with substituent group γ, non-aromatic heterocyclylcarbonyl which may be substituted with substituent group γ′, aromatic carbocyclyloxycarbonyl which may be substituted with substituent group γ, non-aromatic carbocyclyloxycarbonyl which may be substituted with substituent group γ′, aromatic heterocyclyloxycarbonyl which may be substituted with substituent group γ, non-aromatic heterocyclyloxycarbonyl which may be substituted with substituent group γ′, aromatic carbocyclylalkyloxy which may be substituted with substituent group γ, non-aromatic carbocyclylalkyloxy which may be substituted with substituent group γ′, aromatic heterocyclylalkyloxy which may be substituted with substituent group γ, non-aromatic heterocyclylalkyloxy which may be substituted with substituent group γ′, aromatic carbocyclylalkyloxycarbonyl which may be substituted with substituent group γ, non-aromatic carbocyclylalkyloxycarbonyl which may be substituted with substituent group γ′, aromatic heterocyclylalkyloxycarbonyl which may be substituted with substituent group γ, non-aromatic heterocyclylalkyloxycarbonyl which may be substituted with substituent group γ′, aromatic carbocyclylsulfanyl which may be substituted with substituent group γ, non-aromatic carbocyclylsulfanyl which may be substituted with substituent group γ′, aromatic heterocyclylsulfanyl which may be substituted with substituent group γ, non-aromatic heterocyclylsulfanyl which may be substituted with substituent group γ′, aromatic carbocyclylsulfinyl which may be substituted with substituent group γ, non-aromatic carbocyclylsulfinyl which may be substituted with substituent group γ′, aromatic heterocyclylsulfinyl which may be substituted with substituent group γ, non-aromatic heterocyclylsulfinyl which may be substituted with substituent group γ′, aromatic carbocyclylsulfonyl which may be substituted with substituent group γ, non-aromatic carbocyclylsulfonyl which may be substituted with substituent group γ′, aromatic heterocyclylsulfonyl which may be substituted with substituent group γ, and non-aromatic heterocyclylsulfonyl which may be substituted with substituent group γ′.

Substituent group α: halogen, hydroxy, carboxy, alkyloxy, haloalkyloxy, alkenyloxy, alkynyloxy, sulfanyl, and cyano.

Substituent group β: halogen, hydroxy, carboxy, cyano, alkyl which may be substituted with substituent group α, alkenyl which may be substituted with substituent group α, alkynyl which may be substituted with substituent group α, alkylcarbonyl which may be substituted with substituent group α, alkenylcarbonyl which may be substituted with substituent group α, alkynylcarbonyl which may be substituted with substituent group α, alkylsulfanyl which may be substituted with substituent group α, alkenylsulfanyl which may be substituted with substituent group α, alkynylsulfanyl which may be substituted with substituent group α, alkylsulfinyl which may be substituted with substituent group α, alkenylsulfinyl which may be substituted with substituent group α, alkynylsulfinyl which may be substituted with substituent group α, alkylsulfonyl which may be substituted with substituent group α, alkenylsulfonyl which may be substituted with substituent group α, alkynylsulfonyl which may be substituted with substituent group α,
aromatic carbocyclyl which may be substituted with substituent group γ, non-aromatic carbocyclyl which may be substituted with substituent group γ′, aromatic heterocyclyl which may be substituted with substituent group γ, non-aromatic heterocyclyl which may be substituted with substituent group γ′, aromatic carbocyclylalkyl which may be substituted with substituent group γ, non-aromatic carbocyclylalkyl which may be substituted with substituent group γ′, aromatic heterocyclylalkyl which may be substituted with substituent group γ, non-aromatic heterocyclylalkyl which may be substituted with substituent group γ′, aromatic carbocyclylcarbonyl which may be substituted with substituent group γ, non-aromatic carbocyclylcarbonyl which may be substituted with substituent group γ′, aromatic heterocyclylcarbonyl which may be substituted with substituent group γ, non-aromatic heterocyclylcarbonyl which may be substituted with substituent group γ′, aromatic carbocyclyloxycarbonyl which may be substituted with substituent group γ, non-aromatic carbocyclyloxycarbonyl which may be substituted with substituent group γ′, aromatic heterocyclyloxycarbonyl which may be substituted with substituent group γ, non-aromatic heterocyclyloxycarbonyl which may be substituted with substituent group γ′, aromatic carbocyclylsulfanyl which may be substituted with substituent group γ, non-aromatic carbocyclylsulfanyl which may be substituted with substituent group γ′, aromatic heterocyclylsulfanyl which may be substituted with substituent group γ, non-aromatic heterocyclylsulfanyl which may be substituted with substituent group γ′, aromatic carbocyclylsulfinyl which may be substituted with substituent group γ, non-aromatic carbocyclylsulfinyl which may be substituted with substituent group γ′, aromatic heterocyclylsulfinyl which may be substituted with substituent group γ, non-aromatic heterocyclylsulfinyl which may be substituted with substituent group γ′, aromatic carbocyclylsulfonyl which may be substituted with substituent group γ, non-aromatic carbocyclylsulfonyl which may be substituted with substituent group γ′, aromatic heterocyclylsulfonyl which may be substituted with substituent group γ, and non-aromatic heterocyclylsulfonyl which may be substituted with substituent group γ′.

Substituent group γ: substituent group α, alkyl, haloalkyl, hydroxyalkyl, alkenyl, alkynyl, alkylcarbonyl, haloalkylcarbonyl, alkenylcarbonyl, and alkynylcarbonyl.

Substituent group γ′: substituent group γ and oxo.

The substituents on the rings of “aromatic carbocycle” and “aromatic heterocycle”, such as “substituted aromatic carbocyclyl”, “substituted aromatic heterocyclyl”, “substituted aromatic carbocyclyloxy”, “substituted aromatic heterocyclyloxy”, “substituted aromatic carbocyclylcarbonyloxy”, “substituted aromatic heterocyclylcarbonyloxy”, “substituted aromatic carbocyclylcarbonyl”, “substituted aromatic heterocyclylcarbonyl”, “substituted aromatic carbocyclyloxycarbonyl”, “substituted aromatic heterocyclyloxycarbonyl”, “substituted aromatic carbocyclylsulfanyl”, “substituted aromatic heterocyclylsulfanyl”, “substituted aromatic carbocyclylsulfinyl”, “substituted aromatic heterocyclylsulfinyl”, “substituted aromatic carbocyclylsulfonyl”, “substituted aromatic heterocyclylsulfonyl”, and “substituted nitrogen-containing aromatic heterocyclyl” include the following substituent group B. An atom at any position on the ring may be bonded to one or more group(s) selected from the following substituent group B.

Substituent group B: halogen, hydroxy, carboxy, formyl, formyloxy, sulfanyl, sulfino, sulfo, thioformyl, thiocarboxy, dithiocarboxy, thiocarbamoyl, cyano, nitro, nitroso, azide, hydrazino, ureido, amidino, guanidino, pentafluorothio, trialkylsilyl,
alkyl which may be substituted with substituent group α, alkenyl which may be substituted with substituent group α, alkynyl which may be substituted with substituent group α, alkyloxy which may be substituted with substituent group α, alkenyloxy which may be substituted with substituent group α, alkynyloxy which may be substituted with substituent group α, alkylcarbonyloxy which may be substituted with substituent group α, alkenylcarbonyloxy which may be substituted with substituent group α, alkynylcarbonyloxy which may be substituted with substituent group α, alkylcarbonyl which may be substituted with substituent group α, alkenylcarbonyl which may be substituted with substituent group α, alkynylcarbonyl which may be substituted with substituent group α, alkyloxycarbonyl which may be substituted with substituent group α, alkenyloxycarbonyl which may be substituted with substituent group α, alkynyloxycarbonyl which may be substituted with substituent group α, alkylsulfanyl which may be substituted with substituent group α, alkenylsulfanyl which may be substituted with substituent group α, alkynylsulfanyl which may be substituted with substituent group α, alkylsulfinyl which may be substituted with substituent group α, alkenylsulfinyl which may be substituted with substituent group α, alkynylsulfinyl which may be substituted with substituent group α, alkylsulfonyl which may be substituted with substituent group α, alkenylsulfonyl which may be substituted with substituent group α, alkynylsulfonyl which may be substituted with substituent group α,
amino which may be substituted with substituent group β, imino which may be substituted with substituent group β, carbamoyl which may be substituted with substituent group β, sulfamoyl which may be substituted with substituent group β,
aromatic carbocyclyl which may be substituted with substituent group γ, non-aromatic carbocyclyl which may be substituted with substituent group γ′, aromatic heterocyclyl which may be substituted with substituent group γ, non-aromatic heterocyclyl which may be substituted with substituent group γ′, aromatic carbocyclyloxy which may be substituted with substituent group γ, non-aromatic carbocyclyloxy which may be substituted with substituent group γ′, aromatic heterocyclyloxy which may be substituted with substituent group γ, non-aromatic heterocyclyloxy which may be substituted with substituent group γ′, aromatic carbocyclylcarbonyloxy which may be substituted with substituent group γ, non-aromatic carbocyclylcarbonyloxy which may be substituted with substituent group γ′, aromatic heterocyclylcarbonyloxy which may be substituted with substituent group γ, and non-aromatic heterocyclylcarbonyloxy which may be substituted with substituent group γ′, aromatic carbocyclylcarbonyl which may be substituted with substituent group γ, non-aromatic carbocyclylcarbonyl which may be substituted with substituent group γ′, aromatic heterocyclylcarbonyl which may be substituted with substituent group γ, non-aromatic heterocyclylcarbonyl which may be substituted with substituent group γ′, aromatic carbocyclyloxycarbonyl which may be substituted with substituent group γ, non-aromatic carbocyclyloxycarbonyl which may be substituted with substituent group γ′, aromatic heterocyclyloxycarbonyl which may be substituted with substituent group γ, non-aromatic heterocyclyloxycarbonyl which may be substituted with substituent group γ′, aromatic carbocyclylalkyl which may be substituted with substituent group γ, non-aromatic carbocyclylalkyl which may be substituted with substituent group γ′, aromatic heterocyclylalkyl which may be substituted with substituent group γ, non-aromatic heterocyclylalkyl which may be substituted with substituent group γ′, aromatic carbocyclylalkyloxy which may be substituted with substituent group γ, non-aromatic carbocyclylalkyloxy which may be substituted with substituent group γ′, aromatic heterocyclylalkyloxy which may be substituted with substituent group γ, non-aromatic heterocyclylalkyloxy which may be substituted with substituent group γ′, aromatic carbocyclylalkyloxycarbonyl which may be substituted with substituent group γ, non-aromatic carbocyclylalkyloxycarbonyl which may be substituted with substituent group γ′, aromatic heterocyclylalkyloxycarbonyl which may be substituted with substituent group γ, non-aromatic heterocyclylalkyloxycarbonyl which may be substituted with substituent group γ′, aromatic carbocyclylalkyloxyalkyl which may be substituted with substituent group γ, non-aromatic carbocyclylalkyloxyalkyl which may be substituted with substituent group γ′, aromatic heterocyclylalkyloxyalkyl which may be substituted with substituent group γ, non-aromatic heterocyclylalkyloxyalkyl which may be substituted with substituent group γ′, aromatic carbocyclylsulfanyl which may be substituted with substituent group γ, non-aromatic carbocyclylsulfanyl which may be substituted with substituent group γ′, aromatic heterocyclylsulfanyl which may be substituted with substituent group γ, non-aromatic heterocyclylsulfanyl which may be substituted with substituent group γ′, aromatic carbocyclylsulfinyl which may be substituted with substituent group γ, non-aromatic carbocyclylsulfinyl which may be substituted with substituent group γ′, aromatic heterocyclylsulfinyl which may be substituted with substituent group γ, non-aromatic heterocyclylsulfinyl which may be substituted with substituent group γ′, aromatic carbocyclylsulfonyl which may be substituted with substituent group γ, non-aromatic carbocyclylsulfonyl which may be substituted with substituent group γ′, aromatic heterocyclylsulfonyl which may be substituted with substituent group γ, and non-aromatic heterocyclylsulfonyl which may be substituted with substituent group γ′.

The substituents on the ring of “non-aromatic carbocycle” and “non-aromatic heterocycle” of “substituted non-aromatic carbocyclyl”, “substituted non-aromatic heterocyclyl”, “substituted non-aromatic carbocyclyloxy”, “substituted non-aromatic heterocyclyloxy”, “substituted non-aromatic carbocyclylcarbonyloxy”, “substituted non-aromatic heterocyclylcarbonyloxy”, “substituted non-aromatic carbocyclylcarbonyl”, “substituted non-aromatic heterocyclylcarbonyl”, “substituted non-aromatic carbocyclyloxycarbonyl”, “substituted non-aromatic heterocyclyloxycarbonyl”, “substituted non-aromatic carbocyclylsulfanyl”, “substituted non-aromatic heterocyclylsulfanyl”, “substituted non-aromatic carbocyclylsulfinyl”, “substituted non-aromatic heterocyclylsulfinyl”, “substituted non-aromatic carbocyclylsulfonyl”, “substituted non-aromatic heterocyclylsulfonyl”, and “substituted nitrogen-containing non-aromatic heterocyclyl” include the following substituent group C: An atom at any position on the ring may be bonded to one or more group(s) selected from the following substituent group C.

Substituent group C: substituent group B and oxo.

When the “non-aromatic carbocycle” and the “non-aromatic heterocycle” are substituted with “oxo”, this means a ring in which two hydrogen atoms on a carbon atom are substituted as follows.

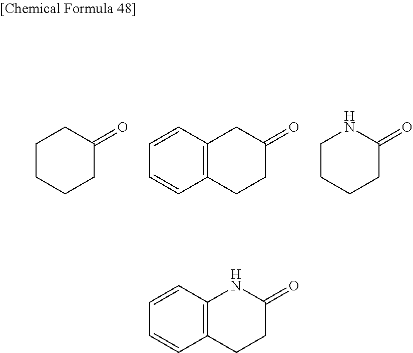

The substituents for “substituted amino”, “substituted imino”, “substituted carbamoyl”, and “substituted sulfamoyl” include the following substituent group D. These moieties may be substituted with one or two group(s) selected from substituent group D.

Substituent group D: halogen, hydroxy, carboxy, cyano, alkyl which may be substituted with substituent group α, alkenyl which may be substituted with substituent group α, alkynyl which may be substituted with substituent group α, alkylcarbonyl which may be substituted with substituent group α, alkenylcarbonyl which may be substituted with substituent group α, alkynylcarbonyl which may be substituted with substituent group α, alkylsulfanyl which may be substituted with substituent group α, alkenylsulfanyl which may be substituted with substituent group α, alkynylsulfanyl which may be substituted with substituent group α, alkylsulfinyl which may be substituted with substituent group α, alkenylsulfinyl which may be substituted with substituent group α, alkynylsulfinyl which may be substituted with substituent group α, alkylsulfonyl which may be substituted with substituent group α, alkenylsulfonyl which may be substituted with substituent group α, alkynylsulfonyl which may be substituted with substituent group α,
amino which may be substituted with substituent group β, imino which may be substituted with substituent group β, carbamoyl which may be substituted with substituent group β, sulfamoyl which may be substituted with substituent group β,
aromatic carbocyclyl which may be substituted with substituent group γ, non-aromatic carbocyclyl which may be substituted with substituent group γ′, aromatic heterocyclyl which may be substituted with substituent group γ, non-aromatic heterocyclyl which may be substituted with substituent group γ′, aromatic carbocyclylalkyl which may be substituted with substituent group γ, non-aromatic carbocyclylalkyl which may be substituted with substituent group γ′, aromatic heterocyclylalkyl which may be substituted with substituent group γ, non-aromatic heterocyclylalkyl which may be substituted with substituent group γ′, aromatic carbocyclylcarbonyl which may be substituted with substituent group γ, non-aromatic carbocyclylcarbonyl which may be substituted with substituent group γ′, aromatic heterocyclylcarbonyl which may be substituted with substituent group γ, non-aromatic heterocyclylcarbonyl which may be substituted with substituent group γ′, aromatic carbocyclyloxycarbonyl which may be substituted with substituent group γ, non-aromatic carbocyclyloxycarbonyl which may be substituted with substituent group γ′, aromatic heterocyclyloxycarbonyl which may be substituted with substituent group γ, non-aromatic heterocyclyloxycarbonyl which may be substituted with substituent group γ′, aromatic carbocyclylsulfanyl which may be substituted with substituent group γ, non-aromatic carbocyclylsulfanyl which may be substituted with substituent group γ′, aromatic heterocyclylsulfanyl which may be substituted with substituent group γ, non-aromatic heterocyclylsulfanyl which may be substituted with substituent group γ′, aromatic carbocyclylsulfinyl which may be substituted with substituent group γ, non-aromatic carbocyclylsulfinyl which may be substituted with substituent group γ′, aromatic heterocyclylsulfinyl which may be substituted with substituent group γ, non-aromatic heterocyclylsulfinyl which may be substituted with substituent group γ′, aromatic carbocyclylsulfonyl which may be substituted with substituent group γ, non-aromatic carbocyclylsulfonyl which may be substituted with substituent group γ′, aromatic heterocyclylsulfonyl which may be substituted with substituent group γ, and non-aromatic heterocyclylsulfonyl which may be substituted with substituent group γ′.

Examples of substituents for the “substituted or unsubstituted non-aromatic heterocyclyl” and the “substituted or unsubstituted nitrogen-containing non-aromatic heterocyclyl” in R¹ include:
oxo; thioxo; halogen; cyano; nitro; carboxy;
substituted or unsubstituted carbamoyl;
substituted or unsubstituted alkyl;
substituted or unsubstituted alkyloxy;
substituted or unsubstituted alkylcarbonyl;
substituted or unsubstituted alkyloxycarbonyl;
substituted or unsubstituted alkylsulfanyl;
substituted or unsubstituted amino;
substituted or unsubstituted aromatic carbocyclyl;
substituted or unsubstituted aromatic heterocyclyl;
substituted or unsubstituted non-aromatic carbocyclyl;
substituted or unsubstituted non-aromatic heterocyclyl;
substituted or unsubstituted non-aromatic heterocyclylcarbonyl.

It may be substituted with one or more group(s) selected from these.

Examples of substituents for the “substituted or unsubstituted non-aromatic heterocyclyl” and the “substituted or unsubstituted nitrogen-containing non-aromatic heterocyclyl” in R¹ include:
oxo; halogen.

It may be substituted with one or more group(s) selected from these.

Examples of substituents for the “substituted or unsubstituted aromatic heterocyclyl” and the “substituted or unsubstituted nitrogen-containing aromatic heterocyclyl” in R¹ include:
halogen; cyano; hydroxy;
substituted or unsubstituted alkyl;
substituted or unsubstituted alkyloxy;
substituted or unsubstituted alkyloxycarbonyl;
substituted or unsubstituted amino;
substituted or unsubstituted aromatic carbocyclyl. It may be substituted with one or more group(s) selected from these.

Examples of substituents for the “substituted or unsubstituted aromatic heterocyclyl” and the “substituted or unsubstituted nitrogen-containing aromatic heterocyclyl” in R¹ include:
halogen; cyano; hydroxy;
substituted or unsubstituted alkyl;
substituted or unsubstituted alkyloxy. It may be substituted with one or more group(s) selected from these.

Examples of substituents for the “substituted or unsubstituted aromatic heterocyclyl” and the “substituted or unsubstituted nitrogen-containing aromatic heterocyclyl” in R¹ include:
halogen; cyano; hydroxy;
substituted alkyl (examples of the substituent include halogen; optionally substituted with one or more group(s) selected from these); unsubstituted alkyl;
unsubstituted alkyloxy. It may be substituted with one or more group(s) selected from these.

Examples of substituents for the “substituted or unsubstituted aromatic heterocyclyl” and the “substituted or unsubstituted nitrogen-containing aromatic heterocyclyl” in R¹ include:
halogen; cyano;
substituted alkyl (examples of the substituent include halogen; optionally substituted with one or more group(s) selected from these); unsubstituted alkyl; substituted alkyloxy (examples of the substituent include halogen; optionally substituted with one or more group(s) selected from these); unsubstituted alkyloxy. It may be substituted with one or more group(s) selected from these.

Examples of the substituent for the “substituted or unsubstituted carbamoyl” in R¹ include:
substituted or unsubstituted alkyl;
substituted or unsubstituted non-aromatic carbocyclyl. It may be substituted with one or more group(s) selected from these.

Examples of the substituent for the “substituted or unsubstituted carbamoyl” in R¹ include
substituted or unsubstituted alkyl. It may be substituted with one or more group(s) selected from these.

Examples of the substituent for the “substituted or unsubstituted carbamoyl” in R¹ include
substituted alkyl (examples of the substituent include halogen, non-aromatic carbocyclyl; optionally substituted with one or more group(s) selected from these); unsubstituted alkyl;
unsubstituted non-aromatic carbocyclyl. It may be substituted with one or more group(s) selected from these.

Examples of the substituent for the “substituted or unsubstituted carbamoyl” in R¹ include
alkyl. It may be substituted with one or more group(s) selected from these.

Examples of the substituent for the “substituted or unsubstituted aromatic carbocyclyl” in R² include:
halogen; cyano;
substituted or unsubstituted alkyl;
substituted or unsubstituted alkynyl;
substituted or unsubstituted alkyloxy.

It may be substituted with one or more group(s) selected from these.

Examples of the substituent for the “substituted or unsubstituted 6-membered aromatic carbocyclyl” in R² include:
halogen; cyano;
substituted or unsubstituted alkyl;
substituted or unsubstituted alkyloxy;
substituted or unsubstituted alkyloxycarbonyl;

It may be substituted with one or more group(s) selected from these.

Examples of the substituent for the “substituted or unsubstituted 6-membered aromatic carbocyclyl” in R² include:
halogen; cyano;
substituted alkyl (examples of the substituent include halogen, alkyloxy; optionally substituted with one or more group(s) selected from these); unsubstituted alkyl;
substituted alkyloxy (examples of the substituent include aromatic carbocyclyl; optionally substituted with one or more group(s) selected from these); unsubstituted alkyloxy;
unsubstituted alkyloxycarbonyl.

It may be substituted with one or more group(s) selected from these.

Examples of the substituent for the “substituted or unsubstituted aromatic heterocyclyl” in R² include:
halogen; cyano;
substituted or unsubstituted alkyl;
substituted or unsubstituted alkynyl;
substituted or unsubstituted alkyloxy;

It may be substituted with one or more group(s) selected from these.

Examples of the substituent for the “substituted or unsubstituted 6-membered aromatic heterocyclyl” in R² include:
halogen.

It may be substituted with one or more group(s) selected from these.

Examples of the substituent for the “substituted or unsubstituted aromatic carbocyclyl” in R³, R^{3a}, R^{3b}, and R^{3b′} include:
halogen; cyano; carboxy;
substituted or unsubstituted alkyl;
substituted or unsubstituted alkenyl;
substituted or unsubstituted alkynyl;
substituted or unsubstituted alkyloxy;
substituted or unsubstituted alkylcarbonyl;
substituted or unsubstituted alkylsulfonyl;
substituted or unsubstituted carbamoyl;
substituted or unsubstituted sulfamoyl;
substituted or unsubstituted amino;
substituted or unsubstituted imino;
substituted or unsubstituted iminosulfino;
substituted or unsubstituted aromatic carbocyclyl;
substituted or unsubstituted aromatic heterocyclyl;
substituted or unsubstituted non-aromatic heterocyclyl.

It may be substituted with one or more group(s) selected from these.

Examples of the substituent for the “substituted or unsubstituted aromatic carbocyclyl” in R³, R^{3a}, R^{3b}, and R^{3b′} include:
halogen; cyano;
substituted or unsubstituted alkyl;
substituted or unsubstituted carbamoyl;
substituted or unsubstituted imino;
substituted or unsubstituted iminosulfino.

It may be substituted with one or more group(s) selected from these.

Examples of the substituent for the “substituted or unsubstituted aromatic carbocyclyl” in R³, R^{3a}, R^{3b}, and R^{3b′} include:
halogen; cyano; hydroxy; carboxy;
substituted or unsubstituted alkyl;
substituted or unsubstituted alkyloxy;
substituted or unsubstituted alkylcarbonyl;
substituted or unsubstituted alkyloxycarbonyl;
substituted or unsubstituted alkylsulfinyl;
substituted or unsubstituted alkylsulfonyl;
substituted or unsubstituted sulfoxyimino;
substituted or unsubstituted iminosulfino;
substituted or unsubstituted amino;
substituted or unsubstituted carbamoyl;
substituted or unsubstituted N-(bis(dialkylamino)methylene)carbamoyl; substituted or unsubstituted sulfamoyl;
substituted or unsubstituted dialkylphosphoryl;
substituted or unsubstituted non-aromatic carbocyclyl;
substituted or unsubstituted aromatic heterocyclyl;
substituted or unsubstituted non-aromatic heterocyclyl;
substituted or unsubstituted non-aromatic heterocyclylcarbonyl.

It may be substituted with one or more group(s) selected from these.

Examples of the substituent for the “substituted or unsubstituted aromatic carbocyclyl” in R³, R^{3a}, R^{3b}, and R^{3b′} include:
halogen; cyano; hydroxy; carboxy;
substituted alkyl (examples of the substituent include halogen, hydroxy, carboxy, carbamoyl, dialkylcarbamoyl, alkylsulfonylalkylcarbamoyl, dialkylaminoalkylcarbamoyl, alkyl non-aromatic heterocyclylcarbamoyl, alkyloxy, alkyloxycarbonyl, dialkylsulfoximinyl, non-aromatic heterocyclyl, alkyl non-aromatic heterocyclyl, amino non-aromatic heterocyclylcarbonyl; optionally substituted with one or more group(s) selected from these); unsubstituted alkyl;
substituted alkyloxy (examples of the substituent include alkyloxy; optionally substituted with one or more group(s) selected from these); unsubstituted alkyloxy;
unsubstituted alkylcarbonyl;
unsubstituted alkyloxycarbonyl;
substituted alkylsulfinyl (examples of the substituent include imino, alkylimino; optionally substituted with one or more group(s) selected from these);
unsubstituted alkylsulfonyl;
substituted sulfoxyimino (examples of the substituent include alkyl; optionally substituted with one or more group(s) selected from these);
substituted iminosulfino (examples of the substituent include alkyl; optionally substituted with one or more group(s) selected from these);
substituted amino (examples of the substituent include alkylcarbonyl, alkylsulfonyl; optionally substituted with one or more group(s) selected from these); unsubstituted amino;
substituted carbamoyl (examples of the substituent include alkyl, hydroxyalkyl, dialkylaminoalkyl, hydroxyalkylaminoalkyl, non-aromatic heterocyclylalkyl, alkyloxy, hydroxyalkyloxy, alkyl non-aromatic heterocyclyl, alkyloxy aromatic heterocyclyl amino non-aromatic carbocyclyl; optionally substituted with one or more group(s) selected from these); unsubstituted carbamoyl;
unsubstituted N-(bis(dialkylamino)methylene)carbamoyl;
substituted sulfamoyl (examples of the substituent include alkyl; optionally substituted with one or more group(s) selected from these); unsubstituted sulfamoyl;
unsubstituted dialkylphosphoryl;
substituted non-aromatic carbocyclyl (examples of the substituent include hydroxy; optionally substituted with one or more group(s) selected from these).
substituted aromatic heterocyclyl (examples of the substituent include amino; alkyl; optionally substituted with one or more group(s) selected from these); unsubstituted aromatic heterocyclyl;
unsubstituted non-aromatic heterocyclyl;
substituted non-aromatic heterocyclylcarbonyl (examples of the substituent include alkyl, alkyloxyalkyl, dialkylcarbamoyl; optionally substituted with one or more group(s) selected from these); unsubstituted non-aromatic heterocyclylcarbonyl.

It may be substituted with one or more group(s) selected from these.

Examples of the substituent for the “substituted or unsubstituted aromatic carbocyclyl” in R³, R^{3a}, R^{3b}, and R^{3b′} include:
halogen, cyano, substituted or unsubstituted alkyl (examples of the substituent include halogen, cyano, hydroxy, carboxy, carbamoyl, dialkylcarbamoyl, alkylsulfonylalkylcarbamoyl, dialkylaminoalkylcarbamoyl, (alkyl non-aromatic heterocyclyl)carbamoyl, alkyloxy, dialkylsulfoxyimino, amino non-aromatic heterocyclylcarbonyl; optionally substituted with one or more group(s) selected from these), unsubstituted alkyloxy, unsubstituted alkylcarbonyl, unsubstituted alkylsulfonyl, substituted sulfoxyimino (examples of the substituent include alkyl; optionally substituted with one or more group(s) selected from these), substituted iminosulfino (examples of the substituent include alkyl; optionally substituted with one or more group(s) selected from these), substituted or unsubstituted amino (examples of the substituent include alkylcarbonyl, alkylsulfonyl; optionally substituted with one or more group(s) selected from these), substituted or unsubstituted carbamoyl (examples of the substituent include alkyl, hydroxyalkyl, dialkylaminoalkyl, hydroxyalkylaminoalkyl, non-aromatic heterocyclylalkyl, alkyloxy, alkyloxyalkyl, hydroxyalkyloxyalkyl, alkyl non-aromatic heterocyclyl, ((alkyloxy aromatic heterocyclyl)amino) non-aromatic carbocyclyl; optionally substituted with one or more group(s) selected from these), substituted or unsubstituted sulfamoyl (examples of the substituent include alkyl; optionally substituted with one or more group(s) selected from these), unsubstituted dialkylphosphoryl, substituted or unsubstituted non-aromatic carbocyclyl (examples of the substituent include hydroxy; optionally substituted with one or more group(s) selected from these), substituted or unsubstituted aromatic heterocyclyl (examples of the substituent include amino, alkyl; optionally substituted with one or more group(s) selected from these), unsubstituted non-aromatic heterocyclyl, and substituted or unsubstituted non-aromatic heterocyclylcarbonyl (examples of the substituent include alkyl; optionally substituted with one or more group(s) selected from these).

It may be substituted with one or more group(s) selected from these.

Examples of the substituent for the “substituted or unsubstituted aromatic heterocyclyl” in R³, R^{3a}, R^{3b}, and R^{3b′} include:
halogen; cyano; carboxy; hydroxy;
substituted or unsubstituted alkyl;
substituted or unsubstituted alkenyl;
substituted or unsubstituted alkynyl;
substituted or unsubstituted alkyloxy;
substituted or unsubstituted alkylcarbonyl;
substituted or unsubstituted alkylsulfonyl;
substituted or unsubstituted carbamoyl;
substituted or unsubstituted sulfamoyl;
substituted or unsubstituted amino;
substituted or unsubstituted imino;
substituted or unsubstituted iminosulfino;
substituted or unsubstituted aromatic carbocyclyl;
substituted or unsubstituted aromatic heterocyclyl;
substituted or unsubstituted non-aromatic heterocyclyl.

It may be substituted with one or more group(s) selected from these.

Examples of the substituent for the “substituted or unsubstituted aromatic heterocyclyl” in R³ include:
halogen; cyano; hydroxy;
substituted or unsubstituted alkyl;
substituted or unsubstituted carbamoyl;
substituted or unsubstituted imino;
substituted or unsubstituted iminosulfino.

It may be substituted with one or more group(s) selected from these.

Examples of the substituent for the “substituted or unsubstituted aromatic heterocyclyl” in R³, R^{3a}, R^{3b}, and R^{3b′} include:
halogen; cyano; hydroxy;
substituted or unsubstituted alkyl;
substituted or unsubstituted alkyloxy;
substituted or unsubstituted amino;
substituted or unsubstituted carbamoyl.

It may be substituted with one or more group(s) selected from these.

Examples of the substituent for the “substituted or unsubstituted aromatic heterocyclyl” in R³, R^{3a}, R^{3b}, and R^{3b′} include:
halogen; cyano; hydroxy;
substituted alkyl (examples of the substituent include halogen, hydroxy; optionally substituted with one or more group(s) selected from these); unsubstituted alkyl;
substituted alkyloxy (examples of the substituent include halogen; optionally substituted with one or more group(s) selected from these); unsubstituted alkyloxy; substituted amino (examples of the substituent include alkyl, carbamoyl, alkylcarbonyl, alkylsulfinyl, alkylsulfonyl; optionally substituted with one or more group(s) selected from these); unsubstituted amino;
substituted carbamoyl (examples of the substituent include alkyl, unsubstituted non-aromatic heterocyclylalkyl, unsubstituted non-aromatic heterocyclyl; optionally substituted with one or more group(s) selected from these); and unsubstituted carbamoyl.

It may be substituted with one or more group(s) selected from these.

Examples of the substituent for the “substituted or unsubstituted non-aromatic heterocyclyl” in R³, R^{3a}, R^{3b}, and R^{3b′} include:
halogen; carboxy; hydroxy; oxo;
substituted or unsubstituted alkyl;
substituted or unsubstituted alkyloxycarbonyl;
substituted or unsubstituted alkenyl;
substituted or unsubstituted alkynyl;
substituted or unsubstituted alkyloxy;
substituted or unsubstituted amino;
substituted or unsubstituted carbamoyl;
substituted or unsubstituted aromatic carbocyclyl;
substituted or unsubstituted aromatic heterocyclyl.

It may be substituted with one or more group(s) selected from these.

Examples of the substituent for the “substituted or unsubstituted non-aromatic heterocyclyl” in R³, R^{3a}, R^{3b}, and R^{3b′} include:
halogen; hydroxy;
substituted or unsubstituted alkyl;
substituted alkyloxy;
substituted or unsubstituted amino;
substituted or unsubstituted carbamoyl.

It may be substituted with one or more group(s) selected from these.

Examples of the substituent for the “substituted or unsubstituted non-aromatic heterocyclyl” in R³, R^{3a}, R^{3b}, and R^{3b′} include:
oxo;
halogen; cyano; hydroxy; carboxy;
substituted or unsubstituted alkyl;
substituted or unsubstituted alkyloxy;
substituted or unsubstituted alkyloxycarbonyl;
substituted or unsubstituted amino;
substituted or unsubstituted carbamoyl;
substituted or unsubstituted alkylsulfonyl;
substituted or unsubstituted aromatic heterocyclyl;
substituted or unsubstituted non-aromatic heterocyclylcarbonyl.

It may be substituted with one or more group(s) selected from these.

Examples of the substituent for the “substituted or unsubstituted non-aromatic heterocyclyl” in R³, R^{3a}, R^{3b}, and R^{3b′} include:
oxo;
halogen; cyano; hydroxy; carboxy;
substituted alkyl (examples of the substituent include halogen, hydroxy, carboxy, alkyloxy, amino, alkylamino, unsubstituted non-aromatic heterocyclyl; optionally substituted with one or more group(s) selected from these); unsubstituted alkyl;
substituted alkyloxy (examples of the substituent include halogen; optionally substituted with one or more group(s) selected from these); unsubstituted alkyloxy; unsubstituted alkyloxycarbonyl;
substituted amino (examples of the substituent include alkyl, alkylcarbonyl, alkyloxycarbonyl; optionally substituted with one or more group(s) selected from these); unsubstituted amino;
substituted carbamoyl (examples of the substituent include alkyl; optionally substituted with one or more group(s) selected from these); unsubstituted carbamoyl;
unsubstituted alkylsulfonyl;
unsubstituted aromatic heterocyclyl;
unsubstituted non-aromatic heterocyclylcarbonyl.

It may be substituted with one or more group(s) selected from these.

Examples of the substituent for the “substituted or unsubstituted non-aromatic heterocyclyl” in R³, R^{3a}, R^{3b}, and R^{3b′} include:
a hydrogen atom, halogen, cyano, hydroxy, carboxy, substituted or unsubstituted alkyl (examples of the substituent include halogen, hydroxy, alkyloxy; optionally substituted with one or more group(s) selected from these), substituted or unsubstituted alkyloxy (examples of the substituent include halogen; optionally substituted with one or more group(s) selected from these), unsubstituted alkyloxycarbonyl, substituted or unsubstituted amino (examples of the substituent include alkyl, alkylcarbonyl; optionally substituted with one or more group(s) selected from these), substituted or unsubstituted carbamoyl (examples of the substituent include alkyl; optionally substituted with one or more group(s) selected from these), unsubstituted alkylsulfonyl, unsubstituted aromatic heterocyclyl, and unsubstituted non-aromatic heterocyclylcarbonyl. It may be substituted with one or more group(s) selected from these.

Examples of the substituent for the “substituted or unsubstituted amino” in R³ and the “substituted amino” in R^{3a}, R^{3b}, and R^{3b′} include:
substituted or unsubstituted alkyl;
substituted or unsubstituted alkylcarbonyl;
substituted or unsubstituted alkylsulfonyl;
substituted or unsubstituted aromatic carbocyclyl;
substituted or unsubstituted non-aromatic carbocyclyl;
substituted or unsubstituted non-aromatic heterocyclyl.

It may be substituted with one or more group(s) selected from these.

Examples of the substituent for the “substituted or unsubstituted amino” in R³ and the “substituted amino” in R^{3a}, R^{3b}, and R^{3b′} include:
substituted alkyl (examples of the substituent include halogen, hydroxy, alkyloxy, non-aromatic carbocyclyl; optionally substituted with one or more group(s) selected from these); unsubstituted alkyl;
substituted alkylcarbonyl (examples of the substituent include halogen, carbamoyl; optionally substituted with one or more group(s) selected from these); unsubstituted alkylcarbonyl;
unsubstituted alkylsulfonyl;
substituted aromatic carbocyclyl (examples of the substituent include haloalkyl, alkyloxy; optionally substituted with one or more group(s) selected from these); unsubstituted aromatic carbocyclyl;
substituted non-aromatic carbocyclyl (examples of the substituent include halogen, hydroxy, alkyloxycarbonyl; optionally substituted with one or more group(s) selected from these); unsubstituted non-aromatic carbocyclyl;
substituted non-aromatic heterocyclyl (examples of the substituent include halogen, alkyl; optionally substituted with one or more group(s) selected from these).

It may be substituted with one or more group(s) selected from these.

Examples of the substituent for the “substituted or unsubstituted amino” in R³ and the “substituted amino” in R^{3a}, R^{3b}, and R^{3b′} include:
substituted or unsubstituted alkyl; substituted or unsubstituted non-aromatic carbocyclyl.

It may be substituted with one or more group(s) selected from these.

Examples of the substituent for the “substituted or unsubstituted amino” in R³ and the “substituted amino” in R^{3a}, R^{3b}, and R^{3b′} include:
substituted alkyl (examples of the substituent include halogen, alkyloxy; optionally substituted with one or more group(s) selected from these); unsubstituted alkyl;
unsubstituted non-aromatic carbocyclyl.

It may be substituted with one or more group(s) selected from these.

Examples of the substituent for the “substituted or unsubstituted alkyloxy” in R³, R^{3a}, R^{3b}, and R^{3b′} include:
halogen; hydroxy; sulfothio;
substituted or unsubstituted non-aromatic carbocycle;
substituted or unsubstituted non-aromatic heterocyclyl.

It may be substituted with one or more group(s) selected from these.

Examples of the substituent for the “substituted or unsubstituted alkyloxy” in R³, R^{3a}, R^{3b}, and R^{3b′} include:
halogen; hydroxy; sulfothio;
substituted non-aromatic carbocycle (examples of the substituent include halogen; optionally substituted with one or more group(s) selected from these); unsubstituted non-aromatic heterocyclyl.

It may be substituted with one or more group(s) selected from these.

Examples of the substituent for the “substituted or unsubstituted carbamoyl” and the “substituted or unsubstituted sulfamoyl” in R³, R^{3a}, R^{3b}, and R^{3b′} include substituted or unsubstituted alkyl.

It may be substituted with one or more group(s) selected from these.

Examples of the substituent for the “substituted or unsubstituted carbamoyl” and the “substituted or unsubstituted sulfamoyl” in R³, R^{3a}, R^{3b}, and R^{3b′} include unsubstituted alkyl.

It may be substituted with one or more group(s) selected from these.

Examples of the substituent for the “substituted or unsubstituted non-aromatic carbocyclyloxy” in R³, R^{3a}, R^{3b}, and R^{3b′} include:
halogen.

It may be substituted with one or more group(s) selected from these.

Examples of the substituent for the “substituted or unsubstituted alkyl” in R³, R^{3b}, and R^{3b′} include:
substituted or unsubstituted aromatic carbocyclyl.

It may be substituted with one or more group(s) selected from these.

Examples of the substituent for the “substituted or unsubstituted alkyl” in R³, R^{3b}, and R^{3b′} include:
substituted or unsubstituted alkyloxy; substituted or unsubstituted aromatic carbocyclyl.

It may be substituted with one or more group(s) selected from these.

Examples of the substituent for the “substituted or unsubstituted alkyl” in R³, R^{3b}, and R^{3b′} include:
unsubstituted aromatic carbocyclyl.

It may be substituted with one or more group(s) selected from these.

Examples of the substituent for the “substituted or unsubstituted alkyl” in R³, R^{3b}, and R^{3b′} include:
unsubstituted alkyloxy; unsubstituted aromatic carbocyclyl.

It may be substituted with one or more group(s) selected from these.

Examples of the substituent for the “substituted or unsubstituted alkyloxy” in R⁶ include:
halogen.

It may be substituted with one or more group(s) selected from these.

Examples of the substituent for the “substituted or unsubstituted alkyl”, the “substituted or unsubstituted alkenyl”, and “substituted or unsubstituted alkynyl” in R⁷ include:
halogen; hydroxy.

It may be substituted with one or more group(s) selected from these.

Examples of the substituent for the “substituted or unsubstituted alkyloxy” in R⁷ include:
halogen; cyano;
substituted or unsubstituted alkyloxy;
substituted or unsubstituted aromatic carbocyclyl.

It may be substituted with one or more group(s) selected from these.

Examples of the substituent for the “substituted or unsubstituted alkyloxy” in R⁷ include:
halogen; cyano;
unsubstituted alkyloxy;
unsubstituted aromatic carbocyclyl.

It may be substituted with one or more group(s) selected from these.

Examples of the substituent for the “substituted or unsubstituted alkylsulfoxy” in R⁷ include
halogen.

It may be substituted with one or more group(s) selected from these.

Examples of the substituent for the “substituted or unsubstituted carbamoyl” in R⁷ include
substituted or unsubstituted alkyl.

It may be substituted with one or more group(s) selected from these.

Examples of the substituent for the “substituted or unsubstituted carbamoyl” in R⁷ include
unsubstituted alkyl.

It may be substituted with one or more group(s) selected from these.

With regard to a compound represented by Formula (I):

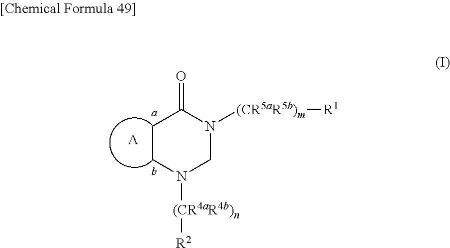

wherein ring A is a ring represented by

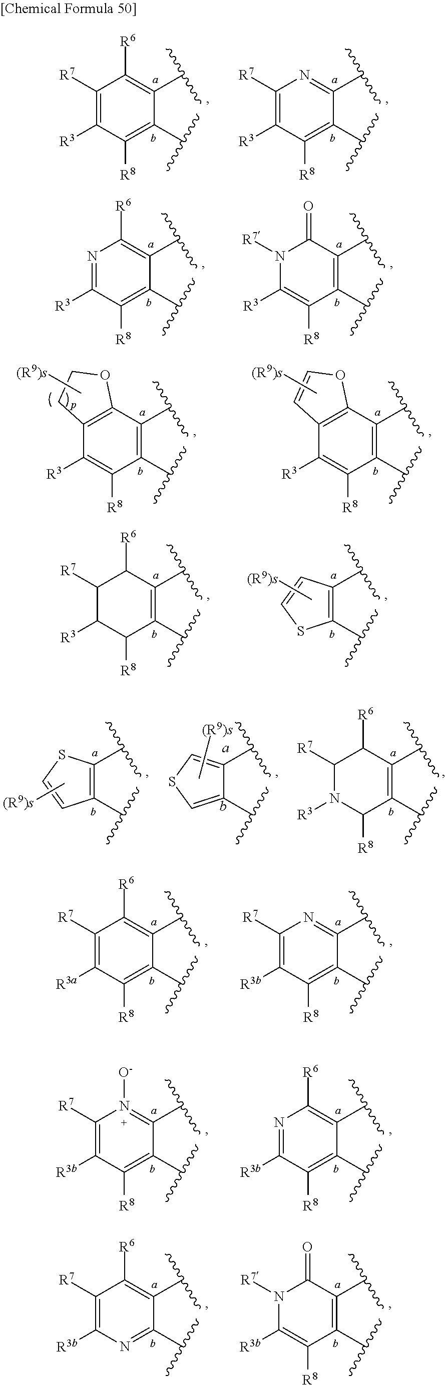

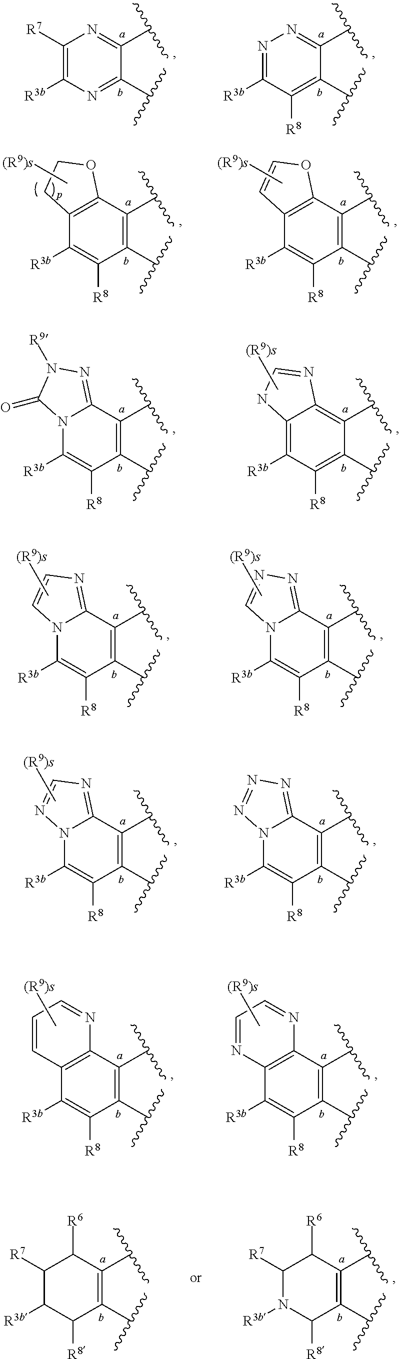

preferred embodiments of R¹, R², R³, R^{4a}, R^{4b}, R^{5a}, R^{5b}, R⁶, R⁷, R⁸, R⁹, m, n, p, s, R^{3a}, R^{3b}, R^{3b′}, R^{7′}, R^{8′}, R^{9′} are shown below. Regarding the compound represented by Formula (I), embodiments of all the combinations of specific examples shown below are mentioned as examples.

R¹ may be substituted or unsubstituted aromatic heterocyclyl, substituted or unsubstituted non-aromatic heterocyclyl, substituted or unsubstituted carbamoyl, or substituted or unsubstituted amino (hereinafter, referred to as A-1).

R¹ may be substituted or unsubstituted nitrogen-containing aromatic heterocyclyl, substituted or unsubstituted nitrogen-containing non-aromatic heterocyclyl, or substituted or unsubstituted carbamoyl (hereinafter, referred to as A-6).

R¹ may be substituted or unsubstituted aromatic heterocyclyl, substituted or unsubstituted non-aromatic heterocyclyl, or substituted or unsubstituted amino (hereinafter, referred to as A-2).

R¹ may be substituted or unsubstituted aromatic heterocyclyl, or substituted or unsubstituted non-aromatic heterocyclyl (hereinafter, referred to as A-3).

R¹ may be substituted or unsubstituted aromatic heterocyclyl (hereinafter, referred to as A-4).

R¹ may be substituted or unsubstituted non-aromatic heterocyclyl (hereinafter, referred to as A-5).

R¹ may be substituted or unsubstituted nitrogen-containing aromatic heterocyclyl, or substituted or unsubstituted nitrogen-containing non-aromatic heterocyclyl (hereinafter, referred to as A-7).

R¹ may be substituted or unsubstituted nitrogen-containing aromatic heterocyclyl (hereinafter, referred to as A-8.

R¹ may be substituted or unsubstituted nitrogen-containing non-aromatic heterocyclyl (hereinafter, referred to as A-9).

R¹ may be substituted or unsubstituted 5- to 9-membered nitrogen-containing aromatic heterocyclyl, or substituted or unsubstituted 6-membered nitrogen-containing non-aromatic heterocyclyl (hereinafter, referred to as A-10).

R¹ may be substituted or unsubstituted 5- to 9-membered nitrogen-containing aromatic heterocyclyl (hereinafter, referred to as A-11).

R¹ may be substituted or unsubstituted 6-membered nitrogen-containing non-aromatic heterocyclyl (hereinafter, referred to as A-12).

R¹ may be substituted or unsubstituted triazolyl, substituted or unsubstituted pyridyl, substituted or unsubstituted dihydropyridyl, or substituted or unsubstituted imidazopyridyl (hereinafter, may be referred to as A-12).

R² may be substituted or unsubstituted aromatic carbocyclyl, substituted or unsubstituted non-aromatic carbocyclyl, substituted or unsubstituted aromatic heterocyclyl, substituted or unsubstituted non-aromatic heterocyclyl, or substituted or unsubstituted alkyl (hereinafter, referred to as B-1).

R² may be substituted or unsubstituted aromatic carbocyclyl, or substituted or unsubstituted aromatic heterocyclyl (hereinafter, referred to as B-2).

R² may be substituted or unsubstituted aromatic carbocyclyl (hereinafter, referred to as B-3).

R² may be substituted or unsubstituted aromatic heterocyclyl (hereinafter, referred to as B-4).

R² may be substituted or unsubstituted 6-membered aromatic carbocyclyl, or substituted or unsubstituted 6-membered aromatic heterocyclyl (hereinafter, referred to as B-7).

R² may be substituted or unsubstituted 6-membered aromatic carbocyclyl (hereinafter, referred to as B-8).

R² may be substituted or unsubstituted 6-membered aromatic heterocyclyl (hereinafter, referred to as B-9).

R² may be 6-membered aromatic carbocyclyl substituted with one, two, three, four, or five substituents selected from substituent group G (substituent group G: halogen, cyano, alkyl, alkenyl, alkynyl, haloalkyl, alkyloxy, alkenyloxy, alkynyloxy, and haloalkyloxy), or 6-membered aromatic heterocyclyl substituted with one or two substituents selected from the substituent group G (hereinafter, referred to as B-10).

R² may be 6-membered aromatic carbocyclyl which is substituted with one halogen and is further substituted with one, two, three, or four substituents selected from substituent group G (substituent group G: halogen, cyano, alkyl, alkenyl, alkynyl, haloalkyl, alkyloxy, alkenyloxy, alkynyloxy, and haloalkyloxy), or 6-membered aromatic heterocyclyl which is substituted with one halogen and is further substituted with one or two substituents selected from the substituent group G (hereinafter, referred to as B-5).

R² may be 6-membered aromatic carbocyclyl which is substituted with one halogen and is further substituted with one, two, three, or four substituents selected from substituent group G (substituent group G: halogen, cyano, alkyl, alkenyl, alkynyl, haloalkyl, alkyloxy, alkenyloxy, alkynyloxy, and haloalkyloxy) (hereinafter, referred to as B-6).

R³ may be a hydrogen atom, substituted or unsubstituted aromatic carbocyclyl, substituted or unsubstituted aromatic heterocyclyl, substituted or unsubstituted non-aromatic heterocyclyl, substituted or unsubstituted alkyloxy, or substituted or unsubstituted amino (hereinafter, referred to as C-1).

R³ may be substituted or unsubstituted aromatic carbocyclyl, substituted or unsubstituted aromatic heterocyclyl, substituted or unsubstituted non-aromatic heterocyclyl, substituted or unsubstituted alkyloxy, or substituted or unsubstituted amino (hereinafter, referred to as C-2).

R³ may be substituted or unsubstituted aromatic carbocyclyl, or substituted or unsubstituted non-aromatic heterocyclyl (hereinafter, referred to as C-3).

R³ may be substituted or unsubstituted aromatic carbocyclyl (hereinafter, referred to as C-4).

R³ may be substituted or unsubstituted non-aromatic heterocyclyl (hereinafter, referred to as C-5).

R³ may be a group represented by Formula:

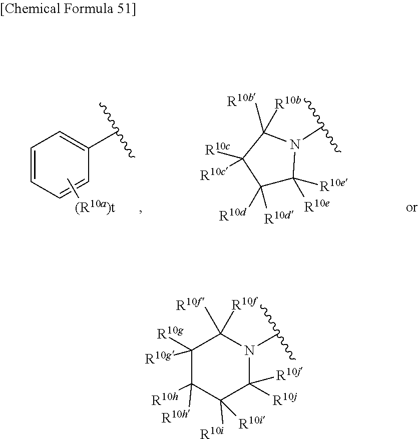

wherein
t is an integer of 0 to 5,
R^{10a}s are each independently halogen, cyano, carboxy, substituted or unsubstituted alkyl, substituted or unsubstituted alkyloxy, substituted or unsubstituted alkylcarbonyl, substituted or unsubstituted alkyloxycarbonyl, substituted or unsubstituted alkylsulfonyl, substituted or unsubstituted sulfamoyl, substituted or unsubstituted carbamoyl, substituted or unsubstituted amino, substituted or unsubstituted aromatic heterocyclyl, substituted or unsubstituted non-aromatic heterocyclyl, substituted or unsubstituted non-aromatic heterocyclylcarbonyl, substituted or unsubstituted sulfoxyimino, or substituted or unsubstituted iminosulfino; or
two R^{10a}s bonded to an adjacent carbon atom may be taken together to form substituted or unsubstituted aromatic heterocycle, or substituted or unsubstituted non-aromatic heterocycle;
R^{10b}, R^{10b′}, R^{10c}, R^{10c′}, R^{10d}, R^{10d′}, R^{10e}, R^{10e′}, R^{10f}, R^{10f′}, R^{10g}, R^{10g′}, R^{10h}, R^{10h′}, R¹⁰ⁱ, R^{10i′}, R^{10j}, and R^{10j′} are each independently a hydrogen atom, hydroxy, halogen, substituted or unsubstituted alkyl, substituted or unsubstituted amino, substituted or unsubstituted carbamoyl, substituted or unsubstituted alkyloxy, substituted or unsubstituted alkyloxycarbonyl, or substituted or unsubstituted aromatic heterocyclyl;
R^{10b} and R^{10b′}, R^{10c} and R^{10c′}, R^{10d} and R^{10d′}, R^{10e} and R^{10e′}, R^{10f} and R^{10f′}, R^{10g} and R^{10g′}, R^{10h} and R^{10h′}, R¹⁰ⁱ and R^{10i′}, and R^{10j} and R^{10j′} may be each independently taken together with a carbon atom to which they are bonded to form oxo, a substituted or unsubstituted non-aromatic carbocycle, or a substituted or unsubstituted non-aromatic heterocycle;
R^{10b} and R^{10c}, R^{10c} and R^{10d}, R^{10d} and R^{10e}, R^{10f} and R^{10g}, R^{10g} and R^{10h}, R^{10h} and R¹⁰ⁱ, and R¹⁰ⁱ and R^{10j} may be each independently taken together with a carbon atom to which they are each bonded to form a substituted or unsubstituted non-aromatic heterocycle, or a substituted or unsubstituted non-aromatic carbocycle;
R^{10b} and R^{10d}, R^{10b} and R^{10e}, R^{10c} and R^{10e}, R^{10f} and R^{10h}, R^{10f} and R¹⁰ⁱ, R^{10f} and R^{10j}, R^{10g} and R¹⁰ⁱ, and R^{10g} and R^{10j} may each independently together each form a (C1-C4) bridge, in which one of the carbon atoms that constitute the bridge may be replaced by an oxygen atom or a nitrogen atom; the carbon atoms constituting the bridge are each independently substituted with a substituent selected from R^{a}, and the nitrogen atom constituting the bridge is substituted with a substituent selected from R^{b};
wherein R^{a} is a hydrogen atom, halogen, substituted or unsubstituted alkyl, or substituted or unsubstituted alkyloxy;
R^{b} is a hydrogen atom, or substituted or unsubstituted alkyl (hereinafter, referred to as C-6).

R^{3a} may be a hydrogen atom, substituted or unsubstituted aromatic carbocyclyl, substituted or unsubstituted aromatic heterocyclyl, substituted or unsubstituted non-aromatic heterocyclyl, substituted or unsubstituted alkyloxy, substituted amino, halogen, substituted or unsubstituted carbamoyl, substituted or unsubstituted sulfamoyl, substituted or unsubstituted non-aromatic carbocyclyloxy, or substituted or unsubstituted non-aromatic heterocyclyloxy (hereinafter, referred to as C-7).

R^{3b} and R^{3b′} may be each independently a hydrogen atom, substituted or unsubstituted aromatic carbocyclyl, substituted or unsubstituted aromatic heterocyclyl, substituted or unsubstituted non-aromatic heterocyclyl, substituted or unsubstituted alkyloxy, substituted amino, halogen, substituted or unsubstituted alkyl, substituted or unsubstituted carbamoyl, substituted or unsubstituted sulfamoyl, substituted or unsubstituted non-aromatic carbocyclyloxy, or substituted or unsubstituted non-aromatic heterocyclyloxy (hereinafter, referred to as C-8).

R^{3a}, R^{3b}, and R^{3b′} may be each independently a hydrogen atom, substituted or unsubstituted aromatic carbocyclyl, substituted or unsubstituted aromatic heterocyclyl, substituted or unsubstituted non-aromatic heterocyclyl, substituted or unsubstituted alkyloxy, substituted amino, halogen, substituted or unsubstituted carbamoyl, substituted or unsubstituted sulfamoyl, substituted or unsubstituted non-aromatic carbocyclyloxy, or substituted or unsubstituted non-aromatic heterocyclyloxy (hereinafter, referred to as C-9).

R^{3a}, R^{3b}, and R^{3b′} may be each independently substituted or unsubstituted aromatic carbocyclyl, or substituted or unsubstituted non-aromatic heterocyclyl (hereinafter, referred to as C-10).

R^{3a}, R^{3b}, and R^{3b′} may be each independently substituted or unsubstituted aromatic carbocyclyl (hereinafter, referred to as C-11).

R^{3a}, R^{3b}, and R^{3b′} may be each independently substituted or unsubstituted non-aromatic heterocyclyl (hereinafter, referred to as C-12).

R^{3a}, R^{3b}, and R^{3b′} may be each independently a group represented by:

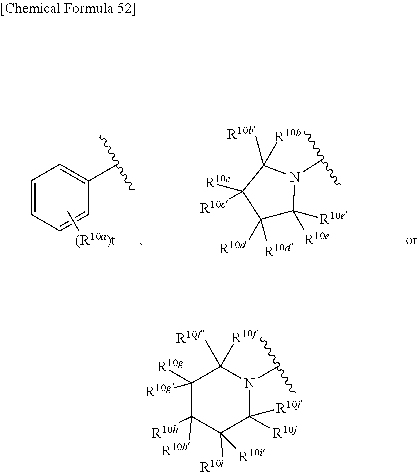

wherein
t is an integer of 0 to 5,
R^{10a}s are each independently halogen, cyano, carboxy, substituted or unsubstituted alkyl, substituted or unsubstituted alkyloxy, substituted or unsubstituted alkylcarbonyl, substituted or unsubstituted alkyloxycarbonyl, substituted or unsubstituted alkylsulfonyl, substituted or unsubstituted sulfamoyl, substituted or unsubstituted carbamoyl, substituted or unsubstituted amino, substituted or unsubstituted aromatic heterocyclyl, substituted or unsubstituted non-aromatic heterocyclyl, substituted or unsubstituted non-aromatic heterocyclylcarbonyl, substituted or unsubstituted sulfoxyimino, or substituted or unsubstituted iminosulfino; or
two R^{10a}s bonded to an adjacent carbon atom may be taken together to form substituted or unsubstituted aromatic heterocycle, or substituted or unsubstituted non-aromatic heterocycle;
R^{10b}, R^{10b′}, R^{10c}, R^{10c′}, R^{10d}, R^{10d′}, R^{10e}, R^{10e′}, R^{10f}, R^{10f′}, R^{10g}, R^{10g′}, R^{10h}, R^{10h′}, R¹⁰ⁱ, R^{10i′}, R^{10j}, and R^{10j′} are each independently a hydrogen atom, hydroxy, halogen, substituted or unsubstituted alkyl, substituted or unsubstituted amino, substituted or unsubstituted carbamoyl, substituted or unsubstituted alkyloxy, substituted or unsubstituted alkyloxycarbonyl, or substituted or unsubstituted aromatic heterocyclyl;
R^{10b} and R^{10b′}, R^{10c} and R^{10c′}, R^{10d} and R^{10d′}, R^{10e} and R^{10e′}, R^{10f} and R^{10f′}, R^{10g} and R^{10g′}, R^{10h} and R^{10h′}, R¹⁰ⁱ and R^{10i′}, and R^{10j} and R^{10j′} may be each independently taken together with a carbon atom to which they are bonded to form oxo, a substituted or unsubstituted non-aromatic carbocycle, or a substituted or unsubstituted non-aromatic heterocycle;
R^{10b} and R^{10c}, R^{10c} and R^{10d}, R^{10d} and R^{10e}, R^{10f} and R^{10g}, R^{10g} and R^{10h}, R^{10h} and R¹⁰ⁱ, and R¹⁰ⁱ and R^{10j} may be each independently taken together with a carbon atom to which they are each bonded to form a substituted or unsubstituted non-aromatic heterocycle, or a substituted or unsubstituted non-aromatic carbocycle; and
R^{10b} and R^{10d}, R^{10b} and R^{10e}, R^{10c} and R^{10e}, R^{10f} and R^{10h}, R^{10f} and R¹⁰ⁱ, R^{10f} and R^{10j}, R^{10g} and R¹⁰ⁱ, and R^{10g} and R^{10j} may each independently together each form a (C1-C4) bridge, in which one of the carbon atoms that constitute the bridge may be replaced by an oxygen atom or a nitrogen atom; the carbon atoms constituting the bridge are each independently substituted with a substituent selected from R^{a}, and the nitrogen atom constituting the bridge is substituted with a substituent selected from R^{b};
wherein R^{a} is a hydrogen atom, halogen, substituted or unsubstituted alkyl, or substituted or unsubstituted alkyloxy;
R^{b} is a hydrogen atom, or substituted or unsubstituted alkyl (hereinafter, referred to as C-13).

R^{3a} and R^{3b} may be each independently a group represented by:

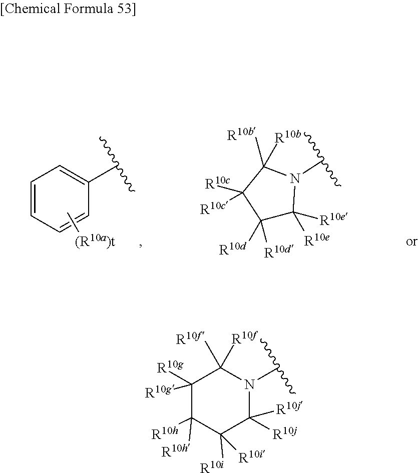

wherein each symbol has the same meaning as that in the above C-13 (hereinafter, referred to as C-14).

R^{3a}, R^{3b}, and R^{3b′} may be each independently a group represented by:

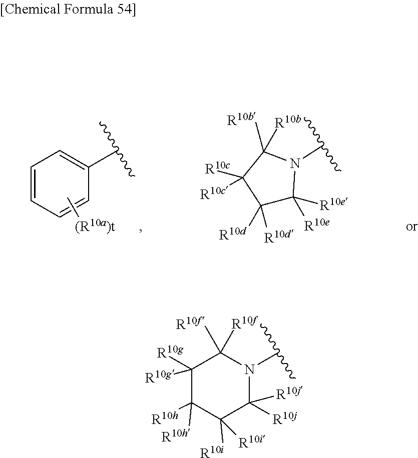

wherein each symbol has the same meaning as that in the above C-13, or
R^{3a}, R^{3b}, and R^{3b′} may be each independently substituted amino (hereinafter referred to as (C-15).

R^{3a} and R^{3b} may be each independently a group represented by:

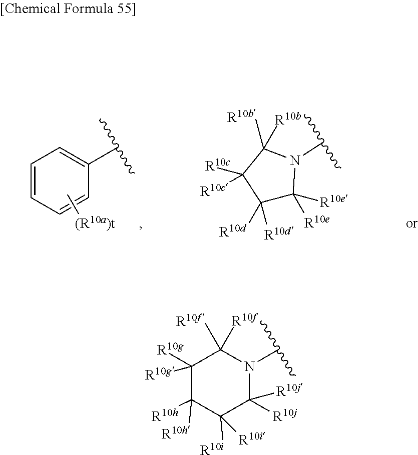

wherein each symbol has the same meaning as that in the above C-13, or
R^{3a}, R^{3b}, and R^{3b′} may be each independently substituted amino (hereinafter referred to as (C-16).

A ring formed by R³ and R⁸ taken together with a carbon atom to which they are each bonded may be a substituted or unsubstituted aromatic carbocycle, or a substituted or unsubstituted aromatic heterocycle (hereinafter, referred to as C′-1).

A ring formed by R^{3a} and R⁸, and R^{3b} and R⁸ taken together with a carbon atom to which they are each bonded may be a substituted or unsubstituted aromatic carbocycle (hereinafter, referred to as C-2′).

R^{4a} may be a hydrogen atom, or substituted or unsubstituted alkyl (hereinafter, referred to as D-1).

R^{4a} may be a hydrogen atom or unsubstituted alkyl (hereinafter, referred to as D-3).

R^{4a} may be a hydrogen atom (hereinafter, referred to as D-2).

R^{4b} may be a hydrogen atom, or substituted or unsubstituted alkyl (hereinafter, referred to as E-1).

R^{4b} may be a hydrogen atom (hereinafter, referred to as E-2).

R^{5a} may be a hydrogen atom, or substituted or unsubstituted alkyl (hereinafter, referred to as F-1).

R^{5a} may be a hydrogen atom (hereinafter, referred to as F-2).

R^{5b} may be a hydrogen atom, or substituted or unsubstituted alkyl (hereinafter, referred to as G-1).

R^{5b} may be a hydrogen atom (hereinafter, referred to as G-2).

R⁶ may be a hydrogen atom, halogen, substituted or unsubstituted alkyloxy, hydroxy, or cyano (hereinafter, referred to as H-2).

R⁶ may be halogen, substituted or unsubstituted alkyloxy, hydroxy, or cyano (hereinafter, referred to as H-3).

R⁶ may be halogen, alkyloxy, or hydroxy (hereinafter, referred to as H-4).

R⁶ may be a hydrogen atom, halogen, or substituted or unsubstituted alkyloxy (hereinafter, referred to as H-1).

R⁷ may be a hydrogen atom, halogen, substituted or unsubstituted alkyl, substituted or unsubstituted alkenyl, substituted or unsubstituted alkynyl, substituted or unsubstituted alkyloxy, substituted or unsubstituted non-aromatic carbocycle, substituted or unsubstituted non-aromatic heterocycle, substituted or unsubstituted non-aromatic carbocyclyloxy, substituted or unsubstituted amino, substituted or unsubstituted alkylsulfoxy, substituted or unsubstituted carbamoyl, hydroxy, carboxy, formyl, or cyano (hereinafter, referred to as I-2).

R⁷ is a hydrogen atom, halogen, substituted or unsubstituted alkyl, substituted or unsubstituted alkyloxy, or substituted or unsubstituted amino (hereinafter referred to as I-1).

R⁷ may be a hydrogen atom, halogen, substituted or unsubstituted alkyl, or substituted or unsubstituted alkyloxy (hereinafter, referred to as I-3).

R^{7′} may be a hydrogen atom, or substituted or unsubstituted alkyl (hereinafter, referred to as I′-1).

R^{9′} may be a hydrogen atom, or substituted or unsubstituted alkyl (hereinafter, referred to as I′-2).

A ring formed by R^{3b} and R^{7′} taken together with an atom to which they are each bonded may be a substituted or unsubstituted non-aromatic heterocycle (hereinafter, referred to as I″-1).

R⁸ and R^{8′} may be each independently a hydrogen atom, halogen, substituted or unsubstituted alkyl, or substituted or unsubstituted alkyloxy (hereinafter, referred to as J-2).

R⁸ and R^{8′} may be each independently a hydrogen atom or halogen (hereinafter, referred to as J-3).

R⁸ may be a hydrogen atom or halogen (hereinafter, referred to as J-1).

R⁸ may be a hydrogen atom (hereinafter, also referred to as J-4).

R⁸ may be halogen (referred to as J-5).

R⁹ may be halogen, hydroxy, substituted or unsubstituted alkyl, or substituted or unsubstituted alkyloxy (hereinafter, referred to as O-2).

R⁹ may be halogen, hydroxy, or alkyl (hereinafter, referred to as O-2).

R⁹ may be substituted or unsubstituted alkyl (hereinafter, referred to as O-1).

m may be 0, 1, or 2 (hereinafter, referred to as K-1).

m may be 0 or 1 (hereinafter, referred to as K-2).

m may be 0 (hereinafter, referred to as K-3).

m may be 1 (hereinafter, referred to as K-4).

n may be 0, 1, or 2 (hereinafter, referred to as L-1).

n may be 0 or 1 (hereinafter, referred to as L-2).

n may be 0 (hereinafter, referred to as L-4).

n may be 1 (hereinafter, referred to as L-3).

p may be 1, 2, or 3 (hereinafter, referred to as M-1).

p may be 1 (hereinafter, referred to as M-2).

s may be 0, 1, or 2 (hereinafter, referred to as N-1).

s may be 0 or 1 (hereinafter, referred to as N-3).

s may be 0 (hereinafter, referred to as N-2).

With regard to a compound represented by Formula (I):

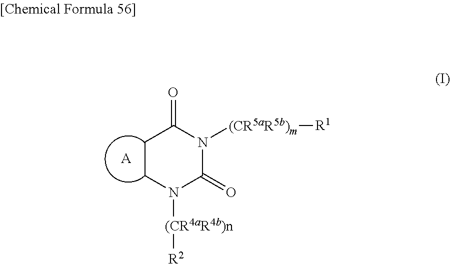

wherein ring A is represented by:

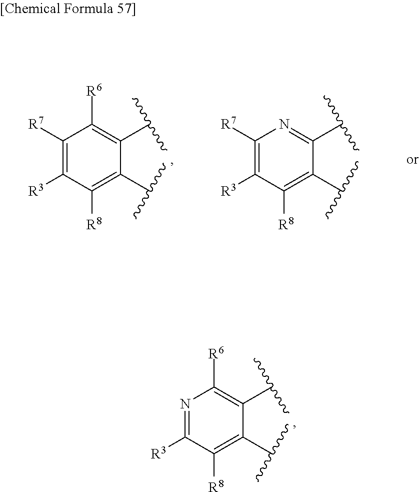

preferred embodiments of R¹, R², R³, R^{4a}, R^{4b}, R^{5a}, R^{5b}, R⁶, R⁷, R⁸, m, and n are shown below. Regarding the compound represented by Formula (I), embodiments of all the combinations of specific examples shown below are mentioned as examples.

R¹ may be A-1, A-2, A-3, A-4, or A-5.

R² may be B-1, B-2, B-3, B-4, B-5, or B-6.

R³ may be C-1, C-2, C-3, C-4, C-5, C-6, or C′-1.

R^{4a} may be D-1 or D-2.

R^{4b} may be E-1 or E-2.

R^{5a} may be F-1 or F-2.

R^{5b} may be G-1 or G-2.

R⁶ may be H-1.

R⁷ may be I-1.

R⁸ may be J-1.

m may be K-1, K-2, K-3, or K-4.

n may be L-1, L-2, or L-3.

With regard to a compound represented by Formula (I):

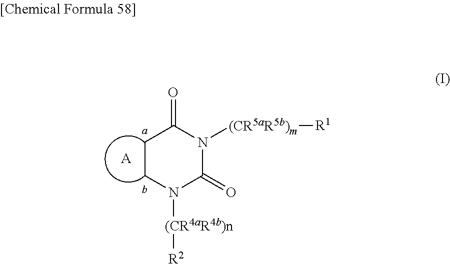

wherein ring A is represented by:

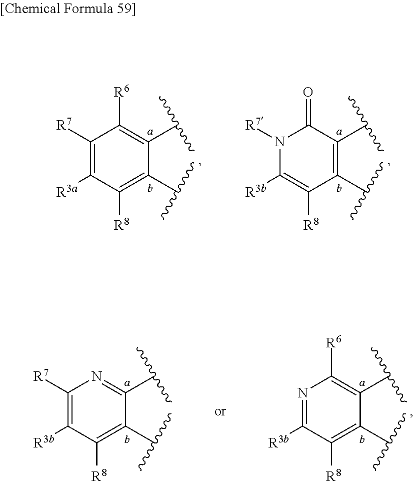

preferred embodiments of R¹, R², R^{3a}, R^{3b}, R^{4a}, R^{4b}, R^{5a}, R^{5b}, R⁶, R⁷, R^{7′}, R⁸, m, and n are shown below. Regarding the compound represented by Formula (I), embodiments of all the combinations of specific examples shown below are mentioned as examples.

R¹ may be A-6, A-7, A-8, or A-9.

R² may be B-5, B-6, B-7, B-8, or B-9.

R^{3a} may be C-7, C-10, C-11, C-12, or C-14.

R^{3b} may be C-8, C-9, C-10, C-11, C-12, or C-14.

R^{3b} may be C-8, C-9, C-10, C-11, C-12, or C-14.

R^{4a} may be D-2 or D-3.

R^{4b} may be E-2.

R^{5a} may be F-2.

R^{5b} may be G-2.

R⁶ may be H-1, H-2, or H-3.

R⁷ may be I-1 or I-2.

R^{7′} may be I′-1, or

R^{3b} and R^{V} may be I″-1,

R⁸ may be J-1 or J-2.

m may be K-2, K-3, or K-4.

n may be L-2, L-3, or L-4.

Another exemplary embodiment of the compound represented by Formula (I) or its pharmaceutically acceptable salt are shown below.

In a compound represented by Formula (I):

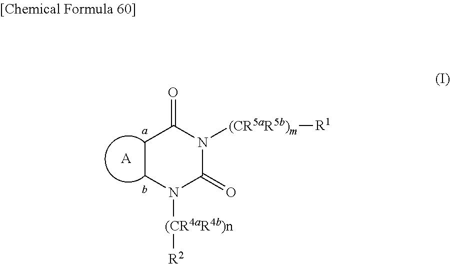

wherein
ring A is a ring represented by:

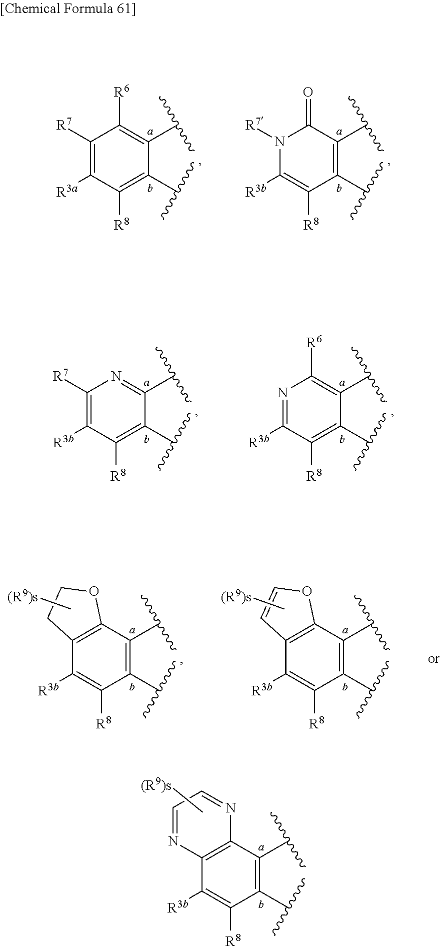

wherein
R^{3a} and R^{3b} may be each independently a group represented by:

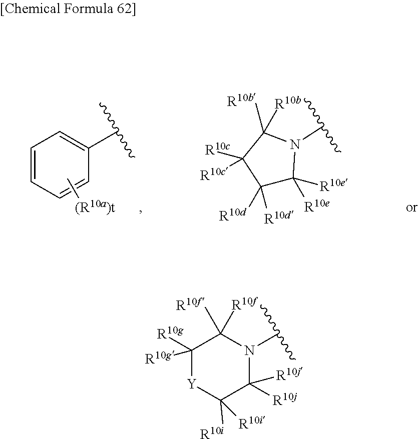

wherein Y is CR^{10h}R^{10h′} or O;
t is an integer of 1 to 5,
R^{10a}s may be each independently halogen, cyano, substituted or unsubstituted alkyl (examples of the substituent include halogen, cyano, hydroxy, carboxy, carbamoyl, dialkylcarbamoyl, alkylsulfonylalkylcarbamoyl, dialkylaminoalkylcarbamoyl, alkyl non-aromatic heterocyclylcarbamoyl, alkyloxy, dialkylsulfoxyimino, amino non-aromatic heterocyclylcarbonyl; optionally substituted with one or more group(s) selected from these), unsubstituted alkyloxy, unsubstituted alkylcarbonyl, unsubstituted alkylsulfonyl, substituted sulfoxyimino (examples of the substituent include alkyl; optionally substituted with one or more group(s) selected from these), substituted iminosulfino (examples of the substituent include alkyl; optionally substituted with one or more group(s) selected from these), substituted or unsubstituted amino (examples of the substituent include alkylcarbonyl, alkylsulfonyl; optionally substituted with one or more group(s) selected from these), substituted or unsubstituted carbamoyl (examples of the substituent include alkyl, hydroxyalkyl, dialkylaminoalkyl, hydroxyalkylaminoalkyl, non-aromatic heterocyclylalkyl, alkyloxy, alkyloxyalkyl, hydroxyalkyloxyalkyl, alkyl non-aromatic heterocyclyl; optionally substituted with one or more group(s) selected from these), substituted or unsubstituted sulfamoyl (examples of the substituent include alkyl; optionally substituted with one or more group(s) selected from these), unsubstituted dialkylphosphoryl, substituted or unsubstituted non-aromatic carbocyclyl (examples of the substituent include hydroxy; optionally substituted with one or more group(s) selected from these), substituted or unsubstituted aromatic heterocyclyl (examples of the substituent include amino, alkyl; optionally substituted with one or more group(s) selected from these), unsubstituted non-aromatic heterocyclyl, or substituted or unsubstituted non-aromatic heterocyclylcarbonyl (examples of the substituent include alkyl; optionally substituted with one or more group(s) selected from these), or
two R^{10a}s bonded to an adjacent carbon atom may be taken together to form substituted or unsubstituted aromatic heterocycle, or substituted or unsubstituted non-aromatic heterocycle;
the substituent on the ring of “substituted or unsubstituted aromatic heterocycle formed by two R^{10a}s bonded to an adjacent carbon atom” is a group selected from cyano, alkyl, haloalkyl, and alkyloxy; and may be substituted with one or more group(s) selected from these;
the substituent on the ring of “substituted or unsubstituted non-aromatic heterocycle formed by two R^{10a}s bonded to an adjacent carbon atom” is a group selected from oxo, alkyl, haloalkyl, and non-aromatic carbocycle; and may be substituted with one or more group(s) selected from these;
R^{10b}, R^{10b′}, R^{10c}, R^{10c′}, R^{10d}, R^{10d′}, R^{10e}, R^{10e′}, R^{10f}, R^{10f′}, R^{10g}, R^{10g′}, R^{10h}, R^{10h′}, R¹⁰ⁱ, R^{10i′}, R^{10j}, and R^{10j′} are each independently a hydrogen atom, halogen, cyano, hydroxy, carboxy, substituted or unsubstituted alkyl (examples of the substituent include halogen, hydroxy, alkyloxy; optionally substituted with one or more group(s) selected from these), substituted or unsubstituted alkyloxy (example of the substituent include halogen; optionally substituted with one or more group(s) selected from these), substituted or unsubstituted amino (examples of the substituent include alkyl, alkylcarbonyl; optionally substituted with one or more group(s) selected from these), substituted or unsubstituted carbamoyl (examples of the substituent include alkyl; optionally substituted with one or more group(s) selected from these), unsubstituted alkylsulfonyl, unsubstituted aromatic heterocyclyl, or unsubstituted non-aromatic heterocyclylcarbonyl;
R^{10b} and R^{10b′}, R^{10c} and R^{10c′}, R^{10d} and R^{10d′}, R^{10e} and R^{10e′}, R^{10f} and R^{10f′}, R^{10g} and R^{10g′}, R^{10h} and R^{10h′}, R¹⁰ⁱ and R^{10i′}, and R^{10j} and R^{10j′} may be each independently taken together with a carbon atom to which they are bonded to form oxo, or a non-aromatic heterocycle;
R^{10b} and R^{10c}, R^{10c} and R^{10d}, R^{10d} and R^{10e}, R^{10f} and R^{10g}, R^{10g} and R^{10h}, R^{10h} and R¹⁰ⁱ, and R¹⁰ⁱ and R^{10j} may be each independently taken together with a carbon atom to which they are each bonded to form a substituted or unsubstituted non-aromatic heterocycle (examples of the substituent include halogen; optionally substituted with one or more group(s) selected from these), or a substituted or unsubstituted non-aromatic carbocycle (examples of the substituent include halogen; optionally substituted with one or more group(s) selected from these);
R^{10b} and R^{10d}, R^{10b} and R^{10e}, R^{10c} and R^{10e}, R^{10f} and R^{10h}, R^{10f} and R¹⁰ⁱ, R^{10f} and R^{10j}, R^{10g} and R¹⁰ⁱ, and R^{10g} and R^{10j} may each independently together each form a (C1-C4) bridge, in which one of the carbon atoms that constitute the bridge may be replaced by an oxygen atom or a nitrogen atom; the carbon atoms constituting the bridge are each independently substituted with a substituent selected from R^{a}, and the nitrogen atom constituting the bridge is substituted with a substituent selected from R^{b};
wherein R^{a} is a hydrogen atom and hydroxy; and R^{b} is a hydrogen atom, or
R^{3a}, R^{3b}, and R^{3b′} may be each independently substituted amino;
the substituent for the “substituted amino” in R^{3a} and R^{3b} is a group selected from substituted alkyl (examples of the substituent include halogen, alkyloxy; optionally substituted with one or more group(s) selected from these), unsubstituted alkyl, and unsubstituted non-aromatic carbocyclyl; optionally substituted with one or more group(s) selected from these;
R⁸ is a hydrogen atom or halogen;
R⁶ is halogen, alkyloxy, or hydroxy;
R⁷ is a hydrogen atom, halogen, alkyl, alkyloxy, cyanoalkyloxy;
R^{7′} is a hydrogen atom or alkyl;
R^{3b} and R^{7′} may be taken together with a carbon atom to which they are each bonded to form a non-aromatic heterocycle;
R⁹s are each independently halogen, hydroxy, or alkyl;
s is 0 or 1;
R¹ is substituted or unsubstituted 5- to 9-membered nitrogen-containing aromatic heterocyclyl, or substituted or unsubstituted 6-membered nitrogen-containing non-aromatic heterocyclyl;
the substituent on the ring of “substituted or unsubstituted 5- to 9-membered nitrogen-containing aromatic heterocyclyl” in R¹ is a group selected from halogen, cyano, alkyl, haloalkyl, alkyloxy, and haloalkyloxy; optionally substituted with one or more group(s) selected from these;
the substituent on the ring of “substituted or unsubstituted 6-membered nitrogen-containing non-aromatic heterocyclyl” in R¹ is a group selected from oxo and halogen; optionally substituted with one or more group(s) selected from these;
R² is 6-membered aromatic carbocyclyl substituted with one, two, three, four, or five substituents selected from substituent group G (substituent group G: halogen, cyano, alkyl, alkenyl, alkynyl, haloalkyl, alkyloxy, alkenyloxy, alkynyloxy, and haloalkyloxy), or 6-membered aromatic heterocyclyl substituted with one or two substituents selected from the substituent group G;
n is 0 or 1;
R^{4a} and R^{4b} are each independently a hydrogen atom or alkyl;
m is 0 or 1;
R^{5a} and R^{5b} are each independently a hydrogen atom.

The compound represented by Formula (I) is not limited to specific isomers, but includes all possible isomers (for example, keto-enol isomer, imine-enamine isomer, diastereoisomer, optical isomer, rotamer, etc.), racemates, or a mixture thereof.

One or more hydrogen atom, carbon atom and/or other atom of the compound represented by Formula (I) may be substituted with an isotope of the hydrogen atom, carbon atom and/or other atom, respectively. Examples of such isotopes include hydrogen, carbon, nitrogen, oxygen, phosphorus, sulfur, fluorine, iodine, and chlorine, as in the cases of ²H, ³H, ¹¹C, ¹³C, ¹⁴C, ¹⁵N, ¹⁸O, ¹⁷O, ³¹P, ³²P, ³⁵S, ¹⁸F, ¹²³I, and ³⁶Cl, respectively. The compound represented by Formula (I) also includes a compound substituted with such an isotope. The compound substituted with the isotope is also useful as a pharmaceutical product and includes all radiolabeled forms of the compound represented by Formula (I). Furthermore, a “radiolabeling method” for producing the “radiolabeled forms” is also included in the present invention, and the “radiolabeled forms” are useful as tools for metabolic pharmacokinetics studies, studies on binding assay, and/or diagnostics.

The radiolabeled form of the compound represented by Formula (I) can be prepared by the method well known in this technical field. For example, a tritium-labeled compound represented by Formula (I) can be prepared by introducing tritium into a specific compound represented by Formula (I) by catalytic dehalogenation reaction using tritium. This method includes reacting an appropriately halogenated precursor of the compound represented by Formula (I) with tritium gas in the presence of an appropriate catalyst, such as Pd/C, and in the presence or absence of a base. Regarding other appropriate methods for preparing tritium-labeled compounds, “Isotopes in the Physical and Biomedical Sciences, Vol. 1, Labeled Compounds (Part A), Chapter 6 (1987)” can be referred to. A ¹⁴C-labeled compound can be prepared by using a raw material having ¹⁴C carbon.

Examples of the pharmaceutically acceptable salt of the compound represented by Formula (I) include salts of the compound represented by Formula (I) with alkali metal (for example, lithium, sodium, potassium, etc.), alkaline earth metal (for example, calcium, barium, etc.), magnesium, transition metal (for example, zinc, iron, etc.), ammonia, organic base (for example, trimethylamine, triethylamine, dicyclohexylamine, ethanolamine, diethanolamine, triethanolamine, meglumine, ethylenediamine, pyridine, picoline, quinoline, etc.) and amino acid or salts of the compound represented by Formula (I) with inorganic acid (for example, hydrochloric acid, sulfuric acid, nitric acid, carbonic acid, hydrobromic acid, phosphoric acid, hydroiodic acid, etc.), and organic acid (for example, formic acid, acetic acid, propionic acid, trifluoroacetic acid, citric acid, lactic acid, tartaric acid, oxalic acid, maleic acid, fumaric acid, succinic acid, mandelic acid, glutaric acid, malic acid, benzoic acid, phthalic acid, ascorbic acid, benzenesulfonic acid, p-toluenesulfonic acid, methanesulfonic acid, ethanesulfonic acid, trifluoroacetic acid, etc.). These salts can be formed according to methods that are conventionally carried out.

The compounds represented by Formula (I) or pharmaceutically acceptable salts thereof according to the present invention may form solvates (e.g., hydrates or the like), cocrystals and/or crystal polymorphs. The present invention encompasses those various solvates, cocrystals and crystal polymorphs. The “solvate” may be one wherein any number of solvent molecules (e.g., water molecules or the like) is coordinated with the compound represented by Formula (I). When the compound represented by Formula (I) or a pharmaceutically acceptable salt thereof is allowed to stand in the atmosphere, the compound may absorb water, resulting in attachment of adsorbed water or formation of a hydrate. Furthermore, crystalline polymorphs may be formed by recrystallizing the compounds represented by Formula (I) or pharmaceutically acceptable salts thereof. The term “co-crystal” means that the compound represented by Formula (I) or a salt thereof and a counter molecule are present in the same crystal lattice, and may contain any number of counter molecules.

The compounds represented by Formula (I) or pharmaceutically acceptable salts thereof may form prodrugs. The present invention also encompasses such various prodrugs. A prodrug is a derivative of a compound of the present invention having a group that can be chemically or metabolically degraded, and is a compound which becomes a pharmaceutically active compound of the present invention in vivo as a result of solvolysis or under physiological conditions. Prodrugs encompass compounds that are converted to the compounds represented by Formula (I) through enzymatic oxidation, reduction, hydrolysis or the like under physiological conditions and in vivo, compounds that are converted to the compounds represented by Formula (I) through hydrolysis by gastric acid etc., and the like. Methods for selecting and producing an appropriate prodrug derivative are described in, for example, “Design of Prodrugs, Elsevier, Amsterdam, 1985”. A prodrug may have activity per se.

When the compounds represented by Formula (I) or pharmaceutically acceptable salts thereof have hydroxy group(s), prodrugs include acyloxy derivatives and sulfonyloxy derivatives that are prepared by, for example, reacting compounds having hydroxy group(s) with suitable acyl halide, suitable acid anhydride, suitable sulfonyl chloride, suitable sulfonyl anhydride and mixed anhydride, or with a condensing agent. Examples include CH₃COO—, C₂H₅COO—, tert-BuCOO—, C₁₅H₃₁COO—, PhCOO—, (m-NaOOCPh)COO—, NaOOCCH₂CH₂COO—, CH₃CH(NH₂)COO—, CH₂N(CH₃)₂COO—, CH₃SO₃—, CH₃CH₂SO₃—, CF₃SO₃—, CH₂FSO₃—, CF₃CH₂SO₃—, p-CH₃O-PhSO₃—, PhSO₃—, and p-CH₃PhSO₃—.

Since the compound according to the present invention has coronavirus 3CL protease inhibitory activity, the compound is useful as a therapeutic and/or prophylactic agent for a disease associated with coronavirus 3CL proteases. When the term “therapeutic agent and/or prophylactic agent” is used in the present invention, this also includes a symptom ameliorating agent. The disease associated with coronavirus 3CL proteases may be viral infections, and preferably coronavirus infections.

According to an aspect, the coronavirus may be a coronavirus that infects human beings. The coronavirus that infects human beings may be HCoV-229E, HCoV-NL63, HCoV-HKU1, HCoV-OC43, SARS-CoV, MERS-CoV, and/or SARS-CoV-2.

As an embodiment, as the coronavirus, Alphacoronavirus and/or Betacoronavirus, more preferably Betacoronavirus, and further preferably Sarbecovirus are exemplified.

According to an aspect, the alphacoronavirus may be HCoV-229E and HCoV-NL63. The alphacoronavirus may be particularly preferably HCoV-229E.

According to an aspect, the betacoronavirus may be HCoV-HKU1, HCoV-OC43, SARS-CoV, MERS-CoV, and/or SARS-CoV-2. The betacoronavirus may be HCoV-OC43 or SARS-CoV-2, and particularly preferably SARS-CoV-2.

According to an aspect, the betacoronavirus may be betacoronavirus lineage A (β-coronavirus lineage A), betacoronavirus lineage B (β-coronavirus lineage B), and betacoronavirus lineage C (β-coronavirus lineage C). The betacoronavirus may be more preferably betacoronavirus lineage A (β-coronavirus lineage A) and betacoronavirus lineage B (β-coronavirus lineage B) and particularly preferably betacoronavirus lineage B (β-coronavirus lineage B).

Examples of the betacoronavirus lineage A (β-coronavirus lineage A) include HCoV-HKU1 and HCoV-OC43, and preferably HCoV-OC43. Examples of the betacoronavirus lineage B (β-coronavirus lineage B) include SARS-CoV and SARS-CoV-2, and preferably SARS-CoV-2. The betacoronavirus lineage C (β-coronavirus lineage C) may be MERS-CoV.

According to an aspect, the coronavirus may be HCoV-229E, HCoV-OC43, and/or SARS-CoV-2, and particularly preferably SARS-CoV-2.

The coronavirus infections may be infections caused by HCoV-229E, HCoV-NL63, HCoV-OC43, HCoV-HKU1, SARS-CoV, MERS-CoV, and/or SARS-CoV-2. Preferably, the coronavirus infections may be infections caused by HCoV-229E, HCoV-OC43, and/or SARS-CoV-2, and particularly preferably infection caused by SARS-CoV-2.

The coronavirus infections may be particularly preferably novel coronavirus infections (COVID-19).

### (Method for Producing Compound of Present Invention)

For example, the compound represented by Formula (I) according to the present invention can be prepared by the general procedure described below. Regarding extraction, purification, and the like, the treatments carried out in ordinary experiments of organic chemistry may be carried out.

The compounds of the present invention can be produced with reference to techniques known in the art. For example, the compounds can be produced with reference to WO2013/184806, U.S. Pat. No. 4,731,106, WO2013/064083, and WO2020/261114.

(Method A) When the ring A is aromatic carbocycle or aromatic heterocycle

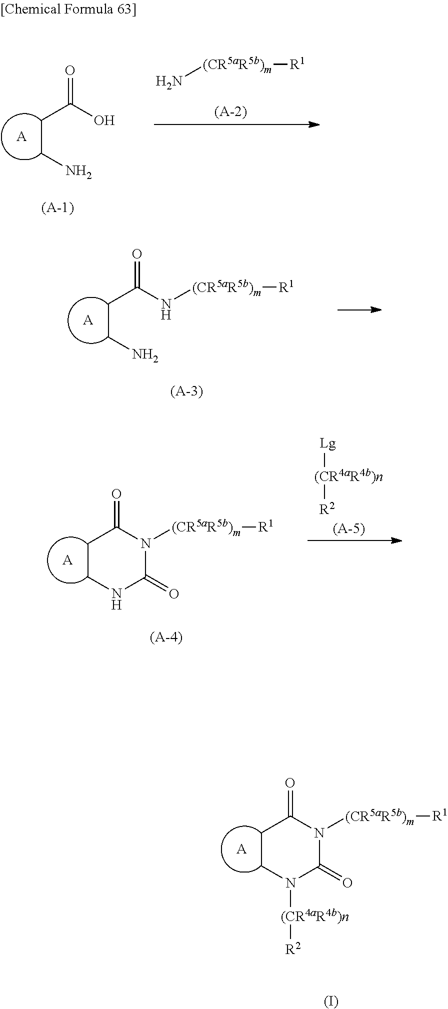

wherein Lg is a leaving group; and other reference symbols have the same meanings as described above.

### (First Step)

In the presence of a solvent such as DMF, DMA, NMP, or THF, or alternatively a mixed solvent thereof, a condensing agent such as HATU, WSC·HCl and HOBt, or PyBOP is added to Compound (A-1), then, Amine (A-2) corresponding to a target, and a tertiary amine such as triethylamine, N-methylmorpholine, or N,N-diisopropylethylamine is added thereto, and the mixture is reacted at 0° C. to 60° C., or preferably 20° C. to 40° C., for 0.1 hours to 24 hours, or preferably 0.5 hours to 12 hours, whereby Compound (A-3) can be obtained.

### (Second Step)

In the presence of a solvent such as DMF, DMA, NMP, THF, toluene, acetonitrile, dimethylsulfoxide, dioxane or dichloromethane or in a mixed solvent thereof, a carbonylating agent such as triphosgene, CDI, di-tert-butyl dicarbonate, urea, 4-nitrophenyl chloroformate, or methyl chloroformate is added to Compound (A-3), and the mixture is reacted at 0° C. to 140° C., or preferably at 20° C. to 100° C., for 0.1 hours to 24 hours, or preferably 0.5 hours to 12 hours, whereby Compound (A-4) can be obtained.

### (Third Step)

In the presence of acetonitrile, acetone, DMF, DMSO, or NMP or in a mixed solvent thereof, in the presence of a base such as potassium carbonate, sodium carbonate, or cesium carbonate, Compound (A-5) corresponding to a target is added to Compound (A-4), and the mixture is reacted at 0° C. to 100° C., or preferably at 20° C. to 60° C., for 0.1 hours to 24 hours, or preferably 0.5 hours to 12 hours, whereby Compound (I) can be obtained.

Examples of the leaving group include halogen and —OSO₂(CₜF₂ₜ₊₁) (wherein t is an integer of 1 to 4). The halogen is preferably chlorine, iodine, and bromine, and the —OSO₂(CₜF₂ₜ₊₁) group is preferably an —OTf group (trifluoromethanesulfonic acid ester).

(Method B) When the ring A is aromatic carbocycle or aromatic heterocycle

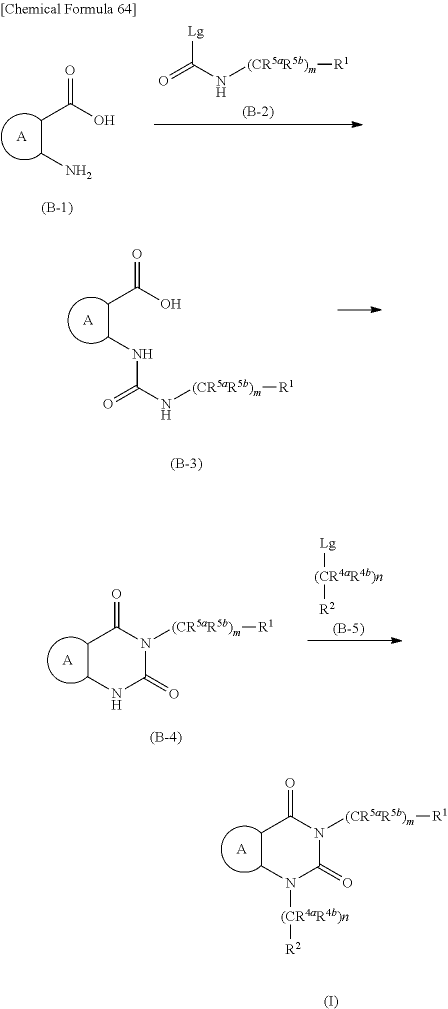

wherein Lg is a leaving group; and other reference symbols have the same meanings as described above.

### (First Step)

In the presence of a solvent such as DMF, DMA, NMP, THF, or toluene, or in a mixed solvent thereof, Compound (B-2) corresponding a target, and a tertiary amine such as triethylamine, N-methylmorpholine, or N,N-diisopropylethylamine, as well as DMAP as required, are added to Compound (B-1), and the mixture is reacted at 0° C. to 140° C., or preferably 20° C. to 100° C., for 0.1 hours to 24 hours, or preferably 0.5 hours to 12 hours, whereby Compound (B-3) can be obtained.

Examples of the leaving group include halogen and alkoxy.

### (Second Step)

An acid such as hydrochloric acid or sulfuric acid is added to Compound (B-3), and the mixture is reacted at 0° C. to 160° C., or preferably at 40° C. to 120° C., for 0.1 hours to 24 hours, or preferably 0.5 hours to 12 hours, whereby Compound (B-4) can be produced.

### (Third Step)

In the presence of acetonitrile, acetone, DMF, DMSO, or NMP or in a mixed solvent thereof, in the presence of a base such as potassium carbonate, sodium carbonate, or cesium carbonate, Compound (B-5) corresponding to a target is added to Compound (B-4), and the mixture is reacted at 0° C. to 100° C., or preferably at 20° C. to 60° C., for 0.1 hours to 24 hours, or preferably 0.5 hours to 12 hours, whereby Compound (B-6) can be obtained.

Examples of the leaving group include halogen and —OSO₂(CₜF₂ₜ₊₁) (wherein t is an integer of 1 to 4). The halogen is preferably chlorine, iodine, and bromine, and the —OSO₂(CₜF₂ₜ₊₁) group is preferably an —OTf group (trifluoromethanesulfonic acid ester).

(Method C) When the ring A is non-aromatic carbocycle or non-aromatic heterocycle

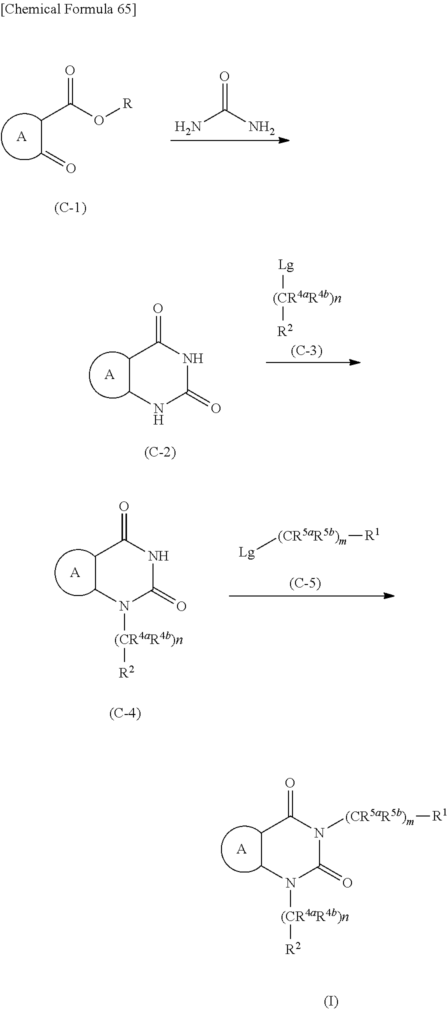

wherein Lg is a leaving group; and other reference symbols have the same meanings as described above.

### (First Step)

In a solvent such as methanol or ethanol, urea and a base such as sodium methoxide or sodium ethoxide are added to Compound (C-1), and the mixture is reacted at 0° C. to 140° C., or preferably at 20° C. to 100° C., for 0.1 hours to 48 hours, or preferably 0.5 hours to 18 hours, whereby Compound (C-2) can be obtained.

### (Second Step)

In the presence of acetonitrile, acetone, DMF, DMSO, NMP or in a mixed solvent thereof, N,O-bis(trimethylsilyl)acetamide and a corresponding compound (C-3) and, if necessary, a quaternary ammonium salt such as tetrabutylammonium bromide are added to Compound (C-2), and the mixture is reacted at 0° C. to 140° C., or preferably at 40° C. to 100° C., for 0.1 hours to 48 hours, or preferably 0.5 hours to 18 hours, whereby Compound (C-4) can be obtained. As another method, in the presence of DMF, DMSO, or NMP or in a mixed solvent thereof, in the presence of a base such as potassium carbonate, sodium carbonate, cesium carbonate, or sodium hydride, Compound (C-3) corresponding to a target is added to Compound (C-2), and the mixture is reacted at 0° C. to 100° C., or preferably at 20° C. to 60° C., for 0.1 hours to 24 hours, or preferably 0.5 hours to 12 hours, whereby Compound (C-4) can be obtained.

Examples of the leaving group include halogen and —OSO₂(CₜF₂ₜ₊₁) (wherein t is an integer of 1 to 4). The halogen is preferably chlorine, iodine, and bromine, and the —OSO₂(CₜF₂ₜ₊₁) group is preferably an —OTf group (trifluoromethanesulfonic acid ester).

### (Third Step)

In the presence of DMF, DMSO, or NMP or in a mixed solvent thereof, in the presence of a base such as potassium carbonate, sodium carbonate, cesium carbonate, or sodium hydride, Compound (C-5) corresponding to a target is added to Compound (C-4), and the mixture is reacted at 0° C. to 100° C., or preferably at 20° C. to 60° C., for 0.1 hours to 24 hours, or preferably 0.5 hours to 12 hours, whereby Compound (C-6) can be obtained.

Examples of the leaving group include halogen and —OSO₂(CₜF₂ₜ₊₁) (wherein t is an integer of 1 to 4). The halogen is preferably chlorine, iodine, and bromine, and the —OSO₂(CₜF₂ₜ₊₁) group is preferably an —OTf group (trifluoromethanesulfonic acid ester).

Since the compound of the present invention has coronavirus 3CL protease inhibitory activity, the compound is useful as a therapeutic and/or prophylactic agent for coronavirus infections.

Furthermore, the compound of the present invention has utility as a medicine, and preferably, the compound of the present invention has any one or a plurality of the following excellent features.
a) Inhibitory activity against CYP enzymes (for example, CYP1A2, CYP2C9, CYP2C19, CYP2D6, and CYP3A4) is weak.
b) Satisfactory pharmacokinetics such as high bioavailability and adequate clearance are exhibited.
c) Metabolic stability is high.
d) Irreversible inhibitory activity is not exhibited against CYP enzymes (for example, CYP3A4) within the concentration range of the measurement conditions described in the present specification.
e) Mutagenicity is not exhibited.
f) The cardiovascular risk is low.
g) High solubility is exhibited.
h) The protein unbinding rate (fu value) is high.
i) High coronavirus 3CL protease selectivity is exhibited.
j) High coronavirus replication inhibitory activity is exhibited. For example, high coronavirus replication inhibitory activity is exhibited when human blood serum (HS) or human serum albumin (HSA) is added.
k) High replication inhibitory activity is exhibited against 3CL protease inhibitor-resistant viruses.

Regarding the coronavirus replication inhibitor, for example, an aspect in which in the CPE effect (SARS-CoV-2) that will be described below, for example, EC₅₀ is 10 μM or less, preferably 1 μM or less, and more preferably 100 nM or less, may be mentioned.

The pharmaceutical composition of the present invention can be administered by either an oral method or a parenteral method. Examples of a parenteral administration method include percutaneous, subcutaneous, intravenous, intra-arterial, intramuscular, intraperitoneal, transmucosal, inhalation, transnasal, ocular instillation, ear instillation, and intravaginal administration.

In the case of oral administration, the pharmaceutical composition may be prepared into any dosage form that is commonly used, such as a solid preparation for internal use (for example, a tablet, a powder preparation, a granular preparation, a capsule, a pill, or a film preparation), or a liquid preparation for internal use (for example, a suspension, an emulsion, an elixir, a syrup, a limonade, a spirit preparation, an aromatic water preparation, an extraction, a decoction, or a tincture) and administered. The tablet may be a dragee, a film-coated tablet, an enteric-coated tablet, a sustained release tablet, a troche, a sublingual tablet, a buccal tablet, a chewable tablet, or an orally disintegrating tablet; the powder preparation and granular preparation may be dry syrups; and the capsule may be a soft capsule, a microcapsule, or a sustained release capsule.

In the case of parenteral administration, the pharmaceutical composition can be suitably administered in any dosage form that is commonly used, such as an injectable preparation, an infusion, or a preparation for external use (for example, an eye drop, a nasal drop, an ear drop, an aerosol, an inhalant, a lotion, an impregnating agent, a liniment, a gargling agent, an enema, an ointment, a plaster, a jelly, a cream, a patch, a poultice, a powder preparation for external use, or a suppository). The injectable preparation may be an O/W, W/O, O/W/O, or W/O/W type emulsion, or the like.

A pharmaceutical composition can be obtained by mixing an effective amount of the compound of the present invention with various pharmaceutical additives appropriate for the dosage form, such as an excipient, a binder, a disintegrating agent, and a lubricating agent, as necessary. Furthermore, the pharmaceutical composition can be prepared into a pharmaceutical composition for use for a child, an elderly, a patient with a serious case, or a surgical operation, by appropriately changing the effective amount of the compound of the present invention, the dosage form, and/or various pharmaceutical additives. For example, a pharmaceutical composition for use for a child may be administered to a neonate (less than 4 weeks after birth), an infant (from 4 weeks after birth to less than 1 year), a preschool child (from 1 year to less than 7 years), a child (from 7 years to less than 15 years), or a patient 15 year to 18 years of age. For example, a pharmaceutical composition for an elderly may be administered to a patient 65 years of age or older.

It is desirable to set the amount of administration of the pharmaceutical composition of the present invention, after considering the age and body weight of the patient, the type and degree of the disease, the route of administration, and the like; however, in the case of oral administration, the amount of administration is usually 0.05 to 200 mg/kg/day and is preferably in the range of 0.1 to 100 mg/kg/day. In the case of parenteral administration, the amount of administration may vary greatly depending on the route of administration; however, the amount of administration is usually 0.005 to 200 mg/kg/day and is preferably in the range of 0.01 to 100 mg/kg/day. This may be administered once a day or several times a day.

The compound of the present invention may be used in combination with, for example, another therapeutic agent for novel coronavirus infections (COVID-19) (the therapeutic agent includes an approved drug and a drug that is under development or to be developed in the future) (hereinafter, referred to as concomitant drug), for the purpose of enhancing the action of the compound, reducing the amount of administration of the compound, or the like. At this time, the timing of administration for the compound of the present invention and the concomitant drug is not limited, and these may be administered simultaneously to the target of administration or may be administered with a time difference. Furthermore, the compound of the present invention and the concomitant drug may be administered as two or more kinds of preparations each including active ingredients, or may be administered as a single preparation including those active ingredients.

The amount of administration of the concomitant drug can be appropriately selected based on the clinically used dosage. Furthermore, the blending ratio of the compound of the present invention and the concomitant drug can be appropriately selected according to the target of administration, the route of administration, the target disease, symptoms, combination, and the like. For example, when the target of administration is a human being, 0.01 to 100 parts by weight of the concomitant drug may be used with respect to 1 part by weight of the compound of the present invention.

### EXAMPLES

Hereinafter, the present invention will be described in more detail by way of Examples, Reference Examples, and Test Examples; however, the present invention is not intended to be limited by these.

Furthermore, abbreviations used in the present specification denote the following meanings.
CDI: Carbonyldiimidazole
DMA: dimethylacetamide
DMAP: 4-dimethylaminopyridine
DMF: N,N-dimethylformamide
ESI: Electrospray ionization
FBS: Fetal bovine serum
NMP: N-methylpyrrolidone
PdCl₂(dppf): [1,1′-bis(diphenylphosphino)ferrocene]palladium (II) dichloride dichloromethane adduct
PyBOP: (benzotriazol-1-yloxy)tripyrrolidinophosphonium hexafluorophosphate
RT: Retention time
RuPhos Pd G3: (2-dicyclohexylphosphino-2′,6′-diisopropoxy-1,1′-biphenyl)[2-(2′-amino-1,1′-biphenyl)]palladium (II) methanesulfonate
mM: mmol/L
μM: μmol/L
nM: nmol/L

### (Method for Identifying Compound)

The NMR analysis obtained in each Example was performed at 400 MHz, and measurement was made using DMSO-d₆ and CDCl₃. Furthermore, when NMR data are shown, there are occasions in which all the measured peaks are not described.

The term RT in the specification indicates retention time in an LC/MS: liquid chromatography/mass analysis, and the retention time was measured under the following conditions.

### (Measurement Conditions 1)

Column: ACQUITY UPLC (registered trademark) BEH C18 (1.7 μm i.d. 2.1×50 mm) (Waters)

Flow rate: 0.8 mL/min

UV detection wavelength: 254 nm

Mobile phase: [A] was 0.1% formic acid-containing aqueous solution, and [B] was 0.1% formic acid-containing acetonitrile solution.

Gradient: A linear gradient of 5% to 100% solvent [B] was carried out for 3.5 minutes, and then 100% solvent [B] was maintained for 0.5 minutes.

### (Measurement Conditions 2)

Column: Shim-pack XR-ODS (2.2 μm, i.d. 3.0×50 mm) (Shimadzu)

Flow rate: 1.6 mL/min

UV detection wavelength: 254 nm

Mobile phase: [A] was 0.1% formic acid-containing aqueous solution, and [B] was 0.1% formic acid-containing acetonitrile solution.

Gradient: A linear gradient of 10% to 100% solvent [B] was carried out for 3 minutes, and then 100% solvent [B] was maintained for 0.5 minutes.

### (Measurement Conditions 3)

Column: ACQUITY UPLC (registered trademark) BEH C18 (1.7 μm i.d. 2.1×50 mm) (Waters)

Flow rate: 0.8 mL/min

UV detection wavelength: 254 nm

Mobile phase: [A] was 10 mM ammonium carbonate in aqueous solution, and [B] was acetonitrile.

Gradient: A linear gradient of 5% to 100% solvent [B] was carried out for 3.5 minutes, and then 100% solvent [B] was maintained for 0.5 minutes.

Incidentally, in the specification, the description of MS (m/z) indicates a value observed by mass analysis.

### (Compound Separation Method)

### (Reverse Phase Separation)

Column: Gemini (registered trademark) 5 μm NX-C18 110 A, LC Column 100×30 mm, AXIA (trademark) Packed (Phenomenex)

Flow rate: 25 mL/min

UV detection wavelength: 254 nm

Mobile phase: [A] was 10 mmol/L ammonium carbonate aqueous solution, and [B] was acetonitrile.

Gradient: Yes

Start (0 min) [A]:[B]=40:60

End (8 min) [A]:[B]=20:80 Wash (8 to 10 min) [A]:[B]=0:100

### Example 1

### Synthesis of Compound (I-0299)

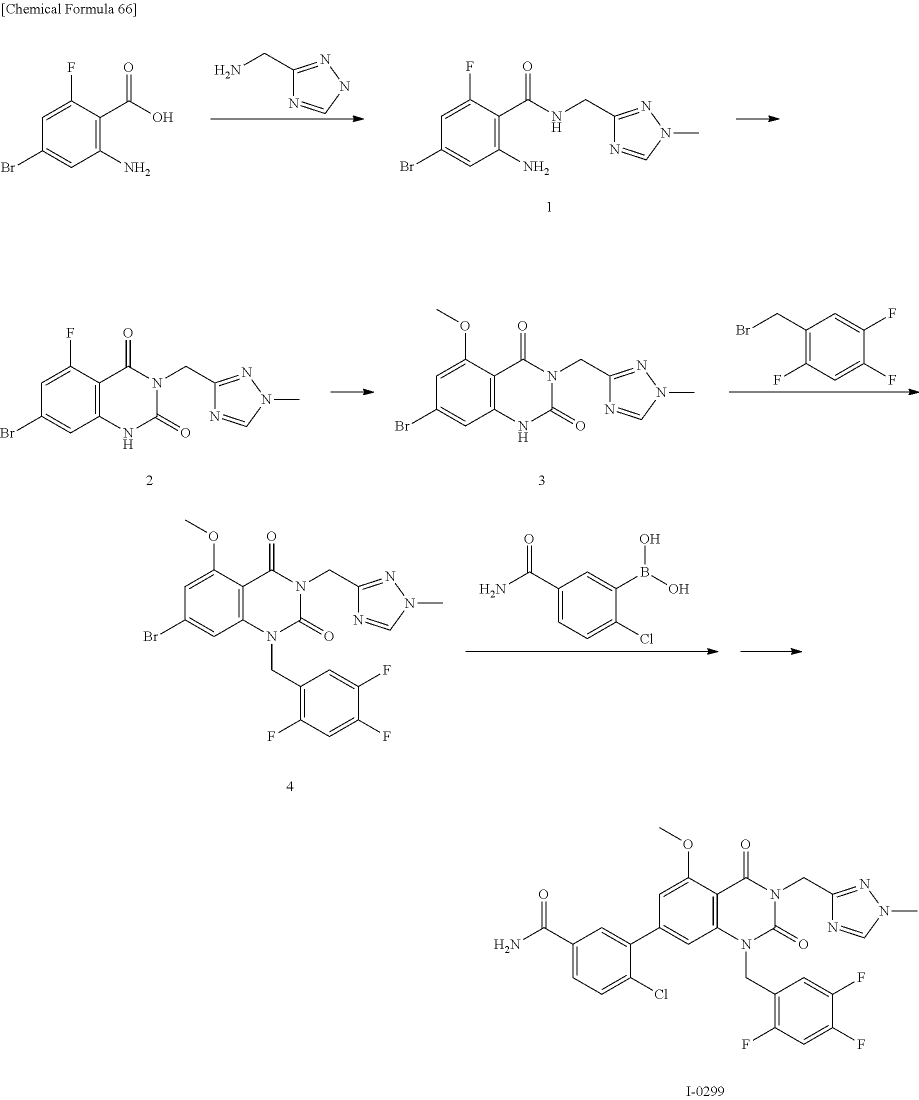

### Step 1Synthesis of Compound 1

2-Amino-4-bromo-6-fluorobenzoic acid (1.00 g, 4.27 mmol) was dissolved in dichloromethane (20 ml), PyBOP (2.67 g, 5.13 mmol) and diisopropylethylamine (2.24 ml, 12.8 mmol) were added thereto, and the mixture was stirred at room temperature for 18 hours. Water (50 ml) was added to the reaction solution, the mixture was separated, and the aqueous layer was extracted with dichloromethane. After the organic layer was washed with water, it was dried over sodium sulfate, and the solvent was distilled off under reduced pressure. The obtained residue was purified by silica gel column chromatography (ethyl acetate/methanol) to give Compound 1 (1.63 g, 4.97 mmol, yield 100%).

LC/MS (ESI): m/z=328, RT=1.41 min, LC/MS measurement conditions 1

### Step 2Synthesis of Compound 2

A mixture of Compound 1 (1.34 g, 4.08 mmol), THF (13 ml), and CDI (1.32 g, 8.17 mmol) was stirred at 70° C. for 4 hours. The reaction solution was cooled to room temperature, water was added thereto, and the precipitated solid was collected by filtration to give Compound 2 (906 mg, 2.56 mmol, yield 63%).

LC/MS (ESI): m/z=354, RT=1.35 min, LC/MS measurement conditions 1

### Step 3Synthesis of Compound 3

Compound 2 (50.0 mg, 0.141 mmol) was dissolved in DMF (1 ml), and a 28% sodium methoxide-methanol solution (0.10³ ml, 0.424 mmol) was added thereto, then the mixture was stirred at 60° C. for 1 hour. The reaction solution was cooled to room temperature, neutralized with a 10% citric acid aqueous solution, and the precipitated solid was collected by filtration to give Compound 3 (51.0 mg, 0.139 mmol, yield 99%).

LC/MS (ESI): m/z=366, RT=1.23 min, LC/MS measurement conditions 1

### Step 4Synthesis of Compound 4

Compound 3 (50.0 mg, 0.137 mmol), DMF (1.0 ml), and potassium carbonate (37.7 g, 0.237 mmol) were mixed, and the mixture was stirred at room temperature for 10 minutes. To the mixture, 1-(bromomethyl)-2,4,5-trifluorobenzene (32.3 mg, 0.143 mmol) was added at 0° C., and the mixture was heated to room temperature and stirred for 2 hours. Water was added to the reaction solution, and the precipitated solid was dissolved in chloroform, washed with water, and then dried over sodium sulfate. The solvent was distilled off under reduced pressure, and the obtained crude product was crystallized from ethyl acetate-diisopropyl ether to give Compound 4 (51 mg, 0.10 mmol, yield 73%).

LC/MS (ESI): m/z=510, RT=2.00 min, LC/MS measurement conditions 1

### Step 5Synthesis of Compound 5

Compound 4 (48.0 mg, 0.0940 mmol), (5-carbamoyl-2-chlorophenyl)boronic acid (24.4 mg, 0.122 mmol), dioxane (0.7 ml), a 2 mol/L aqueous potassium carbonate solution (0.0940 ml, 0.188 mmol), and PdCl₂ (dppf) (6.88 mg, 0.00941 mmol) were mixed, and the mixture was stirred at 90° C. for 4 hours under a nitrogen atmosphere. The reaction solution was ice-cooled, water was added thereto, and was extracted with ethyl acetate. After the organic layer was washed with water, it was dried over sodium sulfate, and the solvent was distilled off under reduced pressure. The obtained residue was purified by silica gel column chromatography (ethyl acetate/methanol) to give Compound (I-0299) (22.0 mg, 0.0380 mmol, yield 40%).

LC/MS (ESI): m/z=585, RT=1.89 min, LC/MS measurement conditions 1

1H-NMR (DMSO-d₆) δ: 8.34 (s, 1H), 8.09 (s, 1H), 7.91 (m, 2H), 7.66 (d, J=8.8 Hz, 1H), 7.63-7.58 (s, 1H), 7.55 (s, 1H), 7.29 (m, 1H), 6.98 (s, 1H), 6.88 (s, 1H), 5.40 (s, 2H), 5.17 (s, 2H), 3.92 (s, 3H), 3.79 (s, 3H).

### Example 2

### Synthesis of Compound (I-0067)

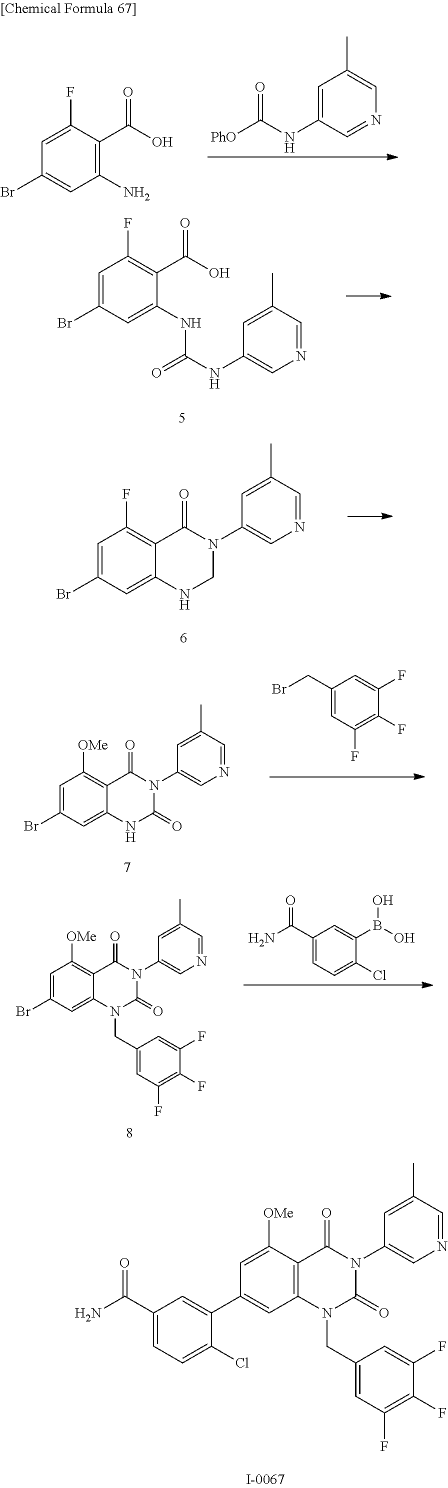

### Step 1Synthesis of Compound 6

2-Amino-4-bromo-6-fluorobenzoic acid (2.00 g, 8.55 mmol) was dissolved in DMA (2.0 ml), phenyl N-(5-methyl-3-pyridyl)carbamate (2.71 g, 10.3 mmol) (see WO2009127948, WO2009127949, and WO2002048152 for the synthesizing method), DMAP (0.104 g, 0.855 mmol), and triethylamine (2.84 ml, 20.5 mmol) were added thereto, and the mixture was stirred at 100° C. for 2 hours. The reaction solution was cooled to room temperature, and water (60 ml) was added thereto, then the mixture was neutralized with 2 mol/L hydrochloric acid. The precipitated solid was collected by filtration and washed with water to give Compound 5 (2.48 g, 6.74 mmol, yield 79%).

LC/MS (ESI): m/z=508, RT=2.17 min, LC/MS measurement conditions 1

### Step 2Synthesis of Compound 6

Compound 5 (2.48 g, 6.74 mmol) was suspended in 2 mol/L hydrochloric acid (25 ml, 50.0 mmol), and the mixture was stirred at 100° C. for 2 hours. The reaction solution was cooled to room temperature and neutralized with saturated aqueous sodium bicarbonate solution. The precipitated solid was collected by filtration and washed with water to give Compound 6 (2.05 g, 5.85 mmol, yield 87%).

LC/MS (ESI): m/z=352, RT=1.45 min, LC/MS measurement conditions 1

### Step 3Synthesis of Compound 7

Compound 7 (79.5 mg, 0.220 mmol, yield 77%) was obtained in the same manner as in Step 3 of Example 1.

LC/MS (ESI): m/z=569, RT=2.07 min, LC/MS measurement conditions 1

### Step 4Synthesis of Compound 8

Compound 8 (99.5 mg, 0.197 mmol, yield 90%) was obtained in the same manner as in Step 4 of Example 1.

LC/MS (ESI): m/z=508, RT=2.17 min, LC/MS measurement conditions 1

### Step 5Synthesis of Compound (I-0067)

Compound (I-0067) (24.0 mg, 0.0410 mmol, yield 42%) was obtained in the same manner as in Step 5 of Example 1.

LC/MS (ESI): m/z=581, RT=1.94 min, LC/MS measurement conditions 1

1H-NMR (DMSO-d₆) δ: 8.46 (d, J=1.4 Hz, 1H), 8.42 (d, J=2.0 Hz, 1H), 8.11 (br s, 1H), 7.90 (dt, J=6.7, 2.2 Hz, 2H), 7.70 (br s, 1H), 7.67-7.65 (m, 1H), 7.57 (br s, 1H), 7.45-7.42 (m, 2H), 6.98 (s, 1H), 6.85 (s, 1H), 5.37 (br s, 2H), 3.91 (s, 3H), 2.38 (s, 3H).

### Example 3

### Synthesis of Compound (I-0176), Compound (I-0177), and Compound (I-0169)

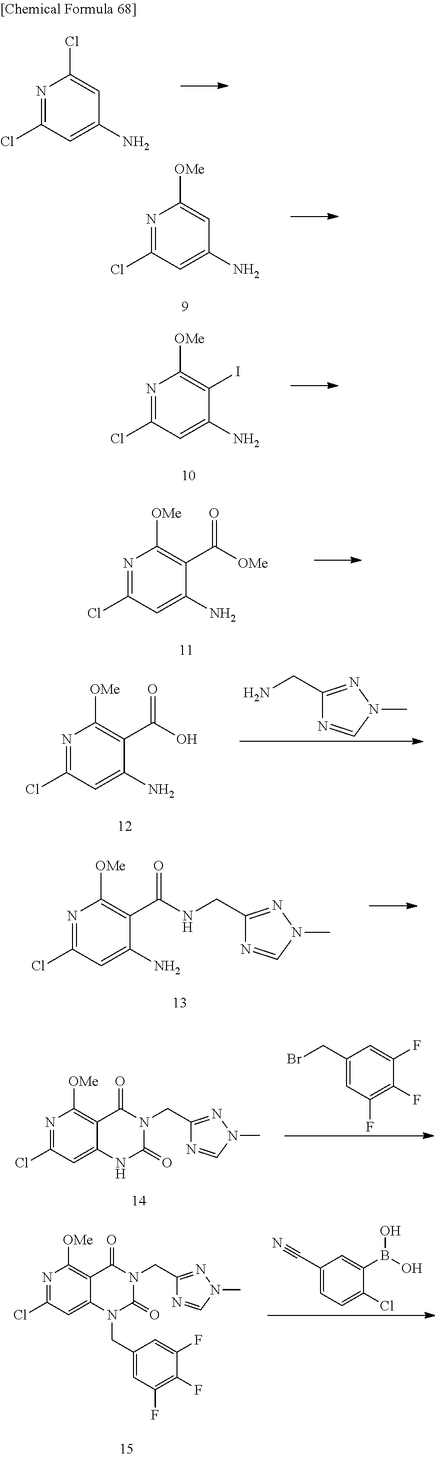

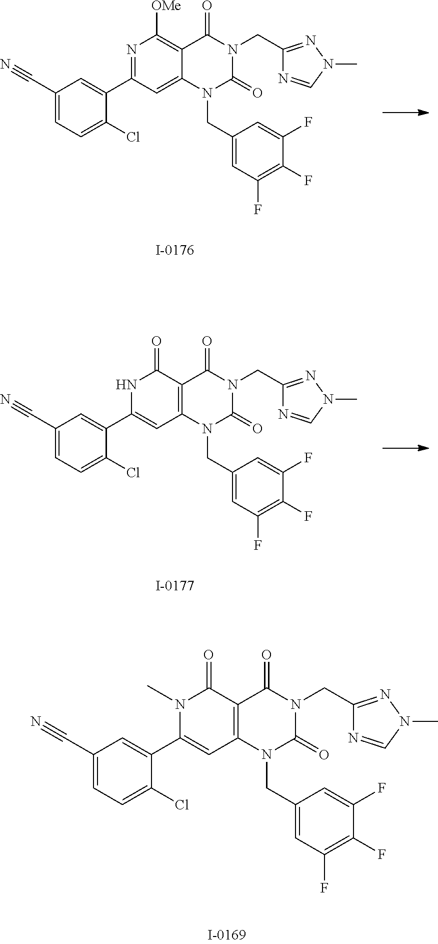 [SMILES: COc1nc(-c2cc(C#N)ccc2Cl)cc2c1c(=O)n(Cc1ncn(C)n1)c(=O)n2Cc1cc(F)c(F)c(F)c1 ; Cn1cnc(Cn2c(=O)c3c(=O)[nH]c(-c4cc(C#N)ccc4Cl)cc3n(Cc3cc(F)c(F)c(F)c3)c2=O)n1 ; Cn1cnc(Cn2c(=O)c3c(=O)n(C)c(-c4cc(C#N)ccc4Cl)cc3n(Cc3cc(F)c(F)c(F)c3)c2=O)n1]

### Step 1Synthesis of Compound 9

Compound 9 (3.52 g, 22.2 mmol, yield 75%) was synthesized by known methods (see WO2017066742 and WO2010093849).

LC/MS (ESI): m/z=159, RT=1.19 min, LC/MS measurement conditions 1

### Step 2Synthesis of Compound 10

Compound 11 (3.32 g, 20.9 mmol) was dissolved in acetic acid (33 ml), and sodium iodide (3.76 g, 25.1 mmol) and chloramine T (7.07 g, 25.1 mmol) were added thereto, then the mixture was stirred at room temperature for 30 minutes. To the reaction solution, a 10 mol/L aqueous sodium hydroxide solution (61.2 ml, 612 mmol), ethyl acetate (30 ml), sodium thiosulfate (3.31 g, 20.9 mmol), and water (30 ml) were added, and the mixture was separated. The water layer was extracted with ethyl acetate, and the organic layer was washed with water and dried over sodium sulfate. The solvent was distilled off under reduced pressure, and the obtained residue was purified by silica gel column chromatography (hexane/ethyl acetate) to give Compound 10 (5.01 g, 17.6 mmol, yield 84%).

LC/MS (ESI): m/z=285, RT=2.02 min, LC/MS measurement conditions 1

### Step 3Synthesis of Compound 11

Compound 10 (4.62 g, 16.2 mmol), methanol (46 ml), triethylamine (4.50 ml, 32.5 mmol), and PdCl₂ (dppf) (1.19 g, 1.62 mmol) were mixed, and the mixture was stirred at 60° C. for 4.5 hours under a carbon monoxide atmosphere. The reaction solution was cooled to room temperature, and the residue obtained by concentration was purified by silica gel column chromatography (hexane/ethyl acetate) to give Compound 11 (3.06 g, 14.1 mmol, yield 87%).

LC/MS (ESI): m/z=217, RT=1.76 min, LC/MS measurement conditions 1

### Step 4Synthesis of Compound 12

Compound 11 (2.86 g, 13.2 mmol) was dissolved in methanol (30 ml) and tetrahydrofuran (15 ml), a 2 mol/L aqueous sodium hydroxide solution (33.0 ml, 66.0 mmol) was added thereto, and then, the mixture was stirred at 60° C. for 45 minutes. After cooled to room temperature, the mixture was neutralized with 2 mol/L hydrochloric acid. The precipitated solid was collected by filtration and washed with water to give Compound 12 (1.47 g, 7.28 mmol, yield 55%).

LC/MS (ESI): m/z=203, RT=1.49 min, LC/MS measurement conditions 1

### Step 5Synthesis of Compound 13

Compound 13 (691 mg, 2.33 mmol, yield 94%) was obtained in the same manner as in Step 1 of Example 1.

LC/MS (ESI): m/z=297, RT=1.34 min, LC/MS measurement conditions 1

### Step 6Synthesis of Compound 14

Compound 13 (610 mg, 2.06 mmol) was added to tetrahydrofuran (60 ml), and 60% sodium hydride (247 mg, 6.17 mmol) and CDI (667 mg, 4.11 mmol) were added at 0° C., then the mixture was stirred at room temperature for 40 minutes. Furthermore, 60% sodium hydride (247 mg, 6.17 mmol) and CDI (667 mg, 4.11 mmol) were added thereto at 0° C., and the mixture was stirred at 70° C. for 30 minutes. Water (30 ml) was added thereto, the mixture was neutralized with 2 mol/L hydrochloric acid, and the precipitated solid was collected by filtration to give Compound 14 (470 mg, 1.46 mmol, yield 71%).

LC/MS (ESI): m/z=323, RT=1.21 min, LC/MS measurement conditions 1

### Step 7Synthesis of Compound 15

Compound 15 (265 mg, 0.568 mmol, yield 92%) was obtained in the same manner as Step 4 of Example 1.

LC/MS (ESI): m/z=467, RT=2.03 min, LC/MS measurement conditions 1

### Step 8Synthesis of Compound (I-0176)

Compound (I-0176) (126 mg, 0.222 mmol, yield 69%) was obtained in the same manner as Step 5 in Example 1.

LC/MS (ESI): m/z=568, RT=2.24 min, LC/MS measurement conditions 1

1H-NMR (DMSO-d6) δ: 8.36 (s, 1H), 8.17 (d, J=1.9 Hz, 1H), 7.98 (dd, J=8.4, 1.9 Hz, 1H), 7.83 (d, J=8.4 Hz), 5.41 (s, 2H), 5.18 (s, 2H), 4.00 (s, 3H), 3.79 (s, 3H).

### Step 9Synthesis of Compound (I-0177)

Compound 18 (111 mg, 0.195 mmol), chlorotrimethylsilane (63.7 mg, 0.586 mmol), sodium iodide (88.0 mg, 0.586 mmol), and acetonitrile (1.1 ml) were mixed, and the mixture was stirred at 70° C. for 30 minutes. The reaction solution was cooled to room temperature, a saturated aqueous sodium bicarbonate solution was added thereto, and the precipitated solid was collected by filtration and washed with water to give Compound 19 (85.7 mg, 0.155 mmol, yield 79%).

LC/MS (ESI): m/z=554, RT=1.87 min, LC/MS measurement conditions 1

1H-NMR (DMSO-d6) δ: 8.37 (s, 1H), 7.92-7.87 (m, 2H), 7.74-7.72 (m, 1H), 7.30-7.26 (m, 2H), 6.13 (br s, 1H), 5.23 (s, 2H), 5.13 (s, 2H), 3.79 (s, 3H).

### Step 10 Synthesis of Compound (I-0169)

Compound (I-0177) (76.4 mg, 0.138 mmol) was dissolved in DMF (1.5 ml), cesium carbonate (225 mg, 0.690 mmol) and iodomethane (98.0 mg, 0.690 mmol) were added thereto, and the mixture was stirred at room temperature for 45 minutes. Water (5 ml) and ethyl acetate (5 ml) were added to the reaction solution, the mixture was separated, and the aqueous layer was extracted with ethyl acetate. The organic layer was washed with water and dried over sodium sulfate. The residue obtained by concentrating the solvent under reduced pressure was purified by silica gel column chromatography (chloroform/methanol) to give Compound (I-0169) (49.0 mg, 0.0860 mmol, yield 63%).

LC/MS (ESI): m/z=568, RT=1.86 min, LC/MS measurement conditions 1

1H-NMR (DMSO-d6) δ: 8.35 (s, 1H), 8.10-8.07 (m, 2H), 7.92 (d, J=8.3 Hz, 1H), 7.27-7.23 (m, 2H), 6.44 (s, 1H), 5.37-5.23 (m, 2H), 5.12 (s, 3H), 3.79 (s, 3H), 3.08 (s, 3H).

### Example 4

### Synthesis of Compound (I-0353)

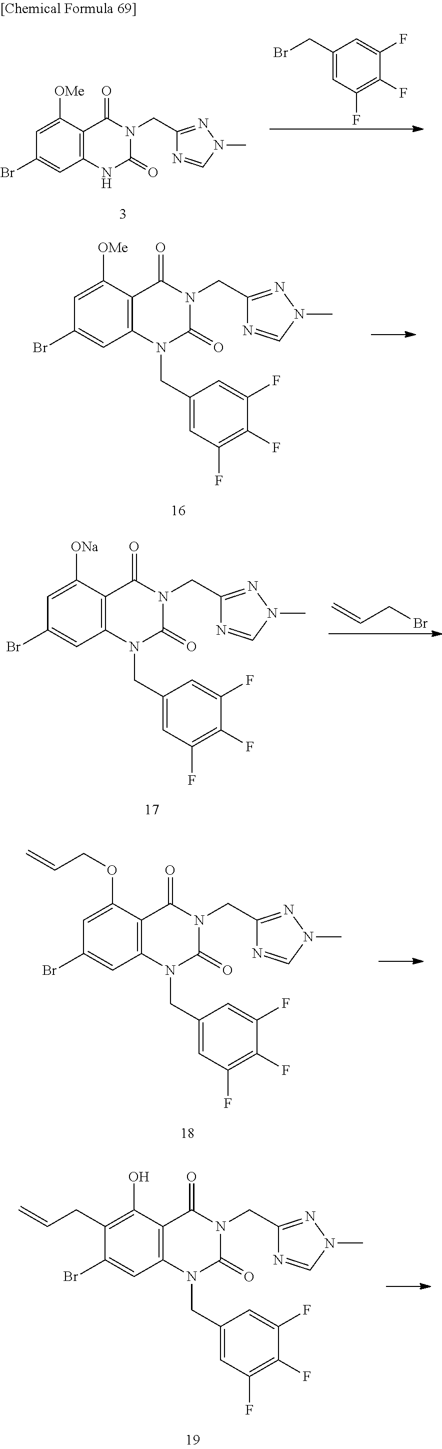

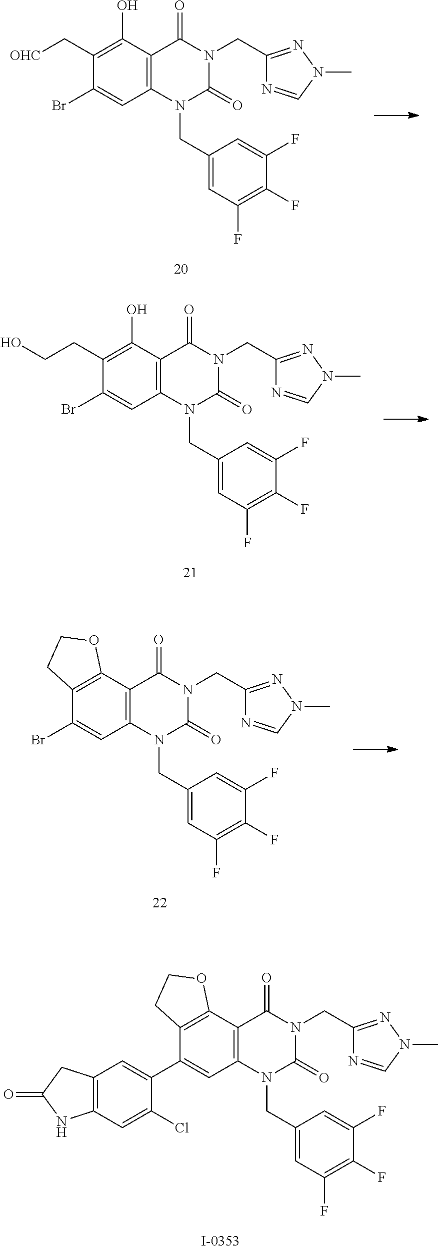 [SMILES: Cn1cnc(Cn2c(=O)c3c(O)c(CC=O)c(Br)cc3n(Cc3cc(F)c(F)c(F)c3)c2=O)n1 ; Cn1cnc(Cn2c(=O)c3c(O)c(CCO)c(Br)cc3n(Cc3cc(F)c(F)c(F)c3)c2=O)n1 ; Cn1cnc(Cn2c(=O)c3c4c(c(Br)cc3n(Cc3cc(F)c(F)c(F)c3)c2=O)CCO4)n1 ; Cn1cnc(Cn2c(=O)c3c4c(c(-c5cc6c(cc5Cl)NC(=O)C6)cc3n(Cc3cc(F)c(F)c(F)c3)c2=O)CCO4)n1]

### Step 1Synthesis of Compound 16

Compound 16 (1.26 g, 2.47 mmol, yield 95%) was obtained in the same manner as Step 4 of Example 1.

LC/MS (ESI): m/z=510, RT=2.05 min, LC/MS measurement conditions 1

### Step 2Synthesis of Compound 17

Dichloromethane (2.0 ml) was added to Compound 16 (200 mg, 0.392 mmol), and a 1.0 mol/L boron tribromide-dichloromethane solution (0.862 ml, 0.862 mmol) was added thereto at 0° C., then the mixture was stirred as it was for 2 hours. Methanol (2.0 ml) and a 2.0 mol/L aqueous sodium hydroxide solution (1.5 ml) were added to the reaction solution, and the mixture was stirred at 50° C. for 1 hour. The reaction solution was cooled to room temperature, and the precipitated solid was collected by filtration and washed with water to give Compound 17 (181 mg, 0.349 mmol, yield 89%).

### Step 3Synthesis of Compound 18

Compound 17 (192 mg, 0.370 mmol) was dissolved in DMF (1.0 ml), and potassium carbonate (51.1 mg, 0.370 mmol) and allyl bromide (0.0680 ml, 0.740 mmol) were added thereto, then the mixture was stirred at 60° C. for 5 hours. The reaction solution was cooled to room temperature, water was added thereto, and the precipitated solid was collected by filtration to give Compound 18 (175 mg, 0.326 mmol, yield 88%).

### Step 4Synthesis of Compound 19

The compound 18 (175 mg, 0.326 mmol) was dissolved in NMP (1.3 mL), and the mixture was stirred at 240° C. for 1 hour. The reaction solution was cooled to room temperature, water was added thereto, and then the mixture was extracted with ethyl acetate. The organic layer was washed with water and dried over sodium sulfate, and then the solvent was distilled off under reduced pressure to give Compound 19 (175 mg, 0.326 mmol, yield 100%).

LC/MS (ESI): m/z=536, RT=2.77 min, LC/MS measurement conditions 1

### Step 5Synthesis of Compound 20

Compound 19 (175 mg, 0.326 mmol) was dissolved in tetrahydrofuran (1.5 ml) and water (0.5 ml), and 2,6-lutidine (0.0760 ml, 0.653 mmol), sodium periodate (209 mg, 0.979 mmol), and potassium osmate (VI) dihydrate (3.61 mg, 0.00979 mmol) were added thereto, then the mixture was stirred at room temperature for 1.5 hours. Water (3 ml) and ethyl acetate (5 ml) were added to the reaction solution, the mixture was separated, and the aqueous layer was extracted with ethyl acetate. The organic layer was washed with water and dried over sodium sulfate. The residue obtained by concentrating the solvent was purified by silica gel column chromatography (chloroform/methanol) to give Compound 20 (116 mg, 0.216 mmol, yield 66%).

LC/MS (ESI): m/z=538, RT=2.19 min, LC/MS measurement conditions 1

### Step 6Synthesis of Compound 21

Compound 20 (116 mg, 0.216 mmol) was dissolved in methanol (1.2 ml), and sodium borohydride (8.15 mg, 0.216 mmol) was added thereto, then the mixture was stirred for 2 hours. A saturated aqueous ammonium chloride solution was added to the reaction solution, and the precipitated solid was collected by filtration to obtain Compound 21 (85.3 mg, 0.158 mmol, yield 73%).

LC/MS (ESI): m/z=540, RT=2.41 min, LC/MS measurement conditions 1

### Step 7Synthesis of Compound 22

Triphenylphosphine (49.7 mg, 0.189 mmol) and diisopropyl azodicarboxylate (47.9 mg, 0.237 mmol) were added to a tetrahydrofuran solution (1.0 ml) of Compound 21 (85.3 mg, 0.158 mmol) at 0° C., and the mixture was stirred at room temperature for 2.5 hours. The reaction solution was concentrated, and the crude product was washed with methanol to obtain Compound 22 (33.2 mg, 0.0640 mmol, yield 40%).

LC/MS (ESI): m/z=522, RT=2.07 min, LC/MS measurement conditions 1

### Step 8Synthesis of Compound (I-0353)

Compound (I-0353) (25.4 mg, 0.0420 mmol, yield 66%) was obtained in the same manner as Step 5 of Example 1, using Compound 22 (33.2 mg, 0.0640 mmol).

LC/MS (ESI): m/z=609, RT=1.83 min, LC/MS measurement conditions 1

1H-NMR (DMSO-d₆) δ: 10.62 (s, 1H), 8.35 (s, 1H), 7.28 (m, 2H), 7.20 (s, 1H), 6.93 (s, 1H), 6.57 (s, 1H), 5.34 (br s, 2H), 5.17 (s, 2H), 4.70 (t, J=8.4 Hz, 2H), 3.79 (s, 3H), 3.51 (s, 2H), 2.98 (br t, J=8.4 Hz, 2H).

### Example 5

### Synthesis of Compound (I-0325)

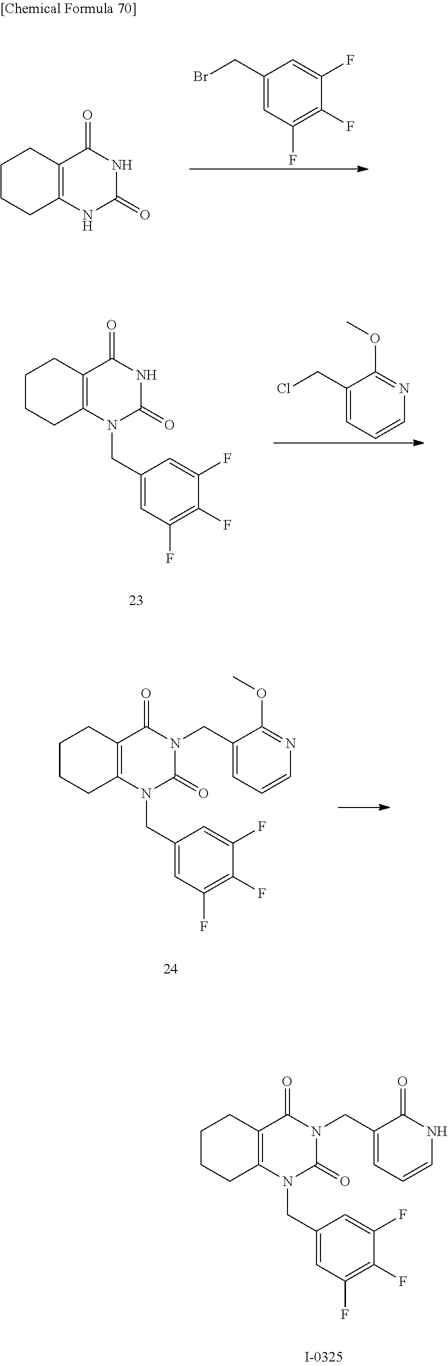

### Step 1Synthesis of Compound 23

A mixture of 5,6,7,8-tetrahydroquinazoline-2,4(1H,3H)-dione (200 mg, 1.20 mmol), acetonitrile (2.0 ml) and N,O-bis(trimethylsilyl)acetamide (367 mg, 1.81 mmol) was stirred at room temperature for 40 minutes. To the reaction solution, 1-(bromomethyl)-3,4,5-trifluorobenzene (406 mg, 1.81 mmol) was added, and the mixture was stirred at 80° C. for 18 hours. Water was added to the reaction solution, the mixture was extracted with chloroform. The organic layer was washed with water and dried over sodium sulfate. The crude product obtained by concentrating the solvent was solidified with dichloromethane-diisopropyl ether to give Compound 23 (283 mg, 0.912 mmol, yield 76%).

LC/MS (ESI): m/z=311, RT=1.90 min, LC/MS measurement conditions 1

### Step 2Synthesis of Compound 24

Compound 23 (50.0 mg, 0.161 mmol) was dissolved in DMF (0.75 ml), and potassium carbonate (44.5 mg, 0.322 mmol) and 3-(chloromethyl)-2-methoxypyridine (33.0 mg, 0.209 mmol) were added, then the mixture was stirred at room temperature for 2 hours. Water was added to the reaction solution, and the mixture was extracted with ethyl acetate. The organic layer was washed with water and dried over sodium sulfate. The residue obtained by concentrating the solvent was purified by silica gel column chromatography (chloroform/methanol) to give Compound 24 (58.0 mg, 0.134 mmol, yield 83%).

LC/MS (ESI): m/z=432, RT=2.45 min, LC/MS measurement conditions 1

### Step 3Synthesis of Compound (I-0325)

Compound 24 (55.0 mg, 0.127 mmol), chlorotrimethylsilane (41.6 mg, 0.382 mmol), sodium iodide (57.3 mg, 0.382 mmol), and acetonitrile (2.2 ml) were mixed, and the mixture was stirred at 70° C. for 2 hours. The reaction solution was cooled to room temperature, an aqueous sodium thiosulfate solution was added thereto, and the mixture was extracted with ethyl acetate. The organic layer was dried over sodium sulfate, and the residue obtained by concentrating the solvent was purified by silica gel column chromatography (ethyl acetate/methanol) to give Compound (I-0325) (12.0 mg, 0.0290 mmol, yield 23%).

LC/MS (ESI): m/z=418, RT=1.84 min, LC/MS measurement conditions 1

1H-NMR (CDCl₃) δ: 11.41 (s, 1H), 7.26-7.19 (m, 2H), 6.83 (t, J=6.9 Hz, 2H), 6.20 (t, J=6.7 Hz, 1H), 5.13 (s, 2H), 5.06 (s, 2H), 2.43 (m, 4H), 1.76 (m, 2H), 1.66 (m, 2H).

### Example 6

### Synthesis of Compound (I-0140)

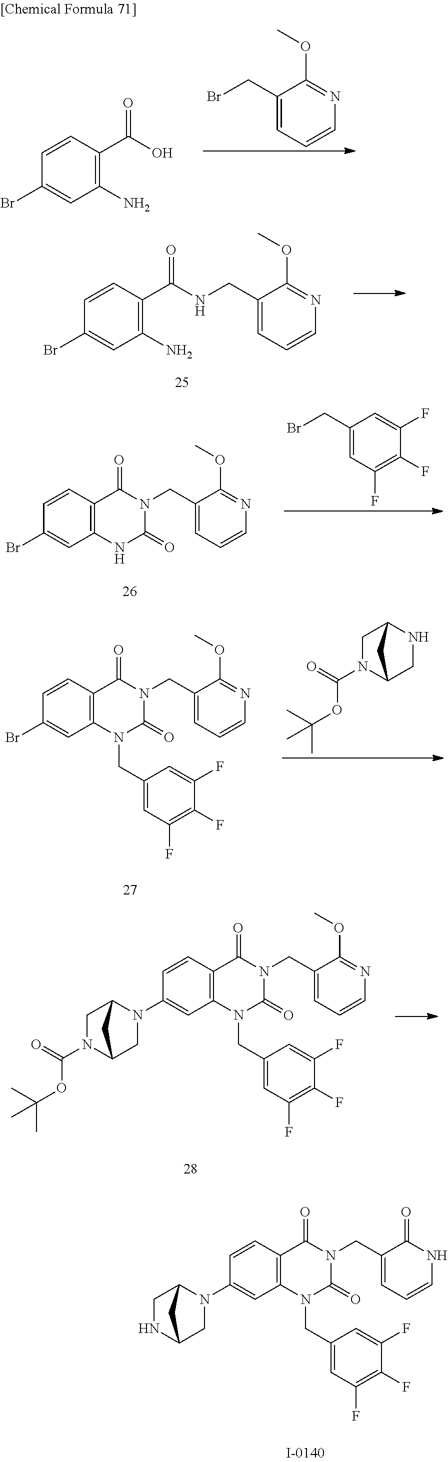

### Step 1Synthesis of Compound 25

Compound 25 (24.0 g, 71.4 mmol, yield 77%) was obtained in the same manner as Step 1 of Example 1.

### Step 2Synthesis of Compound 26

Compound 26 (20.0 g, 55.2 mmol, yield 77%) was obtained in the same manner as Step 2 of Example 1.

### Step 3Synthesis of Compound 27

Compound 27 (19.4 g, 38.1 mmol, yield 93%) was obtained in the same manner as Step 4 of Example 1.

### Step 4Synthesis of Compound 28

Compound 27 (30.0 mg, 0.0590 mmol), 2,5-diazabicyclo[2.2.1]heptane-2-carboxylic acid (1R,4R)-TERT-butyl hydrochloride (24.0 mg, 0.119 mmol), DMF (0.30 ml), cesium carbonate (57.9 mg, 0.178 mmol), and RuPhos Pd G3 (4.96 mg, 0.00593 mmol) were mixed, and the mixture was stirred at 100° C. for 18 hours. The reaction solution was cooled to room temperature, chloroform and water were added, and the mixture was separated. The aqueous layer was extracted with chloroform, and the combined organic layers were concentrated. The obtained residue was purified by PLCMS to obtain Compound 28 (34.4 mg, 0.0552 mmol, yield 94%).

LC/MS (ESI): m/z=624, RT=2.79 min, LC/MS measurement conditions 1

### Step 4Synthesis of Compound (I-0140)

Compound (I-0140) (8.10 mg, 0.0159 mmol, 29% yield) was obtained in the same manner as Step 3 in Example 5.

LC/MS (ESI): m/z=512, RT=1.41 min, LC/MS measurement conditions 1

1H-NMR (CDCl₃) δ: 11.6 (s, 1H), 8.62 (s, 1H), 7.86 (d, J=8.8 Hz, 1H), 7.38 (d, J=8.4 Hz, 1H), 7.36 (d, J=8.4 Hz, 1H), 7.28 (d, J=6.4 Hz, 1H), 6.99 (d, J=4.8 Hz, 1H), 6.62 (d, J=8.4 Hz, 1H), 6.16 (s, 1H), 6.10 (dd, J=6.8 Hz, 1H), 5.41-5.31 (m, 2H), 4.90 (s, 2H), 4.78 (s, 1H), 4.50 (s, 1H), 3.63 (d, J=9.2 Hz, 1H), 3.27-3.16 (m, 4H), 2.97 (d, J=10.4 Hz, 1H), 2.15 (d, J=11.2 Hz, 1H), 1.95 (d, J=10.8 Hz, 1H).

### Example 7

### Synthesis of Compound (I-0978) and Compound (I-1065)

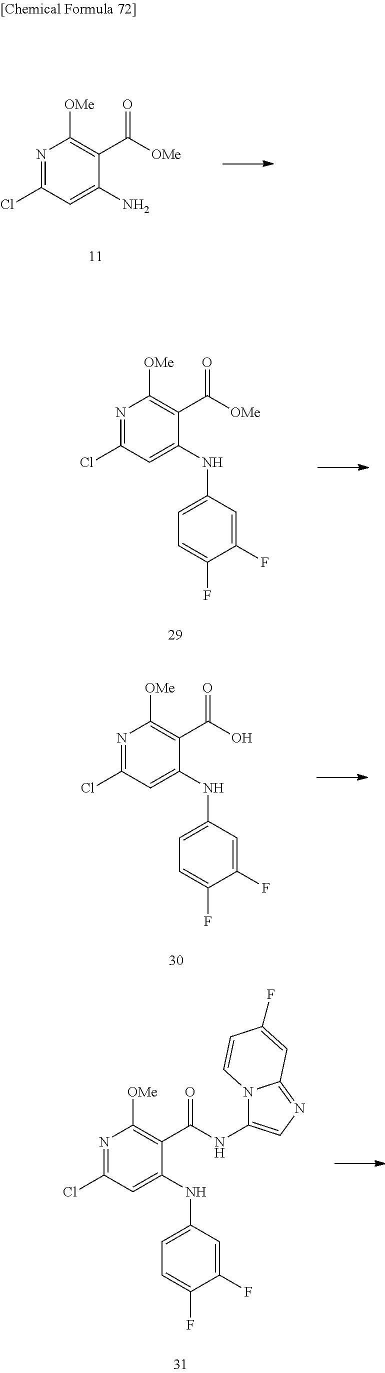 [SMILES: COC(=O)c1c(N)cc(Cl)nc1OC ; COC(=O)c1c(Nc2ccc(F)c(F)c2)cc(Cl)nc1OC ; COc1nc(Cl)cc(Nc2ccc(F)c(F)c2)c1C(=O)O ; COc1nc(Cl)cc(Nc2ccc(F)c(F)c2)c1C(=O)Nc1cnc2cc(F)ccn12]

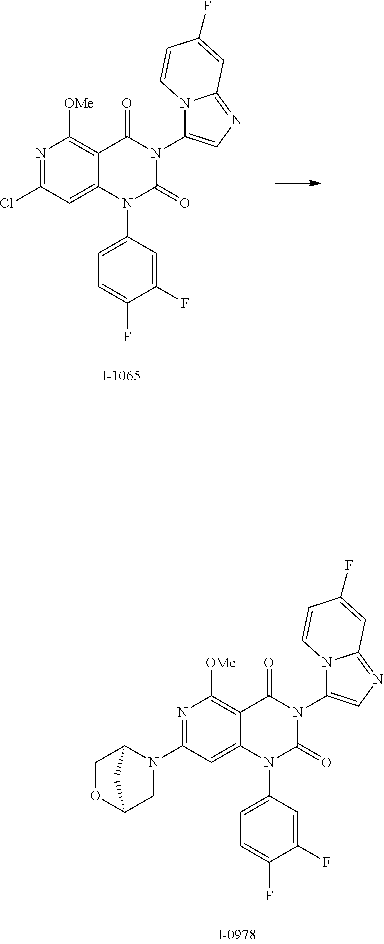 [SMILES: COc1nc(Cl)cc2c1c(=O)n(-c1cnc3cc(F)ccn13)c(=O)n2-c1ccc(F)c(F)c1 ; COc1nc(N2C[C@@H]3C[C@H]2CO3)cc2c1c(=O)n(-c1cnc3cc(F)ccn13)c(=O)n2-c1ccc(F)c(F)c1]

### Step 1Synthesis of Compound 29

Compound 11 (5.00 g, 23.1 mmol) was dissolved in 1,4 dioxane (25 mL), and potassium phosphate (14.7 g, 69.2 mmol), 1,2-difluoro-4-iodobenzene (8.31 g, 34.6 mmol), trans-N,N-dimethylcyclohexane-1,2-diamine (1.31 g, 9.23 mmol), and copper (I) iodide (1.76 g, 9.23 mmol) were added thereto. The reaction solution was stirred at 110° C. for 7 hours under a nitrogen atmosphere. The reaction solution was cooled, diluted by adding chloroform, and then filtered through Celite (registered trademark). The filtrate was concentrated and the obtained residue was purified by silica gel column chromatography (n-hexane/ethyl acetate) to give Compound 29 (3.04 g, 9.25 mol, yield 40%).

LC/MS (ESI): m/z=329, RT=2.74 min, LC/MS measurement conditions 1

### Step 2Synthesis of Compound 30

Compound 29 (3.04 g, 9.25 mmol) was dissolved in methanol (15 mL) and tetrahydrofuran (15 mL), and a 2 mol/L aqueous sodium hydroxide solution (23.1 mL, 46.2 mmol) was added, then the mixture was stirred at 60° C. for 1 hour. After cooled to room temperature, the mixture was neutralized with 2 mol/L hydrochloric acid. The precipitated solid was collected by filtration and washed with water to give Compound 30 (2.53 g, 8.04 mmol, yield 87%).

LC/MS (ESI): m/z=315, RT=2.42 min, LC/MS measurement conditions 1

### Step 3Synthesis of Compound 31

Compound 30 (1.10 g, 3.50 mmol) was dissolved in DMF (11 mL), and 7-fluoroimidazo[1,2-a]pyridine-3-amine (634 mg, 4.19 mmol), PyBOP (2.37 g, 4.54 mmol), and diisopropylethylamine (1.53 mL, 8.74 mmol) were added, then the mixture was stirred at room temperature for 3 hours. Water (50 mL) was added to the reaction solution, the mixture was separated, and the aqueous layer was extracted with ethyl acetate. After the organic layer was washed with water, it was dried over sodium sulfate, and the solvent was distilled off under reduced pressure. The obtained residue was purified by silica gel column chromatography (ethyl acetate/methanol) to give Compound 31 (1.20 g, 2.68 mmol, yield 77%). LC/MS (ESI): m/z=448, RT=2.23 min, LC/MS measurement conditions 1

### Step 4Synthesis of Compound (I-1065)

Compound 31 (1.20 g, 2.68 mmol), 1,3,5-trifluoro-2-nitrobenzene (949 mg, 5.36 mmol), and CDI (1.74 g, 10.7 mmol) were dissolved in DMA (24 mL), and 60% sodium hydride (322 mg, 8.04 mmol) was added thereto at 0° C., then the mixture was stirred at the same temperature for 30 minutes. The reaction solution was added to a mixed liquid of water (50 mL) and 2 mol/L hydrochloric acid (2 mL), and the mixture was extracted with ethyl acetate. After the organic layer was washed with water, it was dried over sodium sulfate, and the solvent was distilled off under reduced pressure. The obtained residue was purified by amino column chromatography (n-hexane/ethyl acetate) to give Compound (I-1065) (283 mg, 0.597 mmol, yield 22%).

LC/MS (ESI): m/z=474, RT=1.91 min, LC/MS measurement conditions 1

### Step 5Synthesis of Compound (I-0978)

Compound (I-1065) (47.0 mg, 0.0990 mmol), (1S,4S)-2-oxa-5-azabicyclo[2,2,1]heptane hydrochloride (14.8 mg, 0.109 mmol) and diisopropylethylamine (0.0518 mL, 0.298 mmol) were dissolved in DMA (0.47 mL), and the mixture was stirred at 110° C. for 2 hours. Water (20 mL) was added to the reaction solution, and the mixture was extracted with ethyl acetate. After the organic layer was washed with water, it was dried over sodium sulfate, and the solvent was distilled off under reduced pressure. The obtained residue was purified by silica gel column chromatography (chloroform/methanol) to give Compound (I-0978) (27.0 mg, 0.0500 mmol, yield 51%).

LC/MS (ESI): m/z=537, RT=1.75 min, LC/MS measurement conditions 1

1H-NMR (CDCl₃) δ: 7.69 (m, 1H), 7.64 (d, J=2.5 Hz, 1H), 7.43-7.12 (m, 511), 6.72 (m, 1H), 4.80 (m, 1H), 4.69 (s, 1H), 4.03 (s, 311), 3.90-3.80 (m, 211), 3.28-3.04 (m, 211), 2.02-1.88 (m, 2H).

### Example 8

### Synthesis of Compound (I-0957)

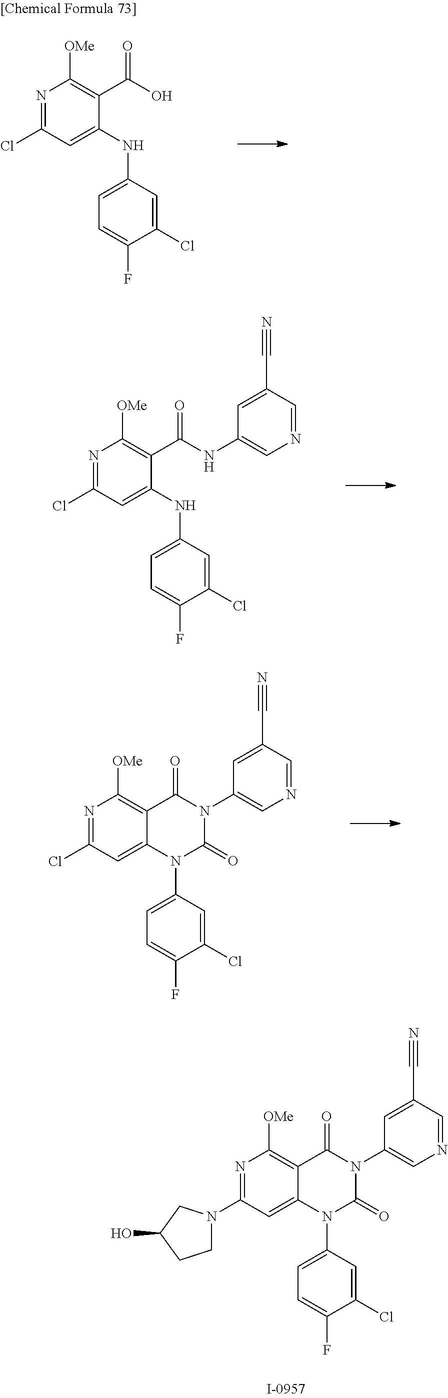 [SMILES: COc1nc(Cl)cc(Nc2ccc(F)c(Cl)c2)c1C(=O)O ; COc1nc(Cl)cc(Nc2ccc(F)c(Cl)c2)c1C(=O)Nc1cncc(C#N)c1 ; COc1nc(Cl)cc2c1c(=O)n(-c1cncc(C#N)c1)c(=O)n2-c1ccc(F)c(Cl)c1 ; COc1nc(N2CC[C@@H](O)C2)cc2c1c(=O)n(-c1cncc(C#N)c1)c(=O)n2-c1ccc(F)c(Cl)c1]

Compound (I-0957) was synthesized in the same manner as in Example 7.

LC/MS (ESI): m/z=509, RT=1.96 min, LC/MS measurement conditions 1

¹H-NMR (CDCl₃) δ: 8.86 (d, J=1.9 Hz, 1H), 8.77 (d, J=2.3 Hz, 1H), 7.97 (t, J=2.1 Hz, 1H), 7.46 (d, J=6.3 Hz, 1H), 7.35 (t, J=8.5 Hz, 1H), 7.26 (m, 2H), 4.84 (br s, 1H), 4.60 (br s, 1H), 4.05 (s, 3H), 3.91-3.00 (m, 4H), 2.08 (m, 2H).

The following compounds were synthesized according to the above general synthesis method and the method described in Examples. The structure and physical properties (LC/MS data) are shown in the tables below.

In the structural formula, the “wedge shape” and “dashed line” indicate the steric configuration.

TABLE 1

| | | LC/MS | Retention | |
|---|---|---|---|---|
| Compound | | measurement | time | |
| No. | Structural formula | conditions | (min) | m/z |
| I-0001 | 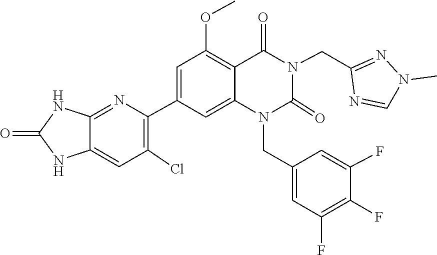 [SMILES: COc1cc(-c2nc3[nH]c(=O)[nH]c3cc2Cl)cc2c1c(=O)n(Cc1ncn(C)n1)c(=O)n2Cc1cc(F)c(F)c(F)c1] | 1 | 1.70 | 599 |
| I-0002 | 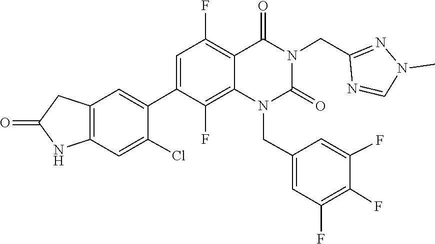 [SMILES: Cn1cnc(Cn2c(=O)c3c(F)cc(-c4cc5c(cc4Cl)NC(=O)C5)c(F)c3n(Cc3cc(F)c(F)c(F)c3)c2=O)n1] | 1 | 1.91 | 603 |
| I-0003 | 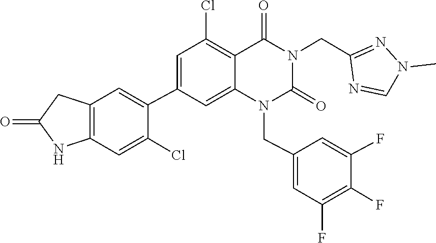 [SMILES: Cn1cnc(Cn2c(=O)c3c(Cl)cc(-c4cc5c(cc4Cl)NC(=O)C5)cc3n(Cc3cc(F)c(F)c(F)c3)c2=O)n1] | 1 | 1.99 | 601 |
| I-0004 | 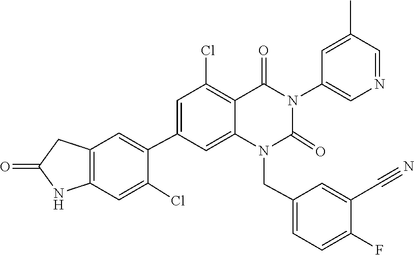 [SMILES: Cc1cncc(-n2c(=O)c3c(Cl)cc(-c4cc5c(cc4Cl)NC(=O)C5)cc3n(Cc3ccc(F)c(C#N)c3)c2=O)c1] | 1 | 2.01 | 586 |
| I-0005 | 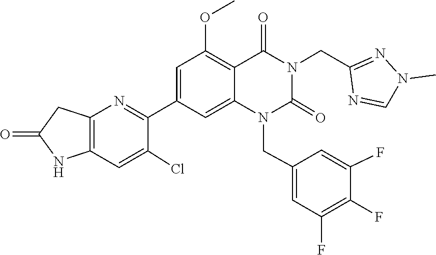 [SMILES: COc1cc(-c2nc3c(cc2Cl)NC(=O)C3)cc2c1c(=O)n(Cc1ncn(C)n1)c(=O)n2Cc1cc(F)c(F)c(F)c1] | 1 | 1.67 | 598 |
| I-0006 | 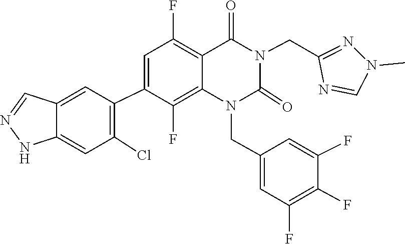 [SMILES: Cn1cnc(Cn2c(=O)c3c(F)cc(-c4cc5cn[nH]c5cc4Cl)c(F)c3n(Cc3cc(F)c(F)c(F)c3)c2=O)n1] | 1 | 2.05 | 588 |

TABLE 2

| | | LC/MS | Retention | |
|---|---|---|---|---|
| Compound | | measurement | time | |
| No. | Structural formula | conditions | (min) | m/z |
| I-0007 | 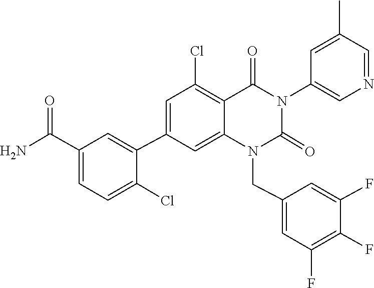 [SMILES: Cc1cncc(-n2c(=O)c3c(Cl)cc(-c4cc(C(N)=O)ccc4Cl)cc3n(Cc3cc(F)c(F)c(F)c3)c2=O)c1] | 1 | 2.13 | 585 |
| I-0008 | 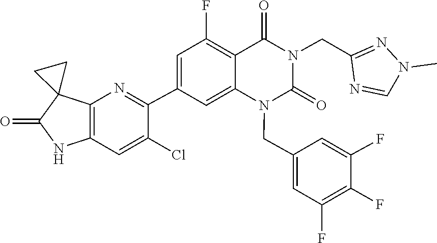 [SMILES: Cn1cnc(Cn2c(=O)c3c(F)cc(-c4nc5c(cc4Cl)NC(=O)C54CC4)cc3n(Cc3cc(F)c(F)c(F)c3)c2=O)n1] | 1 | 1.99 | 612 |
| I-0009 | 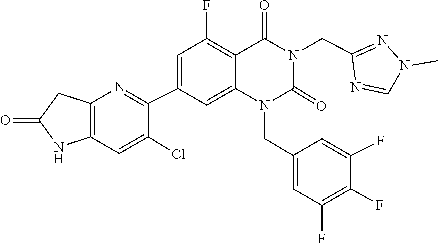 [SMILES: Cn1cnc(Cn2c(=O)c3c(F)cc(-c4nc5c(cc4Cl)NC(=O)C5)cc3n(Cc3cc(F)c(F)c(F)c3)c2=O)n1] | 1 | 1.81 | 586 |
| I-0010 | 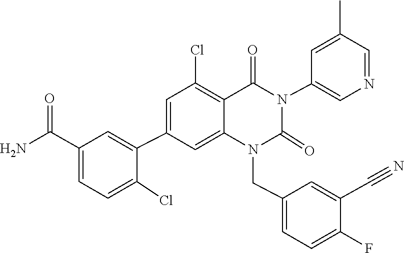 [SMILES: Cc1cncc(-n2c(=O)c3c(Cl)cc(-c4cc(C(N)=O)ccc4Cl)cc3n(Cc3ccc(F)c(C#N)c3)c2=O)c1] | 1 | 2.00 | 574 |
| I-0011 | 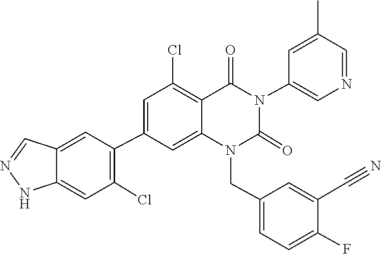 [SMILES: Cc1cncc(-n2c(=O)c3c(Cl)cc(-c4cc5cn[nH]c5cc4Cl)cc3n(Cc3ccc(F)c(C#N)c3)c2=O)c1] | 1 | 2.11 | 571 |
| I-0012 | 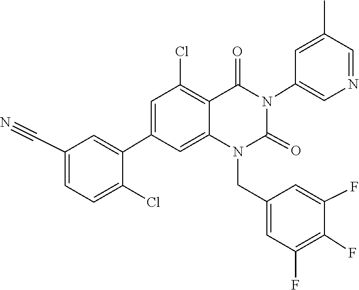 [SMILES: Cc1cncc(-n2c(=O)c3c(Cl)cc(-c4cc(C#N)ccc4Cl)cc3n(Cc3cc(F)c(F)c(F)c3)c2=O)c1] | 1 | 2.56 | 567 |

TABLE 3

| | | LC/MS | Retention | |
|---|---|---|---|---|
| Compound | | measurement | time | |
| No. | Structural formula | conditions | (min) | m/z |
| I-0013 | 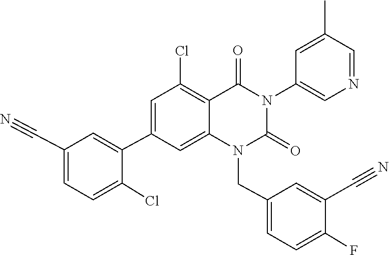 [SMILES: Cc1cncc(-n2c(=O)c3c(Cl)cc(-c4cc(C#N)ccc4Cl)cc3n(Cc3ccc(F)c(C#N)c3)c2=O)c1] | 1 | 2.38 | 556 |
| I-0014 | 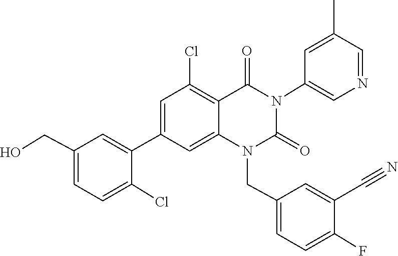 [SMILES: Cc1cncc(-n2c(=O)c3c(Cl)cc(-c4cc(CO)ccc4Cl)cc3n(Cc3ccc(F)c(C#N)c3)c2=O)c1] | 1 | 2.15 | 561.05 |
| I-0015 | 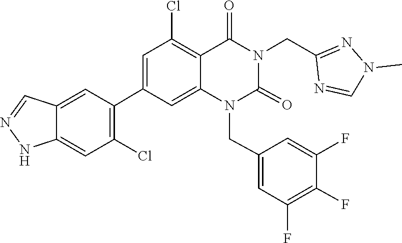 [SMILES: Cn1cnc(Cn2c(=O)c3c(Cl)cc(-c4cc5cn[nH]c5cc4Cl)cc3n(Cc3cc(F)c(F)c(F)c3)c2=O)n1] | 1 | 2.12 | 586 |
| I-0016 | 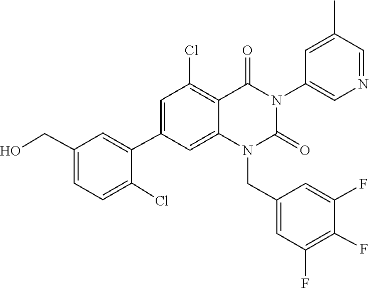 [SMILES: Cc1cncc(-n2c(=O)c3c(Cl)cc(-c4cc(CO)ccc4Cl)cc3n(Cc3cc(F)c(F)c(F)c3)c2=O)c1] | 1 | 2.33 | 573.05 |
| I-0026 | 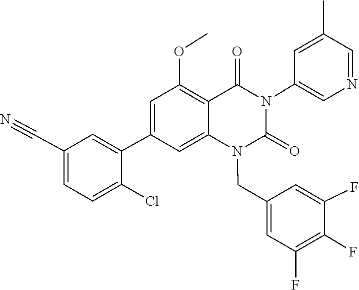 [SMILES: COc1cc(-c2cc(C#N)ccc2Cl)cc2c1c(=O)n(-c1cncc(C)c1)c(=O)n2Cc1cc(F)c(F)c(F)c1] | 1 | 2.32 | 563.1 |
| I-0027 | 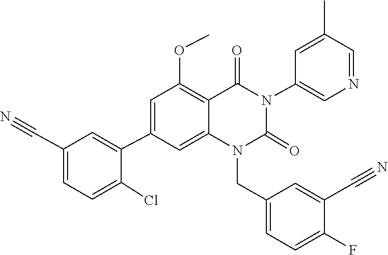 [SMILES: COc1cc(-c2cc(C#N)ccc2Cl)cc2c1c(=O)n(-c1cncc(C)c1)c(=O)n2Cc1ccc(F)c(C#N)c1] | 1 | 2.12 | 552.05 |

TABLE 4

| | | LC/MS | | |
|---|---|---|---|---|
| Compound | | measurement | Retention time | |
| No. | Structural formula | conditions | (min) | m/z |
| I-0028 | 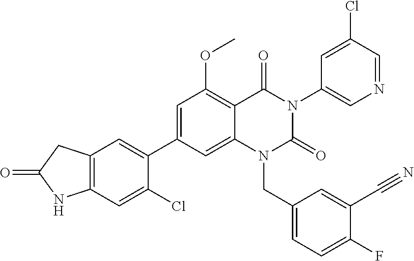 [SMILES: COc1cc(-c2cc3c(cc2Cl)NC(=O)C3)cc2c1c(=O)n(-c1cncc(Cl)c1)c(=O)n2Cc1ccc(F)c(C#N)c1] | 1 | 2.06 | 602.05 |
| I-0029 | 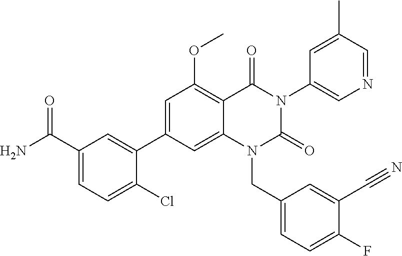 [SMILES: COc1cc(-c2cc(C(N)=O)ccc2Cl)cc2c1c(=O)n(-c1cncc(C)c1)c(=O)n2Cc1ccc(F)c(C#N)c1] | 1 | 1.78 | 570.1 |
| I-0030 | 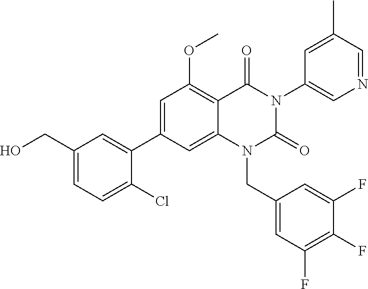 [SMILES: COc1cc(-c2cc(CO)ccc2Cl)cc2c1c(=O)n(-c1cncc(C)c1)c(=O)n2Cc1cc(F)c(F)c(F)c1] | 1 | 2.09 | 568.1 |
| I-0031 | 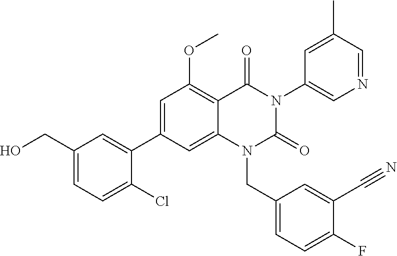 [SMILES: COc1cc(-c2cc(CO)ccc2Cl)cc2c1c(=O)n(-c1cncc(C)c1)c(=O)n2Cc1ccc(F)c(C#N)c1] | 1 | 1.90 | 557.05 |
| I-0032 | 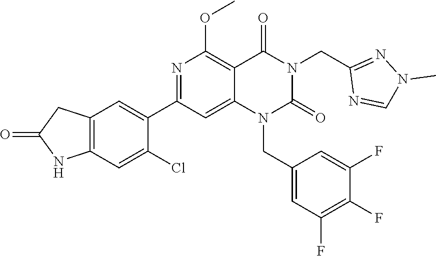 [SMILES: COc1nc(-c2cc3c(cc2Cl)NC(=O)C3)cc2c1c(=O)n(Cc1ncn(C)n1)c(=O)n2Cc1cc(F)c(F)c(F)c1] | 1 | 1.86 | 598.1 |
| I-0033 | 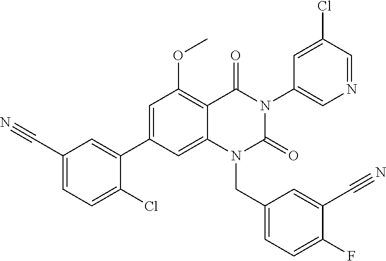 [SMILES: COc1cc(-c2cc(C#N)ccc2Cl)cc2c1c(=O)n(-c1cncc(Cl)c1)c(=O)n2Cc1ccc(F)c(C#N)c1] | 1 | 2.39 | 572 |

TABLE 5

| | | LC/MS | | |
|---|---|---|---|---|
| Compound | | measurement | Retention time | |
| No. | Structural formula | conditions | (min) | m/z |
| I-0034 | 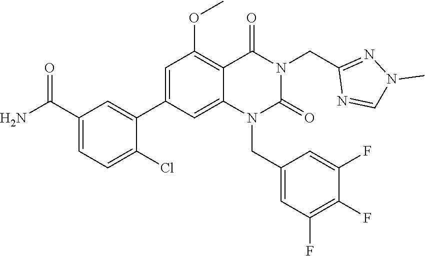 [SMILES: COc1cc(-c2cc(C(N)=O)ccc2Cl)cc2c1c(=O)n(Cc1ncn(C)n1)c(=O)n2Cc1cc(F)c(F)c(F)c1] | 1 | 1.86 | 585.05 |
| I-0035 | 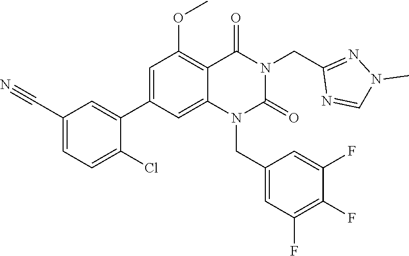 [SMILES: COc1cc(-c2cc(C#N)ccc2Cl)cc2c1c(=O)n(Cc1ncn(C)n1)c(=O)n2Cc1cc(F)c(F)c(F)c1] | 1 | 2.18 | 567.1 |
| I-0036 | 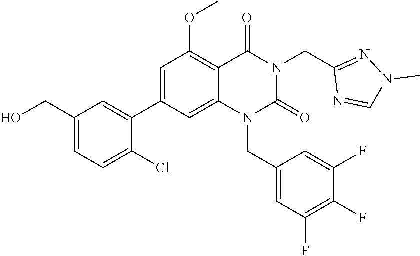 [SMILES: COc1cc(-c2cc(CO)ccc2Cl)cc2c1c(=O)n(Cc1ncn(C)n1)c(=O)n2Cc1cc(F)c(F)c(F)c1] | 1 | 1.94 | 572.1 |
| I-0047 | 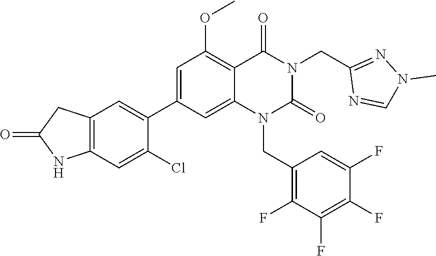 [SMILES: COc1cc(-c2cc3c(cc2Cl)NC(=O)C3)cc2c1c(=O)n(Cc1ncn(C)n1)c(=O)n2Cc1cc(F)c(F)c(F)c1F] | 1 | 1.82 | 615.05 |
| I-0048 | 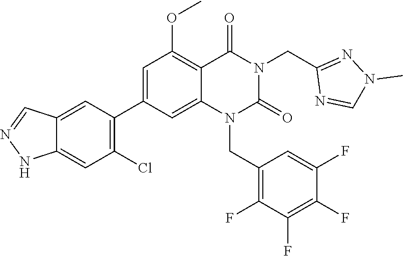 [SMILES: COc1cc(-c2cc3cn[nH]c3cc2Cl)cc2c1c(=O)n(Cc1ncn(C)n1)c(=O)n2Cc1cc(F)c(F)c(F)c1F] | 1 | 1.92 | 600.05 |
| I-0049 | 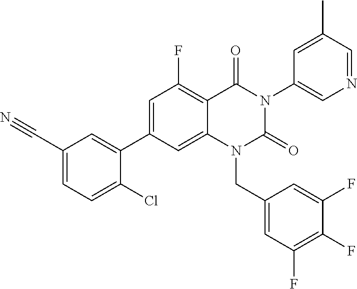 [SMILES: Cc1cncc(-n2c(=O)c3c(F)cc(-c4cc(C#N)ccc4Cl)cc3n(Cc3cc(F)c(F)c(F)c3)c2=O)c1] | 1 | 2.44 | 551.05 |

TABLE 6

| | | LC/MS | | |
|---|---|---|---|---|
| Compound | | measurement | Retention time | |
| No. | Structural formula | conditions | (min) | m/z |
| I-0050 | 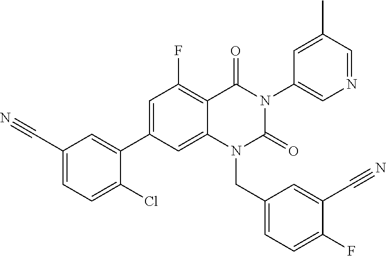 [SMILES: Cc1cncc(-n2c(=O)c3c(F)cc(-c4cc(C#N)ccc4Cl)cc3n(Cc3ccc(F)c(C#N)c3)c2=O)c1] | 1 | 2.28 | 540.05 |
| I-0051 | 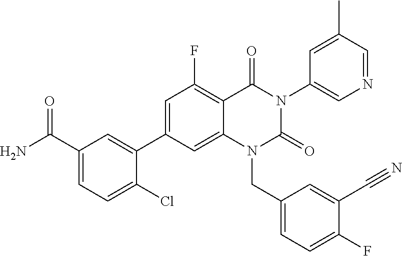 [SMILES: Cc1cncc(-n2c(=O)c3c(F)cc(-c4cc(C(N)=O)ccc4Cl)cc3n(Cc3ccc(F)c(C#N)c3)c2=O)c1] | 1 | 1.91 | 558.05 |
| I-0052 | 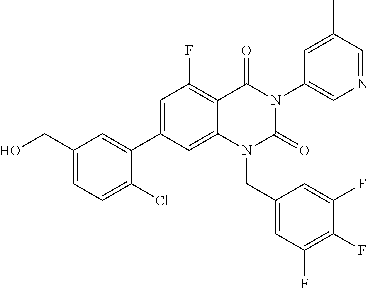 [SMILES: Cc1cncc(-n2c(=O)c3c(F)cc(-c4cc(CO)ccc4Cl)cc3n(Cc3cc(F)c(F)c(F)c3)c2=O)c1] | 1 | 2.23 | 556.05 |
| I-0053 | 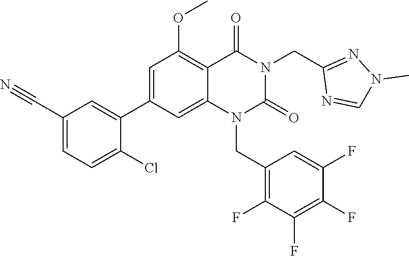 [SMILES: COc1cc(-c2cc(C#N)ccc2Cl)cc2c1c(=O)n(Cc1ncn(C)n1)c(=O)n2Cc1cc(F)c(F)c(F)c1F] | 1 | 2.25 | 585.05 |
| I-0054 | 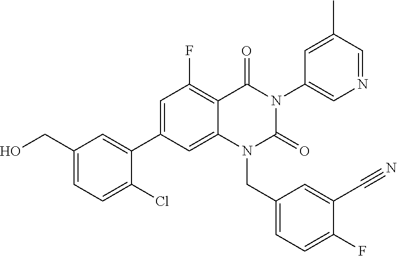 [SMILES: Cc1cncc(-n2c(=O)c3c(F)cc(-c4cc(CO)ccc4Cl)cc3n(Cc3ccc(F)c(C#N)c3)c2=O)c1] | 1 | 2.05 | 545.05 |
| I-0055 | 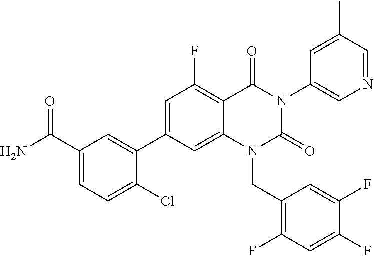 [SMILES: Cc1cncc(-n2c(=O)c3c(F)cc(-c4cc(C(N)=O)ccc4Cl)cc3n(Cc3cc(F)c(F)cc3F)c2=O)c1] | 1 | 2.03 | 569.05 |

TABLE 7

| | | LC/MS | | |
|---|---|---|---|---|
| Compound | | measurement | Retention time | |
| No. | Structural formula | conditions | (min) | m/z |
| I-0058 | 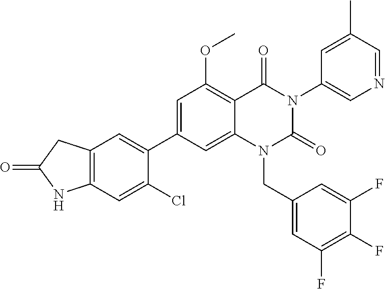 [SMILES: COc1cc(-c2cc3c(cc2Cl)NC(=O)C3)cc2c1c(=O)n(-c1cncc(C)c1)c(=O)n2Cc1cc(F)c(F)c(F)c1] | | 1.88 | 593.05 |
| I-0059 | 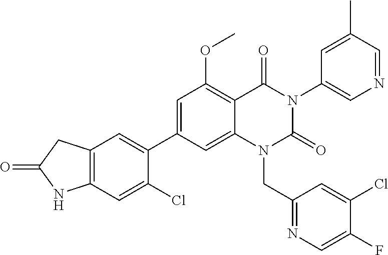 [SMILES: COc1cc(-c2cc3c(cc2Cl)NC(=O)C3)cc2c1c(=O)n(-c1cncc(C)c1)c(=O)n2Cc1cc(Cl)c(F)cn1] | 1 | 1.71 | 592 |
| I-0060 | 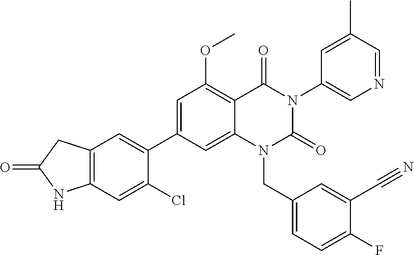 [SMILES: COc1cc(-c2cc3c(cc2Cl)NC(=O)C3)cc2c1c(=O)n(-c1cncc(C)c1)c(=O)n2Cc1ccc(F)c(C#N)c1] | 1 | 1.73 | 582.05 |
| I-0061 | 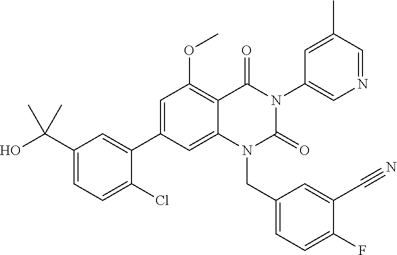 [SMILES: COc1cc(-c2cc(C(C)(C)O)ccc2Cl)cc2c1c(=O)n(-c1cncc(C)c1)c(=O)n2Cc1ccc(F)c(C#N)c1] | 1 | 2.02 | 585.1 |
| I-0062 | 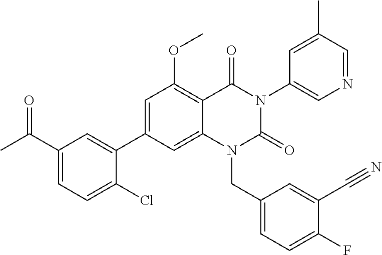 [SMILES: COc1cc(-c2cc(C(C)=O)ccc2Cl)cc2c1c(=O)n(-c1cncc(C)c1)c(=O)n2Cc1ccc(F)c(C#N)c1] | 1 | 2.07 | 569.1 |
| I-0063 | 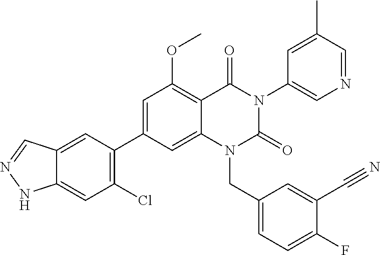 [SMILES: COc1cc(-c2cc3cn[nH]c3cc2Cl)cc2c1c(=O)n(-c1cncc(C)c1)c(=O)n2Cc1ccc(F)c(C#N)c1] | 1 | 2.18 | 567.05 |

TABLE 8

| | | LC/MS | | |
|---|---|---|---|---|
| Compound | | measurement | Retention time | |
| No. | Structural formula | conditions | (min) | m/z |
| I-0064 | 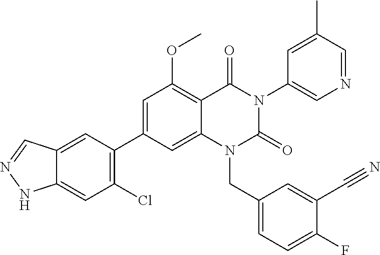 [SMILES: COc1cc(-c2cc3cn[nH]c3cc2Cl)cc2c1c(=O)n(-c1cncc(C)c1)c(=O)n2Cc1ccc(F)c(C#N)c1] | 1 | 1.93 | 605.05 |
| I-0065 | 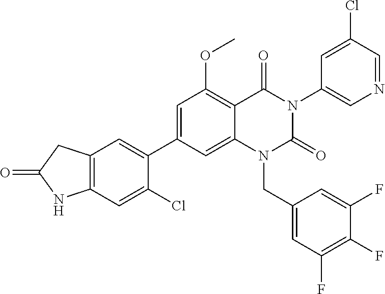 [SMILES: COc1cc(-c2cc3c(cc2Cl)NC(=O)C3)cc2c1c(=O)n(-c1cncc(Cl)c1)c(=O)n2Cc1cc(F)c(F)c(F)c1] | 1 | 2.19 | 613 |
| I-0066 | 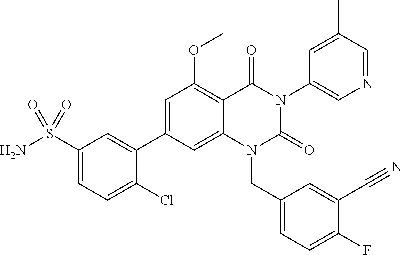 [SMILES: COc1cc(-c2cc(S(N)(=O)=O)ccc2Cl)cc2c1c(=O)n(-c1cncc(C)c1)c(=O)n2Cc1ccc(F)c(C#N)c1] | 1 | 1.83 | 606 |
| I-0067 | 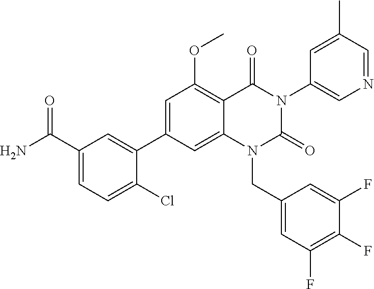 [SMILES: COc1cc(-c2cc(C(N)=O)ccc2Cl)cc2c1c(=O)n(-c1cncc(C)c1)c(=O)n2Cc1cc(F)c(F)c(F)c1] | 1 | 1.95 | 581.05 |
| I-0068 | 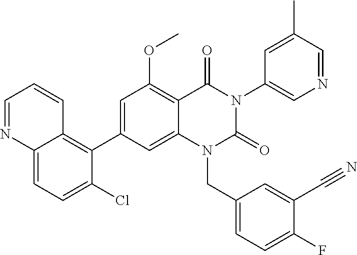 [SMILES: COc1cc(-c2c(Cl)ccc3ncccc23)cc2c1c(=O)n(-c1cncc(C)c1)c(=O)n2Cc1ccc(F)c(C#N)c1] | 1 | 1.90 | 578.05 |
| I-0069 | 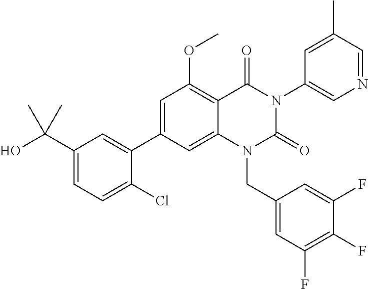 [SMILES: COc1cc(-c2cc(C(C)(C)O)ccc2Cl)cc2c1c(=O)n(-c1cncc(C)c1)c(=O)n2Cc1cc(F)c(F)c(F)c1] | 1 | 2.22 | 596.1 |

TABLE 9

| | | LC/MS | | |
|---|---|---|---|---|
| Compound | | measurement | Retention time | |
| No. | Structural formula | conditions | (min) | m/z |
| I-0070 | 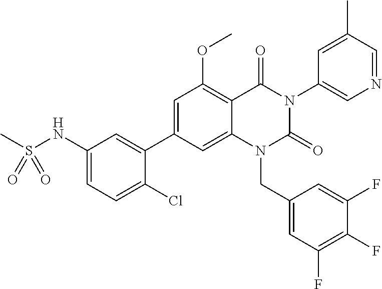 [SMILES: COc1cc(-c2cc(NS(C)(=O)=O)ccc2Cl)cc2c1c(=O)n(-c1cncc(C)c1)c(=O)n2Cc1cc(F)c(F)c(F)c1] | 1 | 2.10 | 631.05 |
| I-0071 | 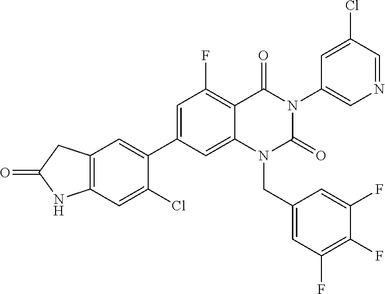 [SMILES: O=C1Cc2cc(-c3cc(F)c4c(=O)n(-c5cncc(Cl)c5)c(=O)n(Cc5cc(F)c(F)c(F)c5)c4c3)c(Cl)cc2N1] | 1 | 2.32 | 600.95 |
| I-0072 | 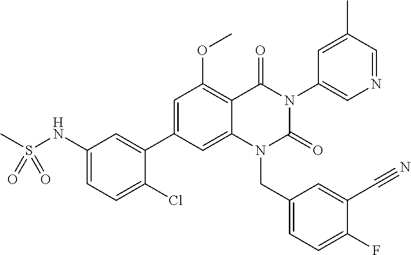 [SMILES: COc1cc(-c2cc(NS(C)(=O)=O)ccc2Cl)cc2c1c(=O)n(-c1cncc(C)c1)c(=O)n2Cc1ccc(F)c(C#N)c1] | 1 | 1.93 | 620 |
| I-0073 | 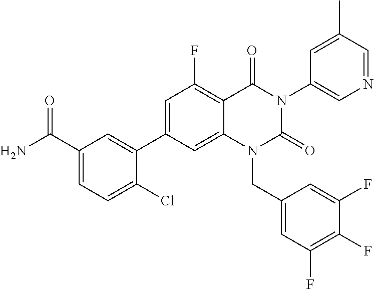 [SMILES: Cc1cncc(-n2c(=O)c3c(F)cc(-c4cc(C(N)=O)ccc4Cl)cc3n(Cc3cc(F)c(F)c(F)c3)c2=O)c1] | 1 | 2.08 | 569.05 |
| I-0074 | 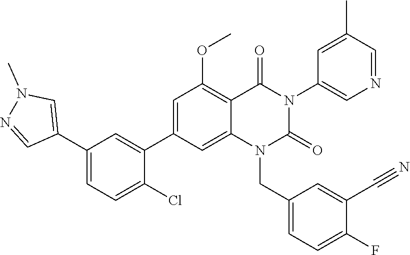 [SMILES: COc1cc(-c2cc(-c3cnn(C)c3)ccc2Cl)cc2c1c(=O)n(-c1cncc(C)c1)c(=O)n2Cc1ccc(F)c(C#N)c1] | 1 | 2.06 | 607.1 |
| I-0075 | 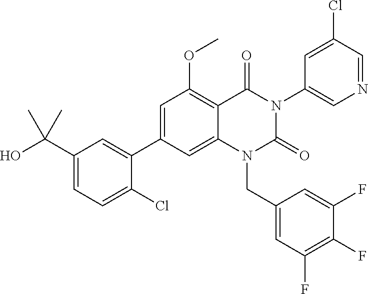 [SMILES: COc1cc(-c2cc(C(C)(C)O)ccc2Cl)cc2c1c(=O)n(-c1cncc(Cl)c1)c(=O)n2Cc1cc(F)c(F)c(F)c1] | 1 | 2.50 | 616.05 |

TABLE 10

| | | LC/MS | | |
|---|---|---|---|---|
| Compound | | measurement | Retention time | |
| No. | Structural formula | conditions | (min) | m/z |
| I-0076 | 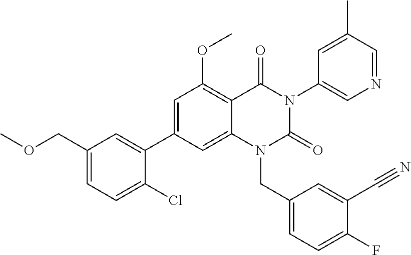 [SMILES: COCc1ccc(Cl)c(-c2cc(OC)c3c(=O)n(-c4cncc(C)c4)c(=O)n(Cc4ccc(F)c(C#N)c4)c3c2)c1] | 1 | 2.17 | 571.1 |
| I-0077 | 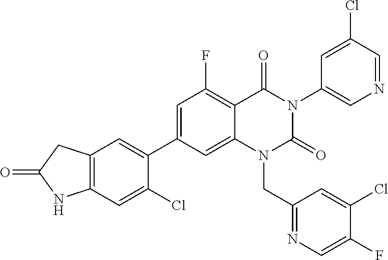 [SMILES: O=C1Cc2cc(-c3cc(F)c4c(=O)n(-c5cncc(Cl)c5)c(=O)n(Cc5cc(Cl)c(F)cn5)c4c3)c(Cl)cc2N1] | 1 | 2.17 | 599.9 |
| I-0078 | 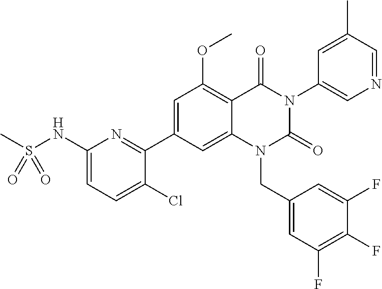 [SMILES: COc1cc(-c2nc(NS(C)(=O)=O)ccc2Cl)cc2c1c(=O)n(-c1cncc(C)c1)c(=O)n2Cc1cc(F)c(F)c(F)c1] | 1 | 2.01 | 632 |
| I-0079 | 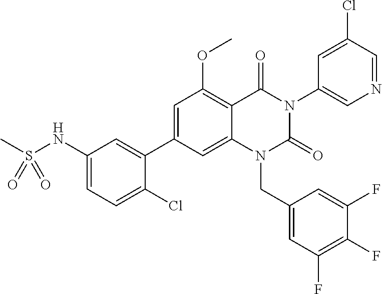 [SMILES: COc1cc(-c2cc(NS(C)(=O)=O)ccc2Cl)cc2c1c(=O)n(-c1cncc(Cl)c1)c(=O)n2Cc1cc(F)c(F)c(F)c1] | 1 | 2.38 | 651.05 |
| I-0080 | 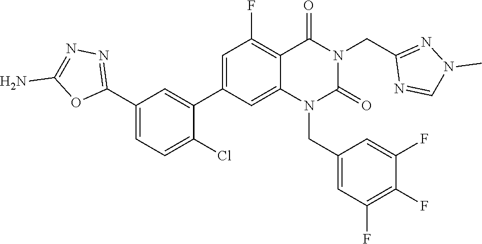 [SMILES: Cn1cnc(Cn2c(=O)c3c(F)cc(-c4cc(-c5nnc(N)o5)ccc4Cl)cc3n(Cc3cc(F)c(F)c(F)c3)c2=O)n1] | 1 | 1.95 | 613.05 |
| I-0110 | 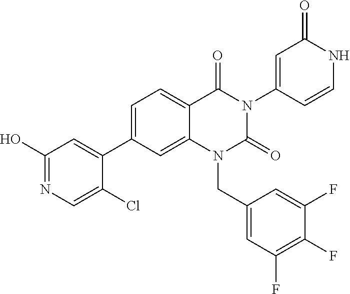 [SMILES: O=c1cc(-n2c(=O)c3ccc(-c4cc(O)ncc4Cl)cc3n(Cc3cc(F)c(F)c(F)c3)c2=O)cc[nH]1] | 1 | 1.64 | 541.1 |

TABLE 11

| | | LC/MS | | |
|---|---|---|---|---|
| Compound | | measurement | Retention time | |
| No. | Structural formula | conditions | (min) | m/z |
| I-0115 | 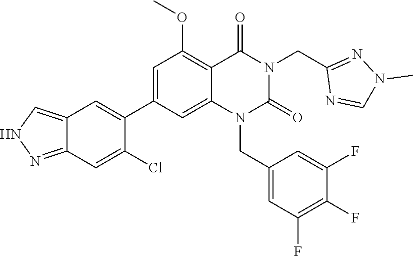 [SMILES: COc1cc(-c2cc3c[nH]nc3cc2Cl)cc2c1c(=O)n(Cc1ncn(C)n1)c(=O)n2Cc1cc(F)c(F)c(F)c1] | 1 | 1.78 | 582.12 |
| I-0116 | 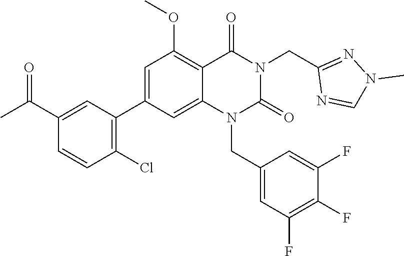 [SMILES: COc1cc(-c2cc(C(C)=O)ccc2Cl)cc2c1c(=O)n(Cc1ncn(C)n1)c(=O)n2Cc1cc(F)c(F)c(F)c1] | 1 | 2.21 | 584.1 |
| I-0117 | 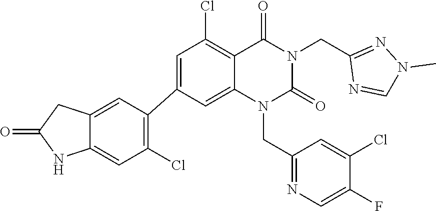 [SMILES: Cn1cnc(Cn2c(=O)c3c(Cl)cc(-c4cc5c(cc4Cl)NC(=O)C5)cc3n(Cc3cc(Cl)c(F)cn3)c2=O)n1] | 1 | 1.86 | 600 |
| I-0118 | 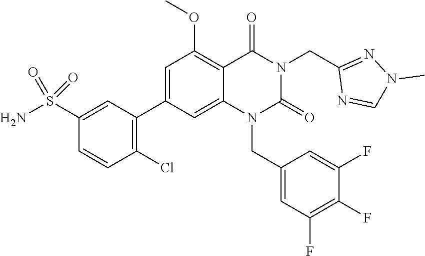 [SMILES: COc1cc(-c2cc(S(N)(=O)=O)ccc2Cl)cc2c1c(=O)n(Cc1ncn(C)n1)c(=O)n2Cc1cc(F)c(F)c(F)c1] | 1 | 1.94 | 621.05 |
| I-0119 | 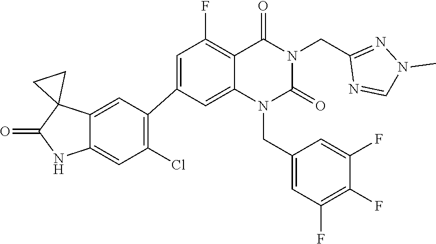 [SMILES: Cn1cnc(Cn2c(=O)c3c(F)cc(-c4cc5c(cc4Cl)NC(=O)C54CC4)cc3n(Cc3cc(F)c(F)c(F)c3)c2=O)n1] | 1 | 2.01 | 611.05 |
| I-0120 | 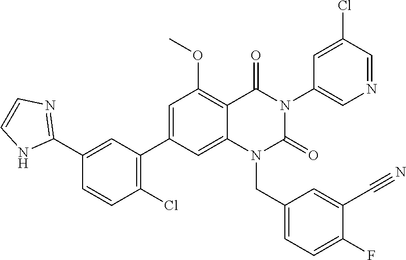 [SMILES: COc1cc(-c2cc(-c3ncc[nH]3)ccc2Cl)cc2c1c(=O)n(-c1cncc(Cl)c1)c(=O)n2Cc1ccc(F)c(C#N)c1] | 1 | 1.78 | 613 |

TABLE 12

| | | LC/MS | | |
|---|---|---|---|---|
| Compound | | measurement | Retention time | |
| No. | Structural formula | conditions | (min) | m/z |
| I-0121 | 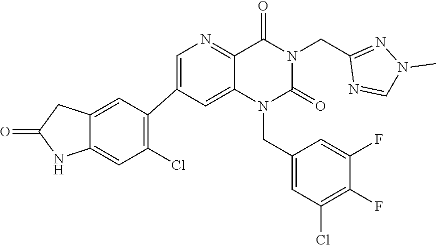 [SMILES: Cn1cnc(Cn2c(=O)c3ncc(-c4cc5c(cc4Cl)NC(=O)C5)cc3n(Cc3cc(F)c(F)c(Cl)c3)c2=O)n1] | 1 | 1.80 | 584 |
| I-0122 | 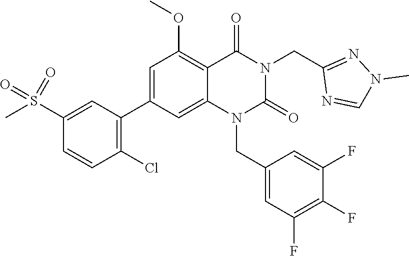 | 1 | 1.93 | 620.09 |
| I-0123 | 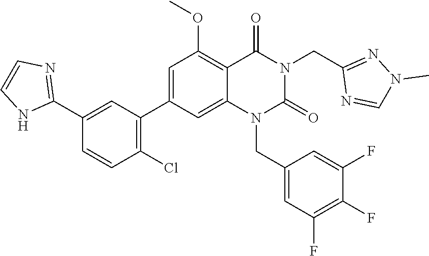 [SMILES: COc1cc(-c2cc(-c3ncc[nH]3)ccc2Cl)cc2c1c(=O)n(Cc1ncn(C)n1)c(=O)n2Cc1cc(F)c(F)c(F)c1] | 1 | 1.61 | 608.1 |
| I-0124 | 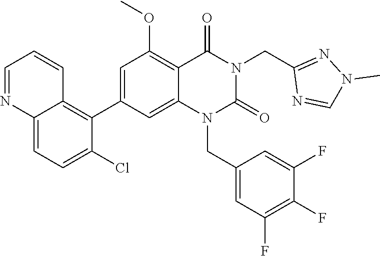 [SMILES: COc1cc(-c2c(Cl)ccc3ncccc23)cc2c1c(=O)n(Cc1ncn(C)n1)c(=O)n2Cc1cc(F)c(F)c(F)c1] | 1 | 1.98 | 593.1 |
| I-0125 | 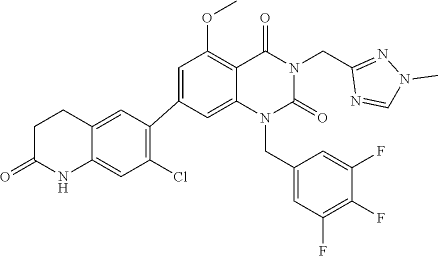 [SMILES: COc1cc(-c2cc3c(cc2Cl)NC(=O)CC3)cc2c1c(=O)n(Cc1ncn(C)n1)c(=O)n2Cc1cc(F)c(F)c(F)c1] | 1 | 1.86 | 6111.1 |
| I-0136 | 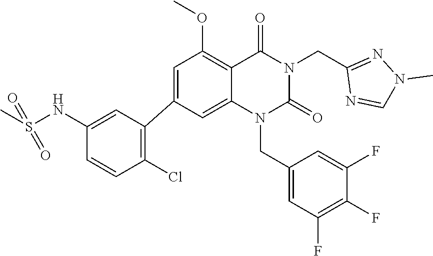 | 1 | 2.05 | 635.05 |

TABLE 13

| | | LC/MS | | |
|---|---|---|---|---|
| Compound | | measurement | Retention time | |
| No. | Structural formula | conditions | (min) | m/z |
| I-0139 | 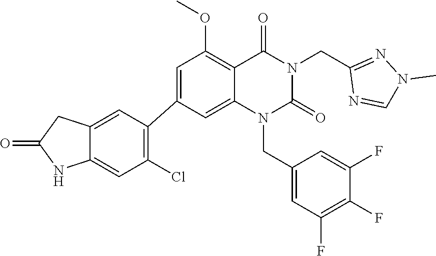 [SMILES: COc1cc(-c2cc3c(cc2Cl)NC(=O)C3)cc2c1c(=O)n(Cc1ncn(C)n1)c(=O)n2Cc1cc(F)c(F)c(F)c1] | 1 | 1.81 | 597.05 |
| I-0140 | 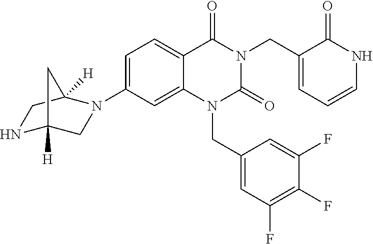 [SMILES: O=c1[nH]cccc1Cn1c(=O)c2ccc(N3C[C@H]4C[C@@H]3CN4)cc2n(Cc2cc(F)c(F)c(F)c2)c1=O] | 1 | 1.29 | 510 |
| I-0141 | 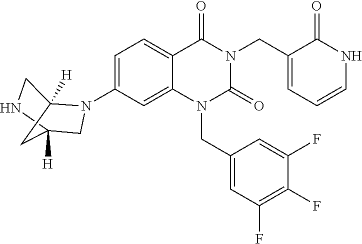 [SMILES: O=c1[nH]cccc1Cn1c(=O)c2ccc(N3C[C@@H]4C[C@H]3CN4)cc2n(Cc2cc(F)c(F)c(F)c2)c1=O] | 1 | 1.30 | 510 |
| I-0142 | 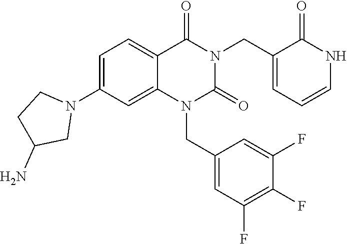 [SMILES: NC1CCN(c2ccc3c(=O)n(Cc4ccc[nH]c4=O)c(=O)n(Cc4cc(F)c(F)c(F)c4)c3c2)C1] | 1 | 1.29 | 498 |
| I-0160 | 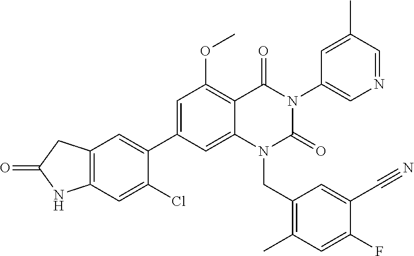 [SMILES: COc1cc(-c2cc3c(cc2Cl)NC(=O)C3)cc2c1c(=O)n(-c1cncc(C)c1)c(=O)n2Cc1cc(C#N)c(F)cc1C] | 1 | 1.80 | 596.15 |
| I-0161 | 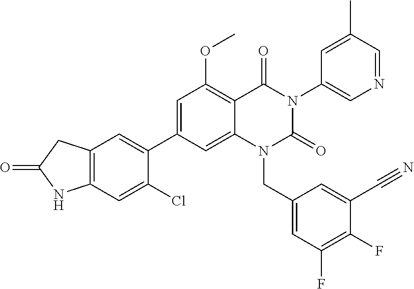 [SMILES: COc1cc(-c2cc3c(cc2Cl)NC(=O)C3)cc2c1c(=O)n(-c1cncc(C)c1)c(=O)n2Cc1cc(F)c(F)c(C#N)c1] | 1 | 1.82 | 600.05 |

TABLE 14

| | | LC/MS | | |
|---|---|---|---|---|
| Compound | | measurement | Retention time | |
| No. | Structural formula | conditions | (min) | m/z |
| I-0162 | 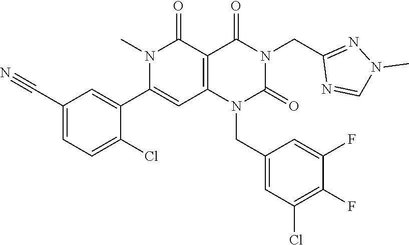 [SMILES: Cn1cnc(Cn2c(=O)c3c(=O)n(C)c(-c4cc(C#N)ccc4Cl)cc3n(Cc3cc(F)c(F)c(Cl)c3)c2=O)n1] | 1 | 2.00 | 584 |
| I-0163 | 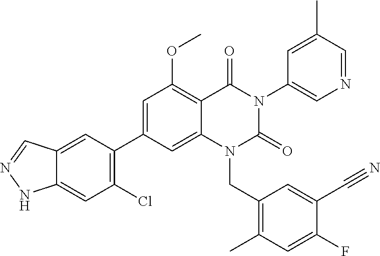 [SMILES: COc1cc(-c2cc3cn[nH]c3cc2Cl)cc2c1c(=O)n(-c1cncc(C)c1)c(=O)n2Cc1cc(C#N)c(F)cc1C] | 1 | 1.84 | 581.1 |
| I-0164 | 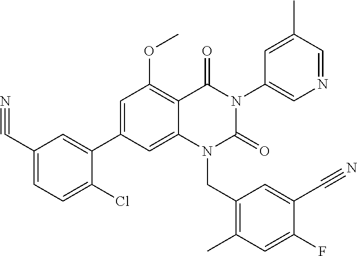 [SMILES: COc1cc(-c2cc(C#N)ccc2Cl)cc2c1c(=O)n(-c1cncc(C)c1)c(=O)n2Cc1cc(C#N)c(F)cc1C] | 1 | 2.14 | 566.1 |
| I-0165 | 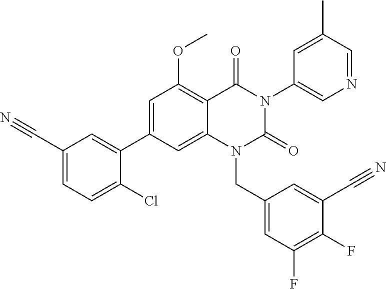 [SMILES: COc1cc(-c2cc(C#N)ccc2Cl)cc2c1c(=O)n(-c1cncc(C)c1)c(=O)n2Cc1cc(F)c(F)c(C#N)c1] | 1 | 2.22 | 570.1 |
| I-0166 | 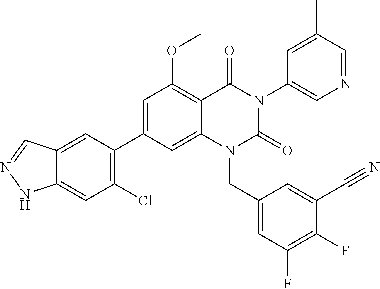 [SMILES: COc1cc(-c2cc3cn[nH]c3cc2Cl)cc2c1c(=O)n(-c1cncc(C)c1)c(=O)n2Cc1cc(F)c(F)c(C#N)c1] | 1 | 1.89 | 585.5 |
| I-0167 | 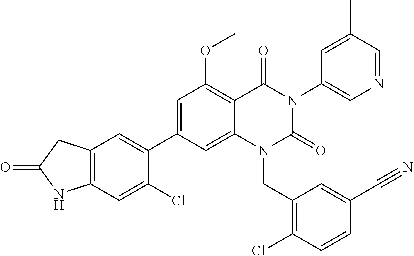 [SMILES: COc1cc(-c2cc3c(cc2Cl)NC(=O)C3)cc2c1c(=O)n(-c1cncc(C)c1)c(=O)n2Cc1cc(C#N)ccc1Cl] | 1 | 1.77 | 598 |

TABLE 15

| | | LC/MS | Retention | |
|---|---|---|---|---|
| Compound | | measurement | time | |
| No. | Structural formula | conditions | (min) | m/z |
| I-0168 | 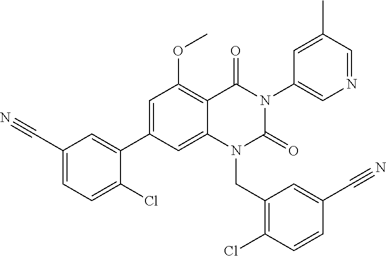 [SMILES: COc1cc(-c2cc(C#N)ccc2Cl)cc2c1c(=O)n(-c1cncc(C)c1)c(=O)n2Cc1cc(C#N)ccc1Cl] | 1 | 2.17 | 568.05 |
| I-0169 | 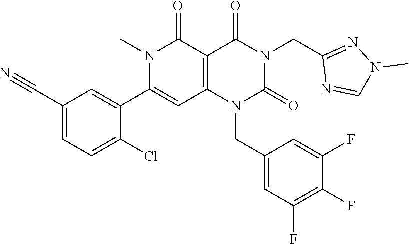 [SMILES: Cn1cnc(Cn2c(=O)c3c(=O)n(C)c(-c4cc(C#N)ccc4Cl)cc3n(Cc3cc(F)c(F)c(F)c3)c2=O)n1] | 1 | 1.86 | 568.05 |
| I-0170 | 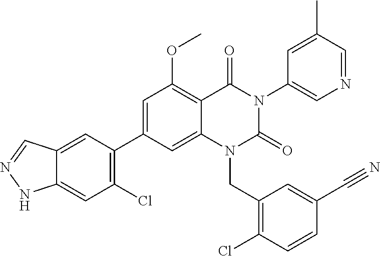 [SMILES: COc1cc(-c2cc3cn[nH]c3cc2Cl)cc2c1c(=O)n(-c1cncc(C)c1)c(=O)n2Cc1cc(C#N)ccc1Cl] | 1 | 1.86 | 583 |
| I-0171 | 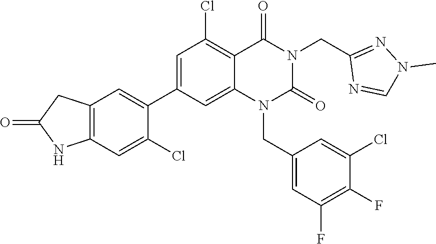 [SMILES: Cn1cnc(Cn2c(=O)c3c(Cl)cc(-c4cc5c(cc4Cl)NC(=O)C5)cc3n(Cc3cc(F)c(F)c(Cl)c3)c2=O)n1] | 1 | 2.10 | 617 |
| I-0172 | 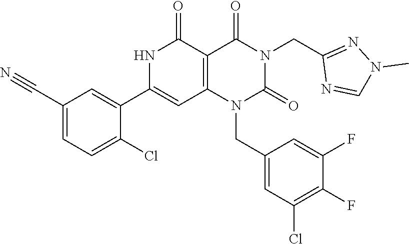 [SMILES: Cn1cnc(Cn2c(=O)c3c(=O)[nH]c(-c4cc(C#N)ccc4Cl)cc3n(Cc3cc(F)c(F)c(Cl)c3)c2=O)n1] | 1 | 1.97 | 570.05 |
| I-0173 | 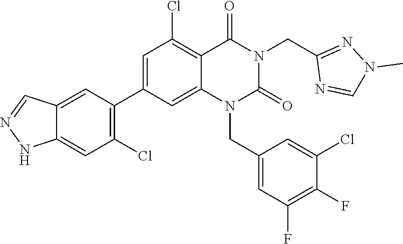 [SMILES: Cn1cnc(Cn2c(=O)c3c(Cl)cc(-c4cc5cn[nH]c5cc4Cl)cc3n(Cc3cc(F)c(F)c(Cl)c3)c2=O)n1] | 1 | 2.17 | 602 |

TABLE 16

| | | LC/MS | Retention | |
|---|---|---|---|---|
| Compound | | measurement | time | |
| No. | Structural formula | conditions | (min) | m/z |
| I-0174 | 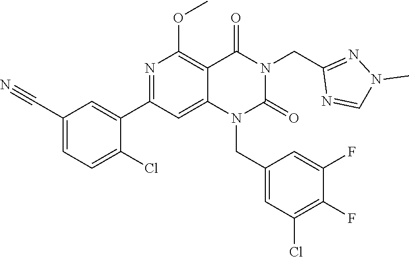 [SMILES: COc1nc(-c2cc(C#N)ccc2Cl)cc2c1c(=O)n(Cc1ncn(C)n1)c(=O)n2Cc1cc(F)c(F)c(Cl)c1] | 1 | 2.39 | 584 |
| I-0175 | 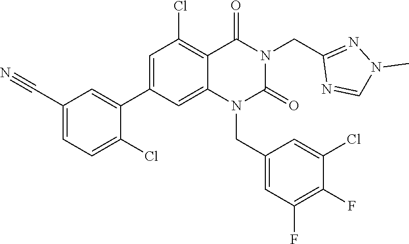 [SMILES: Cn1cnc(Cn2c(=O)c3c(Cl)cc(-c4cc(C#N)ccc4Cl)cc3n(Cc3cc(F)c(F)c(Cl)c3)c2=O)n1] | 1 | 2.50 | 586.95 |
| I-0176 | 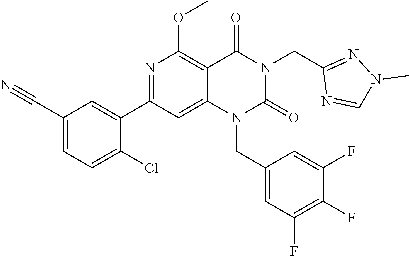 [SMILES: COc1nc(-c2cc(C#N)ccc2Cl)cc2c1c(=O)n(Cc1ncn(C)n1)c(=O)n2Cc1cc(F)c(F)c(F)c1] | 1 | 2.25 | 568.1 |
| I-0177 | 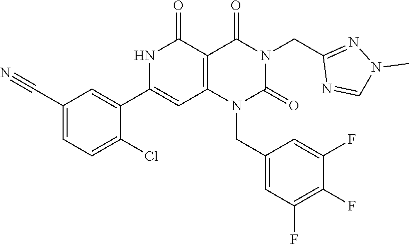 [SMILES: Cn1cnc(Cn2c(=O)c3c(=O)[nH]c(-c4cc(C#N)ccc4Cl)cc3n(Cc3cc(F)c(F)c(F)c3)c2=O)n1] | 1 | 1.86 | 554.05 |
| I-0178 | 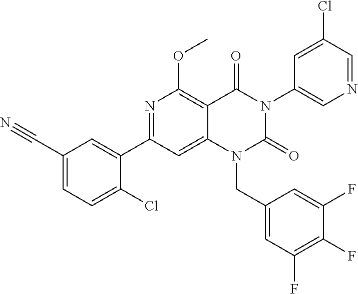 [SMILES: COc1nc(-c2cc(C#N)ccc2Cl)cc2c1c(=O)n(-c1cncc(Cl)c1)c(=O)n2Cc1cc(F)c(F)c(F)c1] | 1 | 2.67 | 584 |
| I-0187 | 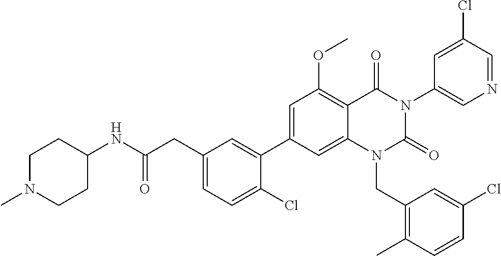 [SMILES: COc1cc(-c2cc(CC(=O)NC3CCN(C)CC3)ccc2Cl)cc2c1c(=O)n(-c1cncc(Cl)c1)c(=O)n2Cc1cc(Cl)ccc1C] | 1 | 1.90 | 706.1 |

TABLE 17

| | | LC/MS | Retention | |
|---|---|---|---|---|
| Compound | | measurement | time | |
| No. | Structural formula | conditions | (min) | m/z |
| I-0188 | 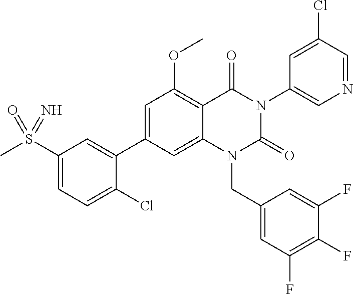 [SMILES: COc1cc(-c2cc(S(C)(=N)=O)ccc2Cl)cc2c1c(=O)n(-c1cncc(Cl)c1)c(=O)n2Cc1cc(F)c(F)c(F)c1] | 1 | 2.18 | 634.95 |
| I-0189 | 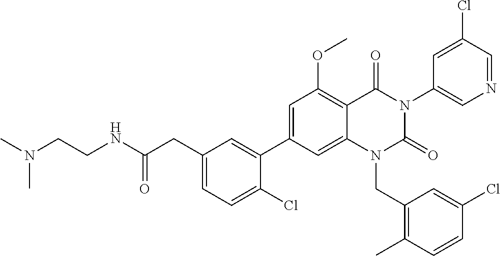 [SMILES: COc1cc(-c2cc(CC(=O)NCCN(C)C)ccc2Cl)cc2c1c(=O)n(-c1cncc(Cl)c1)c(=O)n2Cc1cc(Cl)ccc1C] | 1 | 1.88 | 680.1 |
| I-0190 | 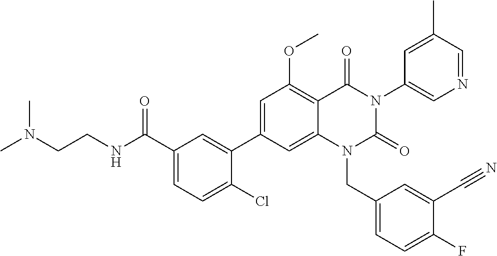 [SMILES: COc1cc(-c2cc(C(=O)NCCN(C)C)ccc2Cl)cc2c1c(=O)n(-c1cncc(C)c1)c(=O)n2Cc1ccc(F)c(C#N)c1] | 1 | 1.54 | 641.1 |
| I-0191 | 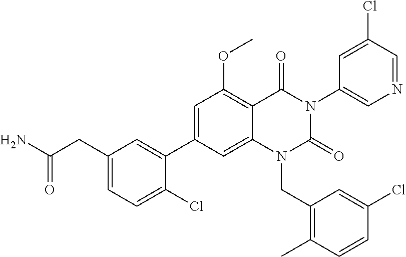 [SMILES: COc1cc(-c2cc(CC(N)=O)ccc2Cl)cc2c1c(=O)n(-c1cncc(Cl)c1)c(=O)n2Cc1cc(Cl)ccc1C] | 1 | 2.24 | 609.05 |
| I-0192 | 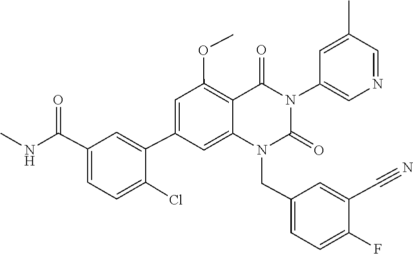 [SMILES: CNC(=O)c1ccc(Cl)c(-c2cc(OC)c3c(=O)n(-c4cncc(C)c4)c(=O)n(Cc4ccc(F)c(C#N)c4)c3c2)c1] | 1 | 1.85 | 584.1 |
| I-0193 | 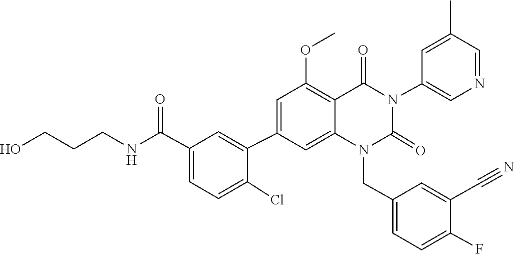 [SMILES: COc1cc(-c2cc(C(=O)NCCCO)ccc2Cl)cc2c1c(=O)n(-c1cncc(C)c1)c(=O)n2Cc1ccc(F)c(C#N)c1] | 1 | 1.76 | 628.1 |

TABLE 18

| | | LC/MS | Retention | |
|---|---|---|---|---|
| Compound | | measurement | time | |
| No. | Structural formula | conditions | (min) | m/z |
| I-0194 | 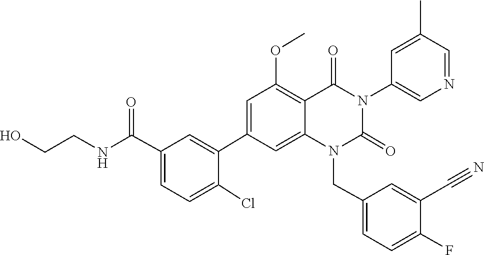 [SMILES: COc1cc(-c2cc(C(=O)NCCO)ccc2Cl)cc2c1c(=O)n(-c1cncc(C)c1)c(=O)n2Cc1ccc(F)c(C#N)c1] | 1 | 1.74 | 614.1 |
| I-0195 | 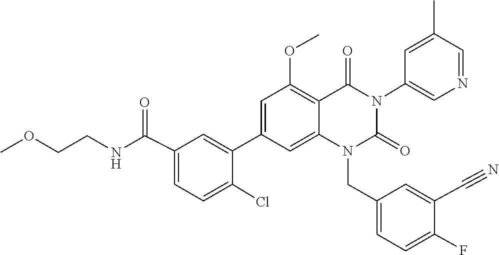 [SMILES: COCCNC(=O)c1ccc(Cl)c(-c2cc(OC)c3c(=O)n(-c4cncc(C)c4)c(=O)n(Cc4ccc(F)c(C#N)c4)c3c2)c1] | 1 | 1.90 | 626.1 |
| I-0196 | 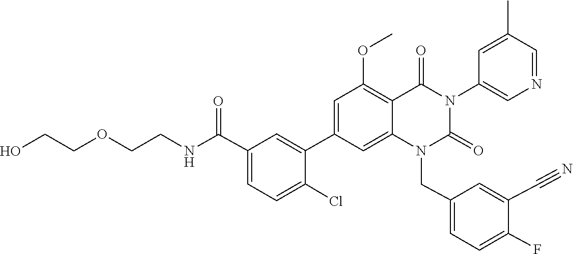 [SMILES: COc1cc(-c2cc(C(=O)NCCOCCO)ccc2Cl)cc2c1c(=O)n(-c1cncc(C)c1)c(=O)n2Cc1ccc(F)c(C#N)c1] | 1 | 1.75 | 658.15 |
| I-0197 | 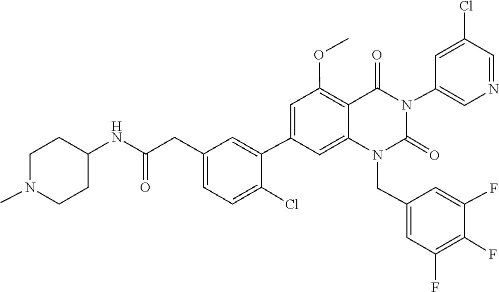 [SMILES: COc1cc(-c2cc(CC(=O)NC3CCN(C)CC3)ccc2Cl)cc2c1c(=O)n(-c1cncc(Cl)c1)c(=O)n2Cc1cc(F)c(F)c(F)c1] | 1 | 1.88 | 712.1 |
| I-0198 | 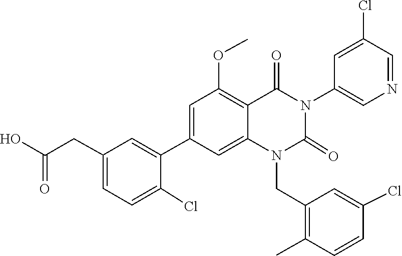 [SMILES: COc1cc(-c2cc(CC(=O)O)ccc2Cl)cc2c1c(=O)n(-c1cncc(Cl)c1)c(=O)n2Cc1cc(Cl)ccc1C] | 1 | 2.41 | 610 |
| I-0199 | 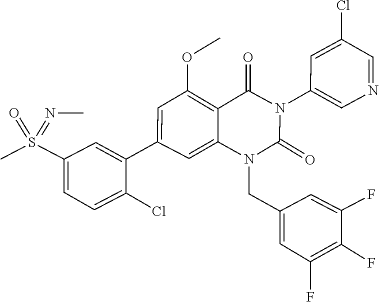 [SMILES: CN=S(C)(=O)c1ccc(Cl)c(-c2cc(OC)c3c(=O)n(-c4cncc(Cl)c4)c(=O)n(Cc4cc(F)c(F)c(F)c4)c3c2)c1] | 1 | 2.24 | 649 |

TABLE 19

| | | LC/MS | Retention | |
|---|---|---|---|---|
| Compound | | measurement | time | |
| No. | Structural formula | conditions | (min) | m/z |
| I-0200 | 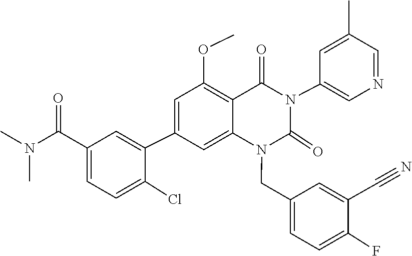 [SMILES: COc1cc(-c2cc(C(=O)N(C)C)ccc2Cl)cc2c1c(=O)n(-c1cncc(C)c1)c(=O)n2Cc1ccc(F)c(C#N)c1] | 1 | 1.87 | 598.1 |
| I-0201 | 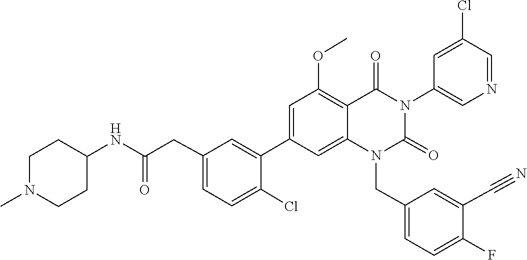 [SMILES: COc1cc(-c2cc(CC(=O)NC3CCN(C)CC3)ccc2Cl)cc2c1c(=O)n(-c1cncc(Cl)c1)c(=O)n2Cc1ccc(F)c(C#N)c1] | 1 | 1.77 | 701.1 |
| I-0202 | 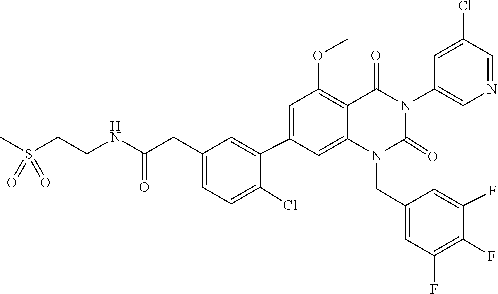 [SMILES: COc1cc(-c2cc(CC(=O)NCCS(C)(=O)=O)ccc2Cl)cc2c1c(=O)n(-c1cncc(Cl)c1)c(=O)n2Cc1cc(F)c(F)c(F)c1] | 1 | 2.25 | 721 |
| I-0203 | 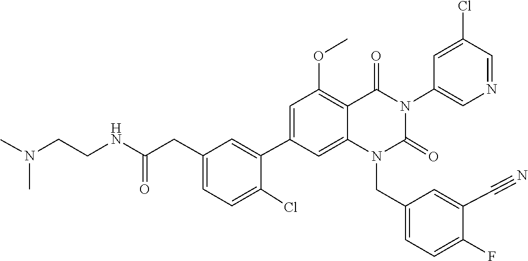 [SMILES: COc1cc(-c2cc(CC(=O)NCCN(C)C)ccc2Cl)cc2c1c(=O)n(-c1cncc(Cl)c1)c(=O)n2Cc1ccc(F)c(C#N)c1] | 1 | 1.77 | 675.1 |
| I-0204 | 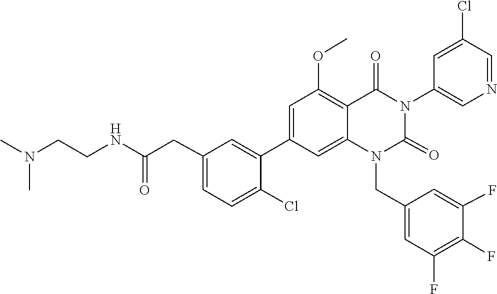 [SMILES: COc1cc(-c2cc(CC(=O)NCCN(C)C)ccc2Cl)cc2c1c(=O)n(-c1cncc(Cl)c1)c(=O)n2Cc1cc(F)c(F)c(F)c1] | 1 | 1.89 | 686.1 |
| I-0205 | 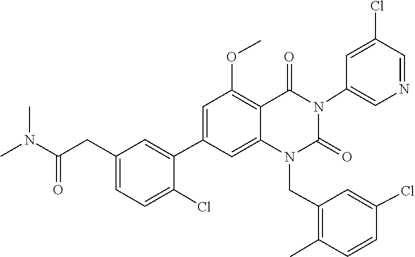 [SMILES: COc1cc(-c2cc(CC(=O)N(C)C)ccc2Cl)cc2c1c(=O)n(-c1cncc(Cl)c1)c(=O)n2Cc1cc(Cl)ccc1C] | 1 | 2.42 | 637.05 |

TABLE 20

| | | LC/MS | Retention | |
|---|---|---|---|---|
| Compound | | measurement | time | |
| No. | Structural formula | conditions | (min) | m/z |
| I-0206 | 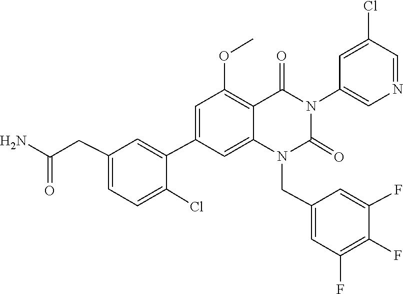 [SMILES: COc1cc(-c2cc(CC(N)=O)ccc2Cl)cc2c1c(=O)n(-c1cncc(Cl)c1)c(=O)n2Cc1cc(F)c(F)c(F)c1] | 1 | 2.18 | 615 |
| I-0207 | 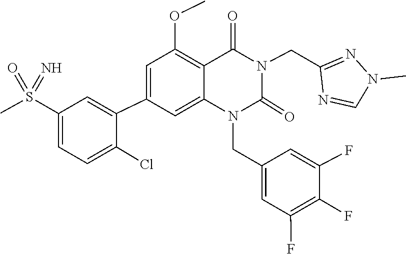 | 1 | 1.81 | 619.05 |
| I-0208 | 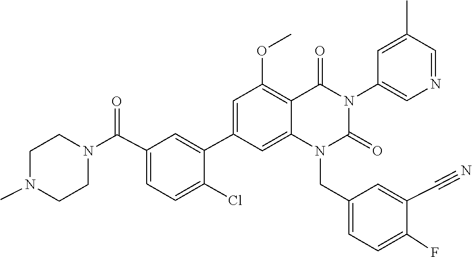 [SMILES: COc1cc(-c2cc(C(=O)N3CCN(C)CC3)ccc2Cl)cc2c1c(=O)n(-c1cncc(C)c1)c(=O)n2Cc1ccc(F)c(C#N)c1] | 1 | 1.55 | 653.15 |
| I-0209 | 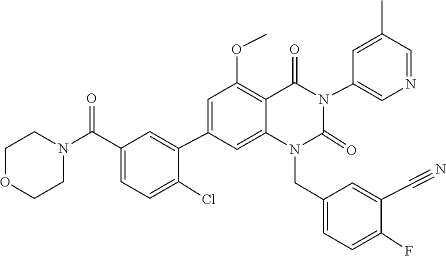 [SMILES: COc1cc(-c2cc(C(=O)N3CCOCC3)ccc2Cl)cc2c1c(=O)n(-c1cncc(C)c1)c(=O)n2Cc1ccc(F)c(C#N)c1] | 1 | 1.89 | 640.1 |
| I-0210 | 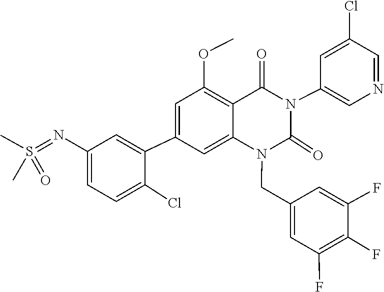 [SMILES: COc1cc(-c2cc(N=S(C)(C)=O)ccc2Cl)cc2c1c(=O)n(-c1cncc(Cl)c1)c(=O)n2Cc1cc(F)c(F)c(F)c1] | 1 | 2.35 | 649 |
| I-0211 | 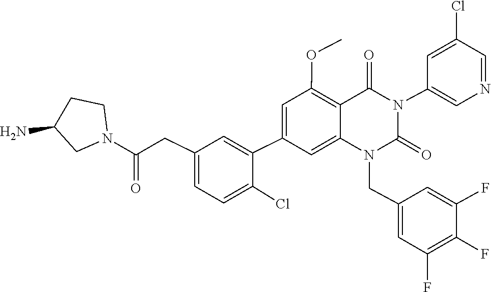 [SMILES: COc1cc(-c2cc(CC(=O)N3CC[C@H](N)C3)ccc2Cl)cc2c1c(=O)n(-c1cncc(Cl)c1)c(=O)n2Cc1cc(F)c(F)c(F)c1] | 1 | 1.86 | 684.05 |

TABLE 21

| | | LC/MS | Retention | |
|---|---|---|---|---|
| Compound | | measurement | time | |
| No. | Structural formula | conditions | (min) | m/z |
| I-0212 | 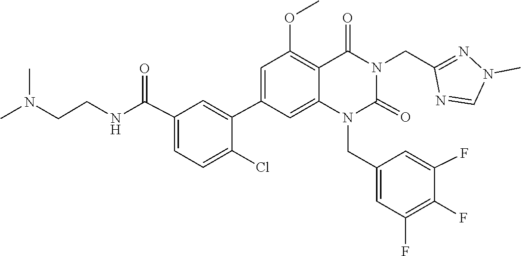 | 1 | 1.62 | 656.15 |
| I-0213 | 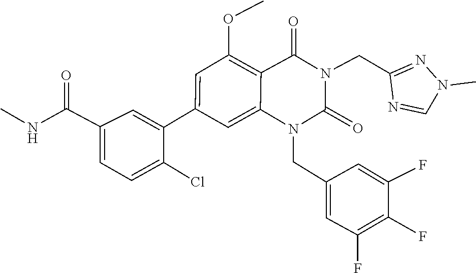 | 1 | 1.93 | 599.05 |
| I-0214 | 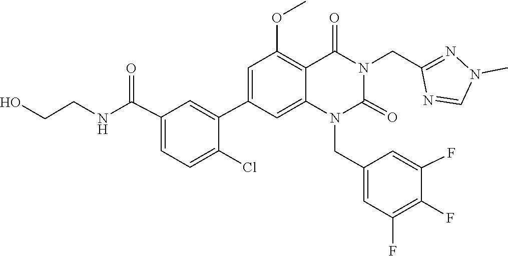 [SMILES: COc1cc(-c2cc(C(=O)NCCO)ccc2Cl)cc2c1c(=O)n(Cc1ncn(C)n1)c(=O)n2Cc1cc(F)c(F)c(F)c1] | 1 | 1.81 | 629.1 |
| I-0215 | 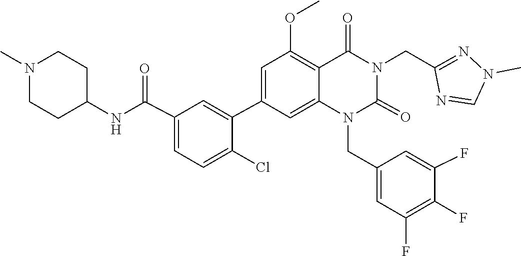 | 1 | 1.64 | 662.1 |
| I-0216 | 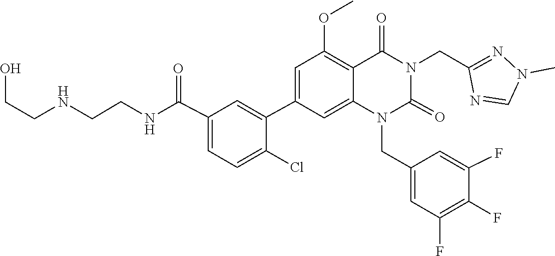 [SMILES: COc1cc(-c2cc(C(=O)NCCNCCO)ccc2Cl)cc2c1c(=O)n(Cc1ncn(C)n1)c(=O)n2Cc1cc(F)c(F)c(F)c1] | 1 | 1.57 | 672.15 |
| I-0217 | 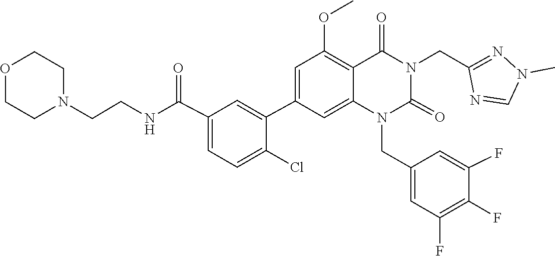 [SMILES: COc1cc(-c2cc(C(=O)NCCN3CCOCC3)ccc2Cl)cc2c1c(=O)n(Cc1ncn(C)n1)c(=O)n2Cc1cc(F)c(F)c(F)c1] | 1 | 1.63 | 698.15 |

TABLE 22

| | | LC/MS | Retention | |
|---|---|---|---|---|
| Compound | | measurement | time | |
| No. | Structural formula | conditions | (min) | m/z |
| I-0248 | 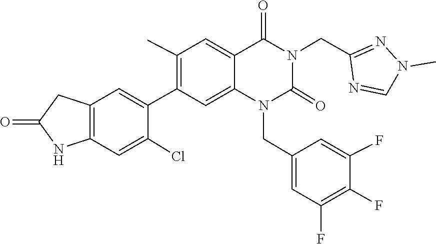 [SMILES: Cc1cc2c(=O)n(Cc3ncn(C)n3)c(=O)n(Cc3cc(F)c(F)c(F)c3)c2cc1-c1cc2c(cc1Cl)NC(=O)C2] | 1 | 1.94 | 581 |
| I-0249 | 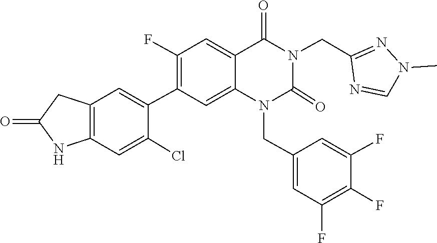 [SMILES: Cn1cnc(Cn2c(=O)c3cc(F)c(-c4cc5c(cc4Cl)NC(=O)C5)cc3n(Cc3cc(F)c(F)c(F)c3)c2=O)n1] | 1 | 1.84 | 585 |
| I-0250 | 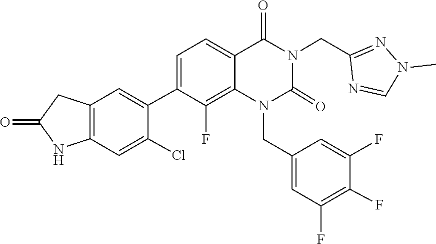 [SMILES: Cn1cnc(Cn2c(=O)c3ccc(-c4cc5c(cc4Cl)NC(=O)C5)c(F)c3n(Cc3cc(F)c(F)c(F)c3)c2=O)n1] | 1 | 1.93 | 585 |
| I-0263 | 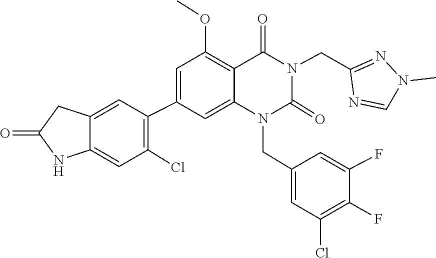 [SMILES: COc1cc(-c2cc3c(cc2Cl)NC(=O)C3)cc2c1c(=O)n(Cc1ncn(C)n1)c(=O)n2Cc1cc(F)c(F)c(Cl)c1] | 1 | 1.85 | 613 |
| I-0264 | 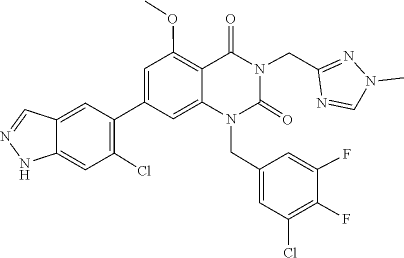 [SMILES: COc1cc(-c2cc3cn[nH]c3cc2Cl)cc2c1c(=O)n(Cc1ncn(C)n1)c(=O)n2Cc1cc(F)c(F)c(Cl)c1] | 1 | 1.93 | 598 |
| I-0265 | 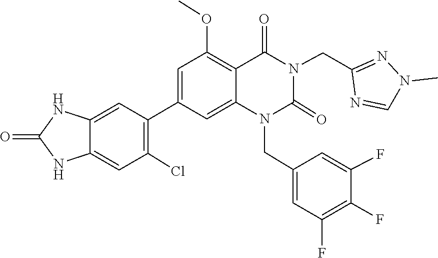 [SMILES: COc1cc(-c2cc3[nH]c(=O)[nH]c3cc2Cl)cc2c1c(=O)n(Cc1ncn(C)n1)c(=O)n2Cc1cc(F)c(F)c(F)c1] | 1 | 1.69 | 598 |

TABLE 23

| | | LC/MS | Retention | |
|---|---|---|---|---|
| Compound | | measurement | time | |
| No. | Structural formula | conditions | (min) | m/z |
| I-0266 | 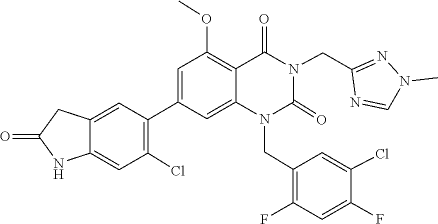 [SMILES: COc1cc(-c2cc3c(cc2Cl)NC(=O)C3)cc2c1c(=O)n(Cc1ncn(C)n1)c(=O)n2Cc1cc(Cl)c(F)cc1F] | 1 | 1.98 | 613 |
| I-0267 | 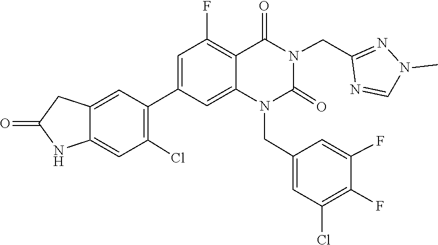 [SMILES: Cn1cnc(Cn2c(=O)c3c(F)cc(-c4cc5c(cc4Cl)NC(=O)C5)cc3n(Cc3cc(F)c(F)c(Cl)c3)c2=O)n1] | 1 | 1.98 | 601 |
| I-0268 | 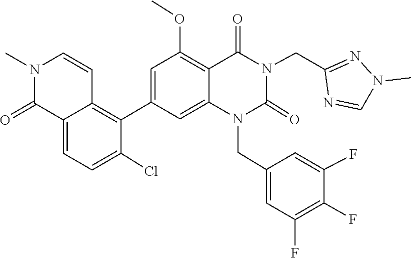 | 1 | 1.95 | 623 |
| I-0269 | 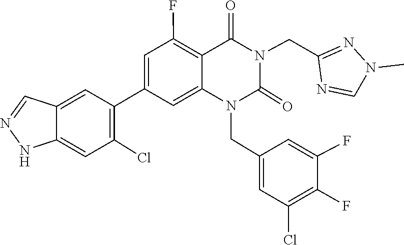 [SMILES: Cn1cnc(Cn2c(=O)c3c(F)cc(-c4cc5cn[nH]c5cc4Cl)cc3n(Cc3cc(F)c(F)c(Cl)c3)c2=O)n1] | 1 | 2.09 | 586 |
| I-0270 | 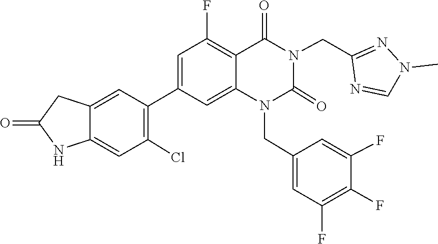 [SMILES: Cn1cnc(Cn2c(=O)c3c(F)cc(-c4cc5c(cc4Cl)NC(=O)C5)cc3n(Cc3cc(F)c(F)c(F)c3)c2=O)n1] | 1 | 1.93 | 585 |
| I-0271 | 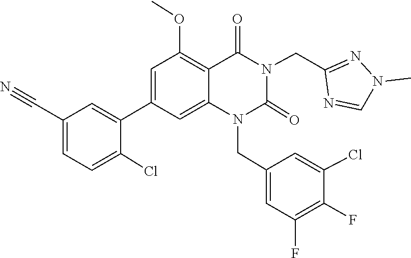 [SMILES: COc1cc(-c2cc(C#N)ccc2Cl)cc2c1c(=O)n(Cc1ncn(C)n1)c(=O)n2Cc1cc(F)c(F)c(Cl)c1] | 1 | 2.27 | 583 |

TABLE 24

| | | LC/MS | Retention | |
|---|---|---|---|---|
| Compound | | measurement | time | |
| No. | Structural formula | conditions | (min) | m/z |
| I-0272 | 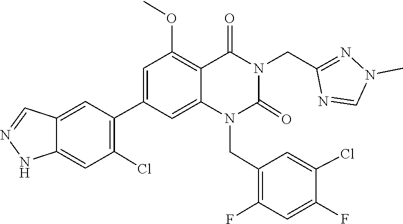 [SMILES: COc1cc(-c2cc3cn[nH]c3cc2Cl)cc2c1c(=O)n(Cc1ncn(C)n1)c(=O)n2Cc1cc(Cl)c(F)cc1F] | 1 | 1.81 | 598 |
| I-0273 | 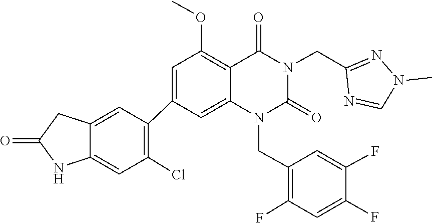 [SMILES: COc1cc(-c2cc3c(cc2Cl)NC(=O)C3)cc2c1c(=O)n(Cc1ncn(C)n1)c(=O)n2Cc1cc(F)c(F)cc1F] | 1 | 1.73 | 597 |
| I-0274 | 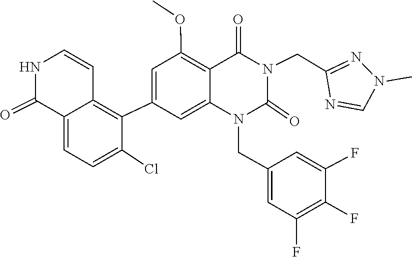 [SMILES: COc1cc(-c2c(Cl)ccc3c(=O)[nH]ccc23)cc2c1c(=O)n(Cc1ncn(C)n1)c(=O)n2Cc1cc(F)c(F)c(F)c1] | 1 | 1.98 | 609 |
| I-0275 | 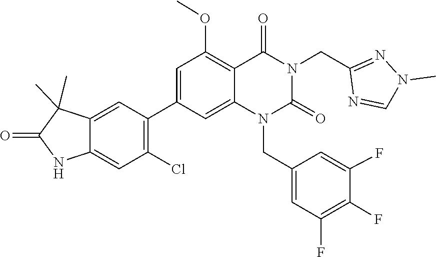 [SMILES: COc1cc(-c2cc3c(cc2Cl)NC(=O)C3(C)C)cc2c1c(=O)n(Cc1ncn(C)n1)c(=O)n2Cc1cc(F)c(F)c(F)c1] | 1 | 1.98 | 625 |
| I-0276 | 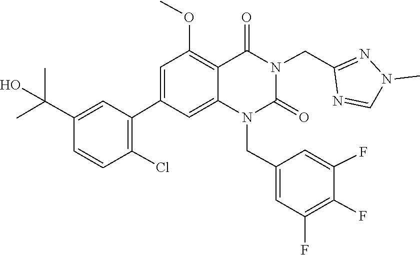 [SMILES: COc1cc(-c2cc(C(C)(C)O)ccc2Cl)cc2c1c(=O)n(Cc1ncn(C)n1)c(=O)n2Cc1cc(F)c(F)c(F)c1] | 1 | 2.11 | 600 |
| I-0277 | 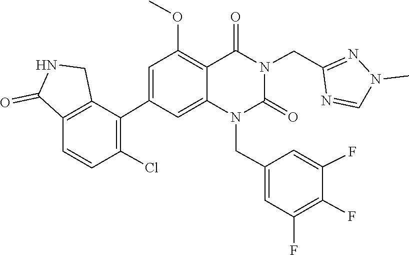 [SMILES: COc1cc(-c2c(Cl)ccc3c2CNC3=O)cc2c1c(=O)n(Cc1ncn(C)n1)c(=O)n2Cc1cc(F)c(F)c(F)c1] | 1 | 1.84 | 597 |

TABLE 25

| | | LC/MS | Retention | |
|---|---|---|---|---|
| Compound | | measurement | time | |
| No. | Structural formula | conditions | (min) | m/z |
| I-0278 | 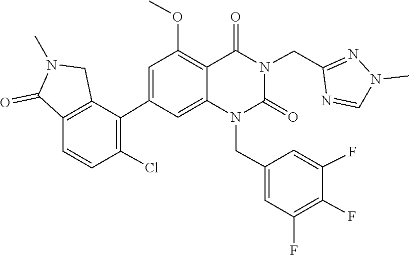 [SMILES: COc1cc(-c2c(Cl)ccc3c2CN(C)C3=O)cc2c1c(=O)n(Cc1ncn(C)n1)c(=O)n2Cc1cc(F)c(F)c(F)c1] | 1 | 1.90 | 611 |
| I-0279 | 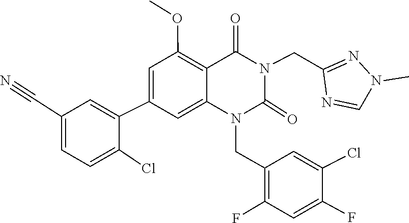 [SMILES: COc1cc(-c2cc(C#N)ccc2Cl)cc2c1c(=O)n(Cc1ncn(C)n1)c(=O)n2Cc1cc(Cl)c(F)cc1F] | 1 | 2.27 | 583 |
| I-0280 | 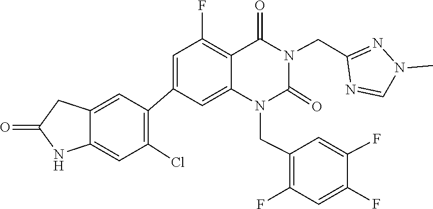 [SMILES: Cn1cnc(Cn2c(=O)c3c(F)cc(-c4cc5c(cc4Cl)NC(=O)C5)cc3n(Cc3cc(F)c(F)cc3F)c2=O)n1] | 1 | 1.85 | 585 |
| I-0281 | 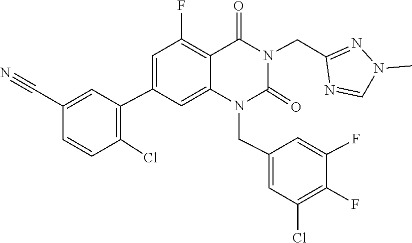 [SMILES: Cn1cnc(Cn2c(=O)c3c(F)cc(-c4cc(C#N)ccc4Cl)cc3n(Cc3cc(F)c(F)c(Cl)c3)c2=O)n1] | 1 | 2.41 | 571 |
| I-0282 | 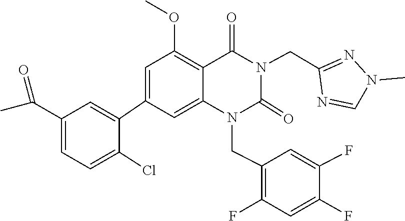 [SMILES: COc1cc(-c2cc(C(C)=O)ccc2Cl)cc2c1c(=O)n(Cc1ncn(C)n1)c(=O)n2Cc1cc(F)c(F)cc1F] | 1 | 2.17 | 584 |
| I-0283 | 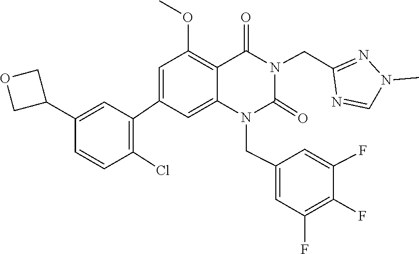 [SMILES: COc1cc(-c2cc(C3COC3)ccc2Cl)cc2c1c(=O)n(Cc1ncn(C)n1)c(=O)n2Cc1cc(F)c(F)c(F)c1] | 1 | 2.14 | 598 |
| I-0284 | 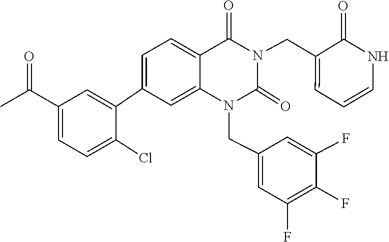 [SMILES: CC(=O)c1ccc(Cl)c(-c2ccc3c(=O)n(Cc4ccc[nH]c4=O)c(=O)n(Cc4cc(F)c(F)c(F)c4)c3c2)c1] | 1 | 2.23 | 566 |

TABLE 26

| | | LC/MS | Retention | |
|---|---|---|---|---|
| Compound | | measurement | time | |
| No. | Structural formula | conditions | (min) | m/z |
| I-0285 | 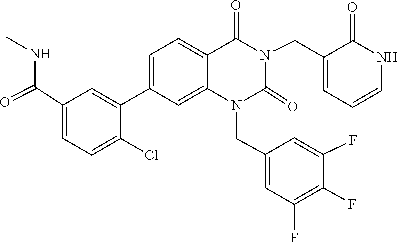 [SMILES: CNC(=O)c1ccc(Cl)c(-c2ccc3c(=O)n(Cc4ccc[nH]c4=O)c(=O)n(Cc4cc(F)c(F)c(F)c4)c3c2)c1] | 1 | 1.95 | 581 |
| I-0295 | 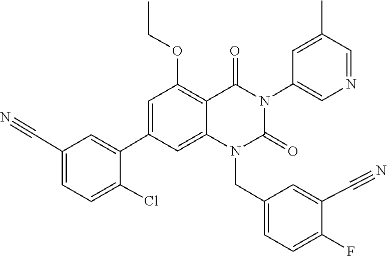 [SMILES: CCOc1cc(-c2cc(C#N)ccc2Cl)cc2c1c(=O)n(-c1cncc(C)c1)c(=O)n2Cc1ccc(F)c(C#N)c1] | 1 | 2.22 | 566.1 |
| I-0296 | 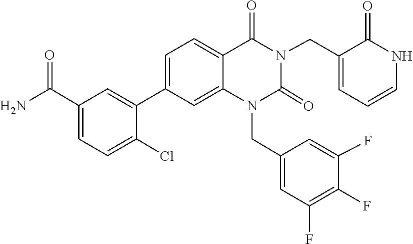 [SMILES: NC(=O)c1ccc(Cl)c(-c2ccc3c(=O)n(Cc4ccc[nH]c4=O)c(=O)n(Cc4cc(F)c(F)c(F)c4)c3c2)c1] | 1 | 1.89 | 567.05 |
| I-0301 | 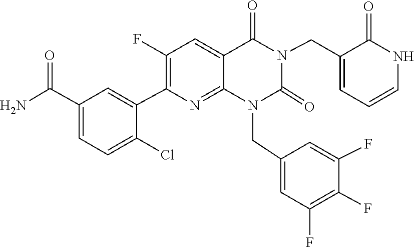 [SMILES: NC(=O)c1ccc(Cl)c(-c2nc3c(cc2F)c(=O)n(Cc2ccc[nH]c2=O)c(=O)n3Cc2cc(F)c(F)c(F)c2)c1] | 1 | 1.93 | 586 |
| I-0315 | 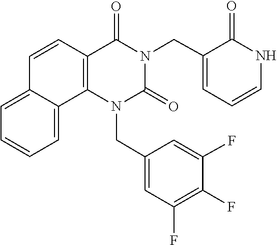 [SMILES: O=c1[nH]cccc1Cn1c(=O)c2ccc3ccccc3c2n(Cc2cc(F)c(F)c(F)c2)c1=O] | 1 | 2.17 | 464.05 |
| I-0325 | 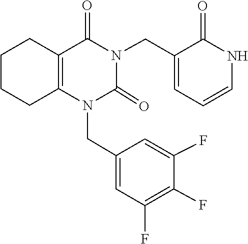 [SMILES: O=c1[nH]cccc1Cn1c(=O)c2c(n(Cc3cc(F)c(F)c(F)c3)c1=O)CCCC2] | 1 | 1.84 | 417.95 |

TABLE 27

| | | LC/MS | Retention | |
|---|---|---|---|---|
| Compound | | measurement | time | |
| No. | Structural formula | conditions | (min) | m/z |
| I-0353 | 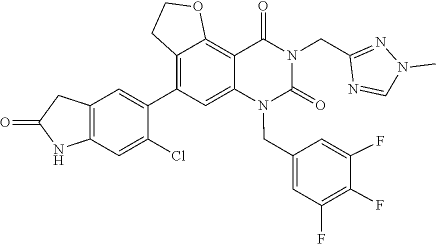 [SMILES: Cn1cnc(Cn2c(=O)c3c4c(c(-c5cc6c(cc5Cl)NC(=O)C6)cc3n(Cc3cc(F)c(F)c(F)c3)c2=O)CCO4)n1] | 1 | 1.83 | 609.05 |
| I-0363 | 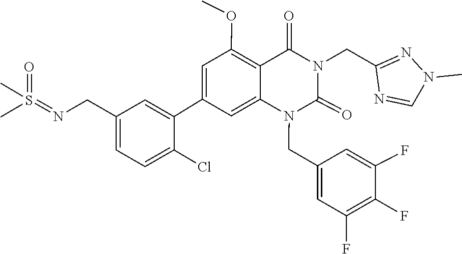 | 1 | 1.78 | 647 |
| I-0378 | 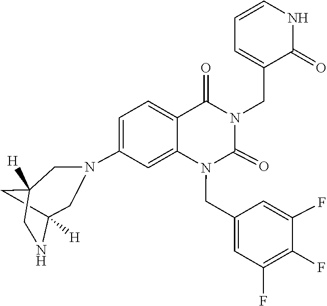 [SMILES: O=c1[nH]cccc1Cn1c(=O)c2ccc(N3C[C@@H]4CN[C@@H](C4)C3)cc2n(Cc2cc(F)c(F)c(F)c2)c1=O] | 1 | 1.34 | 524 |
| I-0379 | 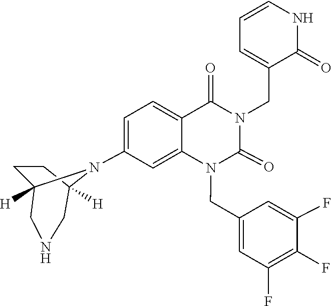 [SMILES: O=c1[nH]cccc1Cn1c(=O)c2ccc(N3[C@@H]4CC[C@H]3CNC4)cc2n(Cc2cc(F)c(F)c(F)c2)c1=O] | 1 | 1.37 | 524 |
| I-0380 | 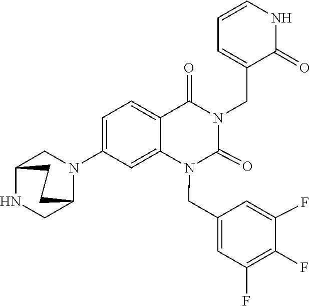 [SMILES: O=c1[nH]cccc1Cn1c(=O)c2ccc(N3C[C@@H]4CC[C@H]3CN4)cc2n(Cc2cc(F)c(F)c(F)c2)c1=O] | 1 | 1.33 | 524 |
| I-0381 | 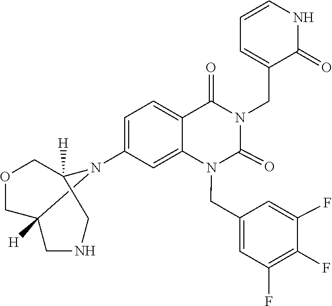 [SMILES: O=c1[nH]cccc1Cn1c(=O)c2ccc(N3[C@@H]4CNC[C@H]3COC4)cc2n(Cc2cc(F)c(F)c(F)c2)c1=O] | 1 | 1.32 | 540 |

TABLE 28

| | | LC/MS | Retention | |
|---|---|---|---|---|
| Compound | | measurement | time | |
| No. | Structural formula | conditions | (min) | m/z |
| I-0388 | 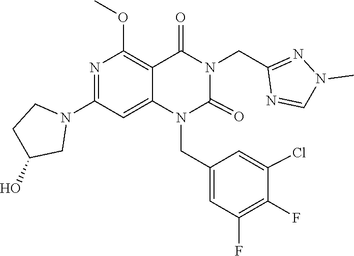 [SMILES: COc1nc(N2CC[C@@H](O)C2)cc2c1c(=O)n(Cc1ncn(C)n1)c(=O)n2Cc1cc(F)c(F)c(Cl)c1] | 1 | 1.76 | 534 |
| I-0389 | 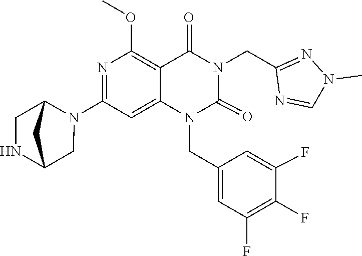 [SMILES: COc1nc(N2C[C@H]3C[C@@H]2CN3)cc2c1c(=O)n(Cc1ncn(C)n1)c(=O)n2Cc1cc(F)c(F)c(F)c1] | 1 | 1.40 | 529 |
| I-0393 | 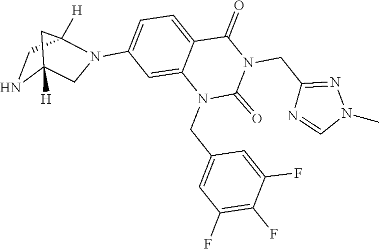 [SMILES: Cn1cnc(Cn2c(=O)c3ccc(N4C[C@H]5C[C@@H]4CN5)cc3n(Cc3cc(F)c(F)c(F)c3)c2=O)n1] | 1 | 1.34 | 498 |
| I-0395 | 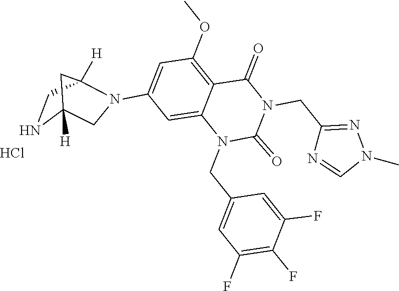 [SMILES: Cl ; COc1cc(N2C[C@H]3C[C@@H]2CN3)cc2c1c(=O)n(Cc1ncn(C)n1)c(=O)n2Cc1cc(F)c(F)c(F)c1] | 1 | 1.92 | 528 |
| I-0396 | 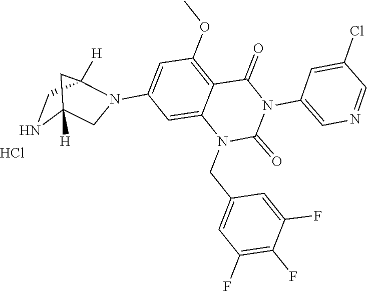 [SMILES: Cl ; COc1cc(N2C[C@H]3C[C@@H]2CN3)cc2c1c(=O)n(-c1cncc(Cl)c1)c(=O)n2Cc1cc(F)c(F)c(F)c1] | 1 | 1.53 | 544 |
| I-0414 | 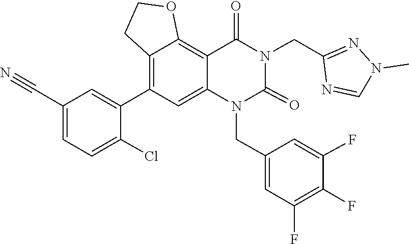 [SMILES: Cn1cnc(Cn2c(=O)c3c4c(c(-c5cc(C#N)ccc5Cl)cc3n(Cc3cc(F)c(F)c(F)c3)c2=O)CCO4)n1] | 1 | 2.20 | 579 |

TABLE 29

| | | LC/MS | Retention | |
|---|---|---|---|---|
| Compound | | measurement | time | |
| No. | Structural formula | conditions | (min) | m/z |
| I-0415 | 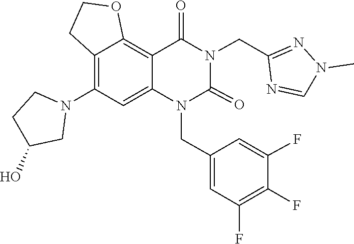 [SMILES: Cn1cnc(Cn2c(=O)c3c4c(c(N5CC[C@@H](O)C5)cc3n(Cc3cc(F)c(F)c(F)c3)c2=O)CCO4)n1] | 1 | 1.65 | 529 |
| I-0429 | 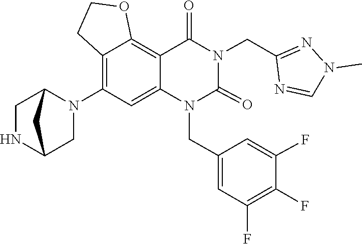 [SMILES: Cn1cnc(Cn2c(=O)c3c4c(c(N5C[C@H]6C[C@@H]5CN6)cc3n(Cc3cc(F)c(F)c(F)c3)c2=O)CCO4)n1] | 1 | 1.36 | 540 |
| I-0431 | 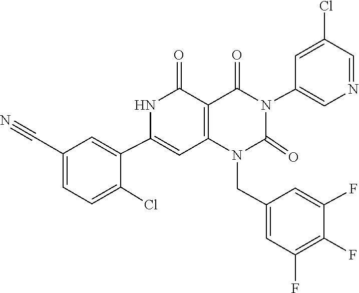 [SMILES: N#Cc1ccc(Cl)c(-c2cc3c(c(=O)[nH]2)c(=O)n(-c2cncc(Cl)c2)c(=O)n3Cc2cc(F)c(F)c(F)c2)c1] | 1 | 2.15 | 569.95 |
| I-0435 | 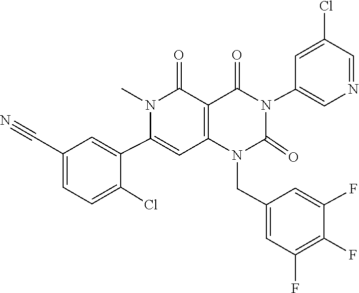 [SMILES: Cn1c(-c2cc(C#N)ccc2Cl)cc2c(c1=O)c(=O)n(-c1cncc(Cl)c1)c(=O)n2Cc1cc(F)c(F)c(F)c1] | 1 | 2.22 | 583.9 |
| I-0437 | 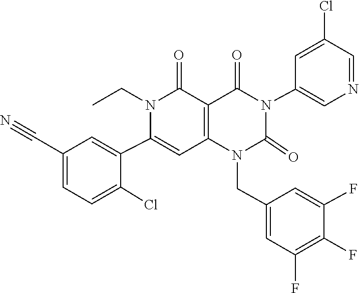 [SMILES: CCn1c(-c2cc(C#N)ccc2Cl)cc2c(c1=O)c(=O)n(-c1cncc(Cl)c1)c(=O)n2Cc1cc(F)c(F)c(F)c1] | 1 | 2.75 | 597.95 |
| I-0438 | 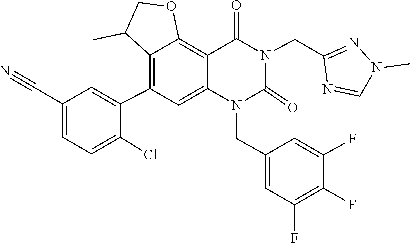 [SMILES: CC1COc2c1c(-c1cc(C#N)ccc1Cl)cc1c2c(=O)n(Cc2ncn(C)n2)c(=O)n1Cc1cc(F)c(F)c(F)c1] | 1 | 2.29 | 593 |

TABLE 30

| | | LC/MS | | |
|---|---|---|---|---|
| Compound | | measurement | Retention time | |
| No. | Structural formula | conditions | (min) | m/z |
| 1-0439 | 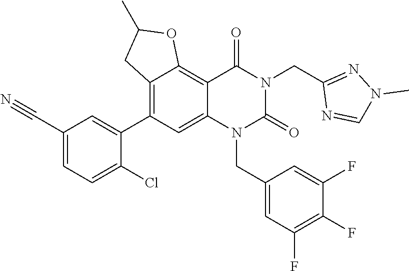 [SMILES: CC1Cc2c(-c3cc(C#N)ccc3Cl)cc3c(c2O1)c(=O)n(Cc1ncn(C)n1)c(=O)n3Cc1cc(F)c(F)c(F)c1] | 1 | 2.29 | 593 |
| 1-0440 | 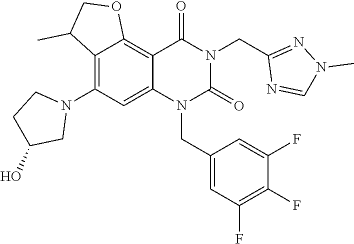 [SMILES: CC1COc2c1c(N1CC[C@@H](O)C1)cc1c2c(=O)n(Cc2ncn(C)n2)c(=O)n1Cc1cc(F)c(F)c(F)c1] | 1 | 1.73 | 543 |
| 1-0442 | 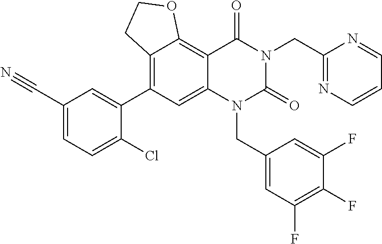 [SMILES: N#Cc1ccc(Cl)c(-c2cc3c(c4c2CCO4)c(=O)n(Cc2ncccn2)c(=O)n3Cc2cc(F)c(F)c(F)c2)c1] | 1 | 2.33 | 576 |
| 1-0443 | 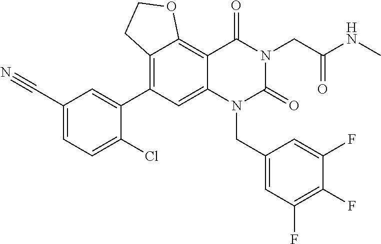 [SMILES: CNC(=O)Cn1c(=O)c2c3c(c(-c4cc(C#N)ccc4Cl)cc2n(Cc2cc(F)c(F)c(F)c2)c1=O)CCO3] | 1 | 2.17 | 555 |
| 1-0445 | 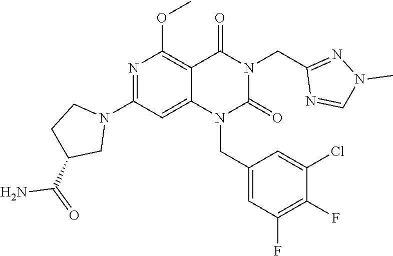 [SMILES: COc1nc(N2CC[C@@H](C(N)=O)C2)cc2c1c(=O)n(Cc1ncn(C)n1)c(=O)n2Cc1cc(F)c(F)c(Cl)c1] | 1 | 1.69 | 561 |
| 1-0446 | 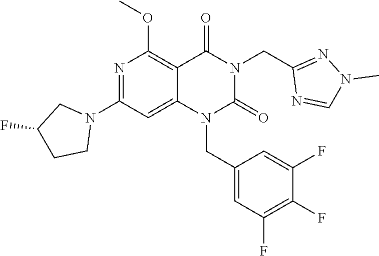 [SMILES: COc1nc(N2CC[C@H](F)C2)cc2c1c(=O)n(Cc1ncn(C)n1)c(=O)n2Cc1cc(F)c(F)c(F)c1] | 1 | 2.05 | 520 |

TABLE 31

| | | LC/MS | | |
|---|---|---|---|---|
| Compound | | measurement | Retention time | |
| No. | Structural formula | conditions | (min) | m/z |
| 1-0449 | 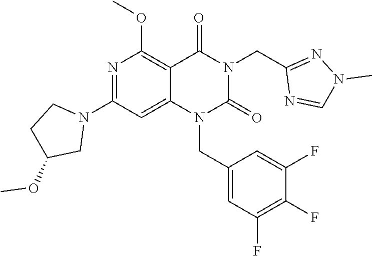 | 1 | 1.78 | 532 |
| 1-0451 | 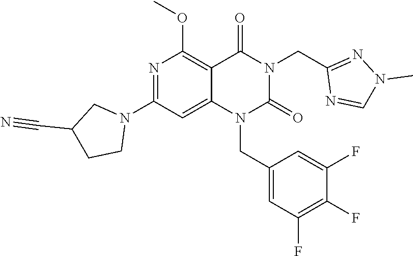 [SMILES: COc1nc(N2CCC(C#N)C2)cc2c1c(=O)n(Cc1ncn(C)n1)c(=O)n2Cc1cc(F)c(F)c(F)c1] | 1 | 2.14 | 527 |
| 1-0453 | 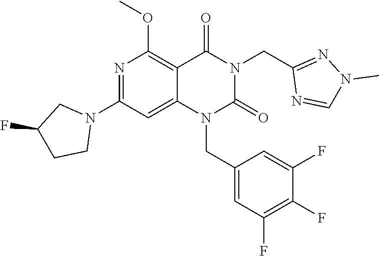 [SMILES: COc1nc(N2CC[C@@H](F)C2)cc2c1c(=O)n(Cc1ncn(C)n1)c(=O)n2Cc1cc(F)c(F)c(F)c1] | 1 | 2.05 | 520 |
| 1-0459 | 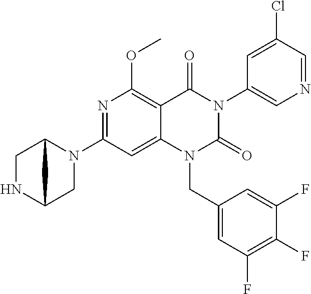 [SMILES: COc1nc(N2C[C@H]3C[C@@H]2CN3)cc2c1c(=O)n(-c1cncc(Cl)c1)c(=O)n2Cc1cc(F)c(F)c(F)c1] | 1 | 1.66 | 545.1 |
| 1-0460 | 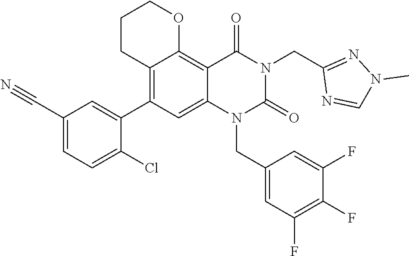 [SMILES: Cn1cnc(Cn2c(=O)c3c4c(c(-c5cc(C#N)ccc5Cl)cc3n(Cc3cc(F)c(F)c(F)c3)c2=O)CCCO4)n1] | 1 | 2.24 | 593 |
| 1-0464 | 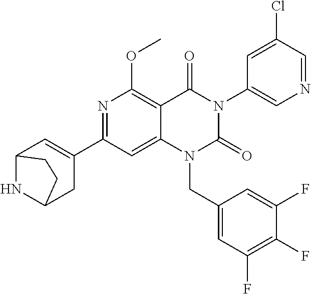 [SMILES: COc1nc(C2=CC3CCC(C2)N3)cc2c1c(=O)n(-c1cncc(Cl)c1)c(=O)n2Cc1cc(F)c(F)c(F)c1] | 1 | 1.76 | 556.05 |

TABLE 32

| | | LC/MS | | |
|---|---|---|---|---|
| Compound | | measurement | Retention time | |
| No. | Structural formula | conditions | (min) | m/z |
| 1-0466 | 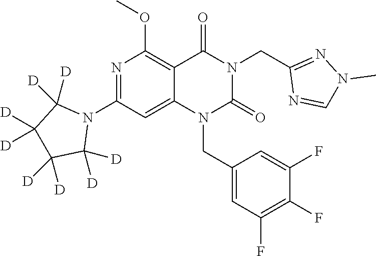 [SMILES: [2H]C1([2H])N(c2cc3c(c(OC)n2)c(=O)n(Cc2ncn(C)n2)c(=O)n3Cc2cc(F)c(F)c(F)c2)C([2H])([2H])C([2H])([2H])C1([2H])[2H]] | 1 | 2.32 | 510 |
| 1-0472 | 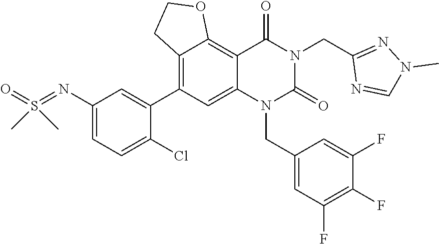 [SMILES: Cn1cnc(Cn2c(=O)c3c4c(c(-c5cc(N=S(C)(C)=O)ccc5Cl)cc3n(Cc3cc(F)c(F)c(F)c3)c2=O)CCO4)n1] | 1 | 1.92 | 645 |
| 1-0473 | 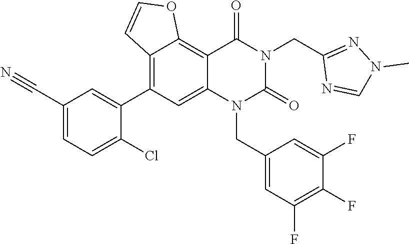 [SMILES: Cn1cnc(Cn2c(=O)c3c4occc4c(-c4cc(C#N)ccc4Cl)cc3n(Cc3cc(F)c(F)c(F)c3)c2=O)n1] | 1 | 2.25 | 577 |
| 1-0484 | 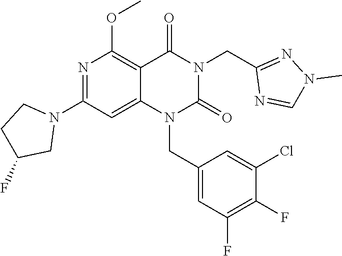 [SMILES: COc1nc(N2CC[C@@H](F)C2)cc2c1c(=O)n(Cc1ncn(C)n1)c(=O)n2Cc1cc(F)c(F)c(Cl)c1] | 1 | 2.15 | 536 |
| 1-0492 | 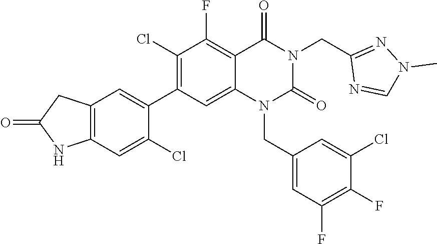 [SMILES: Cn1cnc(Cn2c(=O)c3c(F)c(Cl)c(-c4cc5c(cc4Cl)NC(=O)C5)cc3n(Cc3cc(F)c(F)c(Cl)c3)c2=O)n1] | 1 | 2.05 | 635 |
| 1-0501 | 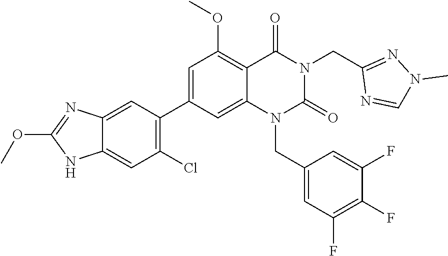 | 1 | 1.85 | 612.2 |

TABLE 33

| | | LC/MS | | |
|---|---|---|---|---|
| Compound | | measurement | Retention time | |
| No. | Structural formula | conditions | (min) | m/z |
| 1-0504 | 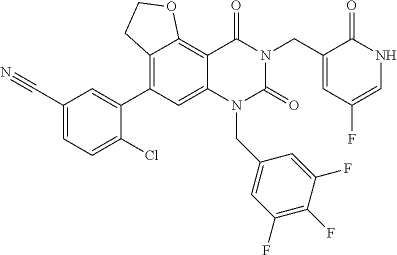 [SMILES: N#Cc1ccc(Cl)c(-c2cc3c(c4c2CCO4)c(=O)n(Cc2cc(F)c[nH]c2=O)c(=O)n3Cc2cc(F)c(F)c(F)c2)c1] | 1 | 2.17 | 609.1 |
| 1-0506 | 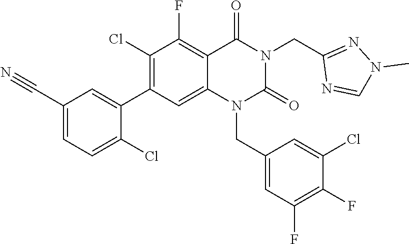 [SMILES: Cn1cnc(Cn2c(=O)c3c(F)c(Cl)c(-c4cc(C#N)ccc4Cl)cc3n(Cc3cc(F)c(F)c(Cl)c3)c2=O)n1] | 1 | 2.49 | 605 |
| 1-0511 | 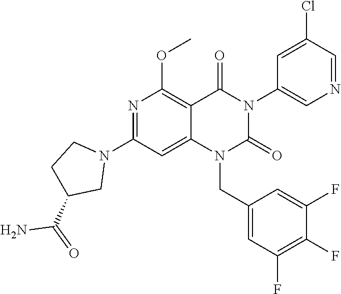 [SMILES: COc1nc(N2CC[C@@H](C(N)=O)C2)cc2c1c(=O)n(-c1cncc(Cl)c1)c(=O)n2Cc1cc(F)c(F)c(F)c1] | 1 | 1.95 | 561 |
| 1-0514 | 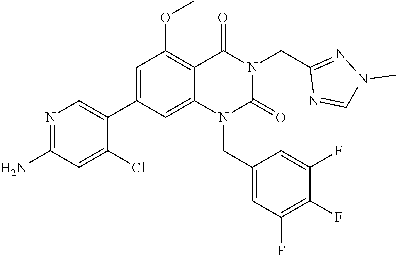 [SMILES: COc1cc(-c2cnc(N)cc2Cl)cc2c1c(=O)n(Cc1ncn(C)n1)c(=O)n2Cc1cc(F)c(F)c(F)c1] | 1 | 1.53 | 558 |
| 1-0516 | 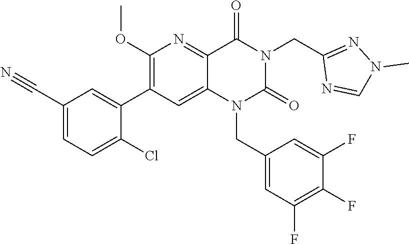 [SMILES: COc1nc2c(=O)n(Cc3ncn(C)n3)c(=O)n(Cc3cc(F)c(F)c(F)c3)c2cc1-c1cc(C#N)ccc1Cl] | 1 | 2.20 | 568.05 |
| 1-0517 | 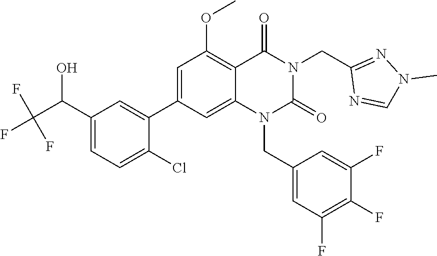 [SMILES: COc1cc(-c2cc(C(O)C(F)(F)F)ccc2Cl)cc2c1c(=O)n(Cc1ncn(C)n1)c(=O)n2Cc1cc(F)c(F)c(F)c1] | 1 | 2.21 | 640 |

TABLE 34

| | | LC/MS | | |
|---|---|---|---|---|
| Compound | | measurement | Retention time | |
| No. | Structural formula | conditions | (min) | m/z |
| 1-0523 | 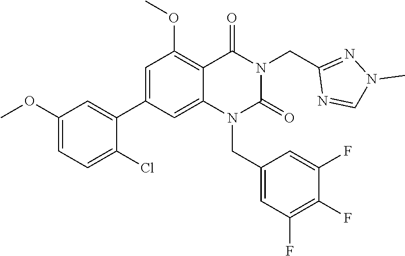 | 1 | 2.35 | 572 |
| 1-0540 | 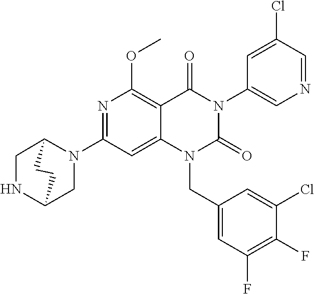 [SMILES: COc1nc(N2C[C@@H]3CC[C@H]2CN3)cc2c1c(=O)n(-c1cncc(Cl)c1)c(=O)n2Cc1cc(F)c(F)c(Cl)c1] | 1 | 1.72 | 599.05 |
| 1-0541 | 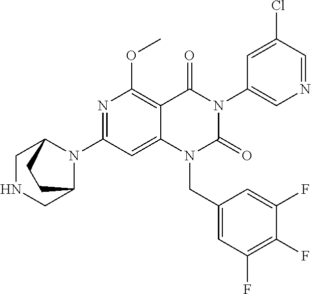 [SMILES: COc1nc(N2[C@@H]3CC[C@H]2CNC3)cc2c1c(=O)n(-c1cncc(Cl)c1)c(=O)n2Cc1cc(F)c(F)c(F)c1] | 1 | 1.77 | 599.05 |
| 1-0542 | 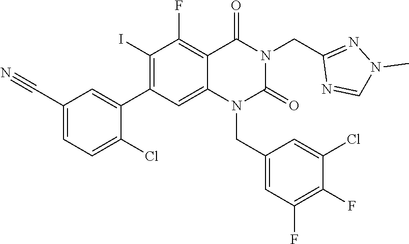 [SMILES: Cn1cnc(Cn2c(=O)c3c(F)c(I)c(-c4cc(C#N)ccc4Cl)cc3n(Cc3cc(F)c(F)c(Cl)c3)c2=O)n1] | 1 | 2.52 | 697 |
| 1-0546 | 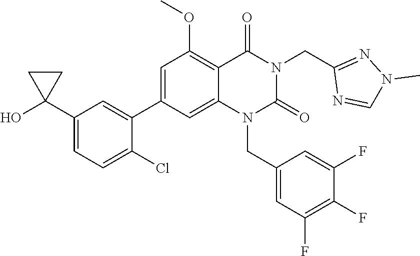 [SMILES: COc1cc(-c2cc(C3(O)CC3)ccc2Cl)cc2c1c(=O)n(Cc1ncn(C)n1)c(=O)n2Cc1cc(F)c(F)c(F)c1] | 1 | 2.28 | 598.1 |
| 1-0547 | 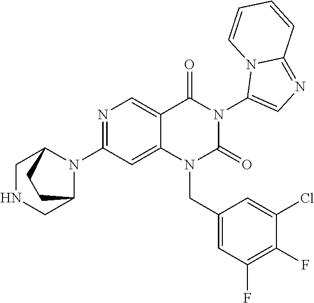 [SMILES: O=c1c2cnc(N3[C@@H]4CC[C@H]3CNC4)cc2n(Cc2cc(F)c(F)c(Cl)c2)c(=O)n1-c1cnc2ccccn12] | 3 | 1.69 | 550 |

TABLE 35

| | | LC/MS | | |
|---|---|---|---|---|
| Compound | | measurement | Retention time | |
| No. | Structural formula | conditions | (min) | m/z |
| 1-0548 | 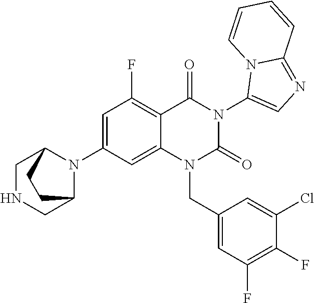 [SMILES: O=c1c2c(F)cc(N3[C@@H]4CC[C@H]3CNC4)cc2n(Cc2cc(F)c(F)c(Cl)c2)c(=O)n1-c1cnc2ccccn12] | 3 | 1.98 | 567 |
| 1-0549 | 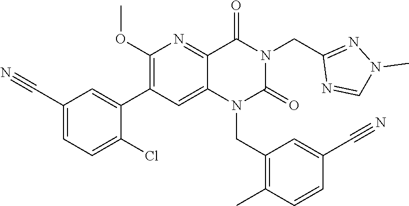 [SMILES: COc1nc2c(=O)n(Cc3ncn(C)n3)c(=O)n(Cc3cc(C#N)ccc3C)c2cc1-c1cc(C#N)ccc1Cl] | 1 | 1.98 | 553.05 |
| 1-0550 | 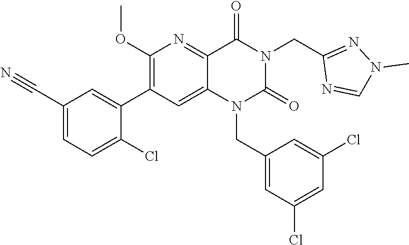 [SMILES: COc1nc2c(=O)n(Cc3ncn(C)n3)c(=O)n(Cc3cc(Cl)cc(Cl)c3)c2cc1-c1cc(C#N)ccc1Cl] | 1 | 2.31 | 583.95 |
| 1-0551 | 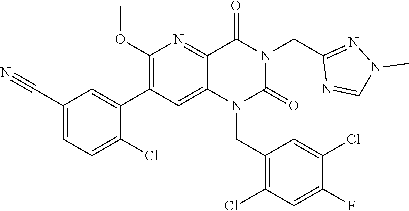 [SMILES: COc1nc2c(=O)n(Cc3ncn(C)n3)c(=O)n(Cc3cc(Cl)c(F)cc3Cl)c2cc1-c1cc(C#N)ccc1Cl] | 1 | 2.41 | 600 |
| 1-0552 | 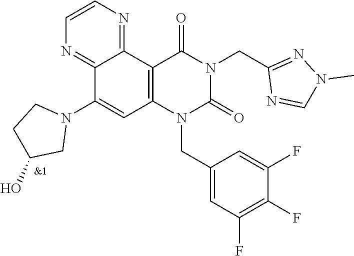 [SMILES: Cn1cnc(Cn2c(=O)c3c4nccnc4c(N4CC[C@@H](O)C4)cc3n(Cc3cc(F)c(F)c(F)c3)c2=O)n1] | 1 | 1.58 | 539 |
| 1-0553 | 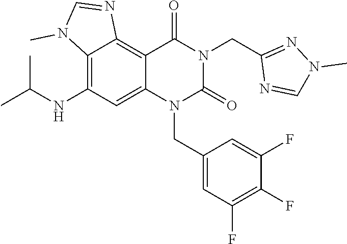 [SMILES: CC(C)Nc1cc2c(c(=O)n(Cc3ncn(C)n3)c(=O)n2Cc2cc(F)c(F)c(F)c2)c2ncn(C)c12] | 1 | 1.75 | 513 |

TABLE 36

| | | LC/MS | | |
|---|---|---|---|---|
| Compound | | measurement | Retention time | |
| No. | Structural formula | conditions | (min) | m/z |
| 1-0554 | 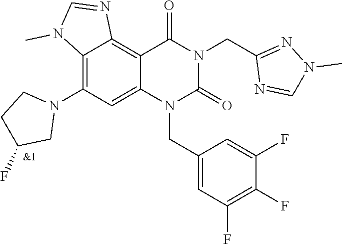 [SMILES: Cn1cnc(Cn2c(=O)c3c4ncn(C)c4c(N4CC[C@@H](F)C4)cc3n(Cc3cc(F)c(F)c(F)c3)c2=O)n1] | 1 | 1.64 | 543 |
| 1-0556 | 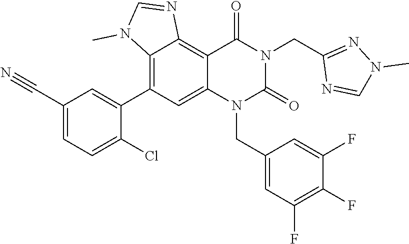 [SMILES: Cn1cnc(Cn2c(=O)c3c4ncn(C)c4c(-c4cc(C#N)ccc4Cl)cc3n(Cc3cc(F)c(F)c(F)c3)c2=O)n1] | 1 | 1.82 | 591 |
| I-0557 | 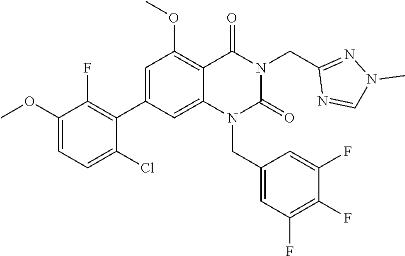 | 1 | 2.36 | 589.95 |
| 1-0561 | 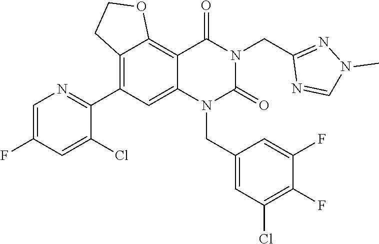 [SMILES: Cn1cnc(Cn2c(=O)c3c4c(c(-c5ncc(F)cc5Cl)cc3n(Cc3cc(F)c(F)c(Cl)c3)c2=O)CCO4)n1] | 1 | 2.37 | 589.07 |
| 1-0563 | 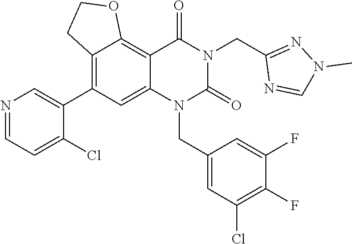 [SMILES: Cn1cnc(Cn2c(=O)c3c4c(c(-c5cnccc5Cl)cc3n(Cc3cc(F)c(F)c(Cl)c3)c2=O)CCO4)n1] | 1 | 2.15 | 571 |
| 1-0565 | 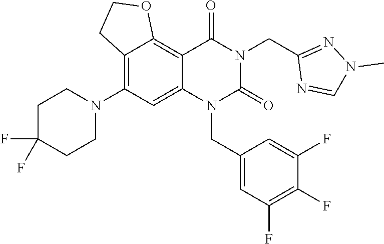 [SMILES: Cn1cnc(Cn2c(=O)c3c4c(c(N5CCC(F)(F)CC5)cc3n(Cc3cc(F)c(F)c(F)c3)c2=O)CCO4)n1] | 1 | 2.16 | 563 |

TABLE 37

| | | LC/MS | | |
|---|---|---|---|---|
| Compound | | measurement | Retention time | |
| No. | Structural formula | conditions | (min) | m/z |
| 1-0570 | 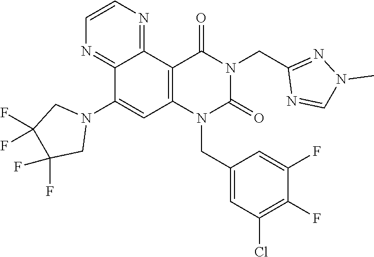 [SMILES: Cn1cnc(Cn2c(=O)c3c4nccnc4c(N4CC(F)(F)C(F)(F)C4)cc3n(Cc3cc(F)c(F)c(Cl)c3)c2=O)n1] | 1 | 2.41 | 611.05 |
| 1-0571 | 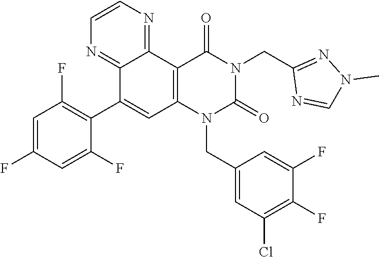 [SMILES: Cn1cnc(Cn2c(=O)c3c4nccnc4c(-c4c(F)cc(F)cc4F)cc3n(Cc3cc(F)c(F)c(Cl)c3)c2=O)n1] | 1 | 2.31 | 600 |
| 1-0572 | 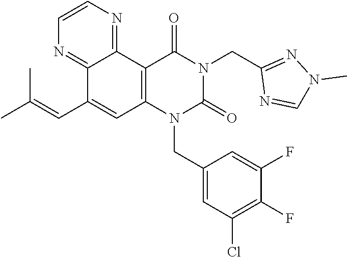 [SMILES: CC(C)=Cc1cc2c(c(=O)n(Cc3ncn(C)n3)c(=O)n2Cc2cc(F)c(F)c(Cl)c2)c2nccnc12] | 1 | 2.28 | 524.1 |
| 1-0573 | 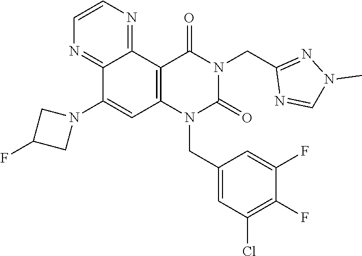 [SMILES: Cn1cnc(Cn2c(=O)c3c4nccnc4c(N4CC(F)C4)cc3n(Cc3cc(F)c(F)c(Cl)c3)c2=O)n1] | 1 | 2.16 | 543.1 |
| 1-0575 | 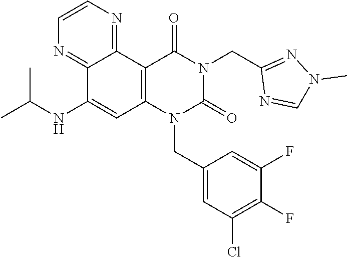 [SMILES: CC(C)Nc1cc2c(c(=O)n(Cc3ncn(C)n3)c(=O)n2Cc2cc(F)c(F)c(Cl)c2)c2nccnc12] | 1 | 2.27 | 527.1 |
| 1-0578 | 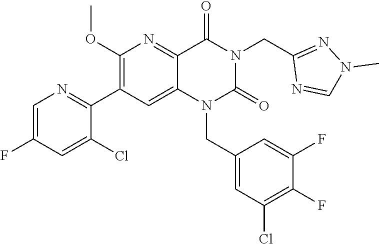 [SMILES: COc1nc2c(=O)n(Cc3ncn(C)n3)c(=O)n(Cc3cc(F)c(F)c(Cl)c3)c2cc1-c1ncc(F)cc1Cl] | 1 | 2.30 | 578.05 |

TABLE 38

| | | LC/MS | | |
|---|---|---|---|---|
| Compound | | measurement | Retention time | |
| No. | Structural formula | conditions | (min) | m/z |
| I-0579 | 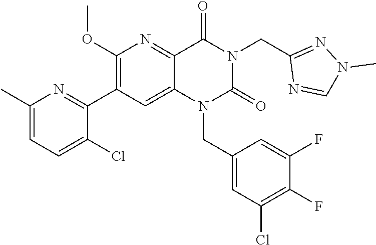 | 1 | 2.27 | 574.05 |
| I-0580 | 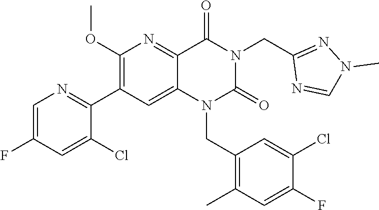 [SMILES: COc1nc2c(=O)n(Cc3ncn(C)n3)c(=O)n(Cc3cc(Cl)c(F)cc3C)c2cc1-c1ncc(F)cc1Cl] | 1 | 2.27 | 574.05 |
| 1-0581 | 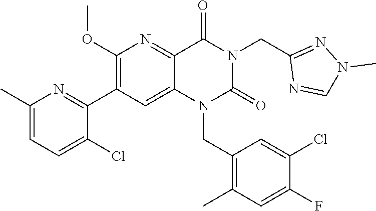 | 1 | 2.25 | 570.05 |
| 1-0586 | 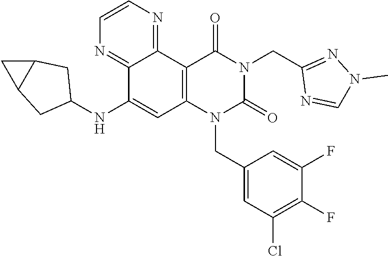 [SMILES: Cn1cnc(Cn2c(=O)c3c4nccnc4c(NC4CC5CC5C4)cc3n(Cc3cc(F)c(F)c(Cl)c3)c2=O)n1] | 1 | 2.30 | 565 |
| I-0595 | 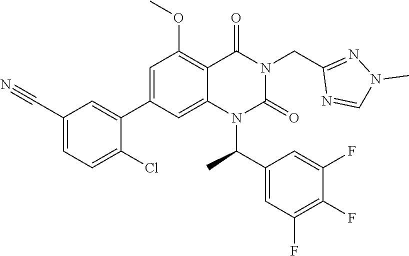 [SMILES: COc1cc(-c2cc(C#N)ccc2Cl)cc2c1c(=O)n(Cc1ncn(C)n1)c(=O)n2[C@H](C)c1cc(F)c(F)c(F)c1] | 1 | 2.24 | 581 |
| 1-0598 | 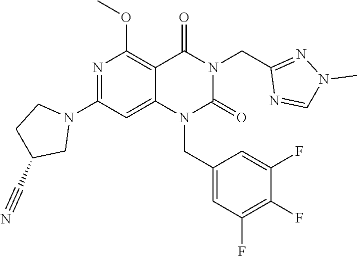 [SMILES: COc1nc(N2CC[C@@H](C#N)C2)cc2c1c(=O)n(Cc1ncn(C)n1)c(=O)n2Cc1cc(F)c(F)c(F)c1] | 1 | 1.89 | 527.05 |
| I-0602 | 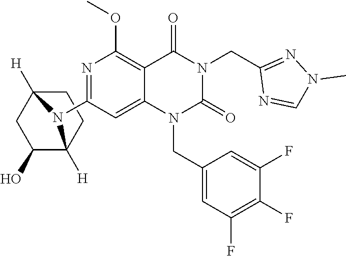 [SMILES: COc1nc(N2[C@H]3CC[C@@H]2[C@@H](O)C3)cc2c1c(=O)n(Cc1ncn(C)n1)c(=O)n2Cc1cc(F)c(F)c(F)c1] | 1 | 1.83 | 544.1 |

TABLE 39

| | | LC/MS | | |
|---|---|---|---|---|
| Compound | | measurement | Retention time | |
| No. | Structural formula | conditions | (min) | m/z |
| 1-0610 | 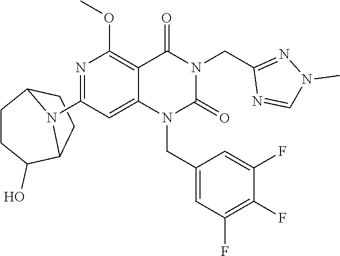 [SMILES: COc1nc(N2C3CCC(O)C2CC3)cc2c1c(=O)n(Cc1ncn(C)n1)c(=O)n2Cc1cc(F)c(F)c(F)c1] | 1 | 1.96 | 558 |
| 1-0623 | 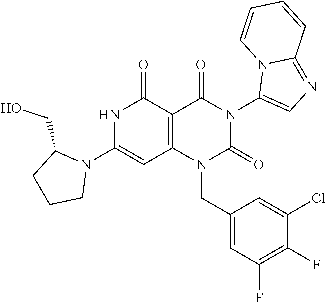 [SMILES: O=c1[nH]c(N2CCC[C@@H]2CO)cc2c1c(=O)n(-c1cnc3ccccn13)c(=O)n2Cc1cc(F)c(F)c(Cl)c1] | 1 | 1.51 | 555.05 |
| 1-0625 | 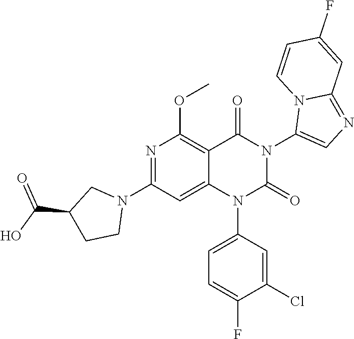 [SMILES: COc1nc(N2CC[C@@H](C(=O)O)C2)cc2c1c(=O)n(-c1cnc3cc(F)ccn13)c(=O)n2-c1ccc(F)c(Cl)c1] | 1 | 1.72 | 569 |
| 1-0626 | 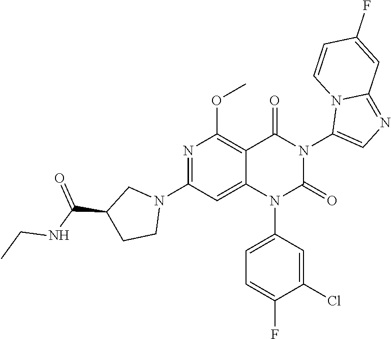 [SMILES: CCNC(=O)[C@@H]1CCN(c2cc3c(c(OC)n2)c(=O)n(-c2cnc4cc(F)ccn24)c(=O)n3-c2ccc(F)c(Cl)c2)C1] | 1 | 1.72 | 596.05 |
| 1-0627 | 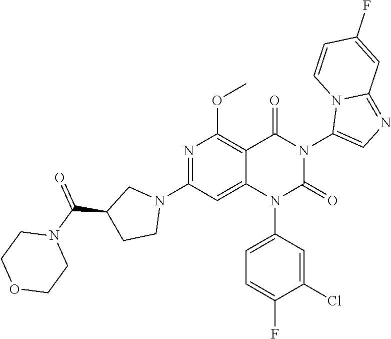 [SMILES: COc1nc(N2CC[C@@H](C(=O)N3CCOCC3)C2)cc2c1c(=O)n(-c1cnc3cc(F)ccn13)c(=O)n2-c1ccc(F)c(Cl)c1] | 1 | 1.75 | 638.1 |

TABLE 40

| | | LC/MS | | |
|---|---|---|---|---|
| Compound | | measurement | Retention time | |
| No. | Structural formula | conditions | (min) | m/z |
| 1-0628 | 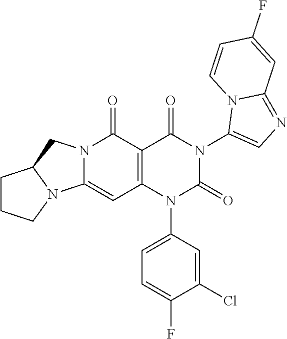 [SMILES: O=c1c2c(=O)n(-c3cnc4cc(F)ccn34)c(=O)n(-c3ccc(F)c(Cl)c3)c2cc2n1C[C@@H]1CCCN21] | 1 | 1.63 | 523 |
| 1-0632 | 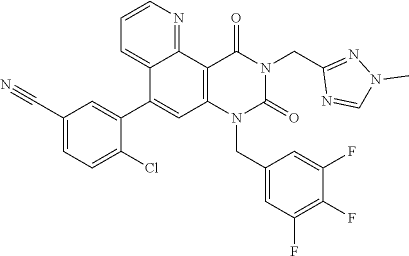 [SMILES: Cn1cnc(Cn2c(=O)c3c4ncccc4c(-c4cc(C#N)ccc4Cl)cc3n(Cc3cc(F)c(F)c(F)c3)c2=O)n1] | 1 | 1.71 | 588 |
| 1-0637 | 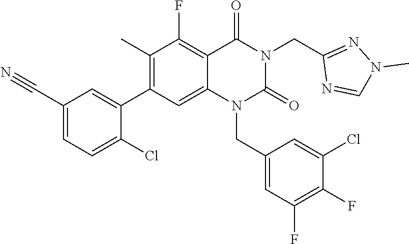 [SMILES: Cc1c(-c2cc(C#N)ccc2Cl)cc2c(c1F)c(=O)n(Cc1ncn(C)n1)c(=O)n2Cc1cc(F)c(F)c(Cl)c1] | 1 | 2.44 | 685 |
| 1-0638 | 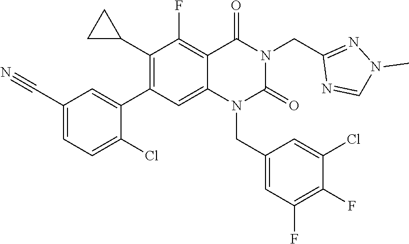 [SMILES: Cn1cnc(Cn2c(=O)c3c(F)c(C4CC4)c(-c4cc(C#N)ccc4Cl)cc3n(Cc3cc(F)c(F)c(Cl)c3)c2=O)n1] | 1 | 2.56 | 611 |
| 1-0639 | 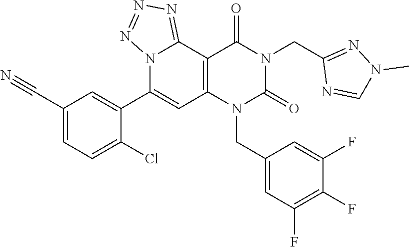 [SMILES: Cn1cnc(Cn2c(=O)c3c(cc(-c4cc(C#N)ccc4Cl)n4nnnc34)n(Cc3cc(F)c(F)c(F)c3)c2=O)n1] | 1 | 2.00 | 759 |
| 1-0642 | 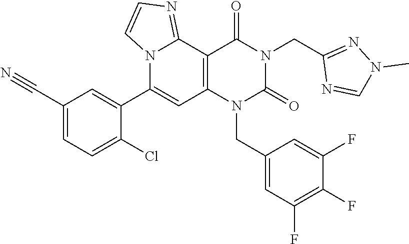 [SMILES: Cn1cnc(Cn2c(=O)c3c(cc(-c4cc(C#N)ccc4Cl)n4ccnc34)n(Cc3cc(F)c(F)c(F)c3)c2=O)n1] | 1 | 1.63 | 577 |

TABLE 41

| | | LC/MS | | |
|---|---|---|---|---|
| Compound | | measurement | Retention time | |
| No. | Structural formula | conditions | (min) | m/z |
| 1-0643 | 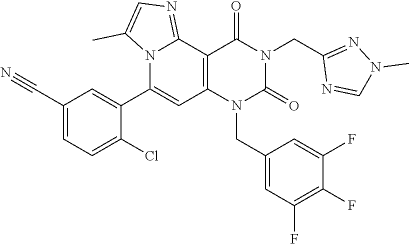 [SMILES: Cc1cnc2c3c(=O)n(Cc4ncn(C)n4)c(=O)n(Cc4cc(F)c(F)c(F)c4)c3cc(-c3cc(C#N)ccc3Cl)n12] | 1 | 1.67 | 591 |
| 1-0644 | 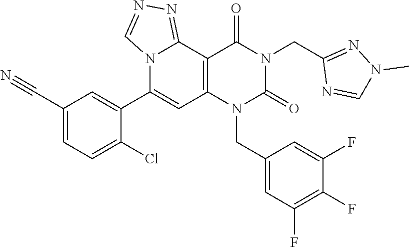 [SMILES: Cn1cnc(Cn2c(=O)c3c(cc(-c4cc(C#N)ccc4Cl)n4cnnc34)n(Cc3cc(F)c(F)c(F)c3)c2=O)n1] | 1 | 1.84 | 578 |
| 1-0645 | 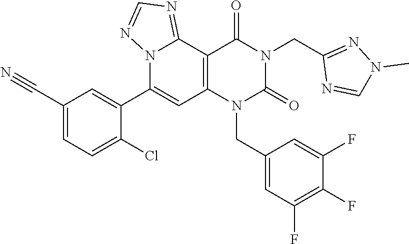 [SMILES: Cn1cnc(Cn2c(=O)c3c(cc(-c4cc(C#N)ccc4Cl)n4ncnc34)n(Cc3cc(F)c(F)c(F)c3)c2=O)n1] | 1 | 2.24 | 578 |
| 1-0646 | 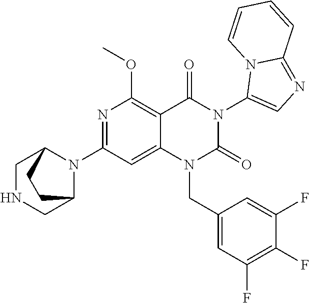 [SMILES: COc1nc(N2[C@@H]3CC[C@H]2CNC3)cc2c1c(=O)n(-c1cnc3ccccn13)c(=O)n2Cc1cc(F)c(F)c(F)c1] | 3 | 1.66 | 564 |
| 1-0647 | 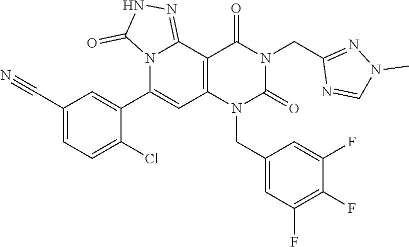 [SMILES: Cn1cnc(Cn2c(=O)c3c(cc(-c4cc(C#N)ccc4Cl)n4c(=O)[nH]nc34)n(Cc3cc(F)c(F)c(F)c3)c2=O)n1] | 1 | 1.81 | 594 |
| 1-0648 | 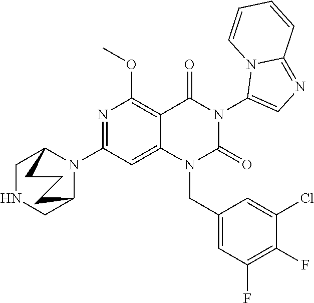 [SMILES: COc1nc(N2[C@@H]3CCC[C@H]2CNC3)cc2c1c(=O)n(-c1cnc3ccccn13)c(=O)n2Cc1cc(F)c(F)c(Cl)c1] | 3 | 1.74 | 594 |

TABLE 42

| | | LC/MS | | |
|---|---|---|---|---|
| Compound | | measurement | Retention time | |
| No. | Structural formula | conditions | (min) | m/z |
| 1-0649 | 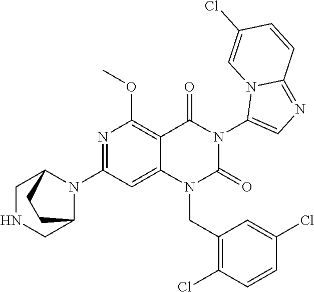 [SMILES: COc1nc(N2[C@@H]3CC[C@H]2CNC3)cc2c1c(=O)n(-c1cnc3ccc(Cl)cn13)c(=O)n2Cc1cc(Cl)ccc1Cl] | 3 | 1.80 | 612 |
| 1-0650 | 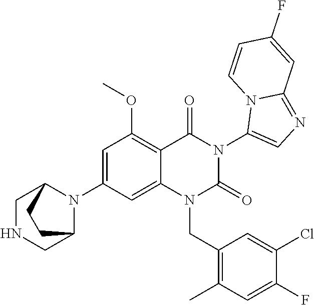 [SMILES: COc1cc(N2[C@@H]3CC[C@H]2CNC3)cc2c1c(=O)n(-c1cnc3cc(F)ccn13)c(=O)n2Cc1cc(Cl)c(F)cc1C] | 3 | 1.66 | 593 |
| 1-0651 | 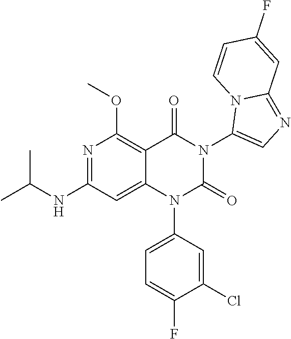 [SMILES: COc1nc(NC(C)C)cc2c1c(=O)n(-c1cnc3cc(F)ccn13)c(=O)n2-c1ccc(F)c(Cl)c1] | 1 | 2.04 | 513 |
| 1-0652 | 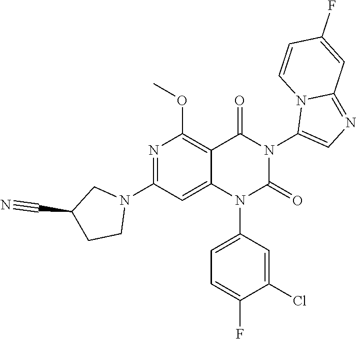 [SMILES: COc1nc(N2CC[C@@H](C#N)C2)cc2c1c(=O)n(-c1cnc3cc(F)ccn13)c(=O)n2-c1ccc(F)c(Cl)c1] | 1 | 1.82 | 550 |
| 1-0653 | 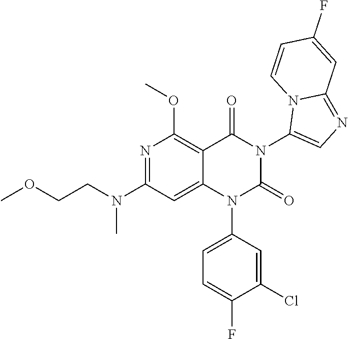 [SMILES: COCCN(C)c1cc2c(c(OC)n1)c(=O)n(-c1cnc3cc(F)ccn13)c(=O)n2-c1ccc(F)c(Cl)c1] | 1 | 1.88 | 543 |

TABLE 43

| | | LC/MS | | |
|---|---|---|---|---|
| Compound | | measurement | Retention time | |
| No. | Structural formula | conditions | (min) | m/z |
| 1-0654 | 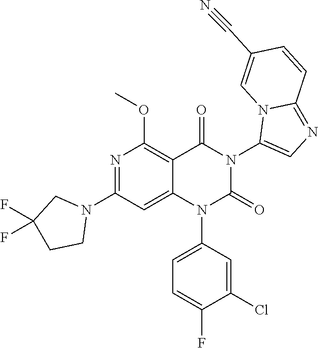 [SMILES: COc1nc(N2CCC(F)(F)C2)cc2c1c(=O)n(-c1cnc3ccc(C#N)cn13)c(=O)n2-c1ccc(F)c(Cl)c1] | 1 | 2.29 | 568 |
| 1-0655 | 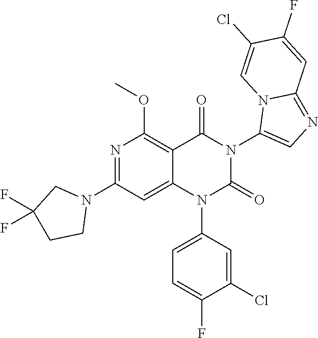 [SMILES: COc1nc(N2CCC(F)(F)C2)cc2c1c(=O)n(-c1cnc3cc(F)c(Cl)cn13)c(=O)n2-c1ccc(F)c(Cl)c1] | 1 | 2.40 | 595 |
| 1-0657 | 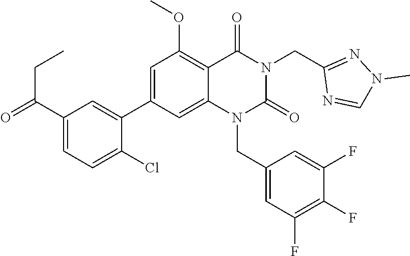 [SMILES: CCC(=O)c1ccc(Cl)c(-c2cc(OC)c3c(=O)n(Cc4ncn(C)n4)c(=O)n(Cc4cc(F)c(F)c(F)c4)c3c2)c1] | 1 | 2.35 | 598 |
| 1-0658 | 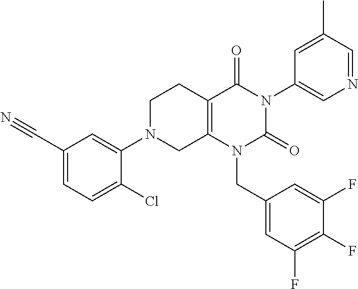 [SMILES: Cc1cncc(-n2c(=O)c3c(n(Cc4cc(F)c(F)c(F)c4)c2=O)CN(c2cc(C#N)ccc2Cl)CC3)c1] | 1 | 2.47 | 538.1 |
| 1-0660 | 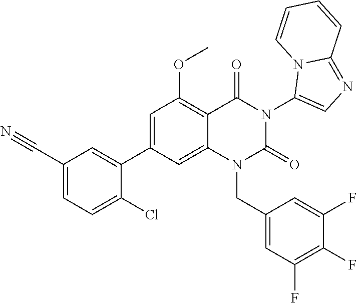 [SMILES: COc1cc(-c2cc(C#N)ccc2Cl)cc2c1c(=O)n(-c1cnc3ccccn13)c(=O)n2Cc1cc(F)c(F)c(F)c1] | 2 | 1.92 | 588.1 |

TABLE 44

| | | LC/MS | | |
|---|---|---|---|---|
| Compound | | measurement | Retention time | |
| No. | Structural formula | conditions | (min) | m/z |
| 1-0661 | 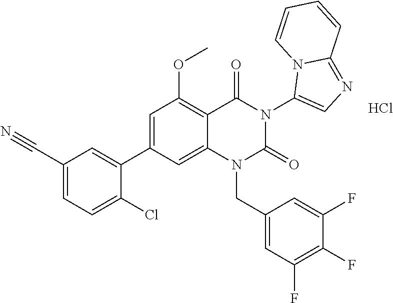 [SMILES: COc1cc(-c2cc(C#N)ccc2Cl)cc2c1c(=O)n(-c1cnc3ccccn13)c(=O)n2Cc1cc(F)c(F)c(F)c1 ; Cl] | 2 | 1.91 | 588.1 |
| 1-0666 | 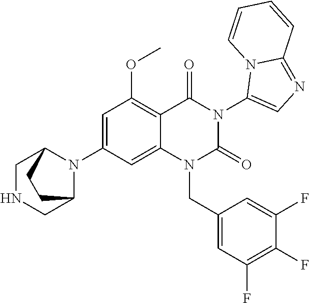 [SMILES: COc1cc(N2[C@@H]3CC[C@H]2CNC3)cc2c1c(=O)n(-c1cnc3ccccn13)c(=O)n2Cc1cc(F)c(F)c(F)c1] | 2 | 1.25 | 563.2 |
| 1-0671 | 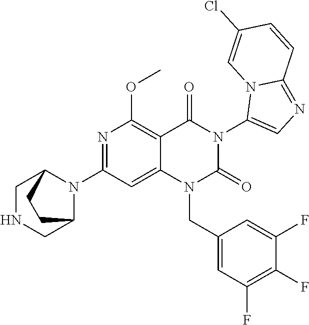 [SMILES: COc1nc(N2[C@@H]3CC[C@H]2CNC3)cc2c1c(=O)n(-c1cnc3ccc(Cl)cn13)c(=O)n2Cc1cc(F)c(F)c(F)c1] | 1 | 1.58 | 598.15 |
| 1-0672 | 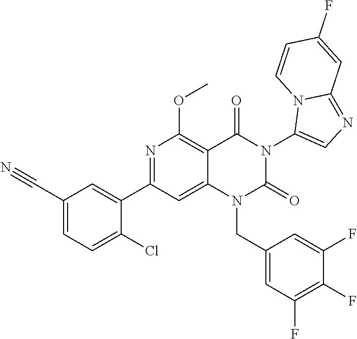 [SMILES: COc1nc(-c2cc(C#N)ccc2Cl)cc2c1c(=O)n(-c1cnc3cc(F)ccn13)c(=O)n2Cc1cc(F)c(F)c(F)c1] | 1 | 2.15 | 607.1 |
| 1-0673 | 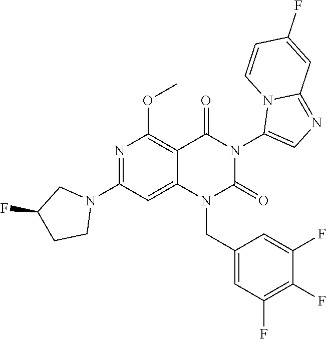 [SMILES: COc1nc(N2CC[C@@H](F)C2)cc2c1c(=O)n(-c1cnc3cc(F)ccn13)c(=O)n2Cc1cc(F)c(F)c(F)c1] | 1 | 1.89 | 559.15 |

TABLE 45

| | | LC/MS | | |
|---|---|---|---|---|
| Compound | | measurement | Retention time | |
| No. | Structural formula | conditions | (min) | m/z |
| 1-0680 | 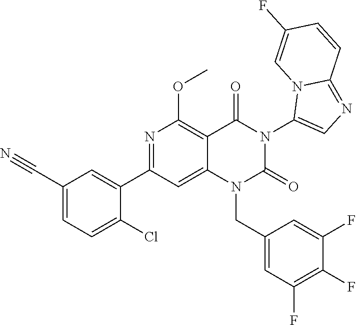 [SMILES: COc1nc(-c2cc(C#N)ccc2Cl)cc2c1c(=O)n(-c1cnc3ccc(F)cn13)c(=O)n2Cc1cc(F)c(F)c(F)c1] | 2 | 2.21 | 607.1 |
| 1-0681 | 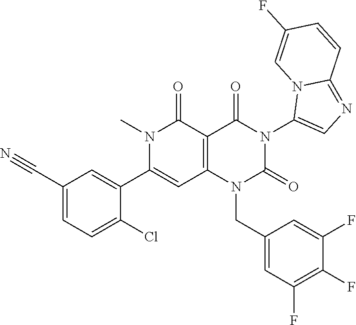 [SMILES: Cn1c(-c2cc(C#N)ccc2Cl)cc2c(c1=O)c(=O)n(-c1cnc3ccc(F)cn13)c(=O)n2Cc1cc(F)c(F)c(F)c1] | 2 | 1.89 | 607.1 |
| 1-0682 | 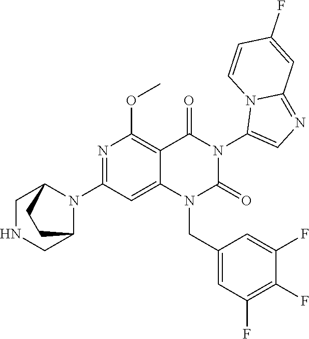 [SMILES: COc1nc(N2[C@@H]3CC[C@H]2CNC3)cc2c1c(=O)n(-c1cnc3cc(F)ccn13)c(=O)n2Cc1cc(F)c(F)c(F)c1] | 2 | 1.37 | 582.2 |
| 1-0683 | 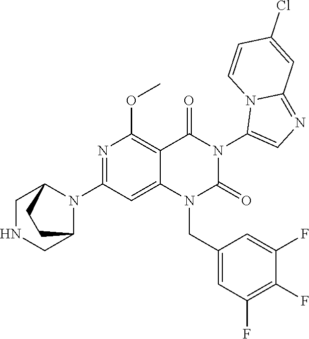 [SMILES: COc1nc(N2[C@@H]3CC[C@H]2CNC3)cc2c1c(=O)n(-c1cnc3cc(Cl)ccn13)c(=O)n2Cc1cc(F)c(F)c(F)c1] | 2 | 1.51 | 598.15 |
| 1-0684 | 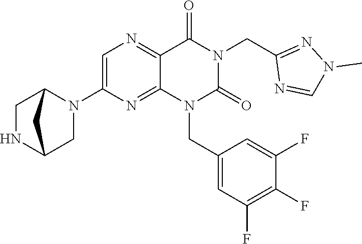 [SMILES: Cn1cnc(Cn2c(=O)c3ncc(N4C[C@H]5C[C@@H]4CN5)nc3n(Cc3cc(F)c(F)c(F)c3)c2=O)n1] | 1 | 1.30 | 500.1 |

TABLE 46

| | | LC/MS | | |
|---|---|---|---|---|
| Compound | | measurement | Retention time | |
| No. | Structural formula | conditions | (min) | m/z |
| 1-0685 | 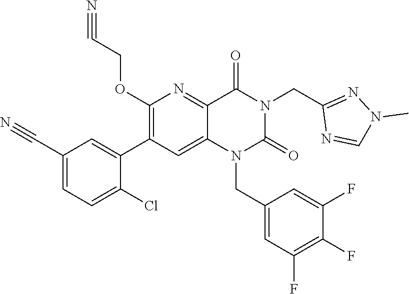 [SMILES: Cn1cnc(Cn2c(=O)c3nc(OCC#N)c(-c4cc(C#N)ccc4Cl)cc3n(Cc3cc(F)c(F)c(F)c3)c2=O)n1] | 1 | 2.17 | 593 |
| 1-0686 | 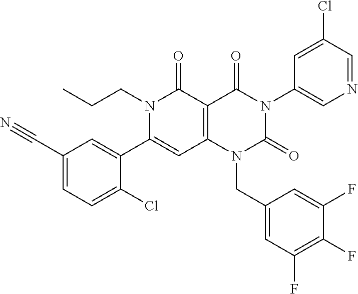 [SMILES: CCCn1c(-c2cc(C#N)ccc2Cl)cc2c(c1=O)c(=O)n(-c1cncc(Cl)c1)c(=O)n2Cc1cc(F)c(F)c(F)c1] | 1 | 2.36 | 611.95 |
| 1-0692 | 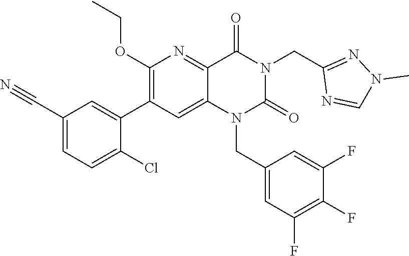 [SMILES: CCOc1nc2c(=O)n(Cc3ncn(C)n3)c(=O)n(Cc3cc(F)c(F)c(F)c3)c2cc1-c1cc(C#N)ccc1Cl] | 1 | 2.28 | 582.05 |
| 1-0697 | 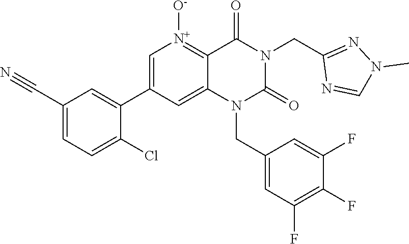 [SMILES: Cn1cnc(Cn2c(=O)c3c(cc(-c4cc(C#N)ccc4Cl)c[n+]3[O-])n(Cc3cc(F)c(F)c(F)c3)c2=O)n1] | 1 | 1.90 | 554 |
| 1-0699 | 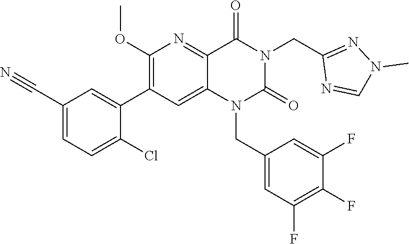 [SMILES: COc1nc2c(=O)n(Cc3ncn(C)n3)c(=O)n(Cc3cc(F)c(F)c(F)c3)c2cc1-c1cc(C#N)ccc1Cl] | 1 | 2.26 | 584 |
| 1-0700 | 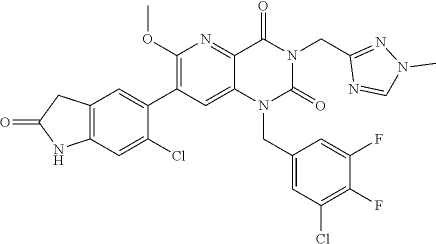 [SMILES: COc1nc2c(=O)n(Cc3ncn(C)n3)c(=O)n(Cc3cc(F)c(F)c(Cl)c3)c2cc1-c1cc2c(cc1Cl)NC(=O)C2] | 1 | 1.84 | 614 |

TABLE 47

| | | LC/MS | | |
|---|---|---|---|---|
| Compound | | measurement | Retention time | |
| No. | Structural formula | conditions | (min) | m/z |
| 1-0701 | 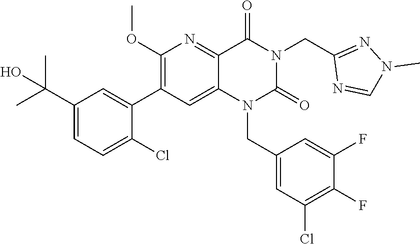 [SMILES: COc1nc2c(=O)n(Cc3ncn(C)n3)c(=O)n(Cc3cc(F)c(F)c(Cl)c3)c2cc1-c1cc(C(C)(C)O)ccc1Cl] | 1 | 2.17 | 617.05 |
| 1-0705 | 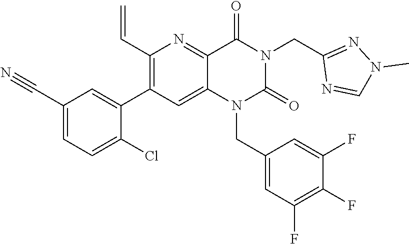 [SMILES: C=Cc1nc2c(=O)n(Cc3ncn(C)n3)c(=O)n(Cc3cc(F)c(F)c(F)c3)c2cc1-c1cc(C#N)ccc1Cl] | 1 | 2.20 | 564.05 |
| 1-0709 | 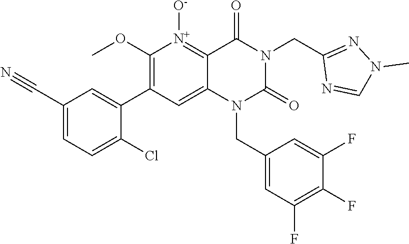 [SMILES: COc1c(-c2cc(C#N)ccc2Cl)cc2c(c(=O)n(Cc3ncn(C)n3)c(=O)n2Cc2cc(F)c(F)c(F)c2)[n+]1[O-]] | 1 | 1.95 | 584.05 |
| 1-0712 | 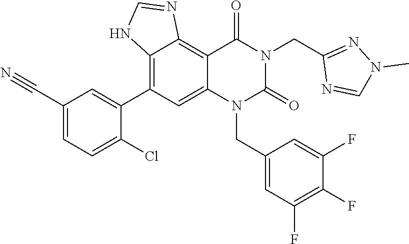 [SMILES: Cn1cnc(Cn2c(=O)c3c4nc[nH]c4c(-c4cc(C#N)ccc4Cl)cc3n(Cc3cc(F)c(F)c(F)c3)c2=O)n1] | 1 | 1.97 | 577 |
| 1-0713 | 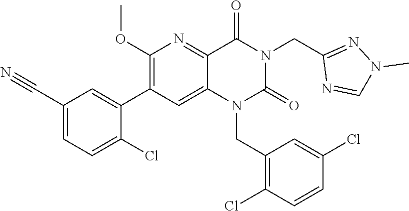 [SMILES: COc1nc2c(=O)n(Cc3ncn(C)n3)c(=O)n(Cc3cc(Cl)ccc3Cl)c2cc1-c1cc(C#N)ccc1Cl] | 1 | 2.24 | 584 |
| 1-0717 | 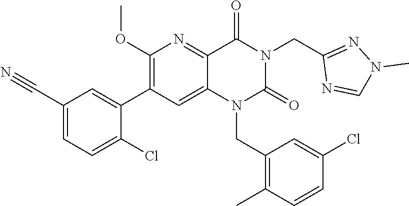 [SMILES: COc1nc2c(=O)n(Cc3ncn(C)n3)c(=O)n(Cc3cc(Cl)ccc3C)c2cc1-c1cc(C#N)ccc1Cl] | 1 | 2.21 | 562.05 |
| 1-0721 | 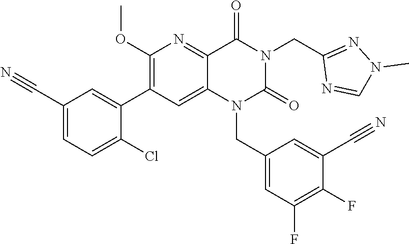 [SMILES: COc1nc2c(=O)n(Cc3ncn(C)n3)c(=O)n(Cc3cc(F)c(F)c(C#N)c3)c2cc1-c1cc(C#N)ccc1Cl] | 1 | 2.09 | 575.05 |

TABLE 48

| | | LC/MS | | |
|---|---|---|---|---|
| Compound | | measurement | Retention time | |
| No. | Structural formula | conditions | (min) | m/z |
| 1-0722 | 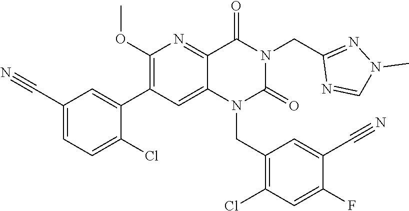 [SMILES: COc1nc2c(=O)n(Cc3ncn(C)n3)c(=O)n(Cc3cc(C#N)c(F)cc3Cl)c2cc1-c1cc(C#N)ccc1Cl] | 1 | 2.11 | 591 |
| 1-0723 | 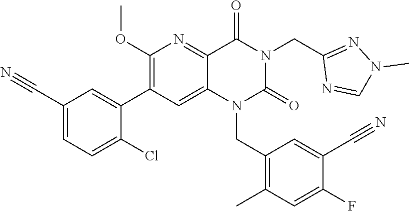 [SMILES: COc1nc2c(=O)n(Cc3ncn(C)n3)c(=O)n(Cc3cc(C#N)c(F)cc3C)c2cc1-c1cc(C#N)ccc1Cl] | 1 | 2.05 | 571.05 |
| 1-0726 | 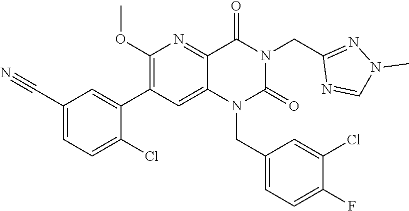 [SMILES: COc1nc2c(=O)n(Cc3ncn(C)n3)c(=O)n(Cc3ccc(F)c(Cl)c3)c2cc1-c1cc(C#N)ccc1Cl] | 1 | 2.18 | 565.95 |
| 1-0727 | 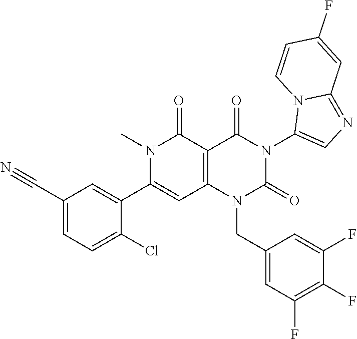 [SMILES: Cn1c(-c2cc(C#N)ccc2Cl)cc2c(c1=O)c(=O)n(-c1cnc3cc(F)ccn13)c(=O)n2Cc1cc(F)c(F)c(F)c1] | 1 | 1.87 | 607 |
| 1-0728 | 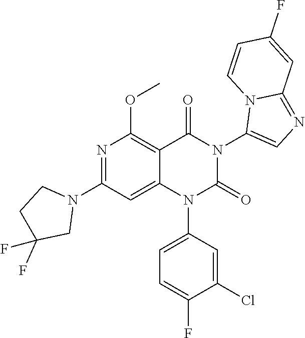 [SMILES: COc1nc(N2CCC(F)(F)C2)cc2c1c(=O)n(-c1cnc3cc(F)ccn13)c(=O)n2-c1ccc(F)c(Cl)c1] | 1 | 2.01 | 561 |
| 1-0729 | 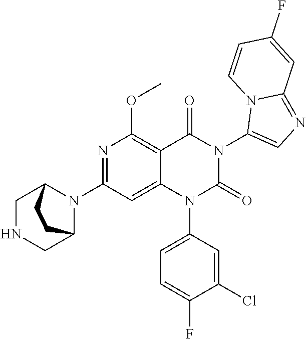 | 1 | 1.38 | 566 |

TABLE 49

| | | LC/MS | | |
|---|---|---|---|---|
| Compound | | measurement | Retention time | |
| No. | Structural formula | conditions | (min) | m/z |
| 1-0730 | 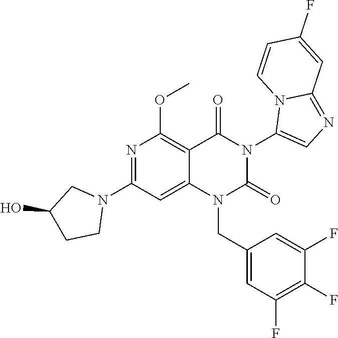 [SMILES: COc1nc(N2CC[C@@H](O)C2)cc2c1c(=O)n(-c1cnc3cc(F)ccn13)c(=O)n2Cc1cc(F)c(F)c(F)c1] | 1 | 1.67 | 557.05 |
| 1-0731 | 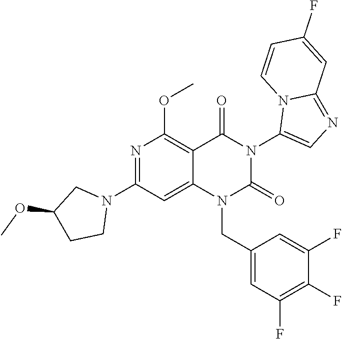 [SMILES: COc1nc(N2CC[C@@H](OC)C2)cc2c1c(=O)n(-c1cnc3cc(F)ccn13)c(=O)n2Cc1cc(F)c(F)c(F)c1] | 1 | 1.91 | 571.1 |
| 1-0732 | 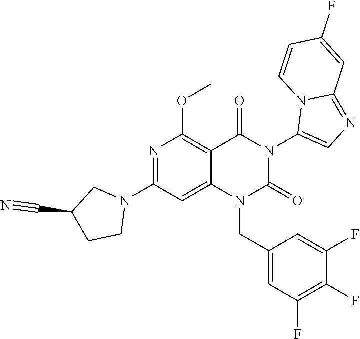 [SMILES: COc1nc(N2CC[C@@H](C#N)C2)cc2c1c(=O)n(-c1cnc3cc(F)ccn13)c(=O)n2Cc1cc(F)c(F)c(F)c1] | 1 | 1.86 | 566.05 |
| 1-0734 | 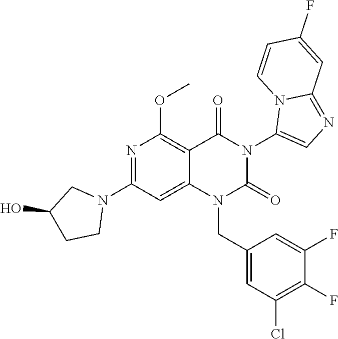 [SMILES: COc1nc(N2CC[C@@H](O)C2)cc2c1c(=O)n(-c1cnc3cc(F)ccn13)c(=O)n2Cc1cc(F)c(F)c(Cl)c1] | 1 | 1.75 | 573.05 |
| 1-0735 | 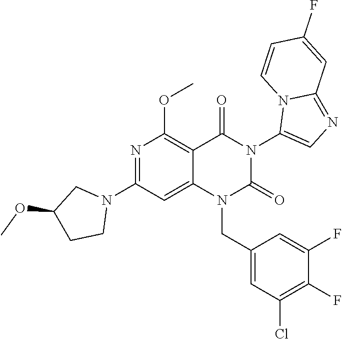 [SMILES: COc1nc(N2CC[C@@H](OC)C2)cc2c1c(=O)n(-c1cnc3cc(F)ccn13)c(=O)n2Cc1cc(F)c(F)c(Cl)c1] | 1 | 2.01 | 587.1 |

TABLE 50

| | | LC/MS | | |
|---|---|---|---|---|
| Compound | | measurement | Retention time | |
| No. | Structural formula | conditions | (min) | m/z |
| 1-0736 | 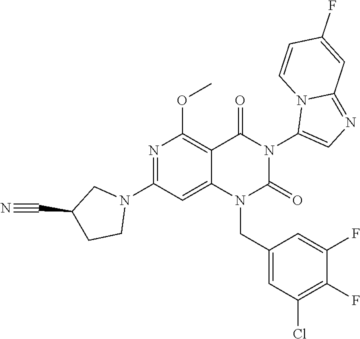 [SMILES: COc1nc(N2CC[C@@H](C#N)C2)cc2c1c(=O)n(-c1cnc3cc(F)ccn13)c(=O)n2Cc1cc(F)c(F)c(Cl)c1] | 1 | 1.92 | 582.05 |
| 1-0739 | 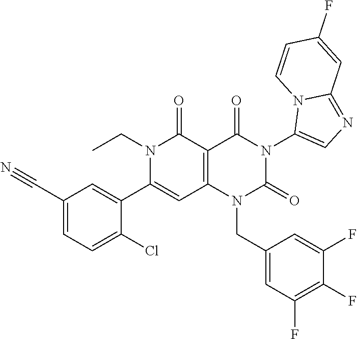 [SMILES: CCn1c(-c2cc(C#N)ccc2Cl)cc2c(c1=O)c(=O)n(-c1cnc3cc(F)ccn13)c(=O)n2Cc1cc(F)c(F)c(F)c1] | 1 | 1.95 | 621 |
| 1-0740 | 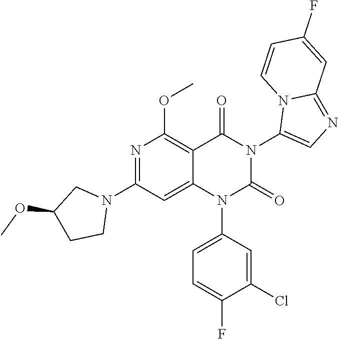 [SMILES: COc1nc(N2CC[C@@H](OC)C2)cc2c1c(=O)n(-c1cnc3cc(F)ccn13)c(=O)n2-c1ccc(F)c(Cl)c1] | 1 | 1.94 | 555.05 |
| 1-0741 | 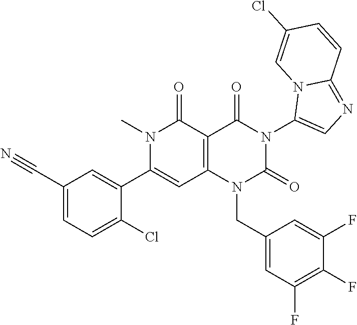 [SMILES: Cn1c(-c2cc(C#N)ccc2Cl)cc2c(c1=O)c(=O)n(-c1cnc3ccc(Cl)cn13)c(=O)n2Cc1cc(F)c(F)c(F)c1] | 1 | 2.08 | 622.95 |
| 1-0742 | 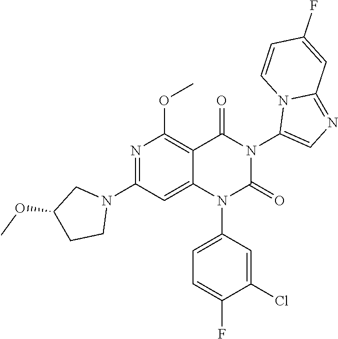 [SMILES: COc1nc(N2CC[C@H](OC)C2)cc2c1c(=O)n(-c1cnc3cc(F)ccn13)c(=O)n2-c1ccc(F)c(Cl)c1] | 1 | 1.92 | 555 |

TABLE 51

| | | LC/MS | | |
|---|---|---|---|---|
| Compound | | measurement | Retention time | |
| No. | Structural formula | conditions | (min) | m/z |
| 1-0743 | 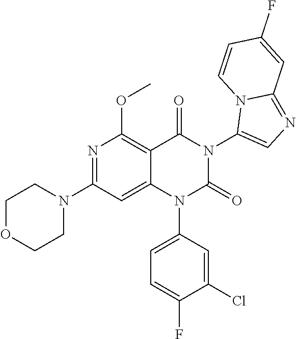 [SMILES: COc1nc(N2CCOCC2)cc2c1c(=O)n(-c1cnc3cc(F)ccn13)c(=O)n2-c1ccc(F)c(Cl)c1] | 1 | 1.72 | 541.05 |
| 1-0744 | 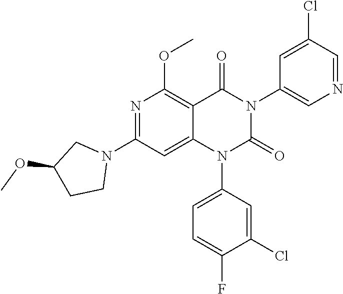 [SMILES: COc1nc(N2CC[C@@H](OC)C2)cc2c1c(=O)n(-c1cncc(Cl)c1)c(=O)n2-c1ccc(F)c(Cl)c1] | 1 | 2.35 | 532 |
| 1-0745 | 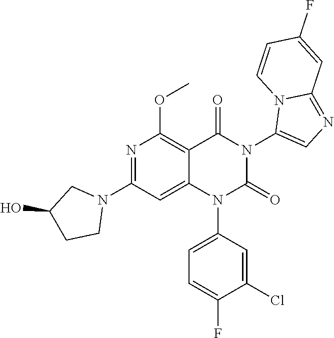 [SMILES: COc1nc(N2CC[C@@H](O)C2)cc2c1c(=O)n(-c1cnc3cc(F)ccn13)c(=O)n2-c1ccc(F)c(Cl)c1] | 1 | 1.67 | 541 |
| 1-0746 | 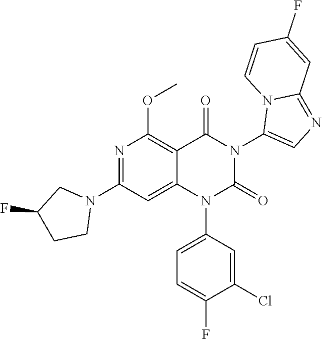 [SMILES: COc1nc(N2CC[C@@H](F)C2)cc2c1c(=O)n(-c1cnc3cc(F)ccn13)c(=O)n2-c1ccc(F)c(Cl)c1] | 1 | 2.00 | 543.05 |
| 1-0747 | 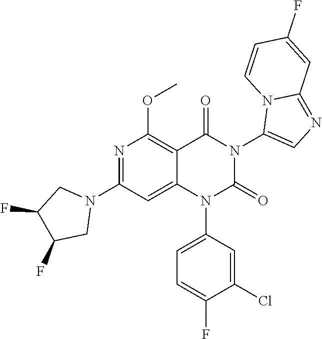 [SMILES: COc1nc(N2C[C@@H](F)[C@@H](F)C2)cc2c1c(=O)n(-c1cnc3cc(F)ccn13)c(=O)n2-c1ccc(F)c(Cl)c1] | 1 | 1.99 | 561.05 |

TABLE 52

| | | LC/MS | | |
|---|---|---|---|---|
| Compound | | measurement | Retention time | |
| No. | Structural formula | conditions | (min) | m/z |
| 1-0748 | 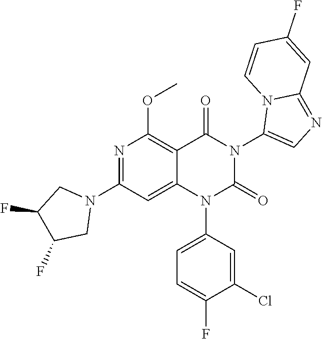 [SMILES: COc1nc(N2C[C@H](F)[C@@H](F)C2)cc2c1c(=O)n(-c1cnc3cc(F)ccn13)c(=O)n2-c1ccc(F)c(Cl)c1] | 1 | 2.00 | 561.05 |
| 1-0749 | 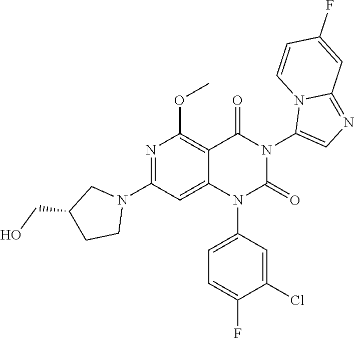 [SMILES: COc1nc(N2CC[C@H](CO)C2)cc2c1c(=O)n(-c1cnc3cc(F)ccn13)c(=O)n2-c1ccc(F)c(Cl)c1] | 1 | 1.69 | 555.05 |
| 1-0750 | 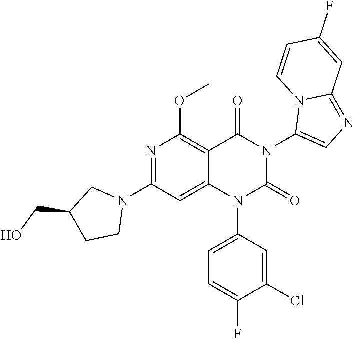 [SMILES: COc1nc(N2CC[C@@H](CO)C2)cc2c1c(=O)n(-c1cnc3cc(F)ccn13)c(=O)n2-c1ccc(F)c(Cl)c1] | 1 | 1.75 | 555.05 |
| 1-0751 | 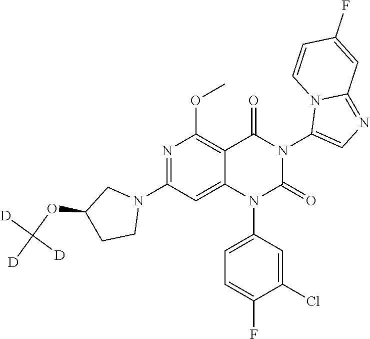 [SMILES: [2H]C([2H])([2H])O[C@@H]1CCN(c2cc3c(c(OC)n2)c(=O)n(-c2cnc4cc(F)ccn24)c(=O)n3-c2ccc(F)c(Cl)c2)C1] | 1 | 1.99 | 558.05 |
| 1-0752 | 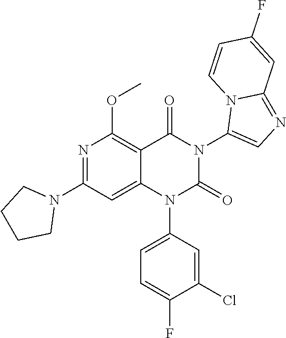 [SMILES: COc1nc(N2CCCC2)cc2c1c(=O)n(-c1cnc3cc(F)ccn13)c(=O)n2-c1ccc(F)c(Cl)c1] | 1 | 2.19 | 525.05 |

TABLE 53

| | | LC/MS | | |
|---|---|---|---|---|
| Compound | | measurement | Retention time | |
| No. | Structural formula | conditions | (min) | m/z |
| 1-0753 | 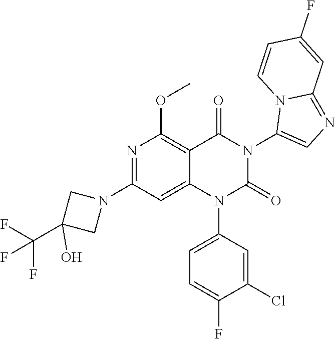 [SMILES: COc1nc(N2CC(O)(C(F)(F)F)C2)cc2c1c(=O)n(-c1cnc3cc(F)ccn13)c(=O)n2-c1ccc(F)c(Cl)c1] | 1 | 1.99 | 595 |
| 1-0755 | 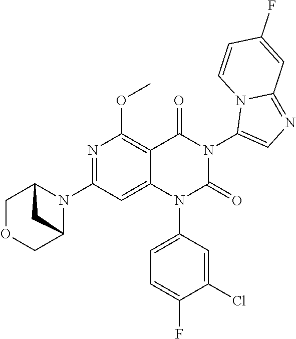 [SMILES: COc1nc(N2[C@@H]3COC[C@H]2C3)cc2c1c(=O)n(-c1cnc3cc(F)ccn13)c(=O)n2-c1ccc(F)c(Cl)c1] | 1 | 1.88 | 553.05 |
| 1-0759 | 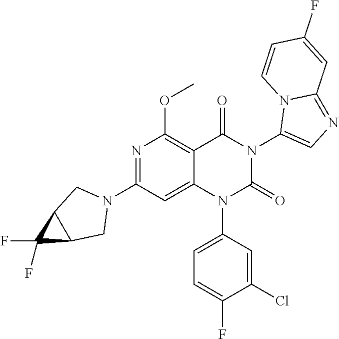 [SMILES: COc1nc(N2C[C@@H]3[C@H](C2)C3(F)F)cc2c1c(=O)n(-c1cnc3cc(F)ccn13)c(=O)n2-c1ccc(F)c(Cl)c1] | 1 | 2.12 | 573 |
| 1-0762 | 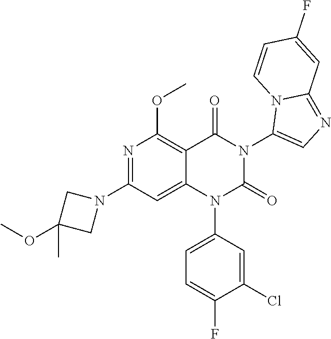 [SMILES: COc1nc(N2CC(C)(OC)C2)cc2c1c(=O)n(-c1cnc3cc(F)ccn13)c(=O)n2-c1ccc(F)c(Cl)c1] | 1 | 2.04 | 555.05 |
| 1-0763 | 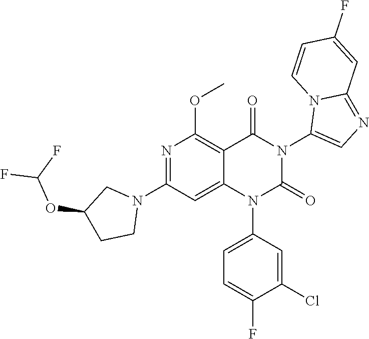 [SMILES: COc1nc(N2CC[C@@H](OC(F)F)C2)cc2c1c(=O)n(-c1cnc3cc(F)ccn13)c(=O)n2-c1ccc(F)c(Cl)c1] | 1 | 2.15 | 591 |

TABLE 54

| | | LC/MS | | |
|---|---|---|---|---|
| Compound | | measurement | Retention time | |
| No. | Structural formula | conditions | (min) | m/z |
| 1-0764 | 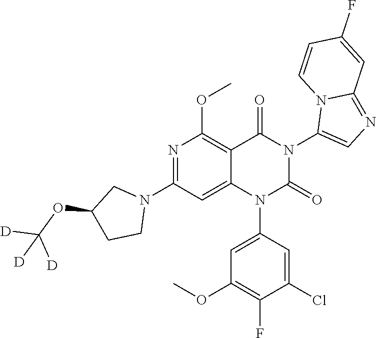 [SMILES: [2H]C([2H])([2H])O[C@@H]1CCN(c2cc3c(c(OC)n2)c(=O)n(-c2cnc4cc(F)ccn24)c(=O)n3-c2cc(Cl)c(F)c(OC)c2)C1] | 1 | 2.10 | 588.05 |
| 1-0784 | 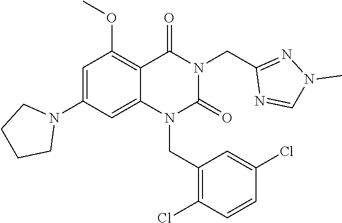 [SMILES: COc1cc(N2CCCC2)cc2c1c(=O)n(Cc1ncn(C)n1)c(=O)n2Cc1cc(Cl)ccc1Cl] | 1 | 2.25 | 515 |
| 1-0786 | 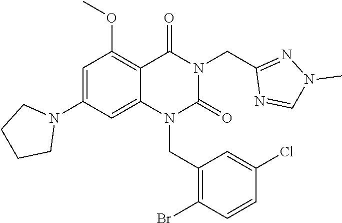 [SMILES: COc1cc(N2CCCC2)cc2c1c(=O)n(Cc1ncn(C)n1)c(=O)n2Cc1cc(Cl)ccc1Br] | 1 | 2.29 | 558.95 |
| 1-0800 | 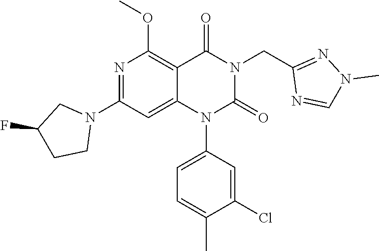 [SMILES: COc1nc(N2CC[C@@H](F)C2)cc2c1c(=O)n(Cc1ncn(C)n1)c(=O)n2-c1ccc(C)c(Cl)c1] | 1 | 2.03 | 500 |
| 1-0802 | 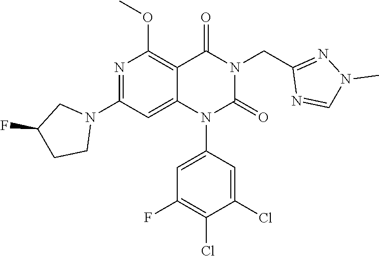 [SMILES: COc1nc(N2CC[C@@H](F)C2)cc2c1c(=O)n(Cc1ncn(C)n1)c(=O)n2-c1cc(F)c(Cl)c(Cl)c1] | 1 | 2.11 | 538 |
| 1-0803 | 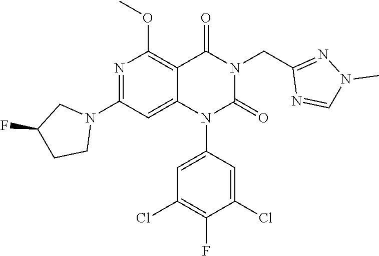 [SMILES: COc1nc(N2CC[C@@H](F)C2)cc2c1c(=O)n(Cc1ncn(C)n1)c(=O)n2-c1cc(Cl)c(F)c(Cl)c1] | 1 | 2.11 | 538 |

TABLE 55

| | | LC/MS | | |
|---|---|---|---|---|
| Compound | | measurement | Retention time | |
| No. | Structural formula | conditions | (min) | m/z |
| 1-0804 | 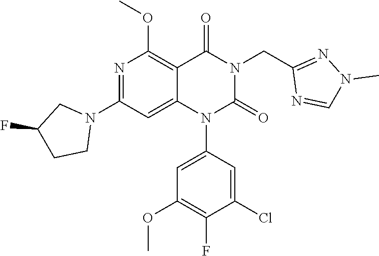 [SMILES: COc1cc(-n2c(=O)n(Cc3ncn(C)n3)c(=O)c3c(OC)nc(N4CC[C@@H](F)C4)cc32)cc(Cl)c1F] | 1 | 2.01 | 534.05 |
| 1-0805 | 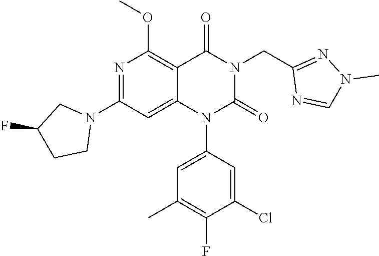 [SMILES: COc1nc(N2CC[C@@H](F)C2)cc2c1c(=O)n(Cc1ncn(C)n1)c(=O)n2-c1cc(C)c(F)c(Cl)c1] | 1 | 2.07 | 518.05 |
| 1-0806 | 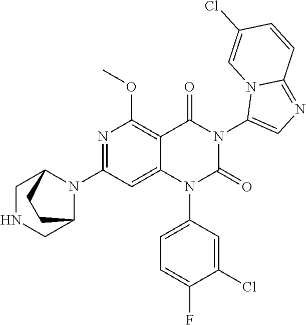 [SMILES: COc1nc(N2[C@@H]3CC[C@H]2CNC3)cc2c1c(=O)n(-c1cnc3ccc(Cl)cn13)c(=O)n2-c1ccc(F)c(Cl)c1] | 1 | 1.52 | 582 |
| 1-0808 | 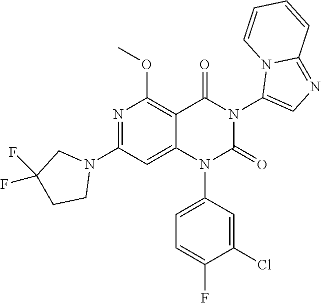 [SMILES: COc1nc(N2CCC(F)(F)C2)cc2c1c(=O)n(-c1cnc3ccccn13)c(=O)n2-c1ccc(F)c(Cl)c1] | 1 | 1.92 | 543 |
| 1-0809 | 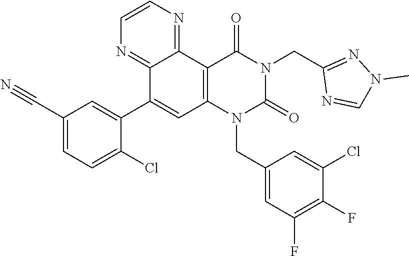 [SMILES: Cn1cnc(Cn2c(=O)c3c4nccnc4c(-c4cc(C#N)ccc4Cl)cc3n(Cc3cc(F)c(F)c(Cl)c3)c2=O)n1] | 2 | 2.26 | 605.05 |
| I-0810 | 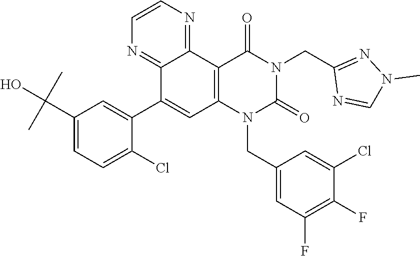 [SMILES: Cn1cnc(Cn2c(=O)c3c4nccnc4c(-c4cc(C(C)(C)O)ccc4Cl)cc3n(Cc3cc(F)c(F)c(Cl)c3)c2=O)n1] | 2 | 2.18 | 638.05 |

TABLE 56

| | | LC/MS | | |
|---|---|---|---|---|
| Compound | | measurement | Retention time | |
| No. | Structural formula | conditions | (min) | m/z |
| 1-0811 | 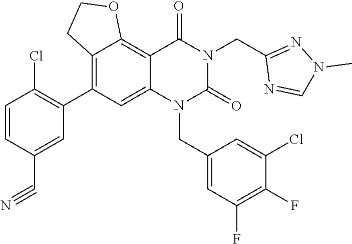 [SMILES: Cn1cnc(Cn2c(=O)c3c4c(c(-c5cc(C#N)ccc5Cl)cc3n(Cc3cc(F)c(F)c(Cl)c3)c2=O)CCO4)n1] | 1 | 2.16 | 595 |
| 1-0812 | 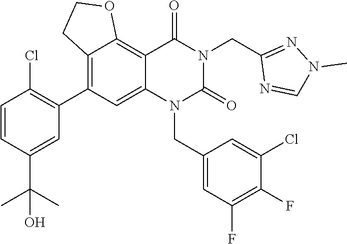 [SMILES: Cn1cnc(Cn2c(=O)c3c4c(c(-c5cc(C(C)(C)O)ccc5Cl)cc3n(Cc3cc(F)c(F)c(Cl)c3)c2=O)CCO4)n1] | 1 | 2.16 | 628 |
| 1-0813 | 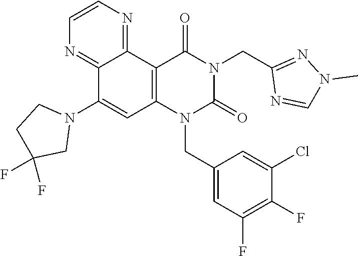 [SMILES: Cn1cnc(Cn2c(=O)c3c4nccnc4c(N4CCC(F)(F)C4)cc3n(Cc3cc(F)c(F)c(Cl)c3)c2=O)n1] | 1 | 2.28 | 575 |
| 1-0814 | 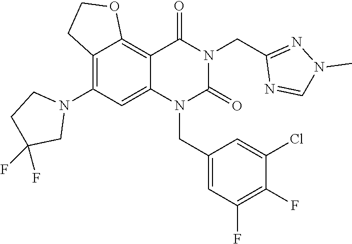 [SMILES: Cn1cnc(Cn2c(=O)c3c4c(c(N5CCC(F)(F)C5)cc3n(Cc3cc(F)c(F)c(Cl)c3)c2=O)CCO4)n1] | 1 | 1.95 | 565 |
| 1-0815 | 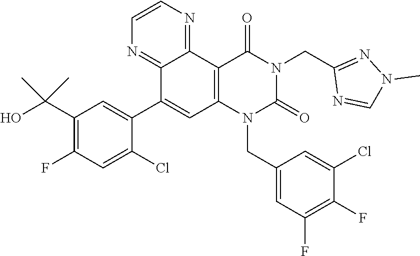 [SMILES: Cn1cnc(Cn2c(=O)c3c4nccnc4c(-c4cc(C(C)(C)O)c(F)cc4Cl)cc3n(Cc3cc(F)c(F)c(Cl)c3)c2=O)n1] | 1 | 2.08 | 656 |
| 1-0816 | 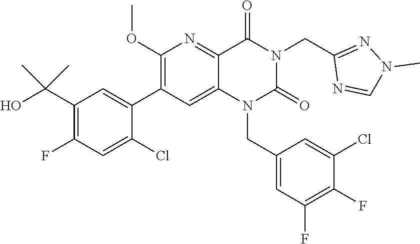 [SMILES: COc1nc2c(=O)n(Cc3ncn(C)n3)c(=O)n(Cc3cc(F)c(F)c(Cl)c3)c2cc1-c1cc(C(C)(C)O)c(F)cc1Cl] | 1 | 2.47 | 635 |

TABLE 57

| | | LC/MS | | |
|---|---|---|---|---|
| Compound | | measurement | Retention time | |
| No. | Structural formula | conditions | (min) | m/z |
| 1-0819 | 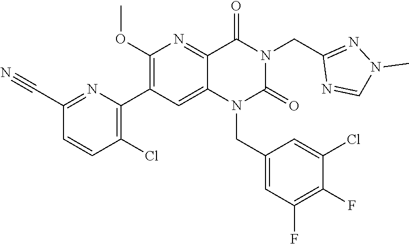 [SMILES: COc1nc2c(=O)n(Cc3ncn(C)n3)c(=O)n(Cc3cc(F)c(F)c(Cl)c3)c2cc1-c1nc(C#N)ccc1Cl] | 1 | 2.26 | 585 |
| 1-0820 | 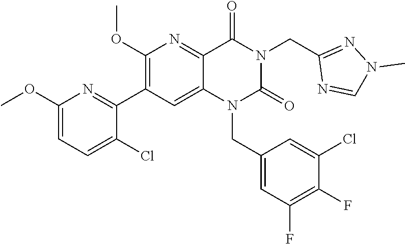 | 1 | 2.44 | 590 |
| 1-0821 | 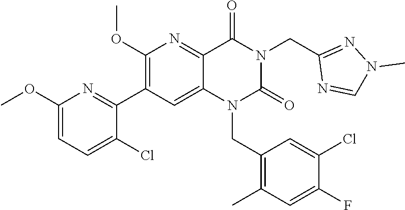 | 1 | 2.12 | 586 |
| 1-0822 | 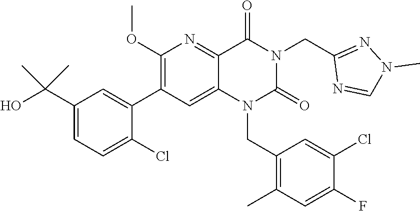 [SMILES: COc1nc2c(=O)n(Cc3ncn(C)n3)c(=O)n(Cc3cc(Cl)c(F)cc3C)c2cc1-c1cc(C(C)(C)O)ccc1Cl] | 1 | 1.96 | 613 |
| 1-0823 | 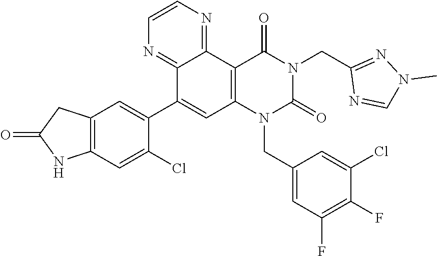 [SMILES: Cn1cnc(Cn2c(=O)c3c4nccnc4c(-c4cc5c(cc4Cl)NC(=O)C5)cc3n(Cc3cc(F)c(F)c(Cl)c3)c2=O)n1] | 1 | 1.94 | 635 |
| 1-0826 | 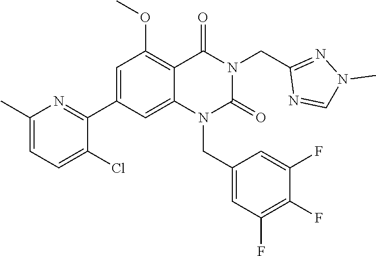 | 1 | 2.11 | 557 |
| 1-0827 | 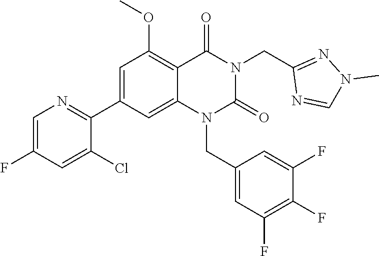 [SMILES: COc1cc(-c2ncc(F)cc2Cl)cc2c1c(=O)n(Cc1ncn(C)n1)c(=O)n2Cc1cc(F)c(F)c(F)c1] | 1 | 2.12 | 561 |

TABLE 58

| | | LC/MS | | |
|---|---|---|---|---|
| Compound | | measurement | Retention time | |
| No. | Structural formula | conditions | (min) | m/z |
| 1-0834 | 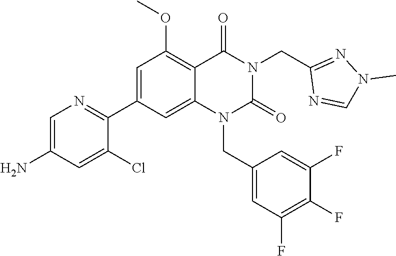 [SMILES: COc1cc(-c2ncc(N)cc2Cl)cc2c1c(=O)n(Cc1ncn(C)n1)c(=O)n2Cc1cc(F)c(F)c(F)c1] | 1 | 1.81 | 558 |
| 1-0838 | 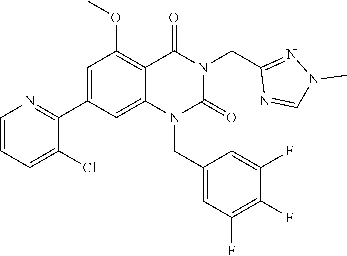 [SMILES: COc1cc(-c2ncccc2Cl)cc2c1c(=O)n(Cc1ncn(C)n1)c(=O)n2Cc1cc(F)c(F)c(F)c1] | 1 | 1.99 | 543 |
| 1-0839 | 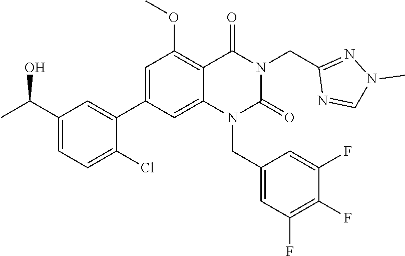 [SMILES: COc1cc(-c2cc([C@@H](C)O)ccc2Cl)cc2c1c(=O)n(Cc1ncn(C)n1)c(=O)n2Cc1cc(F)c(F)c(F)c1] | 1 | 2.22 | 586 |
| 1-0840 | 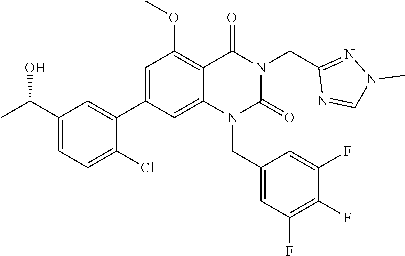 [SMILES: COc1cc(-c2cc([C@H](C)O)ccc2Cl)cc2c1c(=O)n(Cc1ncn(C)n1)c(=O)n2Cc1cc(F)c(F)c(F)c1] | 1 | 2.22 | 586 |
| 1-0842 | 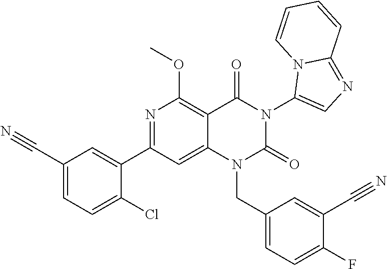 [SMILES: COc1nc(-c2cc(C#N)ccc2Cl)cc2c1c(=O)n(-c1cnc3ccccn13)c(=O)n2Cc1ccc(F)c(C#N)c1] | 1 | 2.03 | 578 |
| 1-0843 | 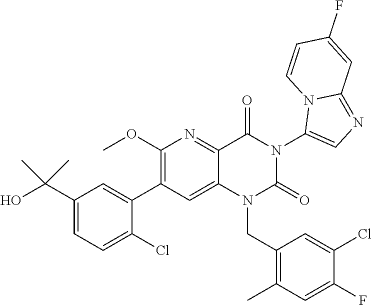 [SMILES: COc1nc2c(=O)n(-c3cnc4cc(F)ccn34)c(=O)n(Cc3cc(Cl)c(F)cc3C)c2cc1-c1cc(C(C)(C)O)ccc1Cl] | 1 | 2.26 | 652 |

TABLE 59

| | | LC/MS | | |
|---|---|---|---|---|
| Compound | | measurement | Retention time | |
| No. | Structural formula | conditions | (min) | m/z |
| 1-0844 | 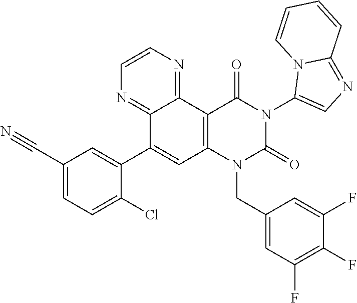 [SMILES: N#Cc1ccc(Cl)c(-c2cc3c(c(=O)n(-c4cnc5ccccn45)c(=O)n3Cc3cc(F)c(F)c(F)c3)c3nccnc23)c1] | 1 | 1.85 | 609.95 |
| 1-0847 | 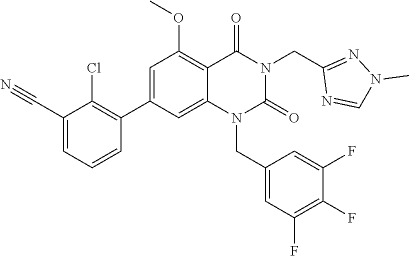 [SMILES: COc1cc(-c2cccc(C#N)c2Cl)cc2c1c(=O)n(Cc1ncn(C)n1)c(=O)n2Cc1cc(F)c(F)c(F)c1] | 1 | 2.09 | 567.15 |
| 1-0848 | 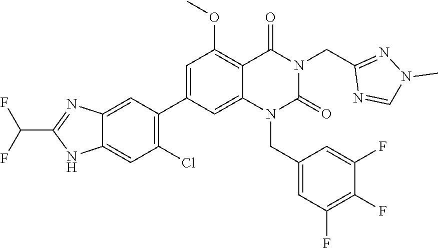 [SMILES: COc1cc(-c2cc3nc(C(F)F)[nH]c3cc2Cl)cc2c1c(=O)n(Cc1ncn(C)n1)c(=O)n2Cc1cc(F)c(F)c(F)c1] | 1 | 1.94 | 632.25 |
| 1-0852 | 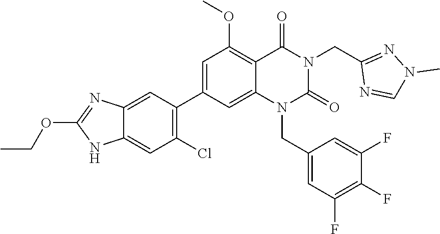 [SMILES: CCOc1nc2cc(-c3cc(OC)c4c(=O)n(Cc5ncn(C)n5)c(=O)n(Cc5cc(F)c(F)c(F)c5)c4c3)c(Cl)cc2[nH]1] | 1 | 1.95 | 626.2 |
| 1-0858 | 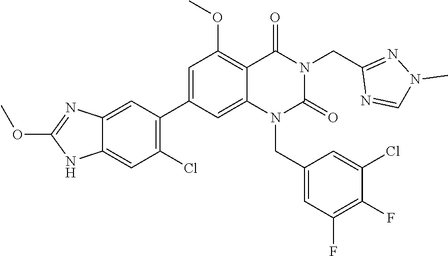 | 1 | 1.91 | 628.15 |
| 1-0859 | 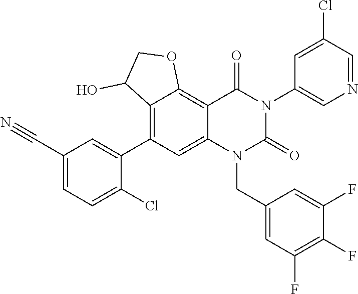 [SMILES: N#Cc1ccc(Cl)c(-c2cc3c(c4c2C(O)CO4)c(=O)n(-c2cncc(Cl)c2)c(=O)n3Cc2cc(F)c(F)c(F)c2)c1] | 1 | 2.28 | 611 |

TABLE 60

| | | LC/MS | | |
|---|---|---|---|---|
| Compound | | measurement | Retention time | |
| No. | Structural formula | conditions | (min) | m/z |
| I-0863 | 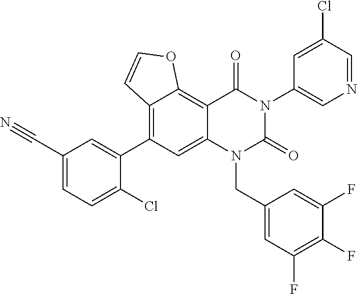 [SMILES: N#Cc1ccc(Cl)c(-c2cc3c(c(=O)n(-c4cncc(Cl)c4)c(=O)n3Cc3cc(F)c(F)c(F)c3)c3occc23)c1] | 1 | 2.53 | 593 |
| I-0864 | 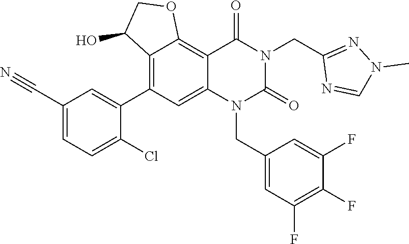 [SMILES: Cn1cnc(Cn2c(=O)c3c4c(c(-c5cc(C#N)ccc5Cl)cc3n(Cc3cc(F)c(F)c(F)c3)c2=O)[C@@H](O)CO4)n1] | 1 | 1.95 | 595.15 |
| I-0865 | 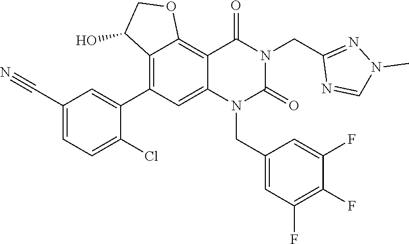 [SMILES: Cn1cnc(Cn2c(=O)c3c4c(c(-c5cc(C#N)ccc5Cl)cc3n(Cc3cc(F)c(F)c(F)c3)c2=O)[C@H](O)CO4)n1] | 1 | 1.95 | 595.1 |
| I-0866 | 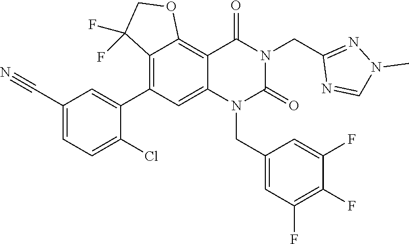 [SMILES: Cn1cnc(Cn2c(=O)c3c4c(c(-c5cc(C#N)ccc5Cl)cc3n(Cc3cc(F)c(F)c(F)c3)c2=O)C(F)(F)CO4)n1] | 1 | 2.23 | 615.15 |
| I-0867 | 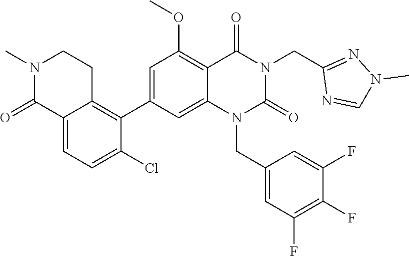 | 1 | 1.92 | 625.2 |
| I-0868 | 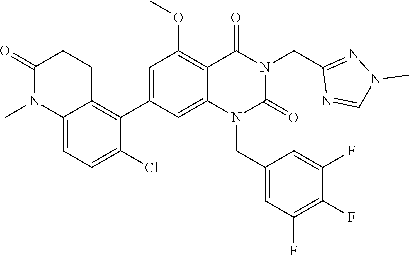 [SMILES: COc1cc(-c2c(Cl)ccc3c2CCC(=O)N3C)cc2c1c(=O)n(Cc1ncn(C)n1)c(=O)n2Cc1cc(F)c(F)c(F)c1] | 1 | 2.02 | 625.25 |

TABLE 61

| | | LC/MS | | |
|---|---|---|---|---|
| Compound | | measurement | Retention time | |
| No. | Structural formula | conditions | (min) | m/z |
| I-0869 | 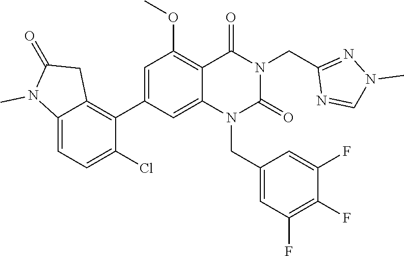 | 1 | 2.01 | 611.15 |
| I-0870 | 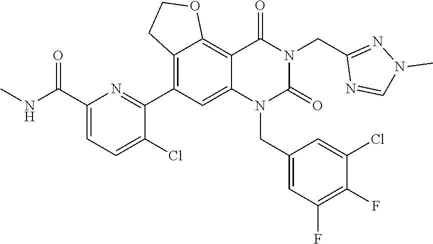 | 1 | 1.96 | 628.1 |
| I-0871 | 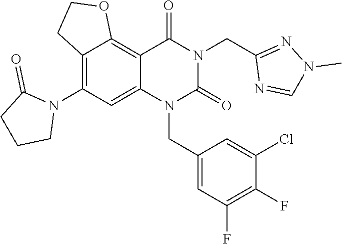 [SMILES: Cn1cnc(Cn2c(=O)c3c4c(c(N5CCCC5=O)cc3n(Cc3cc(F)c(F)c(Cl)c3)c2=O)CCO4)n1] | 1 | 1.76 | 543.1 |
| I-0874 | 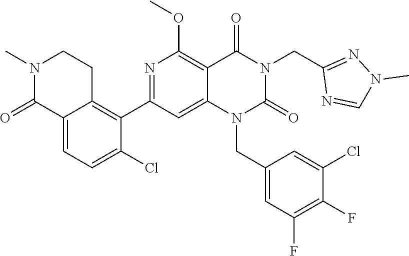 | 1 | 2.01 | 642.15 |
| I-0875 | 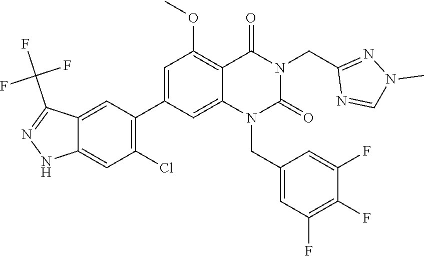 [SMILES: COc1cc(-c2cc3c(C(F)(F)F)n[nH]c3cc2Cl)cc2c1c(=O)n(Cc1ncn(C)n1)c(=O)n2Cc1cc(F)c(F)c(F)c1] | 1 | 2.15 | 650.15 |
| I-0876 | 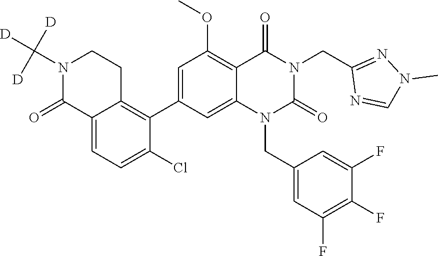 [SMILES: [2H]C([2H])([2H])N1CCc2c(ccc(Cl)c2-c2cc(OC)c3c(=O)n(Cc4ncn(C)n4)c(=O)n(Cc4cc(F)c(F)c(F)c4)c3c2)C1=O] | 1 | 1.92 | 628.2 |

TABLE 62

| | | LC/MS | | |
|---|---|---|---|---|
| Compound | | measurement | Retention time | |
| No. | Structural formula | conditions | (min) | m/z |
| I-0878 | 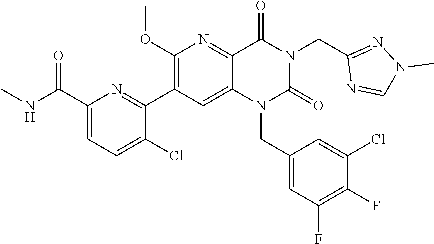 | 1 | 1.93 | 617.15 |
| I-0879 | 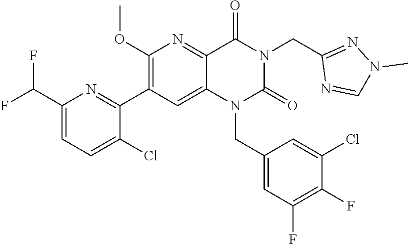 [SMILES: COc1nc2c(=O)n(Cc3ncn(C)n3)c(=O)n(Cc3cc(F)c(F)c(Cl)c3)c2cc1-c1nc(C(F)F)ccc1Cl] | 1 | 2.19 | 610.1 |
| I-0880 | 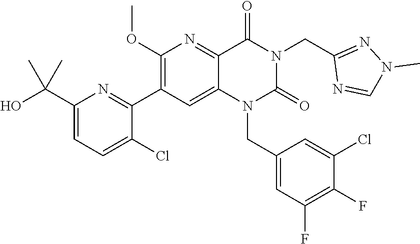 [SMILES: COc1nc2c(=O)n(Cc3ncn(C)n3)c(=O)n(Cc3cc(F)c(F)c(Cl)c3)c2cc1-c1nc(C(C)(C)O)ccc1Cl] | 1 | 2.01 | 618.25 |
| I-0881 | 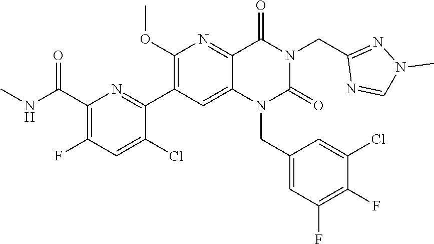 | 1 | 1.91 | 635.1 |
| I-0882 | 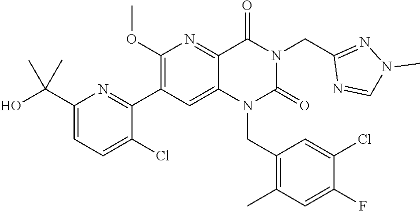 [SMILES: COc1nc2c(=O)n(Cc3ncn(C)n3)c(=O)n(Cc3cc(Cl)c(F)cc3C)c2cc1-c1nc(C(C)(C)O)ccc1Cl] | 2 | 1.98 | 614.15 |
| I-0883 | 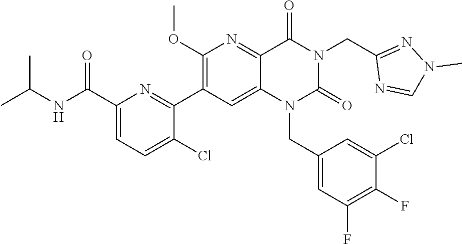 [SMILES: COc1nc2c(=O)n(Cc3ncn(C)n3)c(=O)n(Cc3cc(F)c(F)c(Cl)c3)c2cc1-c1nc(C(=O)NC(C)C)ccc1Cl] | 2 | 2.10 | 645.15 |
| I-0884 | 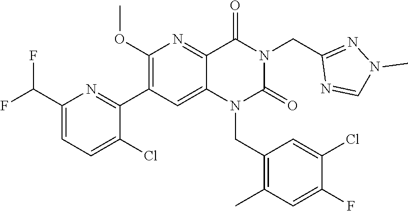 [SMILES: COc1nc2c(=O)n(Cc3ncn(C)n3)c(=O)n(Cc3cc(Cl)c(F)cc3C)c2cc1-c1nc(C(F)F)ccc1Cl] | 2 | 2.18 | 606.05 |

TABLE 63

| | | LC/MS | | |
|---|---|---|---|---|
| Compound | | measurement | Retention time | |
| No. | Structural formula | conditions | (min) | m/z |
| I-0885 | 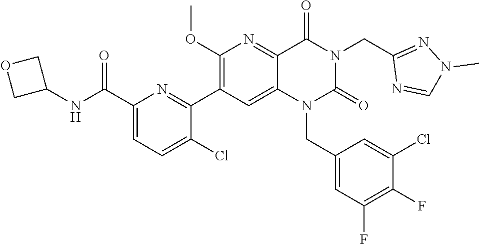 [SMILES: COc1nc2c(=O)n(Cc3ncn(C)n3)c(=O)n(Cc3cc(F)c(F)c(Cl)c3)c2cc1-c1nc(C(=O)NC2COC2)ccc1Cl] | 2 | 1.90 | 659.1 |
| I-0886 | 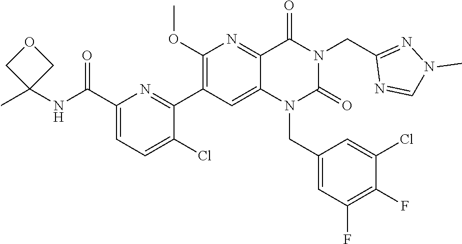 | 1 | 1.97 | 673.1 |
| I-0887 | 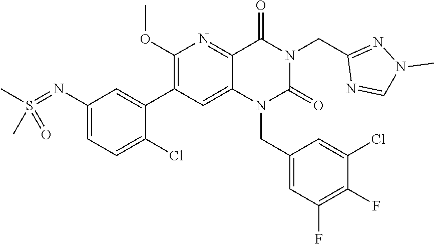 | 1 | 2.00 | 650 |
| I-0891 | 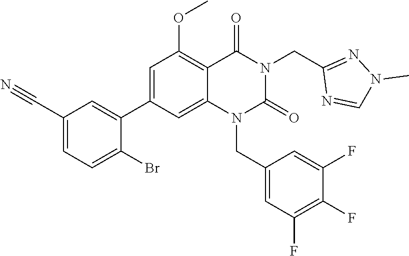 [SMILES: COc1cc(-c2cc(C#N)ccc2Br)cc2c1c(=O)n(Cc1ncn(C)n1)c(=O)n2Cc1cc(F)c(F)c(F)c1] | 1 | 2.20 | 611 |
| I-0893 | 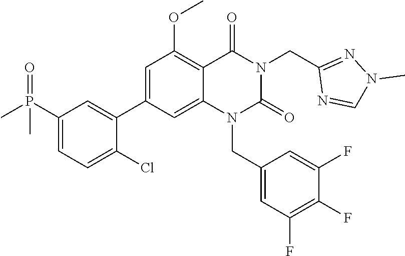 | 1 | 1.74 | 618 |
| I-0895 | 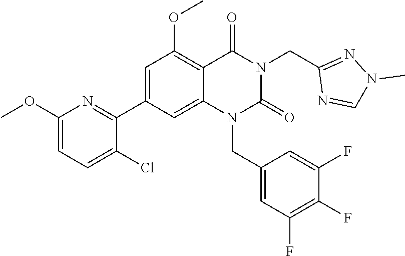 | 1 | 2.30 | 573 |

TABLE 64

| | | LC/MS | | |
|---|---|---|---|---|
| Compound | | measurement | Retention time | |
| No. | Structural formula | conditions | (min) | m/z |
| I-0901 | 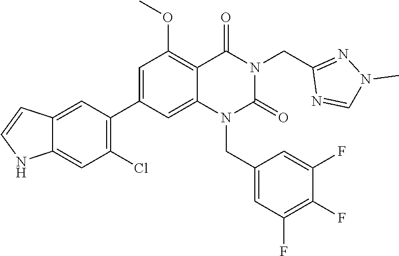 [SMILES: COc1cc(-c2cc3cc[nH]c3cc2Cl)cc2c1c(=O)n(Cc1ncn(C)n1)c(=O)n2Cc1cc(F)c(F)c(F)c1] | 1 | 2.10 | 581 |
| I-0902 | 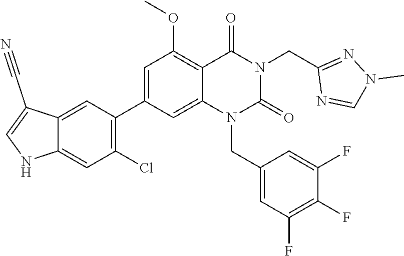 [SMILES: COc1cc(-c2cc3c(C#N)c[nH]c3cc2Cl)cc2c1c(=O)n(Cc1ncn(C)n1)c(=O)n2Cc1cc(F)c(F)c(F)c1] | 1 | 2.00 | 606 |
| I-0907 | 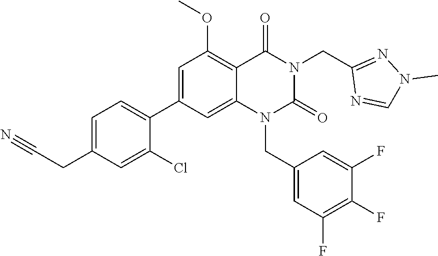 [SMILES: COc1cc(-c2ccc(CC#N)cc2Cl)cc2c1c(=O)n(Cc1ncn(C)n1)c(=O)n2Cc1cc(F)c(F)c(F)c1] | 1 | 2.09 | 581 |
| I-0914 | 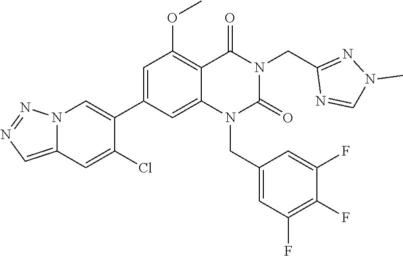 [SMILES: COc1cc(-c2cn3nncc3cc2Cl)cc2c1c(=O)n(Cc1ncn(C)n1)c(=O)n2Cc1cc(F)c(F)c(F)c1] | 1 | 1.92 | 583 |
| I-0917 | 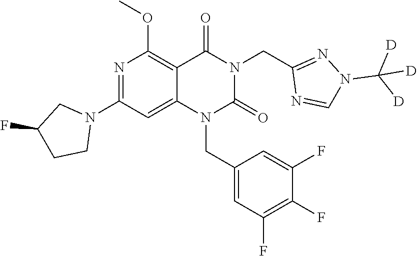 [SMILES: [2H]C([2H])([2H])n1cnc(Cn2c(=O)c3c(OC)nc(N4CC[C@@H](F)C4)cc3n(Cc3cc(F)c(F)c(F)c3)c2=O)n1] | 1 | 2.04 | 523 |
| I-0926 | 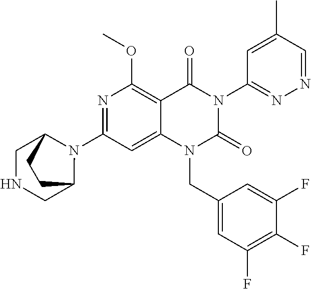 [SMILES: COc1nc(N2[C@@H]3CC[C@H]2CNC3)cc2c1c(=O)n(-c1cc(C)cnn1)c(=O)n2Cc1cc(F)c(F)c(F)c1] | 1 | 1.49 | 540 |

TABLE 65

| | | LC/MS | | |
|---|---|---|---|---|
| Compound | | measurement | Retention time | |
| No. | Structural formula | conditions | (min) | m/z |
| I-0927 | 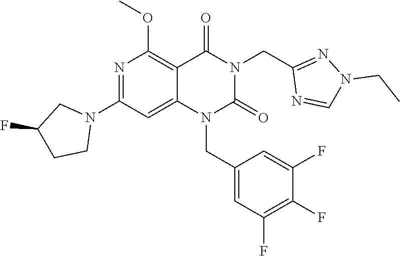 [SMILES: CCn1cnc(Cn2c(=O)c3c(OC)nc(N4CC[C@@H](F)C4)cc3n(Cc3cc(F)c(F)c(F)c3)c2=O)n1] | 1 | 2.12 | 534 |
| I-0929 | 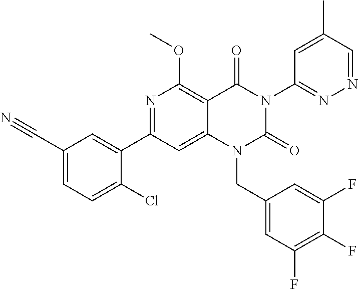 [SMILES: COc1nc(-c2cc(C#N)ccc2Cl)cc2c1c(=O)n(-c1cc(C)cnn1)c(=O)n2Cc1cc(F)c(F)c(F)c1] | 1 | 2.33 | 565 |
| I-0930 | 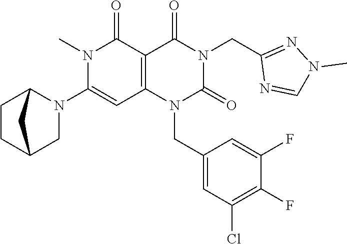 [SMILES: Cn1cnc(Cn2c(=O)c3c(=O)n(C)c(N4C[C@@H]5CC[C@H]4C5)cc3n(Cc3cc(F)c(F)c(Cl)c3)c2=O)n1] | 1 | 1.91 | 544 |
| I-0931 | 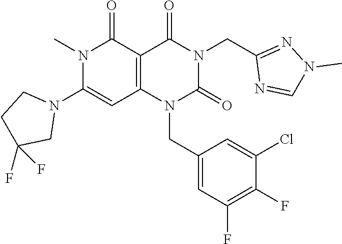 [SMILES: Cn1cnc(Cn2c(=O)c3c(=O)n(C)c(N4CCC(F)(F)C4)cc3n(Cc3cc(F)c(F)c(Cl)c3)c2=O)n1] | 1 | 1.87 | 554 |
| I-0932 | 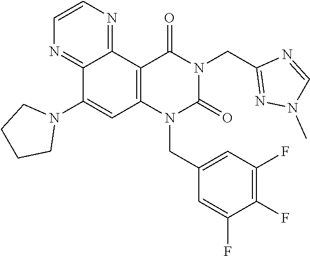 [SMILES: Cn1cnc(Cn2c(=O)c3c4nccnc4c(N4CCCC4)cc3n(Cc3cc(F)c(F)c(F)c3)c2=O)n1] | 1 | 1.95 | 523 |
| I-0933 | 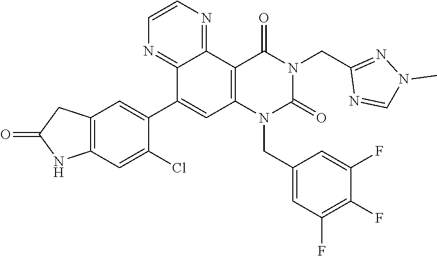 [SMILES: Cn1cnc(Cn2c(=O)c3c4nccnc4c(-c4cc5c(cc4Cl)NC(=O)C5)cc3n(Cc3cc(F)c(F)c(F)c3)c2=O)n1] | 1 | 1.65 | 619 |

TABLE 66

| | | LC/MS | | |
|---|---|---|---|---|
| Compound | | measurement | Retention time | |
| No. | Structural formula | conditions | (min) | m/z |
| I-0934 | 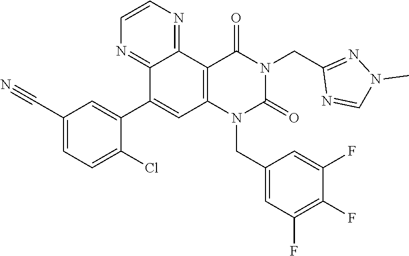 [SMILES: Cn1cnc(Cn2c(=O)c3c4nccnc4c(-c4cc(C#N)ccc4Cl)cc3n(Cc3cc(F)c(F)c(F)c3)c2=O)n1] | 1 | 2.12 | 589 |
| I-0935 | 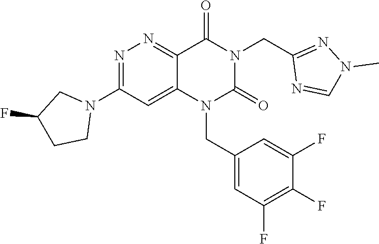 [SMILES: Cn1cnc(Cn2c(=O)c3nnc(N4CC[C@@H](F)C4)cc3n(Cc3cc(F)c(F)c(F)c3)c2=O)n1] | 2 | 1.72 | 491.1 |

TABLE 67

| | | LC/MS | Retention | |
|---|---|---|---|---|
| Compound | | measurement | time | |
| No. | Structural formula | conditions | (min) | m/z |
| I-0936 | 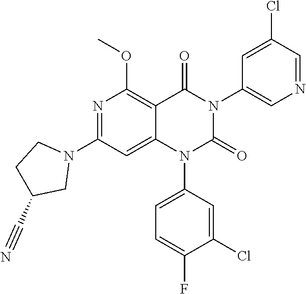 [SMILES: COc1nc(N2CC[C@@H](C#N)C2)cc2c1c(=O)n(-c1cncc(Cl)c1)c(=O)n2-c1ccc(F)c(Cl)c1] | 1 | 2.29 | 527 |
| I-0937 | 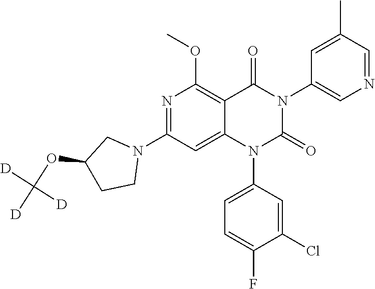 [SMILES: [2H]C([2H])([2H])O[C@@H]1CCN(c2cc3c(c(OC)n2)c(=O)n(-c2cncc(C)c2)c(=O)n3-c2ccc(F)c(Cl)c2)C1] | 1 | 2.13 | 515 |
| I-0938 | 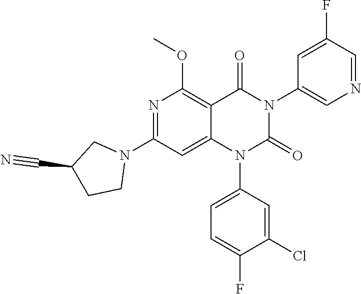 [SMILES: COc1nc(N2CC[C@@H](C#N)C2)cc2c1c(=O)n(-c1cncc(F)c1)c(=O)n2-c1ccc(F)c(Cl)c1] | 1 | 2.10 | 511 |
| I-0939 | 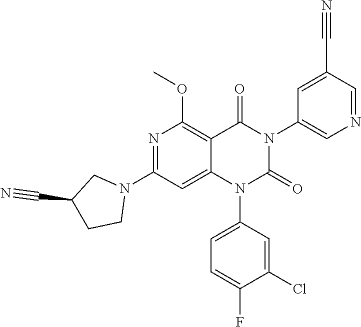 [SMILES: COc1nc(N2CC[C@@H](C#N)C2)cc2c1c(=O)n(-c1cncc(C#N)c1)c(=O)n2-c1ccc(F)c(Cl)c1] | 1 | 2.18 | 518 |
| I-0940 | 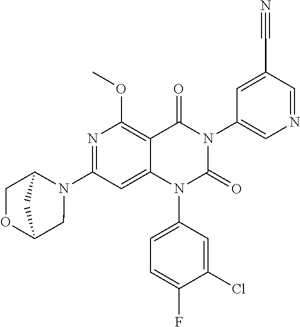 [SMILES: COc1nc(N2C[C@@H]3C[C@H]2CO3)cc2c1c(=O)n(-c1cncc(C#N)c1)c(=O)n2-c1ccc(F)c(Cl)c1] | 1 | 2.05 | 521 |
| I-0941 | 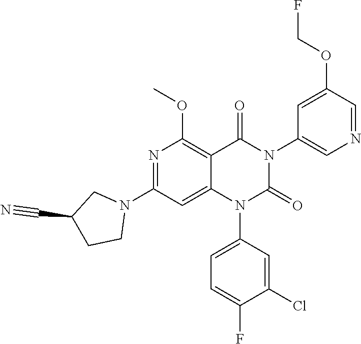 [SMILES: COc1nc(N2CC[C@@H](C#N)C2)cc2c1c(=O)n(-c1cncc(OCF)c1)c(=O)n2-c1ccc(F)c(Cl)c1] | 1 | 2.04 | 541 |

TABLE 68

| | | LC/MS | Retention | |
|---|---|---|---|---|
| Compound | | measurement | time | |
| No. | Structural formula | conditions | (min) | m/z |
| I-0942 | 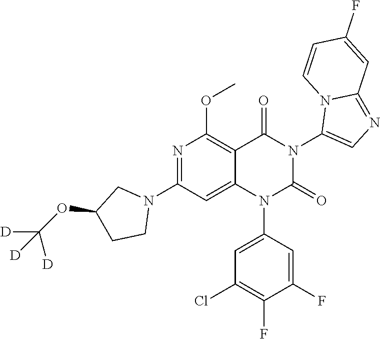 [SMILES: [2H]C([2H])([2H])O[C@@H]1CCN(c2cc3c(c(OC)n2)c(=O)n(-c2cnc4cc(F)ccn24)c(=O)n3-c2cc(F)c(F)c(Cl)c2)C1] | 1 | 2.06 | 576.05 |
| I-0943 | 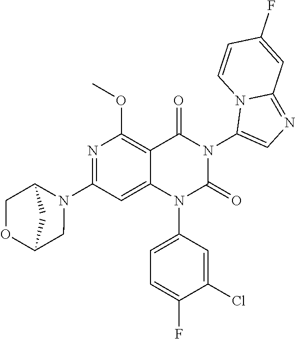 [SMILES: COc1nc(N2C[C@@H]3C[C@H]2CO3)cc2c1c(=O)n(-c1cnc3cc(F)ccn13)c(=O)n2-c1ccc(F)c(Cl)c1] | 3 | 2.05 | 553 |
| I-0944 | 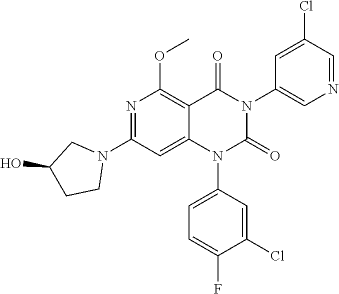 [SMILES: COc1nc(N2CC[C@@H](O)C2)cc2c1c(=O)n(-c1cncc(Cl)c1)c(=O)n2-c1ccc(F)c(Cl)c1] | 1 | 2.00 | 518 |
| I-0945 | 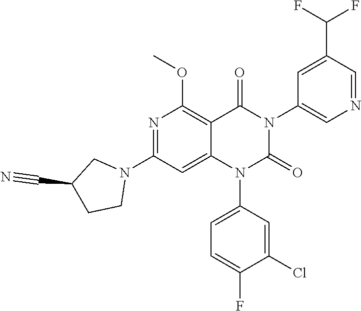 [SMILES: COc1nc(N2CC[C@@H](C#N)C2)cc2c1c(=O)n(-c1cncc(C(F)F)c1)c(=O)n2-c1ccc(F)c(Cl)c1] | 1 | 2.23 | 543 |
| I-0946 | 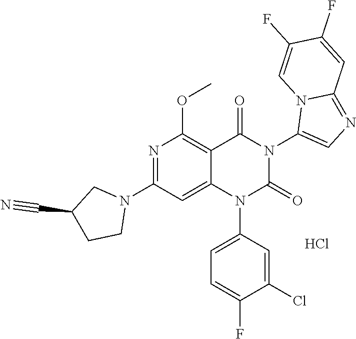 [SMILES: COc1nc(N2CC[C@@H](C#N)C2)cc2c1c(=O)n(-c1cnc3cc(F)c(F)cn13)c(=O)n2-c1ccc(F)c(Cl)c1 ; Cl] | 1 | 2.07 | 568 |
| I-0947 | 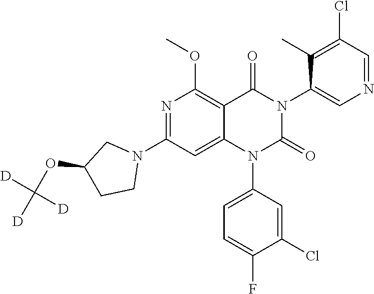 | 1 | 2.43 | 549 |

TABLE 69

| | | LC/MS | Retention | |
|---|---|---|---|---|
| Compound | | measurement | time | |
| No. | Structural formula | conditions | (min) | m/z |
| I-0948 | 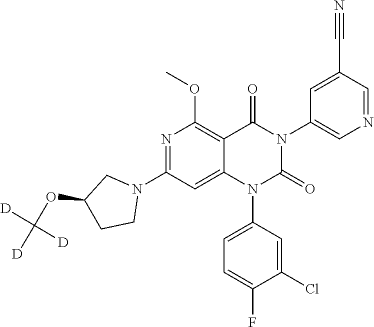 [SMILES: [2H]C([2H])([2H])O[C@@H]1CCN(c2cc3c(c(OC)n2)c(=O)n(-c2cncc(C#N)c2)c(=O)n3-c2ccc(F)c(Cl)c2)C1] | 1 | 2.31 | 526 |
| I-0949 | 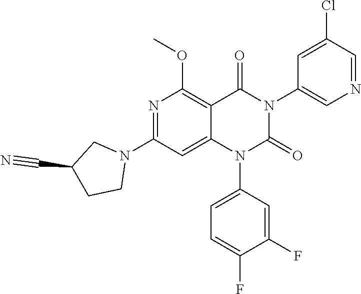 [SMILES: COc1nc(N2CC[C@@H](C#N)C2)cc2c1c(=O)n(-c1cncc(Cl)c1)c(=O)n2-c1ccc(F)c(F)c1] | 1 | 2.13 | 511 |
| I-0950 | 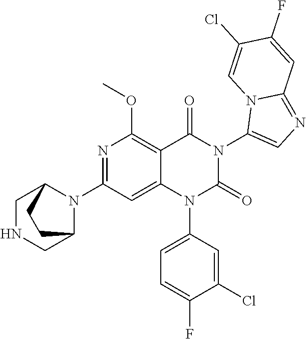 [SMILES: COc1nc(N2[C@@H]3CC[C@H]2CNC3)cc2c1c(=O)n(-c1cnc3cc(F)c(Cl)cn13)c(=O)n2-c1ccc(F)c(Cl)c1] | 3 | 1.86 | 600 |
| I-0951 | 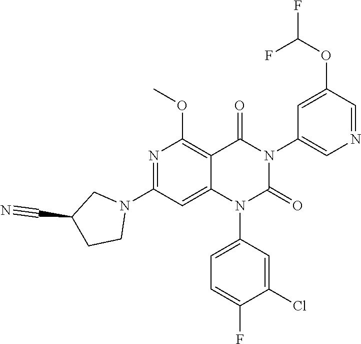 [SMILES: COc1nc(N2CC[C@@H](C#N)C2)cc2c1c(=O)n(-c1cncc(OC(F)F)c1)c(=O)n2-c1ccc(F)c(Cl)c1] | 1 | 2.16 | 559 |
| I-0952 | 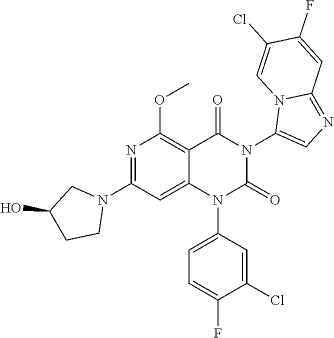 [SMILES: COc1nc(N2CC[C@@H](O)C2)cc2c1c(=O)n(-c1cnc3cc(F)c(Cl)cn13)c(=O)n2-c1ccc(F)c(Cl)c1] | 1 | 1.96 | 575 |
| I-0953 | 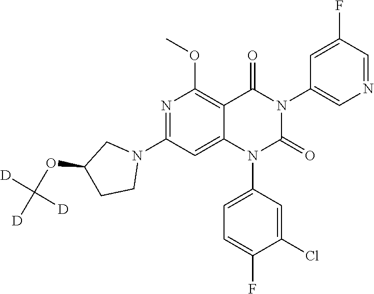 [SMILES: [2H]C([2H])([2H])O[C@@H]1CCN(c2cc3c(c(OC)n2)c(=O)n(-c2cncc(F)c2)c(=O)n3-c2ccc(F)c(Cl)c2)C1] | 1 | 2.28 | 519 |

TABLE 70

| | | IC/MS | Retention | |
|---|---|---|---|---|
| Compound | | measurement | time | |
| No. | Structural formula | conditions | (min) | m/z |
| I-0954 | 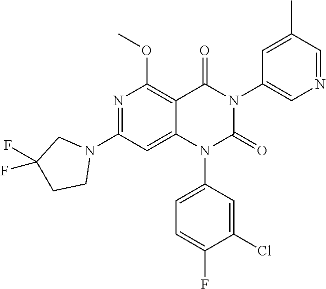 [SMILES: COc1nc(N2CCC(F)(F)C2)cc2c1c(=O)n(-c1cncc(C)c1)c(=O)n2-c1ccc(F)c(Cl)c1] | 1 | 2.26 | 518 |
| I-0955 | 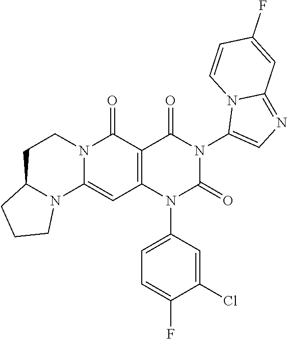 [SMILES: O=c1c2c(=O)n(-c3cnc4cc(F)ccn34)c(=O)n(-c3ccc(F)c(Cl)c3)c2cc2n1CC[C@@H]1CCCN21] | 1 | 1.77 | 537.05 |
| I-0956 | 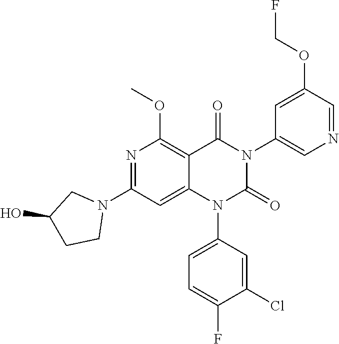 [SMILES: COc1nc(N2CC[C@@H](O)C2)cc2c1c(=O)n(-c1cncc(OCF)c1)c(=O)n2-c1ccc(F)c(Cl)c1] | 1 | 1.93 | 532 |
| I-0957 | 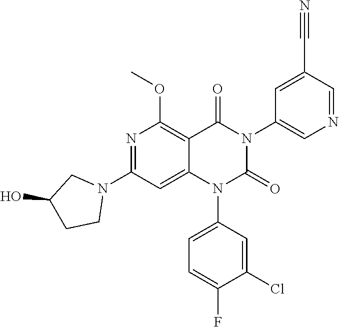 [SMILES: COc1nc(N2CC[C@@H](O)C2)cc2c1c(=O)n(-c1cncc(C#N)c1)c(=O)n2-c1ccc(F)c(Cl)c1] | 1 | 1.96 | 509 |
| I-0958 | 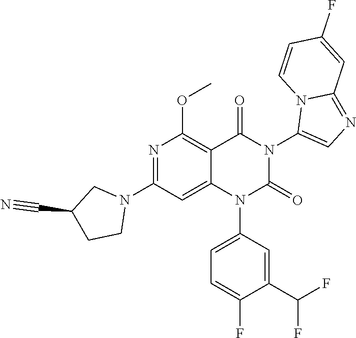 [SMILES: COc1nc(N2CC[C@@H](C#N)C2)cc2c1c(=O)n(-c1cnc3cc(F)ccn13)c(=O)n2-c1ccc(F)c(C(F)F)c1] | 1 | 1.81 | 566.05 |
| I-0959 | 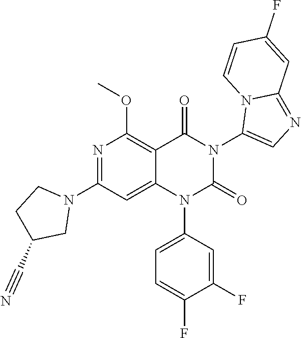 [SMILES: COc1nc(N2CC[C@@H](C#N)C2)cc2c1c(=O)n(-c1cnc3cc(F)ccn13)c(=O)n2-c1ccc(F)c(F)c1] | 1 | 1.64 | 534.1 |

TABLE 71

| | | LC/MS | Retention | |
|---|---|---|---|---|
| Compound | | measurement | time | |
| No. | Structural formula | conditions | (min) | m/z |
| I-0960 | 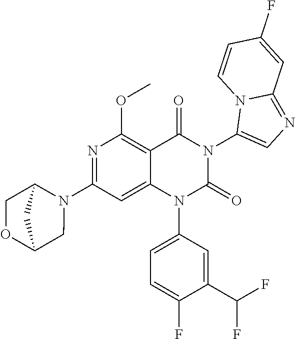 [SMILES: COc1nc(N2C[C@@H]3C[C@H]2CO3)cc2c1c(=O)n(-c1cnc3cc(F)ccn13)c(=O)n2-c1ccc(F)c(C(F)F)c1] | 1 | 1.72 | 569.1 |
| I-0961 | 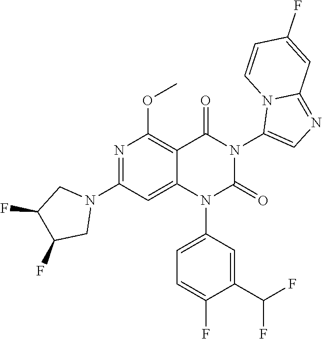 [SMILES: COc1nc(N2C[C@@H](F)[C@@H](F)C2)cc2c1c(=O)n(-c1cnc3cc(F)ccn13)c(=O)n2-c1ccc(F)c(C(F)F)c1] | 1 | 1.94 | 577 |
| I-0962 | 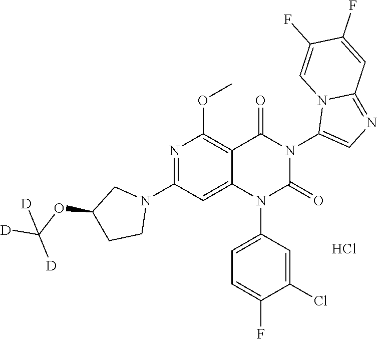 [SMILES: [2H]C([2H])([2H])O[C@@H]1CCN(c2cc3c(c(OC)n2)c(=O)n(-c2cnc4cc(F)c(F)cn24)c(=O)n3-c2ccc(F)c(Cl)c2)C1 ; Cl] | 1 | 2.19 | 576 |
| I-0963 | 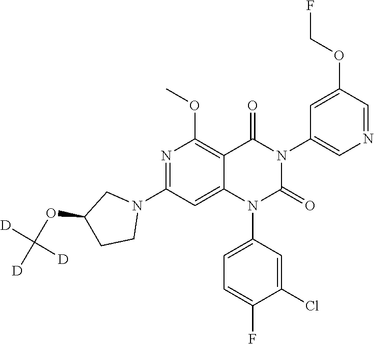 [SMILES: [2H]C([2H])([2H])O[C@@H]1CCN(c2cc3c(c(OC)n2)c(=O)n(-c2cncc(OCF)c2)c(=O)n3-c2ccc(F)c(Cl)c2)C1] | 1 | 2.23 | 549 |
| I-0964 | 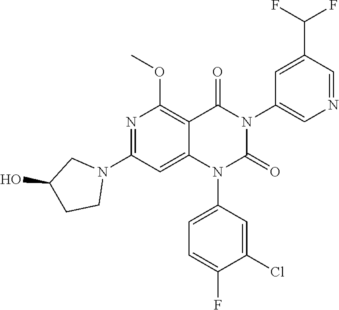 [SMILES: COc1nc(N2CC[C@@H](O)C2)cc2c1c(=O)n(-c1cncc(C(F)F)c1)c(=O)n2-c1ccc(F)c(Cl)c1] | 1 | 1.96 | 534 |
| I-0965 | 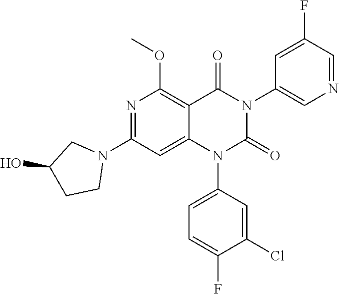 [SMILES: COc1nc(N2CC[C@@H](O)C2)cc2c1c(=O)n(-c1cncc(F)c1)c(=O)n2-c1ccc(F)c(Cl)c1] | 1 | 1.95 | 502 |

TABLE 72

| | | LC/MS | Retention | |
|---|---|---|---|---|
| Compound | | measurement | time | |
| No. | Structural formula | conditions | (min) | m/z |
| I-0966 | 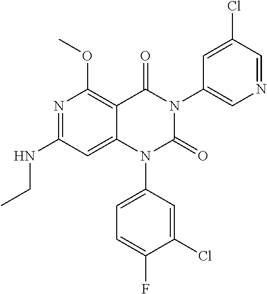 [SMILES: CCNc1cc2c(c(OC)n1)c(=O)n(-c1cncc(Cl)c1)c(=O)n2-c1ccc(F)c(Cl)c1] | 1 | 2.34 | 475.9 |
| I-0967 | 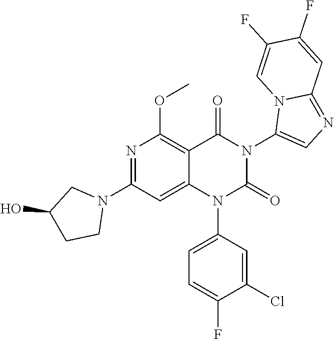 [SMILES: COc1nc(N2CC[C@@H](O)C2)cc2c1c(=O)n(-c1cnc3cc(F)c(F)cn13)c(=O)n2-c1ccc(F)c(Cl)c1] | 1 | 1.86 | 559 |
| I-0968 | 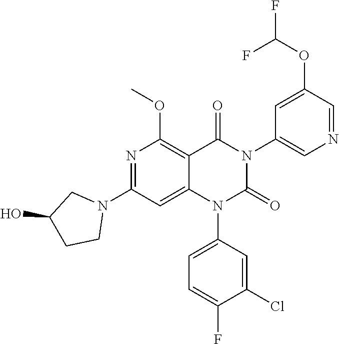 [SMILES: COc1nc(N2CC[C@@H](O)C2)cc2c1c(=O)n(-c1cncc(OC(F)F)c1)c(=O)n2-c1ccc(F)c(Cl)c1] | 1 | 2.04 | 550 |
| I-0969 | 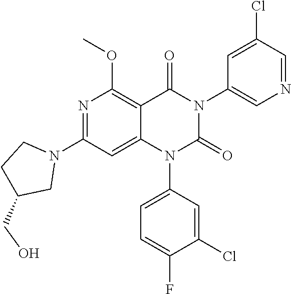 [SMILES: COc1nc(N2CC[C@@H](CO)C2)cc2c1c(=O)n(-c1cncc(Cl)c1)c(=O)n2-c1ccc(F)c(Cl)c1] | 1 | 2.12 | 532 |
| I-0970 | 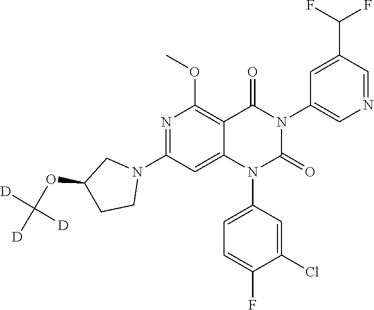 [SMILES: [2H]C([2H])([2H])O[C@@H]1CCN(c2cc3c(c(OC)n2)c(=O)n(-c2cncc(C(F)F)c2)c(=O)n3-c2ccc(F)c(Cl)c2)C1] | 1 | 2.35 | 551 |
| I-0971 | 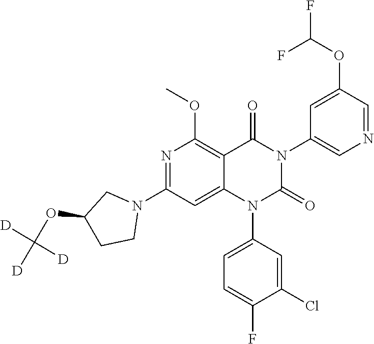 [SMILES: [2H]C([2H])([2H])O[C@@H]1CCN(c2cc3c(c(OC)n2)c(=O)n(-c2cncc(OC(F)F)c2)c(=O)n3-c2ccc(F)c(Cl)c2)C1] | 1 | 2.35 | 567 |

TABLE 73

| | | LC/MS | Retention | |
|---|---|---|---|---|
| Compound | | measurement | time | |
| No. | Structural formula | conditions | (min) | m/z |
| I-0972 | 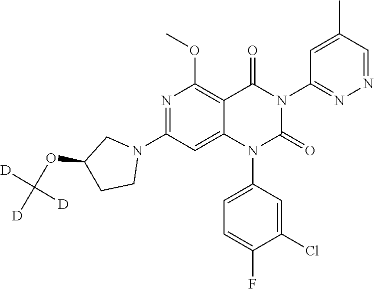 [SMILES: [2H]C([2H])([2H])O[C@@H]1CCN(c2cc3c(c(OC)n2)c(=O)n(-c2cc(C)cnn2)c(=O)n3-c2ccc(F)c(Cl)c2)C1] | 1 | 2.04 | 516 |
| I-0973 | 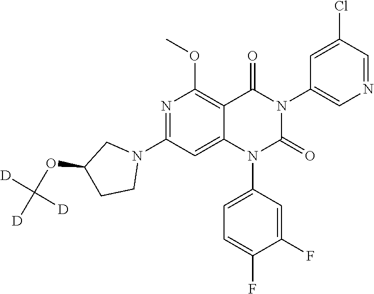 [SMILES: [2H]C([2H])([2H])O[C@@H]1CCN(c2cc3c(c(OC)n2)c(=O)n(-c2cncc(Cl)c2)c(=O)n3-c2ccc(F)c(F)c2)C1] | 1 | 2.23 | 519 |
| I-0974 | 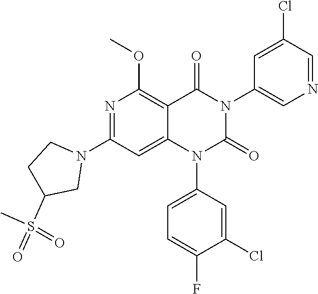 [SMILES: COc1nc(N2CCC(S(C)(=O)=O)C2)cc2c1c(=O)n(-c1cncc(Cl)c1)c(=O)n2-c1ccc(F)c(Cl)c1] | 1 | 2.11 | 579.9 |
| I-0975 | 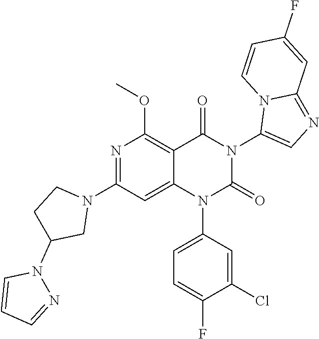 [SMILES: COc1nc(N2CCC(n3cccn3)C2)cc2c1c(=O)n(-c1cnc3cc(F)ccn13)c(=O)n2-c1ccc(F)c(Cl)c1] | 1 | 2.02 | 591 |
| I-0976 | 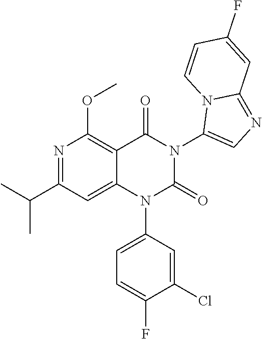 [SMILES: COc1nc(C(C)C)cc2c1c(=O)n(-c1cnc3cc(F)ccn13)c(=O)n2-c1ccc(F)c(Cl)c1] | 1 | 2.27 | 498 |
| I-0977 | 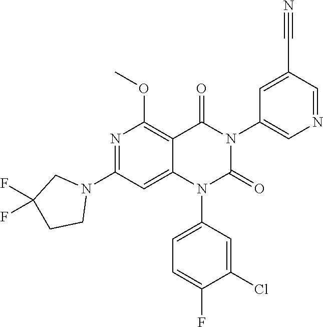 [SMILES: COc1nc(N2CCC(F)(F)C2)cc2c1c(=O)n(-c1cncc(C#N)c1)c(=O)n2-c1ccc(F)c(Cl)c1] | 1 | 2.42 | 529 |

TABLE 74

| | | LC/MS | Retention | |
|---|---|---|---|---|
| Compound | | measurement | time | |
| No. | Structural formula | conditions | (min) | m/z |
| I-0978 | 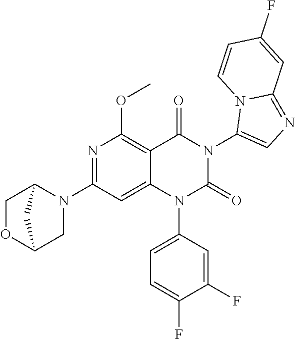 [SMILES: COc1nc(N2C[C@@H]3C[C@H]2CO3)cc2c1c(=O)n(-c1cnc3cc(F)ccn13)c(=O)n2-c1ccc(F)c(F)c1] | 1 | 1.67 | 537.1 |
| I-0979 | 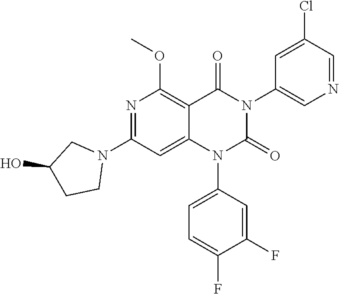 [SMILES: COc1nc(N2CC[C@@H](O)C2)cc2c1c(=O)n(-c1cncc(Cl)c1)c(=O)n2-c1ccc(F)c(F)c1] | 1 | 1.90 | 502 |
| I-0980 | 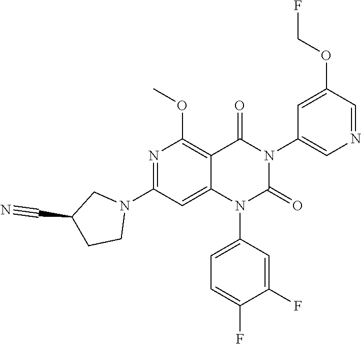 [SMILES: COc1nc(N2CC[C@@H](C#N)C2)cc2c1c(=O)n(-c1cncc(OCF)c1)c(=O)n2-c1ccc(F)c(F)c1] | 1 | 1.96 | 525 |
| I-0981 | 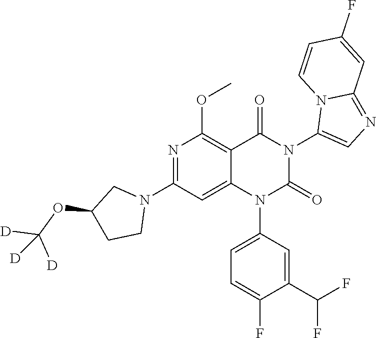 [SMILES: [2H]C([2H])([2H])O[C@@H]1CCN(c2cc3c(c(OC)n2)c(=O)n(-c2cnc4cc(F)ccn24)c(=O)n3-c2ccc(F)c(C(F)F)c2)C1] | 1 | 1.94 | 574.15 |
| I-0982 | 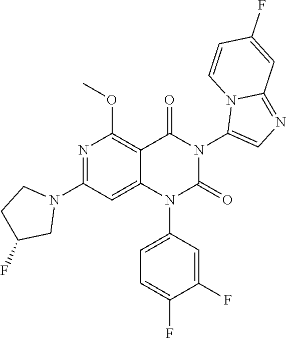 [SMILES: COc1nc(N2CC[C@@H](F)C2)cc2c1c(=O)n(-c1cnc3cc(F)ccn13)c(=O)n2-c1ccc(F)c(F)c1] | 1 | 1.76 | 527 |
| I-0983 | 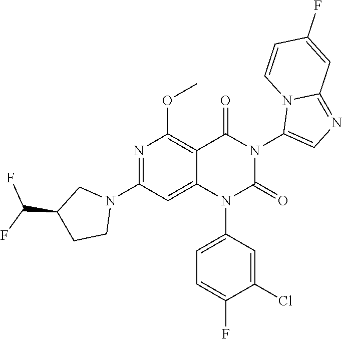 [SMILES: COc1nc(N2CC[C@@H](C(F)F)C2)cc2c1c(=O)n(-c1cnc3cc(F)ccn13)c(=O)n2-c1ccc(F)c(Cl)c1] | 1 | 2.14 | 575 |

TABLE 75

| | | LC/MS | Retention | |
|---|---|---|---|---|
| Compound | | measurement | time | |
| No. | Structural formula | conditions | (min) | m/z |
| I-0984 | 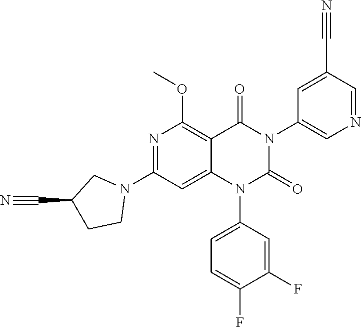 [SMILES: COc1nc(N2CC[C@@H](C#N)C2)cc2c1c(=O)n(-c1cncc(C#N)c1)c(=O)n2-c1ccc(F)c(F)c1] | 1 | 2.03 | 502 |
| I-0985 | 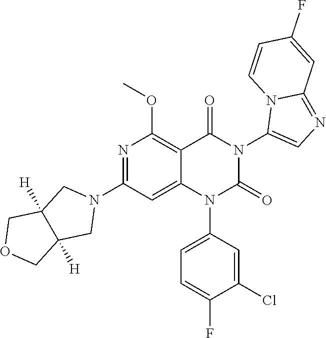 [SMILES: COc1nc(N2C[C@H]3COC[C@H]3C2)cc2c1c(=O)n(-c1cnc3cc(F)ccn13)c(=O)n2-c1ccc(F)c(Cl)c1] | 1 | 1.92 | 567 |
| I-0986 | 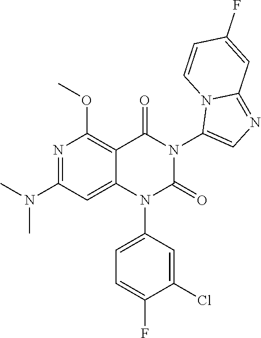 [SMILES: COc1nc(N(C)C)cc2c1c(=O)n(-c1cnc3cc(F)ccn13)c(=O)n2-c1ccc(F)c(Cl)c1] | 3 | 2.17 | 499.1 |
| I-0987 | 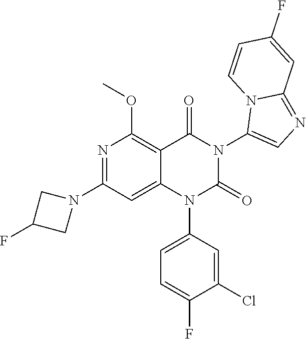 [SMILES: COc1nc(N2CC(F)C2)cc2c1c(=O)n(-c1cnc3cc(F)ccn13)c(=O)n2-c1ccc(F)c(Cl)c1] | 1 | 1.79 | 529 |
| I-0988 | 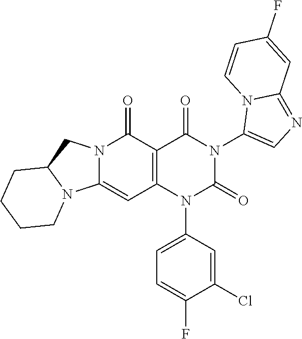 [SMILES: O=c1c2c(=O)n(-c3cnc4cc(F)ccn34)c(=O)n(-c3ccc(F)c(Cl)c3)c2cc2n1C[C@@H]1CCCCN21] | 1 | 1.74 | 537.05 |
| I-0989 | 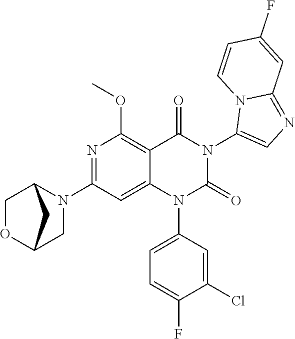 [SMILES: COc1nc(N2C[C@H]3C[C@@H]2CO3)cc2c1c(=O)n(-c1cnc3cc(F)ccn13)c(=O)n2-c1ccc(F)c(Cl)c1] | 3 | 2.05 | 553 |

TABLE 76

| | | LC/MS | Retention | |
|---|---|---|---|---|
| Compound | | measurement | time | |
| No. | Structural formula | conditions | (min) | m/z |
| I-0990 | 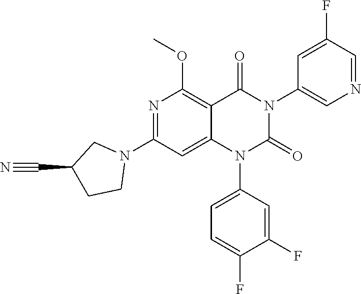 [SMILES: COc1nc(N2CC[C@@H](C#N)C2)cc2c1c(=O)n(-c1cncc(F)c1)c(=O)n2-c1ccc(F)c(F)c1] | 1 | 2.04 | 495 |
| I-0991 | 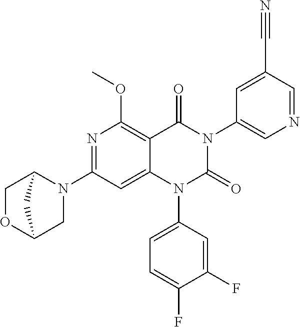 [SMILES: COc1nc(N2C[C@@H]3C[C@H]2CO3)cc2c1c(=O)n(-c1cncc(C#N)c1)c(=O)n2-c1ccc(F)c(F)c1] | 1 | 1.94 | 505 |
| I-0992 | 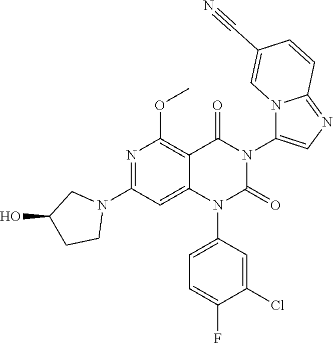 [SMILES: COc1nc(N2CC[C@@H](O)C2)cc2c1c(=O)n(-c1cnc3ccc(C#N)cn13)c(=O)n2-c1ccc(F)c(Cl)c1] | 1 | 1.87 | 548.05 |
| I-0993 | 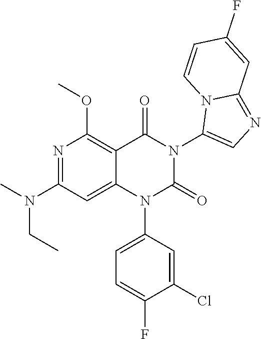 [SMILES: CCN(C)c1cc2c(c(OC)n1)c(=O)n(-c1cnc3cc(F)ccn13)c(=O)n2-c1ccc(F)c(Cl)c1] | 3 | 2.29 | 513 |
| I-0994 | 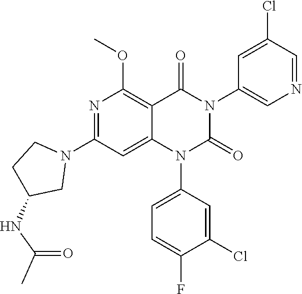 [SMILES: COc1nc(N2CC[C@@H](NC(C)=O)C2)cc2c1c(=O)n(-c1cncc(Cl)c1)c(=O)n2-c1ccc(F)c(Cl)c1] | 1 | 2.00 | 559 |
| I-0995 | 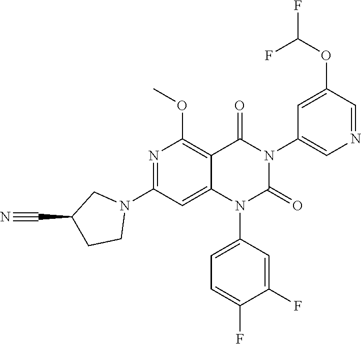 [SMILES: COc1nc(N2CC[C@@H](C#N)C2)cc2c1c(=O)n(-c1cncc(OC(F)F)c1)c(=O)n2-c1ccc(F)c(F)c1] | 1 | 2.08 | 543 |

TABLE 77

| | | LC/MS | Retention | |
|---|---|---|---|---|
| Compound | | measurement | time | |
| No. | Structural formula | conditions | (min) | m/z |
| I-0996 | 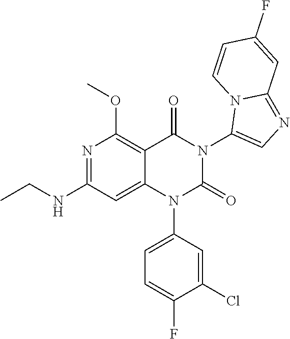 [SMILES: CCNc1cc2c(c(OC)n1)c(=O)n(-c1cnc3cc(F)ccn13)c(=O)n2-c1ccc(F)c(Cl)c1] | 3 | 2.12 | 499.1 |
| I-0997 | 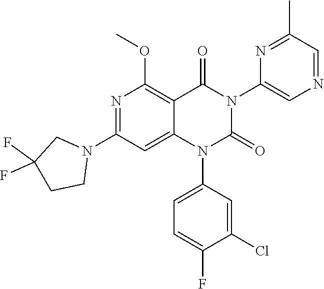 [SMILES: COc1nc(N2CCC(F)(F)C2)cc2c1c(=O)n(-c1cncc(C)n1)c(=O)n2-c1ccc(F)c(Cl)c1] | 1 | 2.59 | 519 |
| I-0998 | 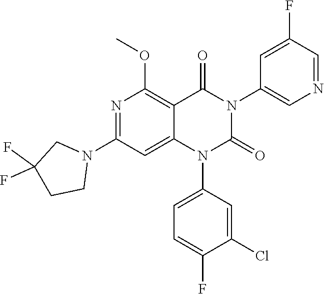 [SMILES: COc1nc(N2CCC(F)(F)C2)cc2c1c(=O)n(-c1cncc(F)c1)c(=O)n2-c1ccc(F)c(Cl)c1] | 1 | 2.41 | 522 |
| I-0999 | 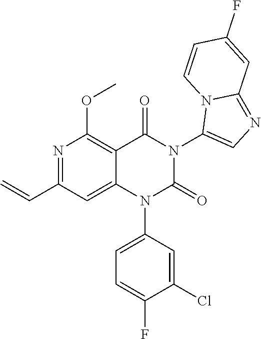 [SMILES: C=Cc1cc2c(c(OC)n1)c(=O)n(-c1cnc3cc(F)ccn13)c(=O)n2-c1ccc(F)c(Cl)c1] | 1 | 2.11 | 482 |
| I-1000 | 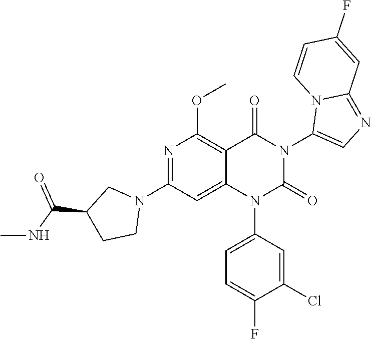 [SMILES: CNC(=O)[C@@H]1CCN(c2cc3c(c(OC)n2)c(=O)n(-c2cnc4cc(F)ccn24)c(=O)n3-c2ccc(F)c(Cl)c2)C1] | 1 | 1.60 | 582.05 |
| I-1001 | 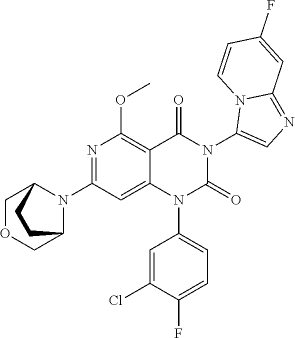 | 3 | 2.19 | 567 |

TABLE 78

| | | LC/MS | Retention | |
|---|---|---|---|---|
| Compound | | measurement | time | |
| No. | Structural formula | conditions | (min) | m/z |
| I-1002 | 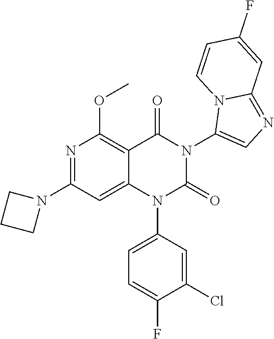 [SMILES: COc1nc(N2CCC2)cc2c1c(=O)n(-c1cnc3cc(F)ccn13)c(=O)n2-c1ccc(F)c(Cl)c1] | 1 | 2.02 | 511 |
| I-1003 | 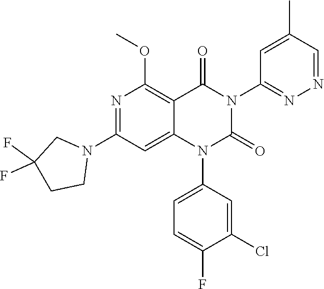 [SMILES: COc1nc(N2CCC(F)(F)C2)cc2c1c(=O)n(-c1cc(C)cnn1)c(=O)n2-c1ccc(F)c(Cl)c1] | 1 | 2.26 | 519 |
| I-1004 | 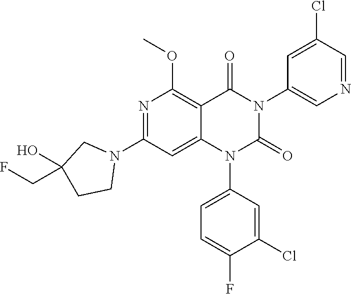 [SMILES: COc1nc(N2CCC(O)(CF)C2)cc2c1c(=O)n(-c1cncc(Cl)c1)c(=O)n2-c1ccc(F)c(Cl)c1] | 1 | 2.16 | 550 |
| I-1005 | 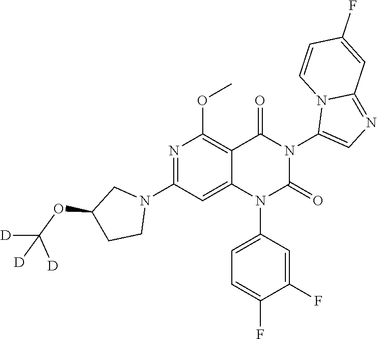 [SMILES: [2H]C([2H])([2H])O[C@@H]1CCN(c2cc3c(c(OC)n2)c(=O)n(-c2cnc4cc(F)ccn24)c(=O)n3-c2ccc(F)c(F)c2)C1] | 1 | 1.95 | 542.1 |
| I-1006 | 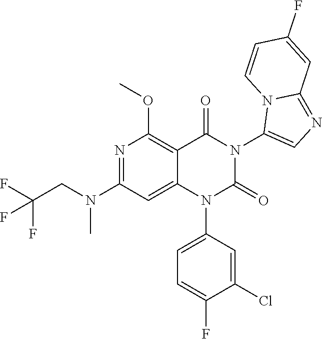 [SMILES: COc1nc(N(C)CC(F)(F)F)cc2c1c(=O)n(-c1cnc3cc(F)ccn13)c(=O)n2-c1ccc(F)c(Cl)c1] | 3 | 2.32 | 567 |
| I-1007 | 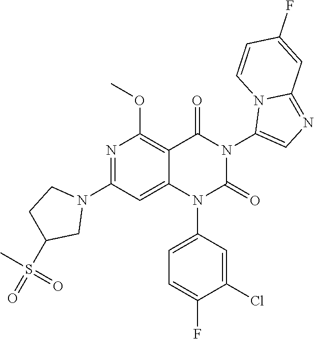 [SMILES: COc1nc(N2CCC(S(C)(=O)=O)C2)cc2c1c(=O)n(-c1cnc3cc(F)ccn13)c(=O)n2-c1ccc(F)c(Cl)c1] | 1 | 1.75 | 603 |

TABLE 79

| | | LC/MS | Retention | |
|---|---|---|---|---|
| Compound | | measurement | time | |
| No. | Structural formula | conditions | (min) | m/z |
| I-1008 | 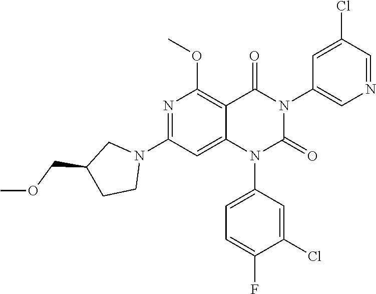 [SMILES: COC[C@@H]1CCN(c2cc3c(c(OC)n2)c(=O)n(-c2cncc(Cl)c2)c(=O)n3-c2ccc(F)c(Cl)c2)C1] | 1 | 2.52 | 546 |
| I-1009 | 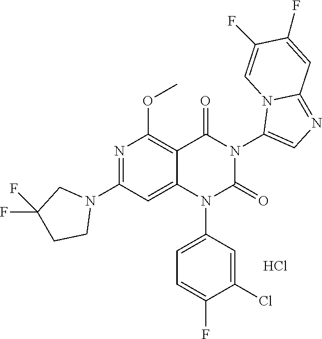 [SMILES: COc1nc(N2CCC(F)(F)C2)cc2c1c(=O)n(-c1cnc3cc(F)c(F)cn13)c(=O)n2-c1ccc(F)c(Cl)c1 ; Cl] | 1 | 2.32 | 579 |
| I-1010 | 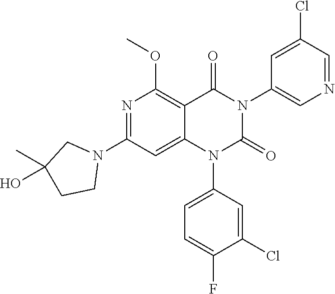 [SMILES: COc1nc(N2CCC(C)(O)C2)cc2c1c(=O)n(-c1cncc(Cl)c1)c(=O)n2-c1ccc(F)c(Cl)c1] | 1 | 2.17 | 532 |
| I-1011 | 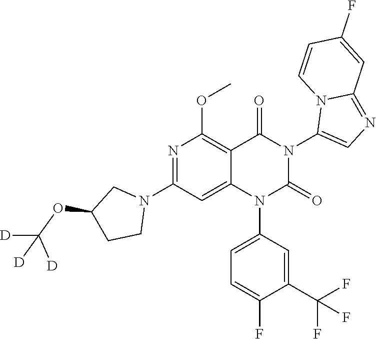 [SMILES: [2H]C([2H])([2H])O[C@@H]1CCN(c2cc3c(c(OC)n2)c(=O)n(-c2cnc4cc(F)ccn24)c(=O)n3-c2ccc(F)c(C(F)(F)F)c2)C1] | 1 | 2.03 | 592.1 |
| I-1012 | 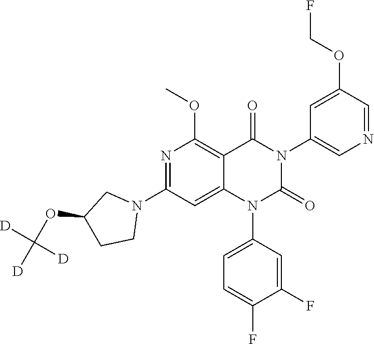 [SMILES: [2H]C([2H])([2H])O[C@@H]1CCN(c2cc3c(c(OC)n2)c(=O)n(-c2cncc(OCF)c2)c(=O)n3-c2ccc(F)c(F)c2)C1] | 1 | 2.13 | 533 |
| I-1013 | 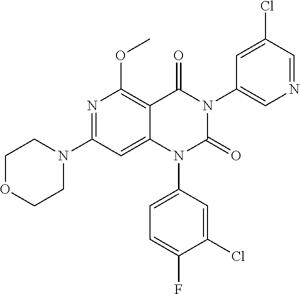 [SMILES: COc1nc(N2CCOCC2)cc2c1c(=O)n(-c1cncc(Cl)c1)c(=O)n2-c1ccc(F)c(Cl)c1] | 1 | 2.19 | 518.05 |

TABLE 80

| | | LC/MS | Retention | |
|---|---|---|---|---|
| Compound | | measurement | time | |
| No. | Structural formula | conditions | (min) | m/z |
| I-1014 | 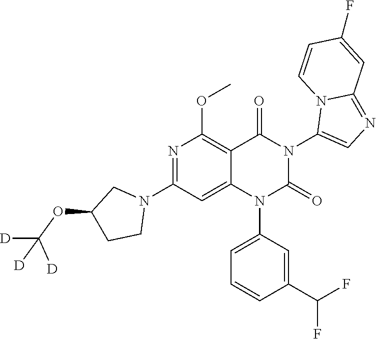 [SMILES: [2H]C([2H])([2H])O[C@@H]1CCN(c2cc3c(c(OC)n2)c(=O)n(-c2cnc4cc(F)ccn24)c(=O)n3-c2cccc(C(F)F)c2)C1] | 1 | 1.86 | 566.15 |
| I-1015 | 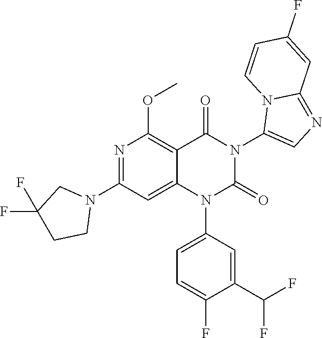 [SMILES: COc1nc(N2CCC(F)(F)C2)cc2c1c(=O)n(-c1cnc3cc(F)ccn13)c(=O)n2-c1ccc(F)c(C(F)F)c1] | 1 | 2.04 | 577 |
| I-1016 | 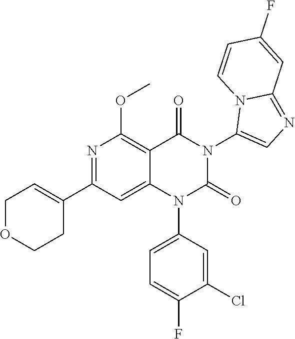 [SMILES: COc1nc(C2=CCOCC2)cc2c1c(=O)n(-c1cnc3cc(F)ccn13)c(=O)n2-c1ccc(F)c(Cl)c1] | 1 | 2.01 | 538 |
| I-1017 | 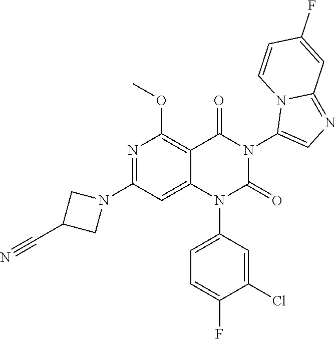 [SMILES: COc1nc(N2CC(C#N)C2)cc2c1c(=O)n(-c1cnc3cc(F)ccn13)c(=O)n2-c1ccc(F)c(Cl)c1] | 1 | 1.68 | 536 |
| I-1018 | 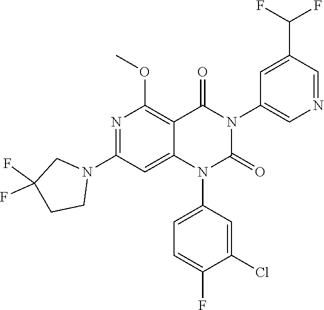 [SMILES: COc1nc(N2CCC(F)(F)C2)cc2c1c(=O)n(-c1cncc(C(F)F)c1)c(=O)n2-c1ccc(F)c(Cl)c1] | 1 | 2.47 | 554 |
| I-1019 | 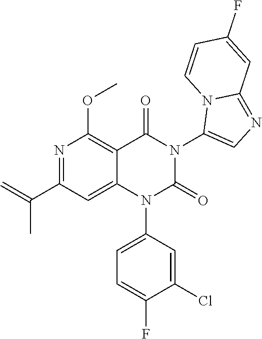 [SMILES: C=C(C)c1cc2c(c(OC)n1)c(=O)n(-c1cnc3cc(F)ccn13)c(=O)n2-c1ccc(F)c(Cl)c1] | 1 | 2.27 | 496.05 |

TABLE 81

| | | LC/MS | Retention | |
|---|---|---|---|---|
| Compound | | measurement | time | |
| No. | Structural formula | conditions | (min) | m/z |
| I-1020 | 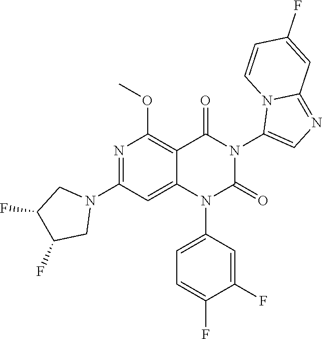 [SMILES: COc1nc(N2C[C@@H](F)[C@@H](F)C2)cc2c1c(=O)n(-c1cnc3cc(F)ccn13)c(=O)n2-c1ccc(F)c(F)c1] | 1 | 1.76 | 545.1 |
| I-1021 | 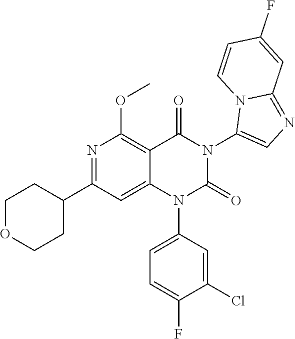 [SMILES: COc1nc(C2CCOCC2)cc2c1c(=O)n(-c1cnc3cc(F)ccn13)c(=O)n2-c1ccc(F)c(Cl)c1] | 1 | 1.91 | 540.1 |
| I-1022 | 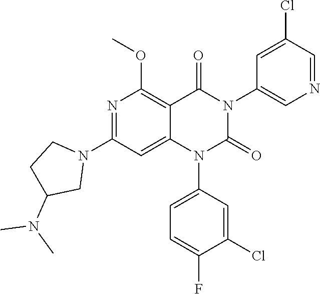 [SMILES: COc1nc(N2CCC(N(C)C)C2)cc2c1c(=O)n(-c1cncc(Cl)c1)c(=O)n2-c1ccc(F)c(Cl)c1] | 1 | 1.75 | 545 |
| I-1023 | 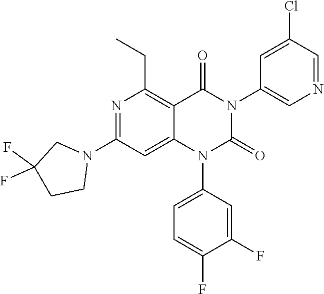 [SMILES: CCc1nc(N2CCC(F)(F)C2)cc2c1c(=O)n(-c1cncc(Cl)c1)c(=O)n2-c1ccc(F)c(F)c1] | 1 | 2.37 | 522 |
| I-1024 | 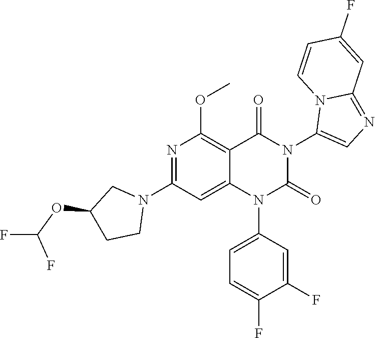 [SMILES: COc1nc(N2CC[C@@H](OC(F)F)C2)cc2c1c(=O)n(-c1cnc3cc(F)ccn13)c(=O)n2-c1ccc(F)c(F)c1] | 1 | 1.98 | 575.1 |
| I-1025 | 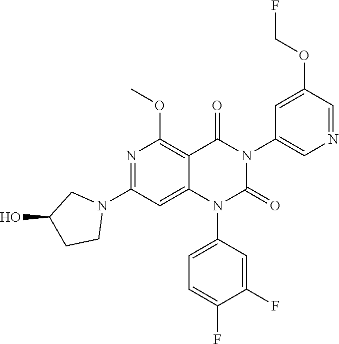 [SMILES: COc1nc(N2CC[C@@H](O)C2)cc2c1c(=O)n(-c1cncc(OCF)c1)c(=O)n2-c1ccc(F)c(F)c1] | 1 | 1.84 | 516 |

TABLE 82

| | | LC/MS | Retention | |
|---|---|---|---|---|
| Compound | | measurement | time | |
| No. | Structural formula | conditions | (min) | m/z |
| I-1026 | 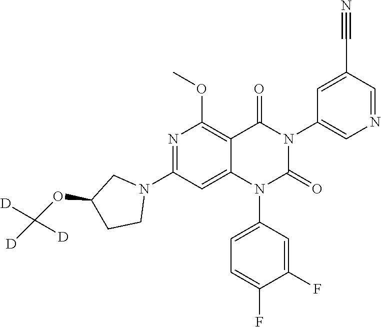 [SMILES: [2H]C([2H])([2H])O[C@@H]1CCN(c2cc3c(c(OC)n2)c(=O)n(-c2cncc(C#N)c2)c(=O)n3-c2ccc(F)c(F)c2)C1] | 1 | 2.14 | 510 |
| I-1027 | 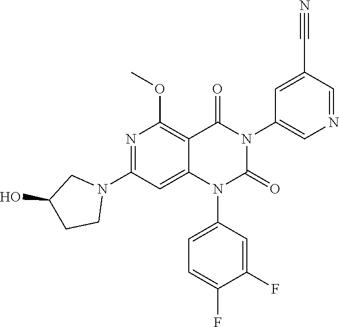 [SMILES: COc1nc(N2CC[C@@H](O)C2)cc2c1c(=O)n(-c1cncc(C#N)c1)c(=O)n2-c1ccc(F)c(F)c1] | 1 | 1.83 | 493 |
| I-1028 | 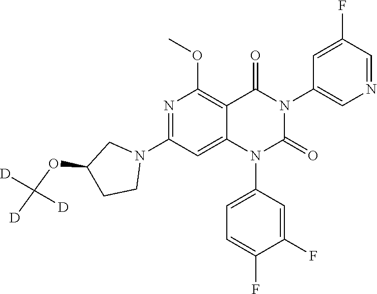 [SMILES: [2H]C([2H])([2H])O[C@@H]1CCN(c2cc3c(c(OC)n2)c(=O)n(-c2cncc(F)c2)c(=O)n3-c2ccc(F)c(F)c2)C1] | 1 | 2.12 | 503 |
| I-1029 | 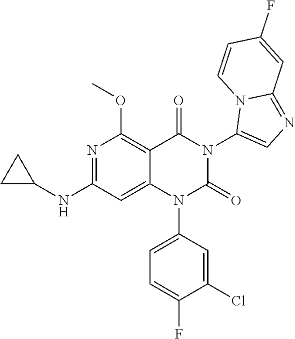 [SMILES: COc1nc(NC2CC2)cc2c1c(=O)n(-c1cnc3cc(F)ccn13)c(=O)n2-c1ccc(F)c(Cl)c1] | 3 | 2.14 | 511.1 |
| I-1030 | 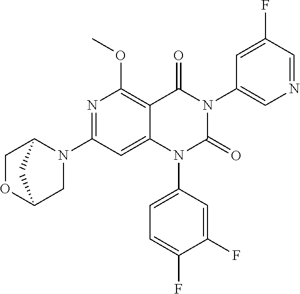 [SMILES: COc1nc(N2C[C@@H]3C[C@H]2CO3)cc2c1c(=O)n(-c1cncc(F)c1)c(=O)n2-c1ccc(F)c(F)c1] | 1 | 1.95 | 498 |
| I-1031 | 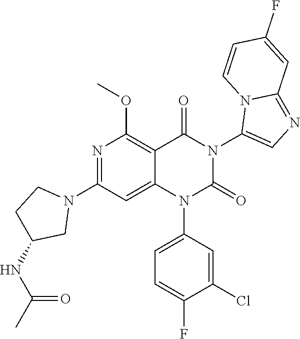 [SMILES: COc1nc(N2CC[C@@H](NC(C)=O)C2)cc2c1c(=O)n(-c1cnc3cc(F)ccn13)c(=O)n2-c1ccc(F)c(Cl)c1] | 1 | 1.65 | 582 |

TABLE 83

| | | LC/MS | Retention | |
|---|---|---|---|---|
| Compound | | measurement | time | |
| No. | Structural formula | conditions | (min) | m/z |
| I-1032 | 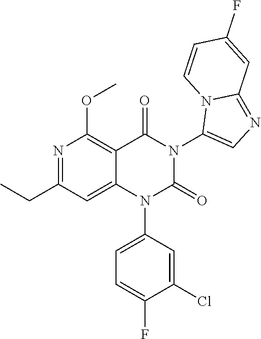 [SMILES: CCc1cc2c(c(OC)n1)c(=O)n(-c1cnc3cc(F)ccn13)c(=O)n2-c1ccc(F)c(Cl)c1] | 1 | 2.11 | 484 |
| I-1033 | 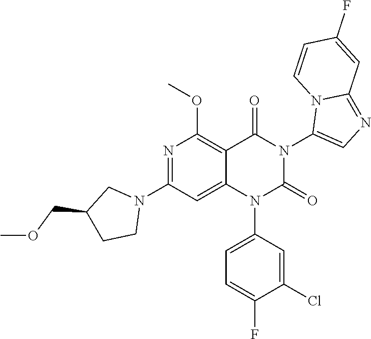 [SMILES: COC[C@@H]1CCN(c2cc3c(c(OC)n2)c(=O)n(-c2cnc4cc(F)ccn24)c(=O)n3-c2ccc(F)c(Cl)c2)C1] | 1 | 2.13 | 569 |
| I-1034 | 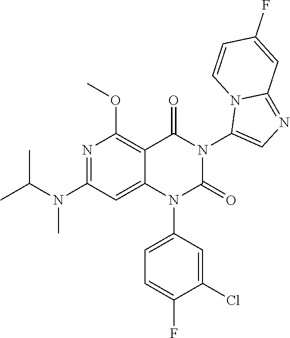 [SMILES: COc1nc(N(C)C(C)C)cc2c1c(=O)n(-c1cnc3cc(F)ccn13)c(=O)n2-c1ccc(F)c(Cl)c1] | 3 | 2.41 | 527.1 |
| I-1035 | 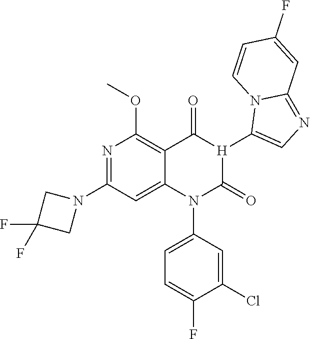 | 1 | 1.92 | 547 |
| I-1036 | 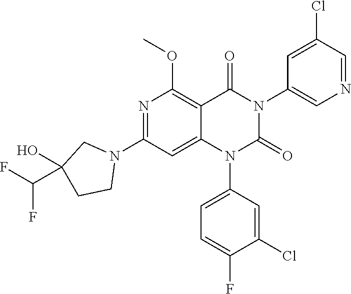 [SMILES: COc1nc(N2CCC(O)(C(F)F)C2)cc2c1c(=O)n(-c1cncc(Cl)c1)c(=O)n2-c1ccc(F)c(Cl)c1] | 1 | 2.24 | 568 |
| I-1037 | 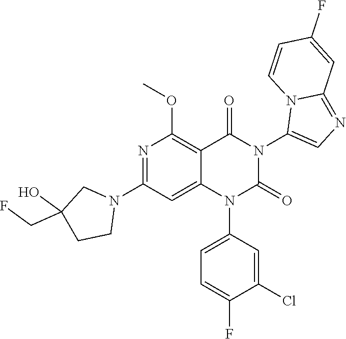 [SMILES: COc1nc(N2CCC(O)(CF)C2)cc2c1c(=O)n(-c1cnc3cc(F)ccn13)c(=O)n2-c1ccc(F)c(Cl)c1] | 1 | 1.79 | 573 |

TABLE 84

| | | LC/MS | Retention | |
|---|---|---|---|---|
| Compound | | measurement | time | |
| No. | Structural formula | conditions | (min) | m/z |
| I-1038 | 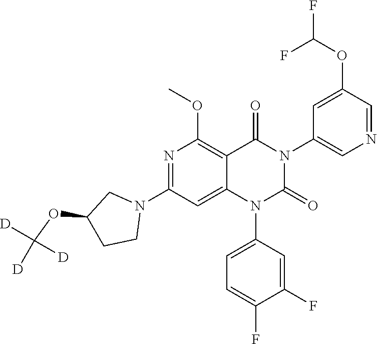 [SMILES: [2H]C([2H])([2H])O[C@@H]1CCN(c2cc3c(c(OC)n2)c(=O)n(-c2cncc(OC(F)F)c2)c(=O)n3-c2ccc(F)c(F)c2)C1] | 1 | 2.16 | 551 |
| I-1039 | 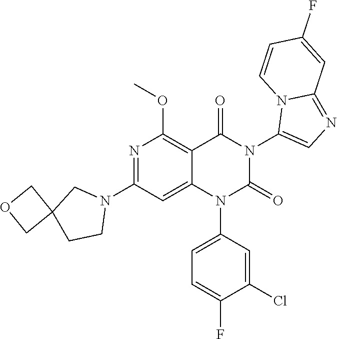 [SMILES: COc1nc(N2CCC3(COC3)C2)cc2c1c(=O)n(-c1cnc3cc(F)ccn13)c(=O)n2-c1ccc(F)c(Cl)c1] | 1 | 1.86 | 567 |
| I-1040 | 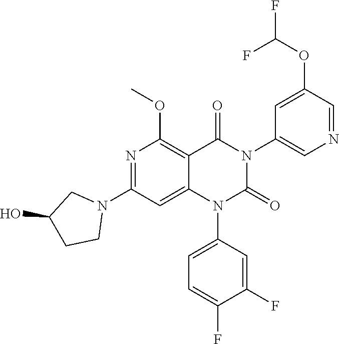 [SMILES: COc1nc(N2CC[C@@H](O)C2)cc2c1c(=O)n(-c1cncc(OC(F)F)c1)c(=O)n2-c1ccc(F)c(F)c1] | 1 | 1.96 | 534 |
| I-1041 | 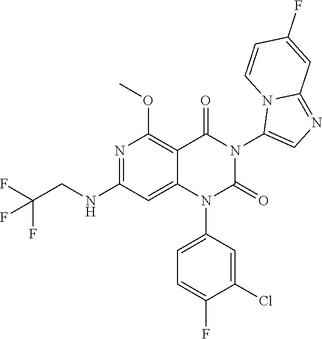 [SMILES: COc1nc(NCC(F)(F)F)cc2c1c(=O)n(-c1cnc3cc(F)ccn13)c(=O)n2-c1ccc(F)c(Cl)c1] | 3 | 2.18 | 553 |
| I-1042 | 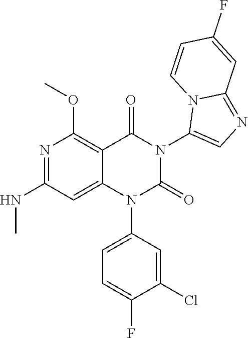 [SMILES: CNc1cc2c(c(OC)n1)c(=O)n(-c1cnc3cc(F)ccn13)c(=O)n2-c1ccc(F)c(Cl)c1] | 3 | 1.99 | 485 |
| I-1043 | 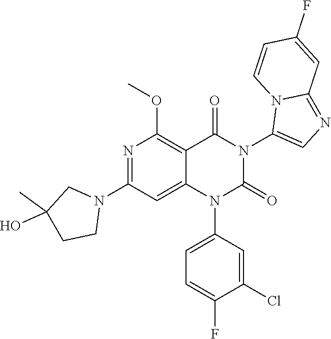 [SMILES: COc1nc(N2CCC(C)(O)C2)cc2c1c(=O)n(-c1cnc3cc(F)ccn13)c(=O)n2-c1ccc(F)c(Cl)c1] | 1 | 1.61 | 555 |

TABLE 85

| | | LC/MS | Retention | |
|---|---|---|---|---|
| Compound | | measurement | time | |
| No. | Structural formula | conditions | (min) | m/z |
| I-1044 | 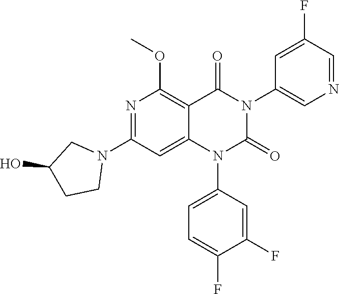 [SMILES: COc1nc(N2CC[C@@H](O)C2)cc2c1c(=O)n(-c1cncc(F)c1)c(=O)n2-c1ccc(F)c(F)c1] | 1 | 1.82 | 486 |
| I-1045 | 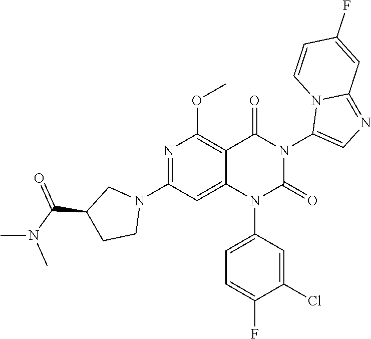 [SMILES: COc1nc(N2CC[C@@H](C(=O)N(C)C)C2)cc2c1c(=O)n(-c1cnc3cc(F)ccn13)c(=O)n2-c1ccc(F)c(Cl)c1] | 1 | 1.79 | 596.05 |
| I-1046 | 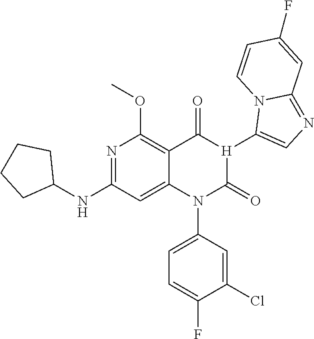 | 3 | 2.44 | 539.1 |
| I-1047 | 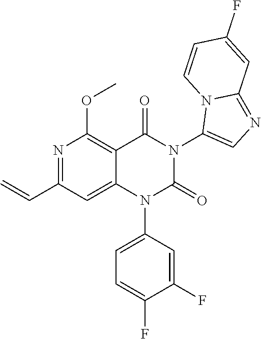 [SMILES: C=Cc1cc2c(c(OC)n1)c(=O)n(-c1cnc3cc(F)ccn13)c(=O)n2-c1ccc(F)c(F)c1] | 1 | 2.05 | 466 |
| I-1048 | 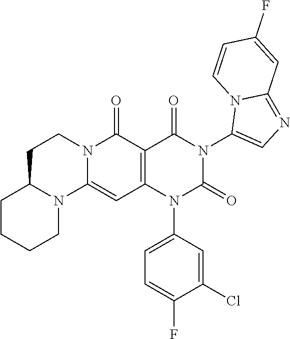 [SMILES: O=c1c2c(=O)n(-c3cnc4cc(F)ccn34)c(=O)n(-c3ccc(F)c(Cl)c3)c2cc2n1CC[C@@H]1CCCCN21] | 1 | 1.81 | 551 |
| I-1049 | 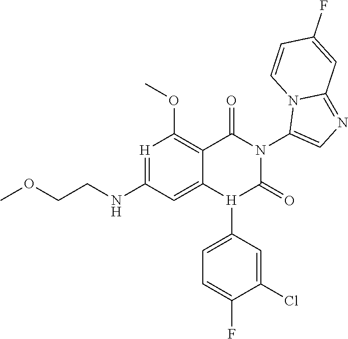 | 3 | 2.00 | 529 |

TABLE 86

| | | LC/MS | Retention | |
|---|---|---|---|---|
| Compound | | measurement | time | |
| No. | Structural formula | conditions | (min) | m/z |
| I-1050 | 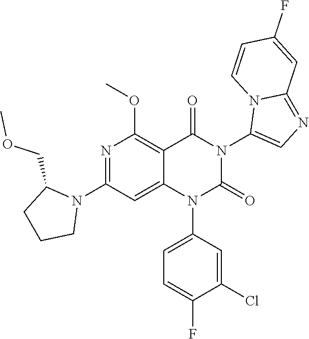 [SMILES: COC[C@H]1CCCN1c1cc2c(c(OC)n1)c(=O)n(-c1cnc3cc(F)ccn13)c(=O)n2-c1ccc(F)c(Cl)c1] | 1 | 2.19 | 569 |
| I-1051 | 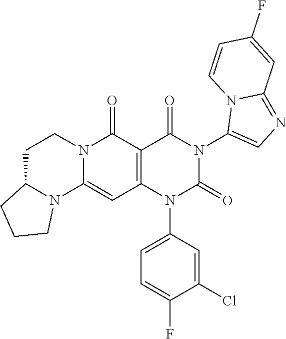 [SMILES: O=c1c2c(=O)n(-c3cnc4cc(F)ccn34)c(=O)n(-c3ccc(F)c(Cl)c3)c2cc2n1CC[C@H]1CCCN21] | 1 | 1.74 | 537.05 |
| I-1052 | 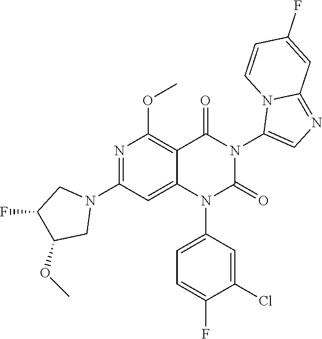 [SMILES: COc1nc(N2C[C@@H](F)[C@@H](OC)C2)cc2c1c(=O)n(-c1cnc3cc(F)ccn13)c(=O)n2-c1ccc(F)c(Cl)c1] | 1 | 1.89 | 573.1 |
| I-1053 | 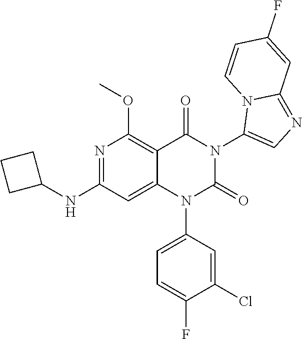 [SMILES: COc1nc(NC2CCC2)cc2c1c(=O)n(-c1cnc3cc(F)ccn13)c(=O)n2-c1ccc(F)c(Cl)c1] | 3 | 2.32 | 525 |
| I-1054 | 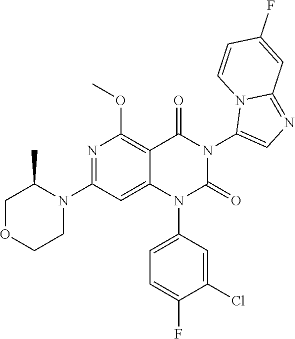 [SMILES: COc1nc(N2CCOC[C@H]2C)cc2c1c(=O)n(-c1cnc3cc(F)ccn13)c(=O)n2-c1ccc(F)c(Cl)c1] | 3 | 2.18 | 555 |
| I-1055 | 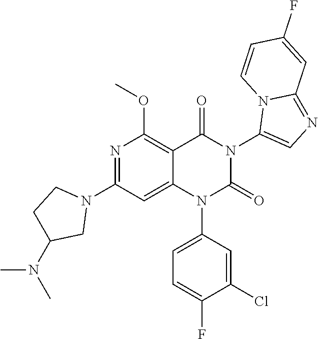 [SMILES: COc1nc(N2CCC(N(C)C)C2)cc2c1c(=O)n(-c1cnc3cc(F)ccn13)c(=O)n2-c1ccc(F)c(Cl)c1] | 1 | 1.37 | 568 |

TABLE 87

| | | LC/MS | Retention | |
|---|---|---|---|---|
| Compound | | measurement | time | |
| No. | Structural formula | conditions | (min) | m/z |
| I-1056 | 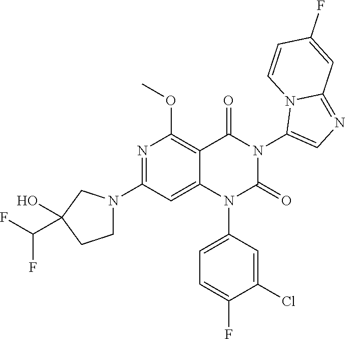 [SMILES: COc1nc(N2CCC(O)(C(F)F)C2)cc2c1c(=O)n(-c1cnc3cc(F)ccn13)c(=O)n2-c1ccc(F)c(Cl)c1] | 1 | 1.88 | 591 |
| I-1057 | 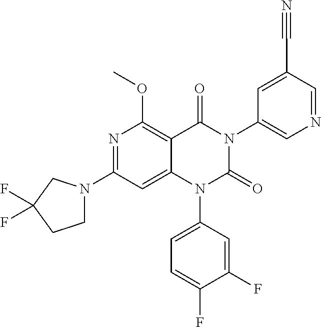 [SMILES: COc1nc(N2CCC(F)(F)C2)cc2c1c(=O)n(-c1cncc(C#N)c1)c(=O)n2-c1ccc(F)c(F)c1] | 1 | 2.29 | 513 |
| I-1058 | 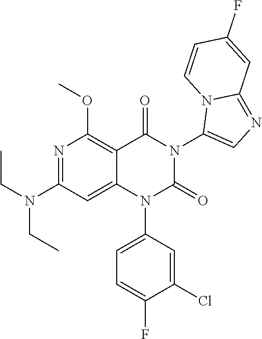 [SMILES: CCN(CC)c1cc2c(c(OC)n1)c(=O)n(-c1cnc3cc(F)ccn13)c(=O)n2-c1ccc(F)c(Cl)c1] | 3 | 2.39 | 527.1 |
| I-1059 | 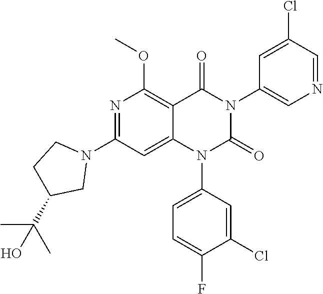 [SMILES: COc1nc(N2CC[C@@H](C(C)(C)O)C2)cc2c1c(=O)n(-c1cncc(Cl)c1)c(=O)n2-c1ccc(F)c(Cl)c1] | 1 | 2.31 | 560 |
| I-1060 | 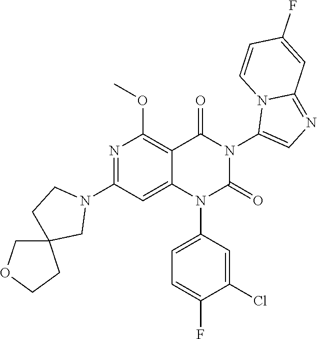 [SMILES: COc1nc(N2CCC3(CCOC3)C2)cc2c1c(=O)n(-c1cnc3cc(F)ccn13)c(=O)n2-c1ccc(F)c(Cl)c1] | 1 | 1.99 | 581 |
| I-1061 | 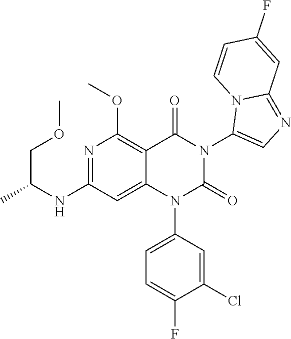 [SMILES: COC[C@@H](C)Nc1cc2c(c(OC)n1)c(=O)n(-c1cnc3cc(F)ccn13)c(=O)n2-c1ccc(F)c(Cl)c1] | 3 | 2.14 | 543.1 |

TABLE 88

| | | LC/MS | Retention | |
|---|---|---|---|---|
| Compound | | measurement | time | |
| No. | Structural formula | conditions | (min) | m/z |
| I-1062 | 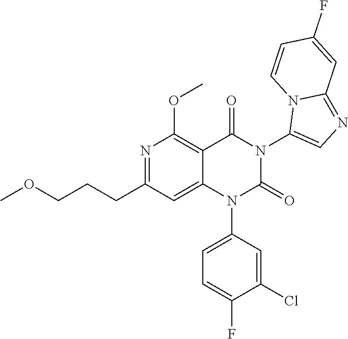 [SMILES: COCCCc1cc2c(c(OC)n1)c(=O)n(-c1cnc3cc(F)ccn13)c(=O)n2-c1ccc(F)c(Cl)c1] | 1 | 2.09 | 528.05 |
| I-1063 | 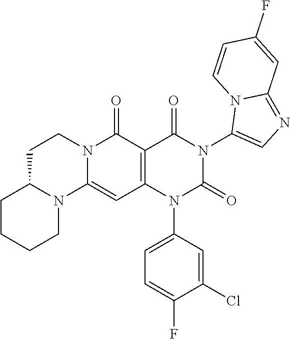 [SMILES: O=c1c2c(=O)n(-c3cnc4cc(F)ccn34)c(=O)n(-c3ccc(F)c(Cl)c3)c2cc2n1CC[C@H]1CCCCN21] | 1 | 1.83 | 551 |
| I-1064 | 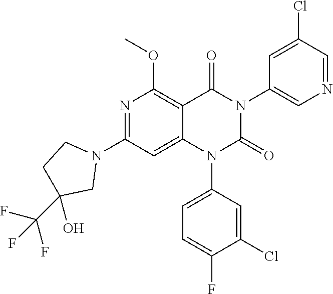 [SMILES: COc1nc(N2CCC(O)(C(F)(F)F)C2)cc2c1c(=O)n(-c1cncc(Cl)c1)c(=O)n2-c1ccc(F)c(Cl)c1] | 1 | 2.38 | 586 |

Incidentally, Compounds (I-0543), (I-0544) and (I-0545) shown below are reference examples.

TABLE 89

| | | LC/MS | Retention | |
|---|---|---|---|---|
| Compound | | measurement | time | |
| No. | Structural formula | conditions | (min) | m/z |
| I-0543 | 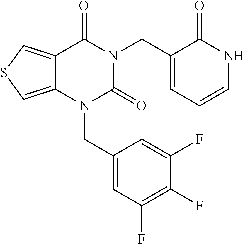 [SMILES: O=c1[nH]cccc1Cn1c(=O)c2cscc2n(Cc2cc(F)c(F)c(F)c2)c1=O] | 1 | 1.85 | 420 |
| I-0544 | 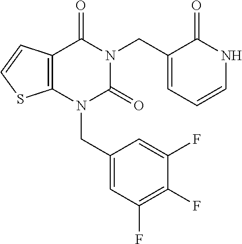 [SMILES: O=c1[nH]cccc1Cn1c(=O)c2ccsc2n(Cc2cc(F)c(F)c(F)c2)c1=O] | 1 | 1.83 | 420 |
| I-0545 | 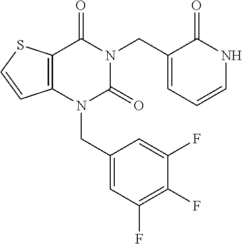 [SMILES: O=c1[nH]cccc1Cn1c(=O)c2sccc2n(Cc2cc(F)c(F)c(F)c2)c1=O] | 1 | 1.79 | 420 |

Biological Test Examples for the compounds of the present invention will be described below.

The compound represented by Formula (I) according to the present invention may have coronavirus 3CL protease inhibitory action and may inhibit coronavirus 3CL protease.

Specifically, in the evaluation method described below, the IC₅₀ is preferably 50 μM or less, more preferably 1 μM or less, and even more preferably 100 nM or less.

### Test Example 1-1: Inhibitory Activity of Cytopathic Effect (CPE) in Vero E6 Cells Expressing Human TMPRSS2 (Vero E6/TMPRSS2 Cells)

### <Operational Procedure>

### Dilution and Dispensing of Sample to be Tested

The sample to be tested is diluted in advance to an appropriate concentration with DMSO, and a 2- to 5-fold series of serial dilutions is prepared and then dispensed into a 384-well plate.

### Dilution and Dispensing of Cells and SARS-CoV-2

VeroE6/TMPRSS2 cells (JCRB1819, 5×10³ cells/well) and SARS-CoV-2 (100 TCID₅₀/well) are mixed in a medium (MEM, 2% FBS, penicillin-streptomycin), the mixture is dispensed into the wells in which the sample to be tested has been introduced, and then the cells are cultured for 3 days in a CO₂ incubator.

### Dispensing of CellTiter-Glo (Registered Trademark) 2.0 and Measurement of Luminescence Signals

The plate that has been cultured for 3 days is returned to room temperature, subsequently CellTiter-Glo (registered trademark) 2.0 is dispensed into each well, and the plate is mixed using a plate mixer. The plate is left to stand for a certain time, and then the luminescence signals (Lum) is measured with a plate reader.

### <Calculation of Each Measurement Item Value>

### Calculation of 50% SARS-CoV-2 Infected Cell Death Inhibitory Concentration (EC₅₀)

When x denotes the logarithmic value of the compound concentration and y denotes % Efficacy, the inhibition curve is approximated by the following Logistic regression equation, and the value of x when y=50(%) is inputted is calculated as EC₅₀.

$$y = min + ( max - min ) / \{ 1 + ( X 50 / x ) \hat{} Hill \}$$

$$\% Efficacy = \{ ( Sample - virus control ) / ( cell control - virus control ) \} ) * 100 \%$$

cell control: the average of Lum of cell control wells
virus control: the average of Lum of virus control wells
min: lower limit value of y-axis, max: upper limit value of y-axis, X50: x-coordinates of inflection point, Hill: slope of curve at midpoint between min and max

The compounds of the present invention were tested essentially as described above. The results are shown in the tables below.

Meanwhile, regarding the EC₅o value, a value of less than 1 μM is denoted by “A”, and a value of 1 μM or more and less than 10 μM is denoted by “B”.

### (Results)

TABLE 90

| Compound No. | EC50 [μM] |
|---|---|
| I-0003 | 0.692 |
| I-0004 | 1.16 |
| I-0007 | 1.14 |
| I-0028 | 10.1 |
| I-0029 | 49.8 |
| I-0033 | 0.217 |
| I-0035 | 0.363 |
| I-0048 | 0.537 |
| I-0053 | 0.497 |
| I-0058 | 0.194 |
| I-0059 | 0.335 |
| I-0064 | 2 |
| I-0066 | 11.1 |
| I-0072 | 2.43 |
| I-0074 | 1.83 |
| I-0076 | 0.489 |
| I-0116 | 1.75 |
| I-0139 | 3.62 |
| I-0160 | 0.670 |
| I-0163 | 0.273 |
| I-0164 | 0.080 |
| I-0167 | 0.528 |
| I-0263 | 0.736 |
| I-0264 | 0.349 |
| I-0295 | 0.426 |
| I-0315 | 0.411 |

TABLE 91

| Compound No. | EC50 |
|---|---|
| I-0010 | B |
| I-0011 | A |
| I-0013 | A |
| I-0015 | A |
| I-0027 | A |
| I-0031 | A |
| I-0034 | B |
| I-0036 | B |
| I-0047 | B |
| I-0049 | A |
| I-0051 | B |
| I-0052 | A |
| I-0054 | A |
| I-0055 | A |
| I-0063 | A |
| I-0065 | A |
| I-0067 | A |
| I-0073 | A |
| I-0166 | A |
| I-0168 | A |
| I-0170 | A |
| I-0190 | B |
| I-0194 | B |
| I-0195 | B |
| I-0200 | B |
| I-0208 | B |
| I-0266 | A |
| I-0273 | B |
| I-0280 | B |
| I-0284 | B |
| I-0285 | B |

### Test Example 1-2: Inhibitory Activity of Cytopathic Effect (CPE) in HEK293T Cells Expressing Human TMPRSS2, ACE2 (HEK293T/ACE2-TMPRSS2 Cells)

### <Operational Procedure>

### Dilution and Dispensing of Sample to be Tested

The sample to be tested is diluted in advance to an appropriate concentration with DMSO, and a 2- to 5-fold series of serial dilutions is prepared and then dispensed into a 384-well plate.

### Dilution and Dispensing of Cells and SARS-CoV-2

HEK293T/ACE2-TMPRSS2 cells (GCP-SL222, 5×10³ cells/well) and SARS-CoV-2 (200-300 TCID₅₀/well) are mixed in a medium (MEM, 2% FBS, penicillin-streptomycin), the mixture is dispensed into the wells in which the sample to be tested has been introduced, and then the cells are cultured for 3 days in a CO₂ incubator.

### Dispensing of CellTiter-Glo (Registered Trademark) 2.0 and Measurement of Luminescence Signals

The plate that has been cultured for 3 days is returned to room temperature, subsequently CellTiter-Glo (registered trademark) 2.0 is dispensed into each well, and the plate is mixed using a plate mixer. The plate is left to stand for a certain time, and then the luminescence signals (Lum) is measured with a plate reader.

### <Calculation of Each Measurement Item Value>

### Calculation of 50% SARS-CoV-2 Infected Cell Death Inhibitory Concentration (EC₅o)

When x denotes the logarithmic value of the compound concentration and y denotes % Efficacy, the inhibition curve is approximated by the following Logistic regression equation, and the value of x when y=50(%) is inputted is calculated as EC₅₀.

$$y = min + ( max - min ) / \{ 1 + ( X 50 / x ) \hat{} Hill \}$$

$$\% Efficacy = \{ ( Sample - virus control ) / ( cell control - virus control ) \} ) * 100 \%$$

cell control: the average of Lum of cell control wells
virus control: the average of Lum of virus control wells
min: lower limit value of y-axis, max: upper limit value of y-axis, X50: x-coordinates of inflection point, Hill: slope of curve at midpoint between min and max

The compounds of the present invention were tested essentially as described above. The results are shown in the tables below.

Meanwhile, regarding the EC₅o value, a value of less than 0.1 μM is denoted by “A”, a value of 0.1 μM or more and less than 1 μM is denoted by “B”, and a value of 1 μM or more and less than 10 μM is denoted by “C”.

TABLE 92

| Compound No. | EC50 [μM] |
|---|---|
| I-0554 | 2.75 |
| I-0556 | 0.376 |
| I-0570 | 0.0247 |
| I-0572 | 0.0283 |
| I-0579 | 0.0121 |
| I-0598 | 0.0136 |
| I-0626 | 0.0552 |
| I-0628 | 0.661 |
| I-0632 | 0.0501 |
| I-0642 | 0.64 |
| I-0643 | 0.52 |
| I-0647 | 13.9 |
| I-0650 | 0.0273 |
| I-0651 | 0.013 |
| I-0652 | 0.00743 |
| I-0653 | 0.0116 |
| I-0657 | 0.0761 |
| I-0671 | 0.021 |
| I-0683 | 0.0454 |
| I-0684 | 4.58 |
| I-0685 | 0.0822 |
| I-0686 | 0.0224 |
| I-0700 | 0.0245 |
| I-0701 | 0.016 |
| I-0727 | 0.102 |
| I-0728 | 0.00765 |
| I-0736 | 0.0201 |
| I-0739 | 0.115 |
| I-0741 | 0.105 |
| I-0743 | 0.0193 |
| I-0744 | 0.00694 |
| I-0746 | 0.0056 |
| I-0750 | 0.0259 |
| I-0753 | 0.0357 |
| I-0755 | 0.00706 |
| I-0802 | 0.0112 |
| I-0803 | 0.0176 |
| I-0806 | 0.0251 |
| I-0810 | 0.0194 |
| I-0812 | 0.0158 |
| I-0821 | 0.0376 |
| I-0823 | 0.197 |
| I-0842 | 0.0882 |
| I-0843 | 0.0723 |
| I-0858 | 0.0441 |
| I-0859 | 0.0187 |
| I-0863 | 0.0525 |
| I-0864 | 0.0462 |
| I-0866 | 0.0154 |
| I-0867 | 0.0479 |
| I-0868 | 0.0214 |
| I-0871 | 0.011 |
| I-0874 | 0.0392 |
| I-0878 | 0.0137 |
| I-0880 | 0.0178 |
| I-0883 | 0.0302 |
| I-0887 | 0.0982 |
| I-0891 | 0.0121 |
| I-0893 | 2.33 |
| I-0902 | 0.104 |
| I-0926 | 0.106 |
| I-0936 | 0.00534 |
| I-0937 | 0.00556 |
| I-0940 | 0.00566 |
| I-0947 | 0.00634 |
| I-0955 | 0.54 |
| I-0957 | 0.00814 |
| I-0961 | 0.00582 |
| I-0966 | 0.00901 |
| I-0978 | 0.0118 |
| I-0986 | 0.0071 |
| I-0990 | 0.00889 |
| I-1005 | 0.00865 |
| I-1019 | 0.57 |
| I-1021 | 0.0161 |
| I-1048 | 0.436 |

TABLE 93

| Compound No. | EC50 |
|---|---|
| I-0546 | A |
| I-0547 | A |
| I-0548 | A |
| I-0549 | B |
| I-0550 | A |
| I-0551 | A |
| I-0552 | B |
| I-0557 | B |
| I-0561 | A |
| I-0563 | A |
| I-0565 | A |
| I-0571 | A |
| I-0573 | A |
| I-0575 | A |
| I-0578 | A |
| I-0580 | A |
| I-0581 | A |
| I-0586 | A |
| I-0595 | A |
| I-0602 | A |
| I-0610 | A |
| I-0623 | A |
| I-0625 | B |
| I-0627 | A |
| I-0637 | A |
| I-0638 | A |
| I-0639 | B |
| I-0645 | B |
| I-0646 | A |
| I-0648 | A |
| I-0649 | A |
| I-0654 | A |
| I-0655 | A |
| I-0658 | B |
| I-0660 | A |
| I-0661 | A |
| I-0666 | B |
| I-0672 | A |
| I-0673 | A |
| I-0680 | A |
| I-0735 | A |
| I-0740 | A |
| I-0742 | A |
| I-0745 | A |
| I-0747 | A |
| I-0748 | A |
| I-0749 | A |
| I-0751 | A |
| I-0752 | A |
| I-0759 | A |
| I-0762 | A |
| I-0763 | A |
| I-0764 | A |
| I-0784 | B |
| I-0786 | B |
| I-0800 | A |
| I-0804 | A |
| I-0805 | A |
| I-0808 | A |
| I-0809 | A |
| I-0811 | A |
| I-0813 | A |
| I-0814 | A |
| I-0815 | A |
| I-0816 | A |
| I-0819 | A |
| I-0820 | A |
| I-0822 | A |
| I-0826 | A |
| I-0827 | B |
| I-0834 | A |
| I-0838 | B |
| I-0839 | B |
| I-0840 | A |
| I-0844 | A |
| I-0847 | B |
| I-0848 | B |
| I-0852 | B |
| I-0865 | B |
| I-0869 | A |
| I-0933 | B |
| I-0934 | A |
| I-0938 | A |
| I-0939 | A |
| I-0941 | A |
| I-0942 | A |
| I-0943 | A |
| I-0944 | A |
| I-0945 | A |
| I-0946 | A |
| I-0948 | A |
| I-0949 | A |
| I-0950 | A |
| I-0951 | A |
| I-0952 | A |
| I-0953 | A |
| I-0954 | A |
| I-0956 | A |
| I-0958 | A |
| I-0959 | A |
| I-0960 | A |
| I-0962 | A |
| I-0963 | A |
| I-0964 | A |
| I-0965 | A |
| I-0967 | A |
| I-0968 | A |
| I-0969 | A |
| I-0970 | A |
| I-0971 | A |
| I-0972 | A |
| I-0973 | A |
| I-0974 | A |
| I-0975 | A |
| I-0976 | A |
| I-0977 | A |
| I-0979 | A |
| I-0980 | A |
| I-0981 | A |
| I-0982 | A |
| I-1004 | A |
| I-1006 | A |
| I-1007 | B |
| I-1008 | A |
| I-1009 | A |
| I-1010 | A |
| I-1011 | A |
| I-1012 | A |
| I-1013 | A |
| I-1014 | A |
| I-1015 | A |
| I-1016 | A |
| I-1017 | A |
| I-1018 | A |
| I-1020 | A |
| I-1022 | A |
| I-1023 | A |
| I-1024 | A |
| I-1025 | A |
| I-1026 | A |
| I-1027 | A |
| I-1028 | A |
| I-1029 | A |
| I-1030 | A |
| I-1031 | B |
| I-1032 | A |
| I-1033 | A |
| I-1034 | A |
| I-1035 | A |
| I-1036 | A |
| I-1037 | A |
| I-1038 | A |
| I-1039 | A |
| I-1040 | A |
| I-1041 | A |
| I-1042 | A |
| I-1043 | A |
| I-1044 | A |
| I-1045 | A |
| I-1046 | A |

TABLE 94

| Compound No. | EC50 |
|---|---|
| I-0681 | A |
| I-0682 | A |
| I-0692 | A |
| I-0697 | C |
| I-0699 | A |
| I-0705 | A |
| I-0709 | C |
| I-0712 | B |
| I-0713 | B |
| I-0717 | A |
| I-0721 | A |
| I-0722 | A |
| I-0723 | A |
| I-0726 | A |
| I-0729 | A |
| I-0730 | A |
| I-0731 | A |
| I-0732 | A |
| I-0734 | A |
| I-0870 | A |
| I-0875 | A |
| I-0876 | A |
| I-0879 | A |
| I-0881 | A |
| I-0882 | A |
| I-0884 | A |
| I-0885 | A |
| I-0886 | A |
| I-0895 | B |
| I-0901 | A |
| I-0907 | B |
| I-0914 | A |
| I-0917 | A |
| I-0927 | A |
| I-0929 | A |
| I-0930 | B |
| I-0931 | B |
| I-0932 | A |
| I-0983 | A |
| I-0984 | A |
| I-0985 | A |
| I-0987 | A |
| I-0988 | B |
| I-0989 | A |
| I-0991 | A |
| I-0992 | A |
| I-0993 | A |
| I-0994 | A |
| I-0995 | A |
| I-0996 | A |
| I-0997 | A |
| I-0998 | A |
| I-0999 | B |
| I-1000 | A |
| I-1001 | A |
| I-1002 | A |
| I-1003 | A |
| I-1047 | B |
| I-1049 | A |
| I-1050 | A |
| I-1051 | B |
| I-1052 | A |
| I-1053 | A |
| I-1054 | A |
| I-1055 | A |
| I-1056 | A |
| I-1057 | A |
| I-1058 | A |
| I-1059 | A |
| I-1060 | A |
| I-1061 | A |
| I-1062 | A |
| I-1063 | B |
| I-1064 | A |

### Test Example 2: Inhibitory Activity Test Against SARS-CoV-2 3CL Proteases

### <Materials>

### Commercially Available Recombinant SARS-CoV-2 3CL Protease

### Commercially Available Substrate Peptide

Dabcyl-Lys-Thr-Ser-Ala-Val-Leu-Gln-Ser-Gly-Phe-Arg-Lys-Met-Glu(Edans)-NH2 (SEQ ID NO: 1)

### Internal Standard Peptide

Dabcyl-Lys-Thr-Ser-Ala-Val-Leu(13C6, 15N)-Gln (SEQ ID NO: 2)

Dabcyl-Lys-Thr-Ser-Ala-Val-Leu(13C6, 15N)-Gln can be synthesized with reference to documents (Atherton, E.; Sheppard, R. C., “In Solid Phase Peptide Synthesis, A Practical Approach”, IRL Press at Oxford University Pres, 1989.; Bioorg. Med. Chem., Vol. 5, No. 9, 1997, pp. 1883-1891; and the like). An example will be shown below.

H-Lys-Thr-Ser-Ala-Val-Leu(13C6, 15N)-Glu(resin)-OaOtBu (the Lys-side chain is Boc-protected, the Thr-side chain is protected with a tert-butyl group, the Ser-side chain is protected with a tert-butyl group, the C-terminal OH of Glu is protected with a tert-butyl group, and the carboxylic acid of the Glu-side chain is condensed into the resin) is synthesized by Fmoc solid-phase synthesis using a Rink amide resin. Regarding the modification of the N-terminal Dabcyl group, 4-dimethylaminoazobenzene-4′-carboxylic acid (Dabcyl-OH) is condensed on the resin using EDC/HOBT. Final deprotection and cleavage from the resin are carried out by treatment with TFA/EDT=95:5. Thereafter, purification is performed by reverse phase HPLC.

### RapidFire Cartridge C4 TypeA

### <Operational Procedure>

### Preparation of Assay Buffer

In the present test, an assay buffer composed of 20 mM Tris-HCl, 100 mM sodium chloride, 1 mM EDTA, 10 mM DTT, and 0.01% BSA is used. For a compound with an IC₅₀ of 10 nM or less, an assay buffer composed of 20 mM Tris-HCl, 1 mM EDTA, 10 mM DTT, and 0.01% BSA is used.

### Dilution and Dispensing of Sample to be Tested

The sample to be tested is diluted in advance to an appropriate concentration with DMSO, and a 2- to 5-fold series of serial dilutions is prepared and then dispensed into a 384-well plate.

### Addition of Enzyme and Substrate, and Enzymatic Reaction

To a prepared compound plate, 8 μM substrate, and a 6 or 0.6 nM enzyme solution are added, and incubation is carried out for 3 to 5 hours at room temperature. Thereafter, a reaction stopping solution (0.067 μM Internal Standard, 0.1% formic acid, and 10% or 25% acetonitrile) is added to stop the enzymatic reaction.

### Measurement of Reaction Product

The plate in which the reaction has been completed is measured using Rapid Fire System 360 and a mass analyzer (Agilent, 6550 iFunnel Q-TOF), or Rapid Fire System 365 and a mass analyzer (Agilent, 6495C Triple Quadrupole). Solution A (75% isopropanol, 15% acetonitrile, 5 mM ammonium formate) and solution B (0.01% trifluoroacetic acid, 0.09% formic acid) are used as a mobile phase at the measurement.

The reaction product detected by the mass analyzer is calculated using RapidFire Integrator or an equivalent program capable of analysis and is taken as Product area value. Furthermore, the Internal Standard that has been detected at the same time is also calculated and is designated as Internal Standard area value.

### <Calculation of Each Measurement Item Value>

### Calculation of P/IS

The area values obtained in the previous items are calculated by the following formula, and P/IS is calculated.

P/IS=Product area value/Internal Standard area value

### Calculation of 50% SARS-CoV-2 3CL Protease Inhibitory Concentration (IC₅₀)

When x denotes the logarithmic value of the compound concentration and y denotes % Inhibition, the inhibition curve is approximated by the following Logistic regression equation, and the value of x obtainable when y=50(%) is inputted is calculated as IC₅₀.

$$y = min + ( max - min ) / \{ 1 + ( X 50 / x ) \hat{} Hill \}$$

$$\% Inhibition = \{ 1 - ( Sample - Control ( - ) ) / Control ( + ) - Control ( - ) ) \} * 100$$

Control(−):the average of P/IS of enzyme inhibited condition wells
Control(+):the average of P/IS of DMSO control wells
min: lower limit value of y-axis, max: upper limit value of y-axis, X50: x-coordinates of inflection point, Hill: slope of curve at midpoint between min and max

The compounds of the present invention were tested essentially as described above. The results are shown in the tables below.

Incidentally, regarding the IC₅₀ value, a value of less than 0.1 μM is denoted by “A”, a value of 0.1 μM or more and less than 1 μM is denoted by “B”, and a value of 1 μM or more and less than 10 μM is denoted by “C”.

### (Results)

TABLE 95

| Compound No. | IC50 [μM] |
|---|---|
| I-0001 | 0.0053 |
| I-0002 | 0.0062 |
| I-0003 | 0.0086 |
| I-0004 | 0.0099 |
| I-0005 | 0.01 |
| I-0007 | 0.019 |
| I-0008 | 0.021 |
| I-0009 | 0.021 |
| I-0014 | 0.03 |
| I-0026 | 0.0065 |
| I-0028 | 0.0075 |
| I-0029 | 0.0078 |
| I-0030 | 0.0085 |
| I-0032 | 0.0094 |
| I-0033 | 0.013 |
| I-0035 | 0.025 |
| I-0048 | 0.011 |
| I-0053 | 0.03 |
| I-0058 | 0.0015 |
| I-0059 | 0.002 |
| I-0060 | 0.0027 |
| I-0061 | 0.0029 |
| I-0062 | 0.0031 |
| I-0064 | 0.0037 |
| I-0066 | 0.005 |
| I-0068 | 0.0058 |
| I-0069 | 0.006 |
| I-0070 | 0.0098 |
| I-0072 | 0.01 |
| I-0074 | 0.012 |
| I-0075 | 0.018 |
| I-0076 | 0.02 |
| I-0080 | 0.034 |
| I-0110 | 0.012 |
| I-0115 | 0.0093 |
| I-0116 | 0.012 |
| I-0119 | 0.023 |
| I-0122 | 0.027 |
| I-0125 | 0.032 |
| I-0139 | 0.0045 |
| I-0140 | 0.0013 |
| I-0142 | 0.025 |
| I-0163 | 0.0011 |
| I-0164 | 0.0012 |
| I-0165 | 0.0012 |
| I-0167 | 0.0019 |
| I-0169 | 0.0027 |
| I-0171 | 0.0032 |
| I-0174 | 0.016 |
| I-0176 | 0.033 |
| I-0177 | 0.034 |
| I-0187 | 0.005 |
| I-0188 | 0.0058 |
| I-0189 | 0.0071 |
| I-0193 | 0.009 |
| I-0196 | 0.011 |
| I-0199 | 0.016 |
| I-0202 | 0.02 |
| I-0207 | 0.023 |
| I-0210 | 0.028 |
| I-0213 | 0.033 |
| I-0248 | 0.0073 |
| I-0249 | 0.033 |
| I-0250 | 0.035 |
| I-0263 | 0.00099 |
| I-0264 | 0.0026 |
| I-0268 | 0.0075 |
| I-0274 | 0.015 |
| I-0275 | 0.019 |
| I-0276 | 0.024 |
| I-0277 | 0.024 |
| I-0278 | 0.026 |
| I-0283 | 0.032 |
| I-0295 | 0.015 |
| I-0301 | 0.098 |
| I-0315 | 0.35 |
| I-0325 | 0.45 |
| I-0353 | 0.00045 |
| I-0363 | 0.035 |
| I-0380 | 0.014 |
| I-0388 | 0.02 |
| I-0389 | 0.0019 |
| I-0395 | 0.0022 |
| I-0396 | 0.0013 |
| I-0437 | 0.0013 |
| I-0438 | 0.0035 |
| I-0439 | 0.0047 |
| I-0440 | 0.01 |
| I-0445 | 0.022 |
| I-0449 | 0.037 |
| I-0453 | 0.0025 |
| I-0460 | 0.0092 |
| I-0464 | 0.011 |
| I-0473 | 0.0041 |
| I-0501 | 0.0064 |
| I-0516 | 0.015 |
| I-0523 | 0.032 |
| I-0541 | 0.0034 |
| I-0554 | 0.33 |
| I-0556 | 0.00094 |
| I-0570 | 0.026 |
| I-0572 | 0.037 |
| I-0579 | 0.0058 |
| I-0598 | 0.018 |
| I-0626 | 0.014 |
| I-0628 | 0.026 |
| I-0632 | 0.0031 |
| I-0642 | 0.003 |
| I-0643 | 0.0043 |
| I-0647 | 0.0057 |
| I-0650 | 0.0012 |
| I-0651 | 0.0085 |
| I-0652 | 0.0014 |
| I-0653 | 0.014 |
| I-0657 | 0.02 |
| I-0671 | 0.0033 |
| I-0683 | 0.0099 |
| I-0684 | 0.37 |
| I-0685 | 0.028 |
| I-0686 | 0.0017 |
| I-0700 | 0.00092 |
| I-0701 | 0.005 |
| I-0727 | 0.0011 |
| I-0728 | 0.0051 |
| I-0736 | 0.012 |
| I-0739 | 0.00098 |
| I-0746 | 0.0014 |
| I-0750 | 0.0061 |
| I-0753 | 0.021 |
| I-0755 | 0.0056 |
| I-0802 | 0.024 |
| I-0803 | 0.032 |
| I-0806 | 0.0039 |
| I-0810 | 0.0013 |
| I-0812 | 0.0011 |
| I-0821 | 0.01 |
| I-0823 | 0.00075 |
| I-0842 | 0.034 |
| I-0843 | 0.0074 |
| I-0858 | 0.0024 |
| I-0859 | 0.0019 |
| I-0863 | 0.0053 |
| I-0864 | 0.0014 |
| I-0866 | 0.0064 |
| I-0867 | 0.0012 |
| I-0868 | 0.0016 |
| I-0871 | 0.0066 |
| I-0874 | 0.0094 |
| I-0878 | 0.0039 |
| I-0880 | 0.0044 |
| I-0883 | 0.0033 |
| I-0887 | 0.0098 |
| I-0891 | 0.01 |
| I-0893 | 0.036 |
| I-0902 | 0.0082 |
| I-0926 | 0.0051 |
| I-0936 | 0.0011 |
| I-0937 | 0.0013 |
| I-0940 | 0.0017 |
| I-0947 | 0.0023 |
| I-0955 | 0.0031 |
| I-0957 | 0.0032 |
| I-0961 | 0.0034 |
| I-0966 | 0.0038 |
| I-0978 | 0.0048 |
| I-0986 | 0.0065 |
| I-0990 | 0.0068 |
| I-1005 | 0.0089 |

TABLE 96

| Compound No. | IC50 [μM] |
|---|---|
| I-0160 | 0.00073 |
| I-0161 | 0.00075 |
| I-0162 | 0.0008 |
| I-0414 | 0.0015 |
| I-0431 | 0.011 |
| I-0435 | 0.001 |
| I-0741 | 0.0043 |
| I-0743 | 0.022 |
| I-0744 | 0.0014 |
| I-1019 | 0.011 |
| I-1021 | 0.012 |
| I-1048 | 0.023 |

TABLE 97

| Compound No. | IC50 |
|---|---|
| I-0006 | A |
| I-0010 | A |
| I-0011 | A |
| I-0012 | A |
| I-0013 | A |
| I-0015 | A |
| I-0016 | A |
| I-0027 | A |
| I-0031 | A |
| I-0034 | A |
| I-0036 | A |
| I-0047 | A |
| I-0049 | A |
| I-0050 | A |
| I-0051 | A |
| I-0052 | A |
| I-0054 | A |
| I-0055 | A |
| I-0063 | A |
| I-0065 | A |
| I-0067 | A |
| I-0071 | A |
| I-0073 | A |
| I-0077 | A |
| I-0078 | A |
| I-0079 | A |
| I-0117 | A |
| I-0118 | A |
| I-0120 | A |
| I-0121 | A |
| I-0123 | A |
| I-0124 | A |
| I-0136 | A |
| I-0141 | A |
| I-0166 | A |
| I-0168 | A |
| I-0170 | A |
| I-0172 | A |
| I-0173 | A |
| I-0175 | A |
| I-0443 | A |
| I-0446 | A |
| I-0451 | A |
| I-0459 | A |
| I-0466 | A |
| I-0472 | A |
| I-0484 | A |
| I-0492 | A |
| I-0504 | A |
| I-0506 | A |
| I-0511 | A |
| I-0514 | A |
| I-0517 | A |
| I-0540 | A |
| I-0542 | A |
| I-0546 | A |
| I-0547 | A |
| I-0548 | A |
| I-0549 | A |
| I-0550 | A |
| I-0551 | A |
| I-0552 | A |
| I-0553 | B |
| I-0557 | A |
| I-0561 | A |
| I-0563 | A |
| I-0565 | A |
| I-0571 | A |
| I-0573 | A |
| I-0575 | A |
| I-0578 | A |
| I-0580 | A |
| I-0581 | A |
| I-0586 | A |
| I-0595 | A |
| I-0602 | A |
| I-0610 | A |
| I-0623 | A |
| I-0625 | A |
| I-0627 | A |
| I-0752 | A |
| I-0759 | A |
| I-0762 | A |
| I-0763 | A |
| I-0764 | A |
| I-0784 | A |
| I-0786 | A |
| I-0800 | A |
| I-0804 | A |
| I-0805 | A |
| I-0808 | A |
| I-0809 | A |
| I-0811 | A |
| I-0813 | A |
| I-0814 | A |
| I-0815 | A |
| I-0816 | A |
| I-0819 | A |
| I-0820 | A |
| I-0822 | A |
| I-0826 | A |
| I-0827 | A |
| I-0834 | A |
| I-0838 | A |
| I-0839 | A |
| I-0840 | A |
| I-0844 | A |
| I-0847 | A |
| I-0848 | A |
| I-0852 | A |
| I-0865 | A |
| I-0869 | A |
| I-0870 | A |
| I-0875 | A |
| I-0876 | A |
| I-0879 | A |
| I-0881 | A |
| I-0882 | A |
| I-0884 | A |
| I-0885 | A |
| I-0974 | A |
| I-0975 | A |
| I-0976 | A |
| I-0977 | A |
| I-0979 | A |
| I-0980 | A |
| I-0981 | A |
| I-0982 | A |
| I-0983 | A |
| I-0984 | A |
| I-0985 | A |
| I-0987 | A |
| I-0988 | A |
| I-0989 | A |
| I-0991 | A |
| I-0992 | A |
| I-0993 | A |
| I-0994 | A |
| I-0995 | A |
| I-0996 | A |
| I-0997 | A |
| I-0998 | A |
| I-0999 | A |
| I-1000 | A |
| I-1001 | A |
| I-1002 | A |
| I-1003 | A |
| I-1004 | A |
| I-1006 | A |
| I-1007 | A |
| I-1008 | A |
| I-1009 | A |
| I-1010 | A |
| I-1011 | A |
| I-1012 | A |
| I-1013 | A |
| I-1014 | A |
| I-1015 | A |
| I-1016 | A |
| I-1017 | A |

TABLE 98

| Compound No. | IC50 |
|---|---|
| I-0178 | A |
| I-0190 | A |
| I-0191 | A |
| I-0192 | A |
| I-0194 | A |
| I-0195 | A |
| I-0197 | A |
| I-0198 | A |
| I-0200 | A |
| I-0201 | A |
| I-0203 | A |
| I-0204 | A |
| I-0205 | A |
| I-0206 | A |
| I-0208 | A |
| I-0209 | A |
| I-0211 | A |
| I-0212 | A |
| I-0214 | A |
| I-0215 | A |
| I-0216 | A |
| I-0217 | A |
| I-0265 | A |
| I-0266 | A |
| I-0267 | A |
| I-0269 | A |
| I-0270 | A |
| I-0271 | A |
| I-0272 | A |
| I-0273 | A |
| I-0279 | A |
| I-0280 | A |
| I-0281 | A |
| I-0282 | A |
| I-0284 | A |
| I-0285 | A |
| I-0296 | A |
| I-0378 | A |
| I-0379 | A |
| I-0381 | A |
| I-0637 | A |
| I-0638 | A |
| I-0639 | A |
| I-0644 | A |
| I-0645 | B |
| I-0646 | A |
| I-0648 | A |
| I-0649 | A |
| I-0654 | A |
| I-0655 | A |
| I-0658 | B |
| I-0660 | A |
| I-0661 | A |
| I-0666 | A |
| I-0672 | A |
| I-0673 | A |
| I-0680 | A |
| I-0681 | A |
| I-0682 | A |
| I-0692 | A |
| I-0697 | B |
| I-0699 | A |
| I-0705 | A |
| I-0709 | B |
| I-0712 | B |
| I-0713 | A |
| I-0717 | A |
| I-0721 | A |
| I-0722 | A |
| I-0723 | A |
| I-0726 | A |
| I-0729 | A |
| I-0730 | A |
| I-0731 | A |
| I-0732 | A |
| I-0734 | A |
| I-0735 | A |
| I-0740 | A |
| I-0742 | A |
| I-0745 | A |
| I-0886 | A |
| I-0895 | A |
| I-0901 | A |
| I-0907 | A |
| I-0914 | A |
| I-0917 | A |
| I-0927 | A |
| I-0929 | A |
| I-0930 | A |
| I-0931 | A |
| I-0932 | A |
| I-0933 | A |
| I-0934 | A |
| I-0935 | C |
| I-0938 | A |
| I-0939 | A |
| I-0941 | A |
| I-0942 | A |
| I-0943 | A |
| I-0944 | A |
| I-0945 | A |
| I-0946 | A |
| I-0948 | A |
| I-0949 | A |
| I-0950 | A |
| I-0951 | A |
| I-0952 | A |
| I-0953 | A |
| I-0954 | A |
| I-0956 | A |
| I-0958 | A |
| I-0959 | A |
| I-0960 | A |
| I-0962 | A |
| I-0963 | A |
| I-0964 | A |
| I-0965 | A |
| I-0967 | A |
| I-0968 | A |
| I-0969 | A |
| I-1018 | A |
| I-1020 | A |
| I-1022 | A |
| I-1023 | A |
| I-1024 | A |
| I-1025 | A |
| I-1026 | A |
| I-1027 | A |
| I-1028 | A |
| I-1029 | A |
| I-1030 | A |
| I-1031 | A |
| I-1032 | A |
| I-1033 | A |
| I-1034 | A |
| I-1035 | A |
| I-1036 | A |
| I-1037 | A |
| I-1038 | A |
| I-1039 | A |
| I-1040 | A |
| I-1041 | A |
| I-1042 | A |
| I-1043 | A |
| I-1044 | A |
| I-1045 | A |
| I-1046 | A |
| I-1047 | A |
| I-1049 | A |
| I-1050 | A |
| I-1051 | A |
| I-1052 | A |
| I-1053 | A |
| I-1054 | A |
| I-1055 | A |
| I-1056 | A |
| I-1057 | A |
| I-1058 | A |
| I-1059 | A |
| I-1060 | A |

TABLE 99

| Compound No. | IC50 |
|---|---|
| I-0393 | A |
| I-0415 | A |
| I-0429 | A |
| I-0442 | A |
| I-0747 | A |
| I-0748 | A |
| I-0749 | A |
| I-0751 | A |
| I-0970 | A |
| I-0971 | A |
| I-0972 | A |
| I-0973 | A |
| I-1061 | A |
| I-1062 | A |
| I-1063 | A |
| I-1064 | A |

### Test Example 3: CYP Inhibition Test

The degrees at which the amounts of respective metabolites produced are inhibited by the compound of the present invention are evaluated in commercially available pooled human liver microsomes by using the O-deethylation of 7-ethoxyresorufin (CYP1A2), the methyl-hydroxylation of tolbutamide (CYP2C9), 4′-hydroxylation of mephenytoin (CYP2C19), the O-demethylation of dextromethorphan (CYP2D6), and the hydroxylation of terfenadine (CYP3A4), which are the typical substrate metabolism reactions of five human major CYP5 molecular species (CYP1A2, CYP2C9, CYP2C19, CYP2D6, and CYP3A4), as indexes.

The reaction conditions were as follows: substrate, 0.5 μmol/L ethoxyresorufin (CYP1A2), 100 μmol/L tolbutamide (CYP2C9), 50 μmol/L S-mephenytoin (CYP2C19), 5 μmol/L dextromethorphan (CYP2D6), and 1 μmol/L terfenadine (CYP3A4); reaction time, 15 minutes; reaction temperature, 37° C.; enzyme, pooled human liver microsomes, 0.2 mg protein/mL; concentrations of the compound of the present invention, 1, 5, 10, and 20 μmol/L (4 points).

Each of the five substrates, the human liver microsomes, and the compound of the present invention are added according to the recipe described above into a 50 mmol/L HEPES buffer solution in a 96-well plate, and a coenzyme NADPH is added thereto to start the metabolism reactions serving as indexes. After reaction at 37° C. for 15 minutes, the reaction is terminated by the addition of a solution of methanol/acetonitrile=1/1 (V/V). After the centrifugation at 3000 rpm for 15 minutes, resorufin (CYP1A2 metabolite) in the supernatant is quantified by a fluorescent multilabel counter or LC/MS/MS and hydroxytolbutamide (CYP2C9 metabolite), 4′ hydroxymephenytoin (CYP2C19 metabolite), dextromethorphan (CYP2D6 metabolite), and terfenadine alcohol metabolite (CYP3A4 metabolite) are quantified by LC/MS/MS. The dilution concentration or the dilution solvent are changed as necessary.

Only a solvent DMSO used for dissolving the compound is added to the reaction solution instead of the compound of the present invention, and the mixture is used as a control (100%). Remaining activity (%) is calculated, and IC₅₀ is calculated by inverse estimation based on a logistic model using the concentrations and the rates of suppression.

The compounds of the present invention were tested essentially as described above.

### Test Example 4: CYP3A4 (MDZ) MBI Test

This test as to the inhibition of CYP3A4 by the compound of the present invention is to evaluate mechanism based inhibition (MBI) ability from enhancement in inhibitory effect, caused by a metabolism reaction, of the compound of the present invention. The inhibition of CYP3A4 is evaluated in pooled human liver microsomes by using the 1-hydroxylation reaction of midazolam (MDZ) as an index.

Reaction conditions are as follows: substrate, 10 μmol/L MDZ; prereaction time, 0 or 30 minutes; substrate metabolism reaction time, 2 minutes; reaction temperature, 37° C.; pooled human liver microsomes, 0.5 mg/mL for the prereaction, and 0.05 mg/mL (10-fold dilution) for the reaction; concentrations of the compound of the present invention for the prereaction, 1, 5, 10, and 20 μmol/L (4 points) or 0.83, 5, 10, and 20 μmol/L (4 points).

The pooled human liver microsomes and a solution of the compound of the present invention are added according to the recipe of the prereaction described above into a K-Pi buffer solution (pH 7.4) as a pre-reaction solution in a 96-well plate. A portion thereof is transferred to another 96-well plate so as to be diluted by 1/10 with a K-Pi buffer solution containing the substrate. A coenzyme NADPH is added thereto to start the reaction serving as an index (0-min preincubation). After reaction for a given time, the reaction is terminated by the addition of a solution of methanol/acetonitrile=1/1 (V/V). NADPH is also added to the remaining pre-reaction solution to start prereaction (30-min preincubation). After prereaction for a given time, a portion thereof is transferred to another plate so as to be diluted by 1/10 with a K-Pi buffer solution containing the substrate, to start the reaction serving as an index. After reaction for a given time, the reaction is terminated by the addition of a solution of methanol/acetonitrile=1/1 (V/V). Each plate where the index reaction has been performed is centrifuged at 3000 rpm for 15 minutes. Then, midazolam 1-hydroxide in the centrifugation supernatants is quantified by LC/MS/MS. The dilution concentration or the dilution solvent are changed as necessary.

Only a solvent DMSO used for dissolving the compound is added to the reaction solution instead of the compound of the present invention, and the mixture is used as a control (100%). Remaining activity (%) at the time of the addition of the compound of the present invention at each concentration is calculated, and IC is calculated by inverse estimation based on a logistic model using the concentrations and the rates of suppression. Shifted IC value is calculated as “IC of preincubation at 0 min/IC of preincubation at 30 min”. When a shifted IC is 1.5 or more, this is defined as positive. When a shifted IC is 1.0 or less, this is defined as negative.

The compounds of the present invention were tested essentially as described above.

### Test Example 5: BA Test

Materials and methods for experiments to evaluate oral absorption
(1) Animals used: SD rats are used.
(2) Rearing conditions: the SD rats are allowed to freely take solid feed and sterilized tap water.
(3) Dose and grouping setting: a given dose is orally administered and intravenously administered. Groups are set as follows: (Dosage can be changed per compound)
Oral administration 1 to 60 μmol/kg or 0.5 to 30 mg/kg (n=2 to 3)
Intravenous administration 0.5 to 20 μmol/kg or 0.1 to 10 mg/kg (n=2 to 3)
(4) Preparation of dosing solution: the test sample is administered as a solution or a suspension for the oral administration. The test sample is solubilized and administered for the intravenous administration.
(5) Administration method: the test sample is forcedly administered into the stomach through an oral probe for the oral administration. The test sample is administered to the tail vein through a syringe with an injection needle for the intravenous administration.
(6) Evaluation item: blood is collected over time, and the concentration of the compound of the present invention in plasma is measured using LC/MS/MS.
(7) Statistical analysis: an area under concentration in plasma-time curve (AUC) is calculated as to change in the concentration of the compound of the present invention in plasma by the moment analysis method, and the bioavailability (BA) of the compound of the present invention is calculated from the dose ratio and AUC ratio between the oral administration group and the intravenous administration group.

The dilution concentration or the dilution solvent are changed as necessary.

The compounds of the present invention were tested essentially as described above.

### Test Example 6: Clearance Evaluation Test

### Materials and Methods for Experiments

(1) Animals used: SD rats are used.
(2) Rearing conditions: the SD rats are allowed to freely take solid feed and sterilized tap water.
(3) Dose and grouping setting: a given dose is intravenously administered. Groups are set as follows: Intravenous administration 0.5 or 1 μmol/kg (n=2)
(4) Preparation of dosing solution: the test sample is solubilized using a solvent of dimethyl sulfoxide/propylene glycol=1/1 and administered.
(5) Administration method: the test sample is administered to the tail vein through a syringe with an injection needle.
(6) Evaluation item: blood is collected over time, and the concentration of the compound of the present invention in plasma is measured using LC/MS/MS.
(7) Statistical analysis: about transition of concentration of a compound of the present invention in plasma, total clearance (CLtot) of a compound according to the present invention is calculated by the moment analysis method. The dilution concentration or the dilution solvent are changed as necessary.

The compounds of the present invention were tested essentially as described above.

### Test Example 7: Metabolism Stability Test

Using pooled human liver microsomes or pooled rat liver microsomes, a compound of the present invention is reacted for a constant time, and a remaining rate is calculated by comparing a reacted sample and an unreacted sample, thereby, a degree of metabolism in liver is assessed.

A reaction is performed (oxidative reaction) at 37° C. for 0 minute or 30 minutes in the presence of 1 mmol/L NADPH in 0.2 mL of a buffer (50 mmol/L Tris-HCl pH 7.4, 150 mmol/L potassium chloride, 10 mmol/L magnesium chloride) containing 0.5 mg protein/mL of human or rat liver microsomes. After the reaction, 50 μL of the reaction solution is added to 100 μL of a methanol/acetonitrile=1/1 (v/v), mixed and centrifuged at 3000 rpm for 15 minutes. The compound of the present invention in the centrifuged supernatant is quantified by LC/MS/MS or solid-phase extraction (SPE)/MS. The ratio of the amount of the compound after the reaction, with respect to the amount of the compound at 0 minutes of the reaction defined as 100%, is shown as the residual rate. Hydrolysis reaction is performed in the absence of NADPH, and glucuronidation reaction is performed in the presence of 5 mmol/L UDP-glucuronic acid instead of NADPH. Then, the same operation is carried out. The dilution concentration or the dilution solvent are changed as necessary.

The compounds of the present invention were tested essentially as described above.

### Test Example 8: Solubility Test

The solubility of the compound of the present invention is determined under 1% DMSO addition conditions. 10 mmol/L solution of the compound is prepared with DMSO. 2 μL of the solution of the compound of the present invention is added to 198 μL each of a JP-1 fluid and a JP-2 fluid. The mixture is shaken for 1 hour or more at a room temperature, and the mixture is filtered. The filtrate is ten-fold diluted with methanol/water=1/1 (V/V), and the compound concentration in the filtrate is measured with LC/MS/MS or Solid-Phase Extraction (SPE)/MS by the absolute calibration method. The dilution concentration or the dilution solvent are changed as necessary.

The recipe of the JP-1 fluid is as follows.

Water is added to 2.0 g of sodium chloride and 7.0 mL of hydrochloric acid to bring the amount to 1000 mL.

The recipe of the JP-2 fluid is as follows.

3.40 g of potassium dihydrogen phosphate and 3.55 g of dibasic sodium phosphate anhydrous are dissolved in water to bring the amount to 1000 mL, and to 1 volume of the resultant, 1 volume of water is added.

The compounds of the present invention were tested essentially as described above.

The preparation examples shown below are only for illustrative purposes and are by no means intended to limit the scope of the invention.

The compound of the present invention can be administered as a pharmaceutical composition by any conventional route, particularly enterally, for example, orally, for example, in the form of a tablet or a capsule; parenterally, for example, in the form of an injectable preparation or a suspension; and topically, for example, in the form of a lotion, a gel, an ointment or a cream, or as a pharmaceutical composition in a transnasal form or a suppository form. A pharmaceutical composition comprising the compound of the present invention in a free form or in the form of a pharmaceutically acceptable salt together with at least one pharmaceutically acceptable carrier or diluent can be produced by a mixing, granulating, or coating method in a conventional manner. For example, the oral composition can be a tablet, a granular preparation, or a capsule, each containing an excipient, a disintegrating agent, a binder, a lubricating agent, and the like, as well as an active ingredient and the like. Furthermore, the composition for injection can be prepared as a solution or a suspension, may be sterilized, and may contain a preservative, a stabilizer, a buffering agent, and the like.

### INDUSTRIAL APPLICABILITY

The compound according to the present invention has coronavirus 3CL protease inhibitory activity, and it is considered that the compound is useful as a therapeutic agent and/or a prophylactic agent for a disease or a condition associated with coronavirus 3CL proteases.

### SEQUENCE LISTING

1. A compound represented by Formula (I): 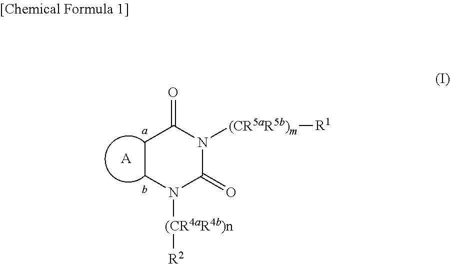
wherein
carbon atom a and carbon atom b are each a carbon atom constituting ring A, the ring A is a ring represented by: 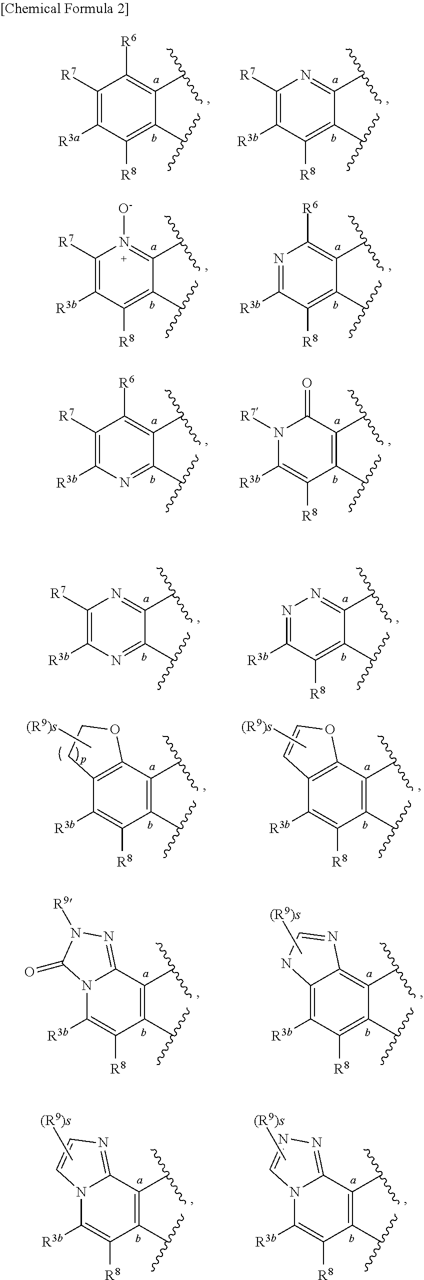 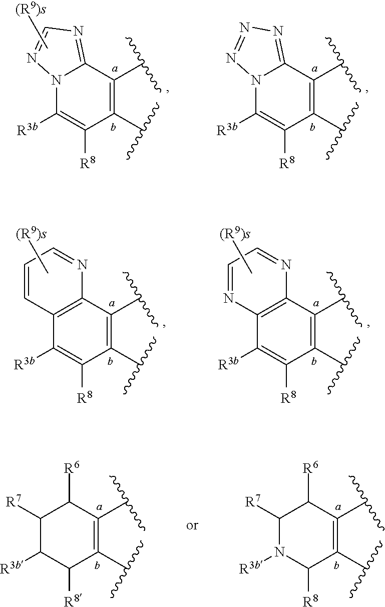
wherein
carbon atom a represents the carbon atom a in Formula (I);
carbon atom b represents the carbon atom b in Formula (I);
R^{3a} is a hydrogen atom, substituted or unsubstituted aromatic carbocyclyl, substituted or unsubstituted aromatic heterocyclyl, substituted or unsubstituted non-aromatic heterocyclyl, substituted or unsubstituted alkyloxy, substituted amino, halogen, substituted or unsubstituted carbamoyl, substituted or unsubstituted sulfamoyl, substituted or unsubstituted non-aromatic carbocyclyloxy, or substituted or unsubstituted non-aromatic heterocyclyloxy;
R^{3b} and R^{3b′} are each independently a hydrogen atom, substituted or unsubstituted aromatic carbocyclyl, substituted or unsubstituted aromatic heterocyclyl, substituted or unsubstituted non-aromatic heterocyclyl, substituted or unsubstituted alkyloxy, substituted amino, halogen, cyano, substituted or unsubstituted alkyl, substituted or unsubstituted alkenyl, substituted or unsubstituted carbamoyl, substituted or unsubstituted sulfamoyl, substituted or unsubstituted non-aromatic carbocyclyloxy, or substituted or unsubstituted non-aromatic heterocyclyloxy;
R⁸ and R^{8′} are each independently a hydrogen atom, halogen, substituted or unsubstituted alkyl, or substituted or unsubstituted alkyloxy; or
R^{3a} and R⁸, and R^{3b} and R^{8′} may be taken together with a carbon atom to which they are each bonded to form a substituted or unsubstituted aromatic carbocycle or a substituted or unsubstituted aromatic heterocycle;
R⁶ is a hydrogen atom, halogen, substituted or unsubstituted alkyloxy, hydroxy, or cyano;
R⁷ is a hydrogen atom, halogen, substituted or unsubstituted alkyl, substituted or unsubstituted alkenyl, substituted or unsubstituted alkynyl, substituted or unsubstituted alkyloxy, substituted or unsubstituted non-aromatic carbocyclyl, substituted or unsubstituted non-aromatic heterocyclyl, substituted or unsubstituted non-aromatic carbocyclyloxy, substituted or unsubstituted amino, substituted or unsubstituted alkylsulfoxy, substituted or unsubstituted carbamoyl, hydroxy, carboxy, formyl, or cyano;
R^{7′} and R^{9′} are each independently a hydrogen atom, or substituted or unsubstituted alkyl;
R^{3b} and R^{7′} may be taken together with an atom to which they are each bonded to form a substituted or unsubstituted non-aromatic heterocycle;
R⁹s are each independently halogen, hydroxy, substituted or unsubstituted alkyl, or substituted or unsubstituted alkyloxy;
s is 0, 1, or 2;
p is 1, 2, or 3;
R¹ is substituted or unsubstituted nitrogen-containing aromatic heterocyclyl, substituted or unsubstituted nitrogen-containing non-aromatic heterocyclyl, or substituted or unsubstituted carbamoyl;
R² is substituted or unsubstituted 6-membered aromatic carbocyclyl or substituted or unsubstituted 6-membered aromatic heterocyclyl;
n is 0 or 1;
R^{4a} is a hydrogen atom, or substituted or unsubstituted alkyl;
R^{4b} is a hydrogen atom or substituted or unsubstituted alkyl; or R^{4a} and R^{4b} may be taken together with a carbon atom to which they are each bonded to form a substituted or unsubstituted non-aromatic carbocycle, or a substituted or unsubstituted non-aromatic heterocycle;
m is 0, 1, or 2;
R^{5a}s are each independently a hydrogen atom or substituted or unsubstituted alkyl;
R^{5b}s are each independently a hydrogen atom or substituted or unsubstituted alkyl,
provided that the compound in which R¹ is substituted or unsubstituted carbamoyl, and R^{3a} and R^{3b} are substituted or unsubstituted oxazolyl or substituted or unsubstituted thiazolyl are excluded,
or a pharmaceutically acceptable salt thereof.

2. The compound according to claim 1,
wherein the ring A is a ring represented by: 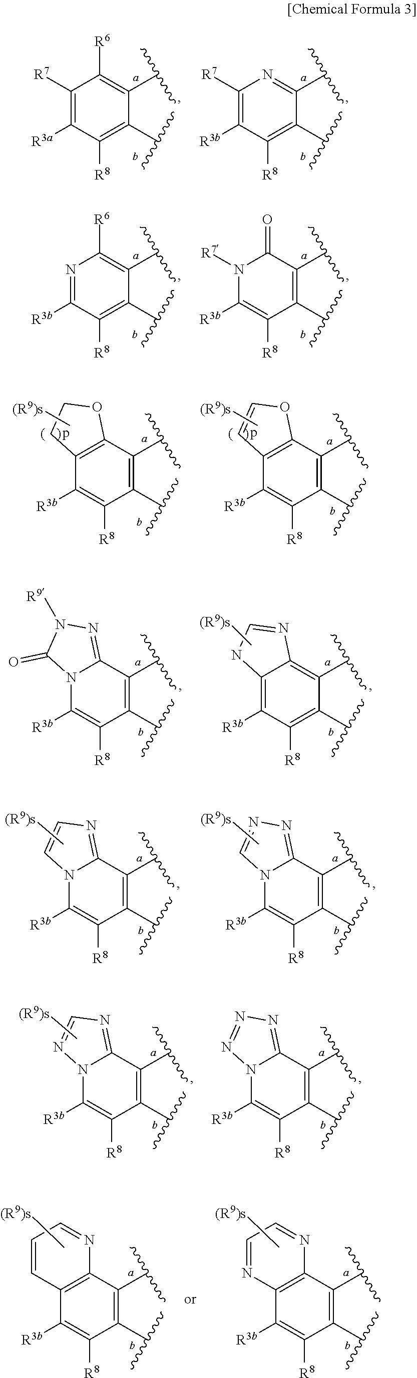
wherein carbon atom a, carbon atom b, R^{3a}, R^{3b}, R⁶, R⁷, R^{7′}, R⁸, R⁹, R⁹, s, and p have the same meanings as those in claim 1,
or a pharmaceutically acceptable salt thereof.

3. The compound according to claim 1,
wherein R^{3a} and R^{3b} are each independently a group represented by: 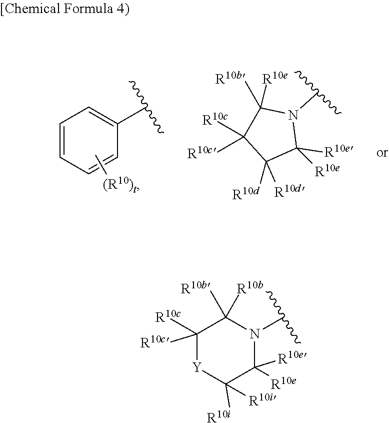
wherein Y is CR^{10h}R^{10h′} or O;
t is an integer of 0 to 5;
R^{10a}s are each independently halogen, cyano, carboxy, substituted or unsubstituted alkyl, substituted or unsubstituted alkyloxy, substituted or unsubstituted alkylcarbonyl, substituted or unsubstituted alkyloxycarbonyl, substituted or unsubstituted alkylsulfonyl, substituted or unsubstituted sulfamoyl, substituted or unsubstituted carbamoyl, substituted or unsubstituted amino, substituted or unsubstituted aromatic heterocyclyl, substituted or unsubstituted non-aromatic heterocyclyl, substituted or unsubstituted non-aromatic heterocyclylcarbonyl, substituted or unsubstituted sulfoxyimino, or substituted or unsubstituted iminosulfino; or
two R^{10a}s bonded to an adjacent carbon atom may be taken together to form a substituted or unsubstituted aromatic heterocycle, or a substituted or unsubstituted non-aromatic heterocycle;
R^{10b}, R^{10b′}, R^{10c}, R^{10c′}, R^{10d}, R^{10d′}, R^{10e}, R^{10e′}, R^{10f}, R^{10f′}, R^{10g}, R^{10g′}, R^{10h}, R^{10h′}, R¹⁰ⁱ, R^{10i′}, R^{10j}, and R^{10j′} are each independently a hydrogen atom, hydroxy, halogen, substituted or unsubstituted alkyl, substituted or unsubstituted amino, substituted or unsubstituted carbamoyl, substituted or unsubstituted alkyloxy, substituted or unsubstituted alkyloxycarbonyl, or substituted or unsubstituted aromatic heterocyclyl;
R^{10b} and R^{10b′}, R^{10c} and R^{10c′}, R^{10d} and R^{10d′}, R^{10e} and R^{10e′}, R^{10f} and R^{10f′}, R^{10g} and R^{10g′}, R^{10h} and R^{10h′}, R¹⁰ⁱ and R^{10i′}, and R^{10j} and R^{10j′} may be each independently taken together with a carbon atom to which they are bonded to form oxo, a substituted or unsubstituted non-aromatic carbocycle, or a substituted or unsubstituted non-aromatic heterocycle;
R^{10b} and R^{10c}, R^{10c} and R^{10d}, R^{10d} and R^{10e}, R^{10f} and R^{10g}, R^{10g} and R^{10h}, R^{10h} and R¹⁰ⁱ, and R¹⁰ⁱ and R^{10j} may be each independently taken together with a carbon atom to which they are each bonded to form a substituted or unsubstituted non-aromatic heterocycle, a substituted or unsubstituted aromatic carbocycle, or a substituted or unsubstituted non-aromatic carbocycle;
R^{10b} and R^{10d}, R^{10b} and R^{10e}, R^{10c} and R^{10e}, R^{10f} and R^{10h}, R^{10f} and R¹⁰ⁱ, R^{10f} and R^{10j}, R^{10g} and R¹⁰ⁱ, and R^{10g} and R^{10j} may each independently together each form a (C1-C4) bridge, in which one of the carbon atoms that constitute the bridge may be replaced by an oxygen atom or a nitrogen atom; the carbon atoms constituting the bridge are each independently substituted with a substituent selected from R^{a}, and the nitrogen atom constituting the bridge is substituted with a substituent selected from R^{b};
wherein R^{a} is a hydrogen atom, halogen, substituted or unsubstituted alkyl, or substituted or unsubstituted alkyloxy; and
R^{b} is a hydrogen atom, or substituted or unsubstituted alkyl, or
R^{3a} and R^{3b} are each independently substituted amino,
or a pharmaceutically acceptable salt thereof.

4. The compound according to claim 1,
wherein R² is 6-membered aromatic carbocyclyl which is substituted with one halogen and is further substituted with one, two, three, or four substituents selected from substituent group G, or 6-membered aromatic heterocyclyl which is substituted with one halogen and is further substituted with one or two substituents selected from substituent group G,
wherein the substituent group G is the group consisting of halogen, cyano, alkyl, alkenyl, alkynyl, haloalkyl, alkyloxy, alkenyloxy, alkynyloxy and haloalkyloxy,
or a pharmaceutically acceptable salt thereof.

5. The compound according to claim 1,
wherein R² is 6-membered aromatic carbocyclyl which is substituted with one halogen and is further substituted with one, two, three, or four substituents selected from the substituent group G,
wherein the substituent group G is the group consisting of halogen, cyano, alkyl, alkenyl, alkynyl, haloalkyl, alkyloxy, alkenyloxy, alkynyloxy and haloalkyloxy,
or a pharmaceutically acceptable salt thereof.

6. The compound according to claim 1,
wherein R¹ is substituted or unsubstituted nitrogen-containing aromatic heterocyclyl or substituted or unsubstituted nitrogen-containing non-aromatic heterocyclyl,
or a pharmaceutically acceptable salt thereof.

7. The compound according to claim 1,
wherein the ring A is a ring represented by: 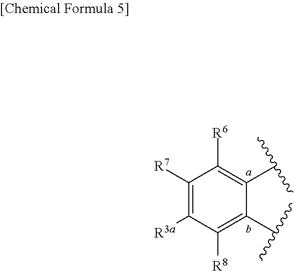
wherein carbon atom a, carbon atom b, R^{3a}, R⁶, and R⁸ have the same meanings as those in claim 1, and
R⁷ is a hydrogen atom, halogen, or substituted or unsubstituted alkyl,
or a pharmaceutically acceptable salt thereof.

8. The compound according to claim 1,
wherein the ring A is a ring represented by: 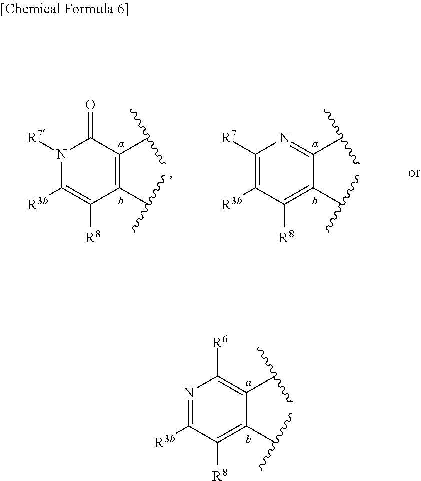
wherein carbon atom a, carbon atom b, R^{3b}, R⁶, R⁷, R^{7′}, and R⁸ have the same meanings as those in claim 1,
or a pharmaceutically acceptable salt thereof.

9. The compound according to claim 1,
wherein m is 0 or 1,
or a pharmaceutically acceptable salt thereof.

10. The compound according to claim 1,
wherein R^{5a}s are each independently a hydrogen atom, and R^{5b}s are each independently a hydrogen atom,
or a pharmaceutically acceptable salt thereof.

11. The compound according to claim 1,
wherein R⁶ is halogen, substituted or unsubstituted alkyloxy, or hydroxy,
or a pharmaceutically acceptable salt thereof.

12. The compound according to claim 1,
wherein R^{4a} is a hydrogen atom, and R^{4b} is a hydrogen atom,
or a pharmaceutically acceptable salt thereof.

13. The compound according to claim 1, selected from the group consisting of compounds I-0174, I-0414, I-0417, I-0437, I-0699, I-0934, I-0955, I-0957, I-0978, I-0990, and I-1005,
or a pharmaceutically acceptable salt thereof.

14. A pharmaceutical composition comprising the compound according to claim 1, or a pharmaceutically acceptable salt thereof.

15. A coronavirus 3CL protease inhibitor comprising the compound according to claim 1, or a pharmaceutically acceptable salt thereof.

16. A coronavirus replication inhibitor comprising the compound according to claim 1, or a pharmaceutically acceptable salt thereof.

17. The coronavirus replication inhibitor according to claim 16, wherein the coronavirus is an alphacoronavirus and/or betacoronavirus.

18. The coronavirus replication inhibitor according to claim 16, wherein the coronavirus is SARS-CoV-2.

19. A method for treating and/or preventing a disease associated with coronavirus 3CL proteases, characterized by administering the compound according to claim 1, or a pharmaceutically acceptable salt thereof.

20. The compound according to claim 1, or a pharmaceutically acceptable salt thereof, for use in treating and/or preventing a disease associated with coronavirus 3CL proteases.


SEQUENCE LISTING (2 sequences)

SEQ ID NO: 1 - protein, 14 aa, synthetic construct
  REGION 1..14: note = Substrate Peptide
KTSAVLQSGFRKME

SEQ ID NO: 2 - protein, 7 aa, synthetic construct
  REGION 1..7: note = Internal Standard Peptide
KTSAVLQ